SEQUENCE LISTING (112 sequences)

SEQ ID NO: 1 - protein, 246 aa, Homo sapiens [sequence omitted]

SEQ ID NO: 2 - protein, 325 aa, Homo sapiens
  misc_feature: Incyte ID No 1294096CD1
MALADSTRGLPNGGGGGGGSGSSSSSAEPPLFPDIVELNVGGQVYVTRRCTVVSVPDSLLWRMFTQQQPQELARDSKGRFFLDRDGFLFRYILDYLRDLQLVLPDYFPERSRLQREAEYFELPELVRRLGAPQQPGPGPPPSRRGVHKEGSLGDELLPLGYSEPEQQEGASAGAPSPTLELASRSPSGGAAGPLLTPSQSLDGSRRSGYITIGYRGSYTIGRDAQADAKFRRVARITVCGKTSLAKEVFGDTLNESRDPDRPPERYTSRYYLKFNFLEQAFDKLSESGFHMVACSSTGTCAFASSTDQSEDKIWTSYTEYVFCRE

SEQ ID NO: 3 - protein, 376 aa, Homo sapiens
  misc_feature: Incyte ID No 7238537CD1
MARGPGPLGRPRPDTVAMPKRGKRLKFRAHDACSGRVTVADYANSDPAVVRSGRVKKAVANAVQQEVKSLCGLEASQVPAEEALSGAGEPCDIIDSSDEMDAQEESIHERTVSRKKKSKRHKEELDGAGGEEYPMDIWLLLASYIRPEDIVNFSLICKNAWTVTCTAAFWTRLYRRHYTLDASLPLRLRPESMEKLRCLRACVIRSLYHMYEPFAARISKNPAIPESTPSTLKNSKCLLFWCRKIVGNRQEPMWEFNFKFKKQSPRLKSKCTGGLQPPVQYEDVHTNPDQDCCLLQVTTLNFIFIPIVMGMIFTLFTINVSTDMRHHRVRLVFQDSPVHGGRKLRSEQGVQVILDPVHSVRLFDWWHPQYPFSLRA

SEQ ID NO: 4 - protein, 461 aa, Homo sapiens
  misc_feature: Incyte ID No 7494391CD1
MKILFVEPAIFLSAFAMTLTGPLTTQYVYRRIWEETGNYTFSSDSNISECEKNKSSPIFAFQEEVQKKVSRFNLQMDISGLIPGLVSTFILLSISDHYGRKFPMILSSVGALATSVWLCLLCYFAFPFQLLIASTFIGAFCGNYTTFWGACFAYIVDQCKEHKQKTIRIAIIDFLLGLVTGLTGLSSGYFIRELGFEWSFLIIAVSLAVNLIYILFFLGDPVKECSSQNVTMSCSEGFKNLFYRTYMLFKNASGKRRFLLCLLLFTVITYFFVVIGIAPIFILYELDSPLCWNEVFIGYGSALGSASFLTSFLGIWLFSYCMEDIHMAFIGIFTTMTGMAMTAFASTTLMMFLARVPFLFTIVPFSVLRSMLSKVVRSTEQGTLFACIAFLETLGGVTAVSTFNGIYSATVAWYPGFTFLLSAGLLLLPAISLCVVKCTSWNEGSYELLIQEESSEDASDR

SEQ ID NO: 5 - protein, 168 aa, Homo sapiens
  misc_feature: Incyte ID No 6451054CD1
MMEEIDRFQVPTAHSEMQPLDPAAASISDGDCDAREEKQRELARKGSLKNGSMGSPVNQQPKKNNVMARTRLVVPNKGYSSLDQSPDEKPLVALDTDSDDDFDMSRYSSSGYSSAEQINQDLNIQLLKDGYRLDEIPDDEDLDLIPPKSVNPTCMCCQATSSTACHIQ

SEQ ID NO: 6 - protein, 832 aa, Homo sapiens
  misc_feature: Incyte ID No 7494592CD1
MMEEEELEFVEELEAVLQLTPEVQLAIEQVFPSQDPLDRADFNAVEYINTLFPTEQSLANIDEVVNKIRLKIRRLDDNIRTVVRGQTNVGQDGRQALEEAQKAIQQLFGKIKDIKDKAEKSEQMVKEITRDIKQLDHAKRHLTTSITTLNHLHMLAGGVDSLEAMTRRRQYGEVANLLQGVMNVLEHFHKYMGIPQIRQLSERVKAAQTELGQQILADFEEAFPSQGTKRPGGPSNVLRDACLVANILDPRIKQEIIKKFIKQHLSEYLVLFQENQDVAWLDKIDRRYAWIKRQLVDYEEKYGRMFPREWCMAERIAVEFCHVTRAELAKIMRTRAKEIEVKLLLFAIQRTTNFEGFLAKRFSGCTLTDGTLKKLESPPPSTNPFLEDEPTPEMEELATEKGDLDQPKKPKAPDNPFHGIVSKCFEPHLYVYIESQDKNLGELIDRFVADFKAQGPPKPNTDEGGAVLPSCADLFVYYKKCMVQCSQLSTGEPMIALTTIFQKYLREYAWKILSGNLPKTTTSSGGLTISSLLKEKEGSEVAKFTLEELCLICNILSTAEYCLATTQQLEEKLKEKVDVSLIERINLTGEMDTFSTVISSSIQLLVQDLDAACDPALTAMSKMQWQNVEHVGDQSPYVTSVILHIKQNVPIIRDNLASTRKYFTQFCVKFANSFIPKFITHLFKCKPISMVGAEQLLLDTHSLKMVLLDLPSISSQVVRKAPASYTKIVVKGMTRAEMILKVVMAPHEPLVVFVDNYIKLLTDCNTETFQKILDMKGLKRSEQSSMLELLRQRLPAPPSGAESSGSLSLTAPTPEQESSRIRKLEKLIKKRL

SEQ ID NO: 7 - protein, 393 aa, Homo sapiens
  misc_feature: Incyte ID No 5202657CD1
MEQCACVERELDKVLQKFLTYGQHCERSLEELLHYVGQLRAELASAALQGTPLSATLSLVMSQCCRKIKDTVQKLASDHKDIHSSVSRVGKAIDRNFDSEICGVVSDAVWDAREQQQQILQMAIVEHLYQQGMLSVAEELCQESTLNVDLDFKQPFLELNRILEALHEQDLGPALEWAVSHRQRLLELNSSLEFKLHRLHFIRLLAGGPAKQLEALSYARHFQPFARLHQREIQVMMGSLVYLRLGLEKSPYCHLLDSSHWAEICETFTRDACSLLGLSVESPLSVSFASGCVALPVLMNIKAVIEQRQCTGVWNHKDELPIEIELGMKCWYHSVFACPILRQQTSDSNPPIKLICGHVISRDALNKLINGGKLKCPYCPMEQNPADGKRIIF

SEQ ID NO: 8 - protein, 280 aa, Homo sapiens
  misc_feature: Incyte ID No 2013529CD1
MATEAPVNIAPPECSTVVSTAVDSLIWQPNSLNMHMIRPKSAKGRTRPSLQKSQGVEVCAHHIPSPPPAIPYELPSSQKPGACAPKSPNQGASDEIPELQQQVPTGASSSLNKYPVLPSINRKNLEEEAVETVAKKASSLQLSSIRALYQDETGTMKTSEEDSRARACAVERKFIVRTKKQGSSRAGNLEEPSDQEPRLLLAVRSPTGQRFVRHFRPTDDLQTIVAVAEQKNKTSYRHCSIETMEVPRRRFSDLTKSLQECRIPHKSVLGISLEDGEGWP

SEQ ID NO: 9 - protein, 344 aa, Homo sapiens
  misc_feature: Incyte ID No 3841351CD1
MDSYSAPESTPSASSRPEDYFIGATPLQKRLESVRKQSSFILTPPRRKIPQCSQLQEDVDPQKVAFLLHKQWTLYSLTPLYKFSYSNLKEYSRLLNAFIVAEKQKGLAVEVGEDFNIKVIFSTLLGMKGTQRDPEAFLVQIVSKSQLPSENREGKVLWTGWFCCVFGDSLLETVSEDFTCLPLFLANGAESNTAIIGTWFQKTFDCYFSPLAINAFNLSWMAAMWTACKMDHYVATTEFLWSVPCSPQSLDISFAIHPEDAKALWDSVHKTPGEVTQEEVDLFMDCLYSHFHRHFKIHLSATRLVRVSTSVASAHTDGKIKILCHKYLIGVLAYLTELAIFQIE

SEQ ID NO: 10 - protein, 405 aa, Homo sapiens
  misc_feature: Incyte ID No 152116CD1
MEPGAGGRNTARAQRAGSPNTPPPREQERKLEQEKLSGVVKSVHRRLRKKYREVGDFDKIWREHCEDEETLCEYAVAMKNLADNHWAKTCEGEGRIEWCCSVCREYFQNGGKRKALEKDEKRAVLATKTTPALNMHESSQLEGHLTNLSFTNPEFITELLQASGKIRLLDVGSCFNPFLKFEEFLTVGIDIVPAVESVYKCDFLNLQLQQPLQLAQDAIDAFLKQLKNPIDSLPGELFHVVVFSLLLSYFPSPYQRWICCKKAHELLVLNGLLLIITPDSSHQNRHAMMMKSWKIAIESLGFKRFKYSKFSHMHLMAFRKISLKTTSDLVSRNYPGMLYIPQDFNSIEDEEYSNPSCYVRSDIEDEQLAYGFTELPDAPYDSDSGESQASSIPFYELEDPILLLS

SEQ ID NO: 11 - protein, 185 aa, Homo sapiens
  misc_feature: Incyte ID No 2381031CD1
MEVHGKPKASPSCSSPTRDSSGVPVSKELLTAGSDGRGGIWDRLLINSQPKSRKTSTLQTVRIERSPLLDQVQTFLPQMARANEKLRKEMAAAPPGRFNIENIDGPHSKVIQMDVALFEMNQSDSKEVDSSEESSQDSSENSSESEDEDDSIPSEVTIDNIKLPNSEGGKGKIEVLDSPASKKKK

SEQ ID NO: 12 - protein, 463 aa, Homo sapiens
  misc_feature: Incyte ID No 2511371CD1
MAQQQTGSRKRKAPAVEADAESSPSQGLAAADGEGPLLLKRQRRPATYRSMAHYLKVREVGGWGPARLQGFDGELRGYAVQRLPELLTERQLELGTVNKVFASQWLNSRQVVCGTKCNTLFVVDVESGHIARIPLLRDSEARLAQDQQGCGIHAIELNPSKTLLATGGENPNSLAIYQLPSLDPLCLGDRHGHKDWIFAVAWLSDTVAVSGSRDGTVALWRMDPDKFDDTVAWHSEVGLPVYAHIRPRDVEAIPRAIINPSNRKVRALACGGKNQELGAVSLDGYFHLWKAGSALSRLLSIRLPYFRDNVCLTYCDDMSVYAVGSHSHVSFLDLRQDQQNIRPLCSREGGTGVRSLSFYRHIITVGTGQGSLLFYDVRAQKFLEERASATLESSSGPARRKLRLACGRGWLNHNDFWVNYFGGMEVFPNALYTHCYNWPEMKLFVAGGPLPAGLHGNYAGLWS

SEQ ID NO: 13 - protein, 403 aa, Homo sapiens
  misc_feature: Incyte ID No 8068623CD1
MALRSAQGDGPTSGHWDGGAEKADFNAKRKKKVAEIHQALNSDPTDVAALRRMAISEGGLLTDEIRRKVWPKLLNVNANDPPPISGKNLRQMSKDYQQVLLDVRRSLRRFPPGMPEEQREGLQEELIDIILLILERNPQLHYYQGYHDIVVTFLLVVGERLATSLVEKLSTHHLRDFMDPTMDNTKHILNYLMPIIDQVNPELHDFMQSAEVGTIFALSWLITWFGHVLSDFRHVVRLYDFFLACHPLMPIYFAAVIVLYREQEVLDCDCDMASVHHLLSQIPQDLPYETLISRAGDLFVQFPPSELAREAAAQQQAERTAASTFKDFELASAQQRPDMVLRQRFRGLLRPEDRTKDVLTKPRTNRFVKLAVMGLTVALGAAALAVVKSALEWAPKFQLQLFP

SEQ ID NO: 14 - protein, 574 aa, Homo sapiens
  misc_feature: Incyte ID No 677977CD1
MGGERKAQTCAATFSVPARACAAGSRTMPTCAGSWSSWAVRWALSARRCGWPTRRRSTAPCTPGSWRCATTGCRCLARSSRRSRGLRAPDLRARVHLQGQPRLVLALAEAPRHLQPALLRRGGPPAPSPAPGPPVKEEPALPSGAGPLPDRAPAPPPPAEGGYGDEQIYSASVTGLYWKLLPEQAAPPGAGDPGAGGCGRRWRGDRVTVLLAANLTGSHKLKPLVIGRLPDPPSLRHHNQDKFPASYRYSPDAWLSRPLLRGWFFEEFVPGVKRYLRRSCLQQKAVLLVAHPPCPSPAASMPALDSEDAPVRCRPEPLGPPEELQTPDGAVRVLFLSKGSSRAHIPEPVEQGVVAAFKQLYKRELLRLAVSCASGSPLDFMRSFMLKDMLYLAGLSWDLVQAGSIERCWLLGLRAAFEPRPGEDSAGQPAQAEEAAEHSRVLSDLTHLAALAYKCLAPEEVAEWLHLDDDGASLPSAMGGGEDEEEATDYGGTSSLPSAIGGGEDEEEATDYGGTSVPTAGEAVRGLETALRWLENQDPREVGPLRLVQLRSLISMARRLGGIGHTPAGPYDGV

SEQ ID NO: 15 - protein, 731 aa, Homo sapiens [sequence omitted]

SEQ ID NO: 16 - protein, 299 aa, Homo sapiens [sequence omitted]

SEQ ID NO: 17 - protein, 620 aa, Homo sapiens
  misc_feature: Incyte ID No 2159545CD1
MASDSMSSKQARNHITKGKRQQQHQQIKNRSSISDGDGEDSFIFEANEAWKDFHGSLLRFYENGELCDVTLKVGSKLISCHKLVLACVIPYFRAMFLSEMAEAKQTLIEIRDFDGDAIEDLVKFVYSSRLTLTVDNVQPLLYAACILQVELVARACCEYMKLHFHPSNCLAVRAFAESHNRIDLMDMADQYACDHFTEVVECEDFVSVSPQHLHKLLSSSDLNIENEKQVYNAAIKWLLANPQHHSKWLDETLAQVRLPLLPVDFLMGVVAKEQIVKQNLKCRDLLDEARNYHLHLSSRAVPDFEYSIRTTPRKHTAGVLFCVGGRGGSGDPFRSIECYSINKNSWFFGPEMNSRRRHVGVISVEGKVYAVGGHDGNEHLGSMEMFDPLTNKWMMKASMNTKRRGIALASLGGPIYAIGGLDDNTCFNDVERYDIESDQWSTVAPMNTPRGGVGSVALVNHVYAVGGNDGMASLSSVERYDPHLDKWIEVKEMGQRRAGNGVSKLHGCLYVVGGFDDNSPLSSVERYDPRSNKWDYVAALTTPRGGVGIATVMGKIFAVGGHNGNAYLNTVEAFDPVLNRWELVGSVSHCRAGAGVAVCSCLTSQIRDVGHGSNNVVDCM

SEQ ID NO: 18 - protein, 218 aa, Homo sapiens
  misc_feature: Incyte ID No 8560269CD1
MALVPYEETTEFGLQKFHKPLATFSFANHTIQIRQDWRHLGVAAVVWDAAIVLSTYLEMGAVELRGRSAVELGAGTGLVGIVAALLGAHVTITDRKVALEFLKSNVQANLPPHIQTKTVVKELTWGQNLGSFSPGEFDLILGADIIYLEETFTDLLQTLEHLCSNHSVILLACRIRYERDNNFLAMLERQFIVRKVHYDPEKDVHIYEAQKRNQKEDL

SEQ ID NO: 19 - protein, 427 aa, Homo sapiens
  misc_feature: Incyte ID No 8710302CD1
MAALSKSIPHNCYEIGHTWHPSCRVSFLQITGGALEESLKIYAPLYLIAAILRKRKLDYYLHKLLPEILQSASFLTANGALYMAFFCILRRGLLTIYMANLATETLFRMGVARGTITTLRNGEVLLFCITAAMYMFFFRCKDGLKGFTFSALRFIVGKEEIPTHSFSPEAAYAKVEQKREQHEEKPRRMNMIGLVRKFVDSICKHGPRHRCCKHYEDNCISYCIKGFIRMFSVGYLIQCCLRIPSAFRHLFTQPSRLLSLFYNKENFQLGAFLGSFVSIYKGTSCFLRWIRNLDDELHAIIAGFLAGISMMFYKSTTISMYLASKLVETMYFKGIEAGKVPYFPHADTIIYSISTAICFQAAVMEVQTLRPSYWKFLLRLTKGKFAVMNRKVLDVFGTGASKHFQDFIPRLDPRYTTVTPELPTEFS

SEQ ID NO: 20 - protein, 612 aa, Homo sapiens
  misc_feature: Incyte ID No 6778214CD1
MEIAPQEAPPVPGADGDIEEAPAEAGSPSPASPPADGRLKAAAKRVTFPSDEDIVSGAVEPKDPWRHAQNVTVDEVIGAYKQACQKLNCRQIPKLLRQLQEFTDLGHRLDCLDLKGEKLDYKTCEALEEVFKRLQFKVVDLEQTNLDEDGASALFDMIEYYESATHLNISFNKHIGTRGWQAAAHMMRKTSCLQYLDARNTPLLDHSAPFVARALRIRSSLAVLHLENASLSGRPLMLLATALKMNMNLRELYLADNKLNGLQDSAQLGNLLKFNCSLQILDLRNNHVLDSGLAYICEGLKEQRKGLVTLVLWNNQLTHTGMAFLGMTLPHTQSLETLNLGHNPIGNEGVRHLKNGLISNRSVLRLGLASTKLTCEGAVAVAEFIAESPRLLRLDLRENEIKTGGLMALSLALKVNHSLLRLDLDREPKKEAVKSFIETQKALLAEIQNGCKRNLVLAREREEKEQPPQLSASMPETTATEPQPDDEPAAGVQNGAPSPAPSPDSDSDSDSDGEEEEEEEGERDETPSGAIDTRDTGSSEPQPPPEPPRSGPPLPNGLKPEFALALPPEPPPGPEVKGGSCGLEHELSCSKNEKELEELLLEASQESGQETL

SEQ ID NO: 21 - protein, 458 aa, Homo sapiens [sequence omitted]

SEQ ID NO: 22 - protein, 1451 aa, Homo sapiens [sequence omitted]

SEQ ID NO: 23 - protein, 184 aa, Homo sapiens
  misc_feature: Incyte ID No 7494915CD1
MMPGEKKQSGSQQQNNGQASKNRTQKEIVTQKRPITSNEIELVVKKKLPREKGPGPDGFIAEFFRTVKEELEPTLLKLFQKIERERILPNTFYGVSITLMPKPEKDTTATTTTTTTNYRPTSLMNVDSKILNKILANQIQPHIKKIIHHNQKLFSLIRSHLSILAFVAIAFGVLDVKPLPIPMH

SEQ ID NO: 24 - protein, 407 aa, Homo sapiens
  misc_feature: Incyte ID No 2073751CD1
MAAEIDFLREQNRRLNEDFRRYQMESFSKYSSVQKAVCQGEGDDTFENLVFDQSFLAPLVTEYDKHLGELNGQLKYYQKQVGEMKLQFENVIKENERLHSELKDAVEKKLEAFPLGTEVGTDIYADDETVRNLQEQLQLANQEKTQAVELWQTVSQELDRLHKLYQEHMTEAQIHVFESQKQKDQLFDFQQLTKQLHVTNENMEVTNQQFLKTVTEQSVIIEQLRKKLRQAKLELRVAVAKVEELTNVTEDLQGQMKKKEKDVVSAHGREEASDRRLQQLQSSIKQLEIRLCVTIQEANQLRTENTHLEKQTRELQAKCNELENERYEAIVRARNSMQLLEEANLQKSQALLEEKQKEEDIEKMKETVSRFVQDATIRTKKEVANTKKQCNIQISRLTEELSALQME

SEQ ID NO: 25 - protein, 261 aa, Homo sapiens
  misc_feature: Incyte ID No 3178841CD1
MTCLAPTMSAELNVPIDPSAPACPEPGHKGMDYRDWVRRSYLELVTSNHHSVQALSWRKLYLSRAKLKASSRTSALLSGFAMVAMVEVQLETQYQYPRPLLIAFSACTTVLVAVHLFALLISTCILPNVEAVSNIHNLNSISESPHERMHPYIELAWGFSTVLGILLFLAEVVLLCWIKFLPVDARRQPGPPPGPGSHTGWQAALVSTIIMVPVGLIFVVFTIHFYRSLVRHKTERHNREIEELHKLKVQLDGHERSLQVL

SEQ ID NO: 26 - protein, 209 aa, Homo sapiens
  misc_feature: Incyte ID No 3674807CD1
MATIAAAAFEALMDGVTCWDVPRGPIPSELLLIGEAAFPVMVNDKGQVLIAASSYGRGRLVVVSHEGYLSHAGLAPFLLNAVSWLCPCPGAPVGVHPSLAPLVNILQDAGLEAQVKPEPGEPLGVYCINAYNDTLTATLIQFVKHGGGLLIGGQAWYWASQHGPDKVLSRFPGNKVTSVAGVYFTDTYGDRDRFKVSKKVPKIPLHVRR

SEQ ID NO: 27 - protein, 333 aa, Homo sapiens
  misc_feature: Incyte ID No 1794922CD1
MKMDVSVRAAGCSDDLSSGEADVDPKLLELTADEEKCRSIRRQYRQLMYCVRQNREDIVSSANNSLTEALEEANVLFDGVSRTREAALDARFLVMASDLGKEKAKQLNSDMNFFNQLAFCDFLFLFVGLNWMEGDPDKLSDCDDSIALSFWKAIEKEATSWMVKAETFHFVFGSFKLERSAPKPRLEHQKKVRKMEENGNMPTKLQKLDLSSYPEATEKNVERILGLLQTYFRKYPDTPVSYFEFVIDPNSFSRTVENIFYVSFIVRDGFARIRLDEDRLPILEPMNVNQMGEGNDSSCHGRKQGVISLTLQEWKNIVAAFEISEAMITYSSY

SEQ ID NO: 28 - protein, 257 aa, Homo sapiens
  misc_feature: Incyte ID No 1795509CD1
MVAEKETLSLNKCPDKMPKRTKLLAQQPLPVHQPHSLVSEGFTVKAMMKNSVVRGPPAAGAFKERPTKPTAFRKFYERGDFPIALEHDSKGNKIAWKVEIEKLDYHHYLPLFFDGLCEMTFPYEFFARQGIHDMLEHGGNKILPVLPQLIIPIKNALNLRNRQVICVTLKVLQHLVVSAEMVGKALVPYYRQILPVLNIFKNMNVNSGDGIDYSQQKRENIGDLIQETLEAFERYGGENAFINIKYVVPTYESCLLN

SEQ ID NO: 29 - protein, 293 aa, Homo sapiens
  misc_feature: Incyte ID No 2017180CD1
MRVDSSADPTMSQEQGPGSSTPPSSPTLLDALLQNLYDFGGTEGETEQKKIIKKRENKKRDVMASAALAAEPSPLPGSLIRGQRKSASSFFKELREERHCAPSGTPTGPEILAAAVPPSSLKNNREQVEVVEFHSNKKRKLTPDHNKNTKQANPSVLERDVDTQEFNLEKARLEVHRFGITGYGKGKERILEQERAIMLGAKPPKKSYVNYKVLQEQIKEKKAAKEEEKRLAQETDIFKKKKRKGQEDRKSKKKSAPSILSNGRIGQVGKFKNGTLILSPVDIKKINSSRVAK

SEQ ID NO: 30 - protein, 598 aa, Homo sapiens
  misc_feature: Incyte ID No 219442CD1
MAASVTNAVPPHNFKSQEVTPACLDGKSLRAGITEVKEPSVTSPTPSDIQQNKGLPKPEFRFKGQSTKSDSAEDYLLWKRLQGVSAACPAPSSAAHQLEHLSAKLQKIDEQLLAIQNIAENIEQDFPKPEMLDLHCDKIGPVDHIEFSSGPEFKKTLASKTISISEEVRFLTHMDEEDQSDKKETSEPEFSITENYSGQKTCVFPTADSAVSLSSSSDQNTTSPGMNSSDELCESVSVHPLQMTGLTDIADIIDDLIIKDGVSSEELGLTEQAMGTSRIQHYSGRHSQRTDKERREIQAWMKRKRKERMAKYLNELAEKRGQEHDPFCPRSNPLYMTSREIRLRQKMKHEKDRLLLSEHYSRRISQAYGLMNELLSESVQLPTLPQKPLPNKPSPTQSSSCQHCPSPRGENQHGHSFLINRPGKVKYMSKPSYIHKRKSFGQPQGSPWPHGTATFTIQKKAGGAKAAVRKATQSPVTFQKGSNAPCHSLQHTKKHGSAGLAPQTKQVCVEYEREETVVSPWTIPSEIHKILHESHNSLLQDLSPTEEEEPEHPFGVGGVDSVSESTGSILSKLDWNAIEDMVASVEDQGLSVHWALDL

SEQ ID NO: 31 - protein, 470 aa, Homo sapiens
  misc_feature: Incyte ID No 2597459CD1
MPSERCLSIQEMLTGQRLCHSESHNDSVLAALNQQRSDGILCDITLIAEEQKFHAHKAVLAACSDYFRAMFSLCMVESGADEVNLHGVTSLGLKQALEFAYTGQILLEPGVIQDVLAAGSHLQLLELLNLCSHYLIQELNSFNYLDLYRLADLFNLTLLEKAVIDFLVKHLSELLKSRPEEVLTLPYCLLQEVLKSDRLTSLSEEQIWQLAVRWLEHNCHYQYMDELLQYIRFGLMDVDTLHTVALSHPLVQASETATALVNEALEYHQSIYAQPVWQTRRTKPRFQSDTLYIIGGKKREVCKVKELRYFNPVDQENALIAAIANWSELAPMPVGRSHHCVAVMGDFLFVAGGEVEHASGRTCAVRTACRYDPRSNSWAEIAPMKNCREHFVLGAMEEYLYAVGGRNELRQVLPTVERYCPKKNKWTFVQSFDRSLSCHAGYVADGLLWISGRTYLMLDLSKHTFIVVYI

SEQ ID NO: 32 - protein, 311 aa, Homo sapiens
  misc_feature: Incyte ID No 2783863CD1
MHQKLLKSAHYIELGSYQYWPVLVPRGIRLYTYEQIPGSLKDNPYITDGYRAYLPSRLCIKSLFILSNETVNIWSHLLGFFLFFTLGIYDMTSVLPSASASREDFVICSICLFCFQVCMLCSVGYHLFSCHRSEKTCRRWMALDYAGISIGILGCYVSGVFYAFYCNNYWRQVYLITVLAMILAVFFAQIHPNYLTQQWQRLRSIIFCSVSGYGVIPTLHWVWLNGGIGAPIVQDFAPRVIVMYMIALLAFLFYISKVPERYFPGQLNYLGSSHQIWHILAVVMLYWWHQSTVYVMQYRHSKPCPDYVSHL

SEQ ID NO: 33 - protein, 894 aa, Homo sapiens
  misc_feature: Incyte ID No 2902971CD1
MATSMAAASGRFESAKSIEERKEQTRNARAEVLRQAKANFEKEERRKELKRLRGEDTWMLPDVNERIEQFSQEHSVKKKKKKDKHSKKAKKEKKKKSKKQKYEKNNESSDSSSSSEDEWVEAVPSQTPDKEKAWKVKDEKSGKDDTQIIKRDEWMTVDFMSVKTVSSSSLKAEKETMRKIEQEKNQALEQSKLMERELNPYWKDGGTGLPPEDCSVSSITKVSVVEDGGLSWLRKSYLRMKEQAEKQSRNFEDIVAERYGSMEIFQSKLEDAEKAASTKEDYRRERWRKPTYSDKAQNCQESRESDLVKYGNSSRDRYATTDTAKNSNNEKFIGDEKDKRPGSLETCRRESNPRQNQEFSFGNLRAKFLRPSDDEELSFHSKGRKFEPLSSSSALVAQGSLCSGFRKPTKNSEERLTSWSRSDGRGDKKHSNQKPSETSTDEYQHVPEDPREKSQDEVLRDDPPKKEHLRDTKSTFAGSPERESIHILSVDEKNKLGAKIIKAEMMGNMELAEQLKVQLEKANKFKETITQIPKKSGVENEDQQEVILVRTDQSGRVWPVNTPGKSLESQGGRRKRQMVSTHEERERVRYFHDDDNLSLNDLVKNEKMGTAENQNKLFMRMASKFMGKTDGDYYTLDDMFVSKAAERERLGEEEENQRKKAIAEHRSLAAQMEKCLYCFDSSQFPKHLIVAIGVKVYLCLPNVRSLTEGHCLIVPLQHHRAATLLDEDIWEEIQMFRKSLVKMFEDKGLDCIFLETNMSMKKQYHMVYECIPLPKEVGDMAPIYFKKAIMESDEEWSMNKKLMDLSSKDIRKSVPRGLPYFSVDFGLHGGFAHVIEDQHKFPHYFGKEIIGGMLDIEPRLWRKGIRESFEDQRKKALQFAQWWKPYDFTKSKNY

SEQ ID NO: 34 - protein, 653 aa, Homo sapiens
  misc_feature: Incyte ID No 368660CD1
MDRDLLRQSLNCHGSSLLSLLRSEQQDNPHFRSLLGSAAEPARGPPPQHPLQGRKEKRVDNIEIQKFISKKADLLFALSWKSDAPATSEINEDSEDHYAIMPPLEQFMEIPSMDRRELFFRDIERGDIVIGRISSIREFGFFMVLICLGSGIMRDIAHLEITALCPLRDVPSHSNHGDPLSYYQTGDIIRAGIKDIDRYHEKLAVSLYSSSLPPHLSGIKLGVISSEELPLYYRRSVELNSNSLESYENVMQSSLGFVNPGVVEFLLEKLGIDESNPPSLMRGLQSKNFSEDDFASALRKKQSASWALKCVKIGVDYFKVGRHVDAMNEYNKALEIDKQNVEALVARGALYATKGSLNKAIEDFELALENCPTHRNARKYLCQTLVERGGQLEEEEKFLNAESYYKKALALDETFKDAEDALQKLHKYMQKSLELREKQAEKEEKQKTKKIETSAEKLRNVLKEEKRLKKKRRKSTSSSSVSSADESVSSSSSSSSSGHKRHKKHKRNRSESSRSSRRHSSRASSNQIDQNRKDECYPVPANTSASFLNHKQEVEKLLGKQDRLQYEKTQIKEKDRCPLSSSSLEIPDDFGVYSYLFKKLTIKQPQAGPSGDIPEEGIVIIDDSSIHVTDPEDLQVGQDMEVEDSGIDDPDHG

SEQ ID NO: 35 - protein, 144 aa, Homo sapiens [sequence omitted]

SEQ ID NO: 36 - protein, 424 aa, Homo sapiens
  misc_feature: Incyte ID No 168571CD1
MSPLCSLCQEGSWTGPAAWMPGPLGPEHQGVQPRTPQAWAPLPAEGLWGARGEASRHGGCPSPSHGLGPHAALCLPQENPRLTEDFVSHLETELEQSRLRETETLGALREMQDKVLDMEKRNSSLPDENNVAQLQEELKALKVREGQAVASTRELKLQLQELSDTWQAHLARGGRWKESPRKLVVGELQDELMSVRLREAQALAEGRELRQRVVELETQDHIHRNLLNRVEAERAALQEKLQYLAAQNKGLQTQLSESRRKQAEAECKSKEEVMAVRLREADSMAAVAEMRQRIAELEIQREEGRIQGQLNHSDSSQYIRELKDQIEELKAEVRLLKGPPPFEDPLAFDGLSLARHLDEDSLPSSDEELLGVGVGAALQDALYPLSPRDARFFRRLERPAKDSEGSSDSDADELAAPYSQGLDN

SEQ ID NO: 37 - protein, 1351 aa, Homo sapiens [sequence omitted]

SEQ ID NO: 38 - protein, 78 aa, Homo sapiens
  misc_feature: Incyte ID No 2007684CD1
MQEMVRELWMWNVEEEEHEVGICTWGGQHCGCPAKSLPGPHPGGVSAPQSASQLMVKLLVWQKSVHKLRKLLEKTENY

SEQ ID NO: 39 - protein, 411 aa, Homo sapiens
  misc_feature: Incyte ID No 2227040CD1
MTEMSEKENEPDDAATHSPPGTVSALQETKLQRFKRSLSLKTILRSKSLENFFLRSGSELKCPTEVLLTPPTPLPPPSPPPTASDRGLATPSPSPCPVPRPLAALKPVTLHSFQEHVFKRASPCELCHQLIVGNSKQGLRCKMCKVSVHLWCSEEISHQQCPGKTSTSFRRNFSSPLLVHEPPPVCATSKESPPTGDSGKVDPVYETLRYGTSLALMNRSSFSSTSESPTRSLSERDELTEDGEGSIRSSEEGPGDSASPVFTAPAESEGPGPEEKSPGQQLPKATLRKDVGPMYSYVALYKFLPQENNDLALQPGDRIMLVDDSNEDWWKGKIGDRVGFFPANFVQRVRPGENVWRCCQPFSGNKEQGYMSLKENQICVGVGRSKDADGFIRVSSGKKRGLVPVDALTEI

SEQ ID NO: 40 - protein, 1704 aa, Homo sapiens
  misc_feature: Incyte ID No 4346130CD1
MSSVSEVNVDIKDFLMSINLEQYLLHFHESGFTTVKDCAAINDSLLQKIGISPTGHRRRILKQLQIILSKMQDIPIYANVHKTKKNDDPSKDYHVPSSDQNICIELSNSGSVQTSSPPQLETVRKNLEDSDASVERSQYPQSDDKLSPPKRDFPTAEEPHLNLGSLNDSLFGSDNIKIESLITKKTVDHTVEEQQTEKVKLITENLSKLPNADSECLSFVGCSTSGTNSGNGTNGLLEGSPPSPFFKFQGEMIVNDLYVPSSPILAPVRSRSKLVSRPSRSFLLRHRPVPEIPGSTKGVSGSYFRERRNVATSTEKSVAWQNSNEENSSSIFPYGETFLFQRLENSKKRSIKNEFLTQGEALKGEAATATNSFIIKSSIYDNRKEKISEDKVEDIWIPREDKNNFLIDTASESEYSTVEECFQSLRRKNSKASKSRTQKALILDSVNRHSYPLSSTSGNADSSAVSSQAISPYACFYGASAKKVKSGWLDKLSPQGKRMFQKRWVKFDGLSISYYNNEKEMYSKGIIPLSAISTVRVQGDNKFEVVTTQRTFVFRVEKEEERNDWISILLNALKSQSLTSQSQAVVTPEKCGYLELRGYKAKIFTVLSGNSVWLCKNEQDFKSGLGITIIPMNVANVKQVDRTVKQSFEIITPYRSFSFTAETEKEKQDWIEAVQQSIAETLSDYEVAEKIWFNESNRSCADCKAPDPDWASINLCVVICKKCAGQHRSLGPKDSKVRSLKMDASIWSNELIELFIVIGNKRANDFWAGNLQKDEELHMDSPVEKRKNFITQKYKEGKFRKTLLASLTKEELNKALCAAVVKPDVLETMALLFSGADVMCATGDPVHSTPYLLAKKAGQSLQMEFLYHNKFSDFPQHDIHSEGVLSQESSQSTFLCDFLYQAPSAASKLSSEKKLLEETNKKWCVLEGGFLSYYENDKSTTPNGTININEVICLAIHKEDFYLNTGPIFIFEIYLPSERVFLFGAETSQAQRKWTEAIAKHFVPLFAENLTEADYDLIGQLFYKDCHALDQWRKGWFAMDKSSLHFCLQMQEVQGDRMHLRRLQELTISTMVQNGEKLDVLLLVEKGRTLYIHGHTKLDFTVWHTAIEKAAGTDGNALQDQQLSKNDVPIIVNSCIAFVTQYGLGCKYIYQKNGDPLHISELLESFKKDARSFKLRAGKHQLEDVTAVLKSFLSDIDDALLTKELYPYWISALDTQDDKERIKKYGAFIRSLPGVNRATLAAIIEHLYRVQKCSEINHMNAHNLALVFSSCLFQTKGQTSEEVNVIEDLINNYVEIFEVKEDQVKQMDIENSFITKWKDTQVSQAGDLLIEVYVERKEPDCSIIIRISPVMEAEELTNDILAIKNIIPTKGDIWATFEVIENEELERPLHYKENVLEQVLRWSSLAEPGSAYLVVKRFLTADTIKHCSDRSTLGSIKEGILKIKEEPSKILSGNKFQDRYFVLRDGFLFLYKDVKSSKHDKMFSLSSMKFYRGVKKKMKPPTSWGLTAYSEKHHWHLCCDSSQTQTEWMTSIFIAQHEYDIWPPAGKERKRSITKNPKIGGLPLIPIQHEGNATLARKNIESARAELERLRLSEKCDKESVDSSLKERASMVAHCLEHKDDKLRNRPRKHRSFNCLEDTEPEAPLGQPKGHKGLKTLRKTEDRNSKATLDSDHKLPSRVIEELNVVLQRSRTLPKELQDEQILK

SEQ ID NO: 41 - protein, 243 aa, Homo sapiens
  misc_feature: Incyte ID No 55117040CD1
MVAEVDSMPAASSVKKPFGLRSKMGKWCCHCFPCCRGSGKSNVGTSGDHNDSSVKTLGSKRCKWCCHCFPCCRGSGKSNVVAWGDYDDSAFMDPRYHVHGEDLDKLHRAAWWGKVPRKDLIVMLRDTDVNKRDKQKRTALHLASANGNSEVVKLVLDRRCQLNVLDNKKRTALTKAVQCQEDECALMLLEHGTDPNIPDEYGNTTLHYAVYNEDKLMAKHCSYTVLISNQKTAWPHTTATWYT

SEQ ID NO: 42 - protein, 248 aa, Homo sapiens
  misc_feature: Incyte ID No 7472392CD1
MDVLHASVRRSTIVCMEETEFLVVDREDFFANKLDQEVQKDAQYRFEFFRKMELFASWSDEKLWQLVAMAKIERFSYGQLISKDFGESPFIMFISKGSCEVLRLLDLGASPSYRRWIWQHLELIDGRPLKTHLSEYSPMERFKEFQIKSYPLQDFSSLKLPHLKKAWGLQGTSFSRKIRTSGDTLPKMLGPKIQSRPAQSIKCAMINIKPGELPKEAAVGAYVKVHTVEQGEILVSVPRALFTMEYVT

SEQ ID NO: 43 - protein, 310 aa, Homo sapiens
  misc_feature: Incyte ID No 4028960CD1
MGKRRCVPPLEPKLAAGCCGVKKPKLSGSGTHSHGNQSTTVPGSSSGPLQNHQHVDSSSGRENVSDLTLGPGNSPITRMNPASGALSPLPRPNGTANTTKNLVVTAEMCCYCFDVLYCHLYGFPQPRLPRFTNDPYPLFVTWKTGRDKRLRGCIGTFSAMNLHSGLREYTLTSALKDSRFPPLTREELPKLFCSVSLLTNFEDASDYLDWEVGVHGIRIEFINEKGVKRTATYLPEVAKEQDWDQIQTIDSLLRKGGFKAPITSEFRKTIKLTRYRSEKVTISYAEYIASRQHCFQNGTLHAPPLYNHYS

SEQ ID NO: 44 - protein, 838 aa, Homo sapiens
  misc_feature: Incyte ID No 8227004CD1
MFWKFDLHSSSHIDTLLEREDVTLKELMDEEDVLQECKAQNRKLIEFLLKAECLEDLVSFIIEEPPQDMDEKIRYKYPNISCELLTSDVSQMNDRLGEDESLLMKLYSFLLNDSPLNPLLASFFSKVLSILISRKPEQIVDFLKKKHDFVDLIIKHIGTSAIMDLLLRLLTCIEPPQPRQDVLNWLNEEKIIQRLVEIVHPSQEEDRHSNASQSLCEIVRLSRDQMLQIQNSTEPDPLLATLEKQEIIEQLLSNIFHKEKNESAIVSAIQILLTLLETRRPTFEGHIEICPPGMSHSACSVNKSVLEAIRGRLGSFHELLLEPPKKSVMKTTWGVLDPPVGNTRLNVIRLISSLLQTNTSSINGDLMELNSIGVILNMFFKYTWNNFLHTQVEICIALILASPFENTENATITDQDSTGDNLLLKHLFQKCQLIERILEAWEMNEKKQAEGGRRHGYMGHLTRIANCIVHSTDKGPNSALVQQLIKDLPDEVRERWETFCTSSLGETNKRNTVDLMQQMTSNFIDQFGFNDEKFADQDDIGNVSFDRVSDINFTLNTNESGNIALFEACCKERIQQFDDGGSDEEDIWEEKHIAFTPESQRRSSSGSTDSEESTDSEEEDGAKQDLFEPSSANTEDKMEVDLSEPPNWSANFDVPMETTHGAPLDSVGSDVWSTEEPMPTKETGWASFSEFTSSLSTKDSLRSNSPVEMETSTEPMDPLTPSAAALAVQPEAAGSVAMEASSDGEEDAESTDKVTETVMNGGMKETLSLTVDAKTETAVFKSEEGKLSTSQDAACKDAEECPETAEAKCAAPRPPSSSPEQRTGQPSAPGDTSVNGPV

SEQ ID NO: 45 - protein, 408 aa, Homo sapiens
  misc_feature: Incyte ID No 3044763CD1
MRTDSGARLEEGHLRPPRALPPVPSQDDIPLSRPKKKKPRTKNTPASASLEGLAQTAGRRPSEGNEPSTKELKEHPEAPVQRRQKKTRLPLELETSSTQKKSSSSSLLRNENGIDAEPAEEAVIQKPRRKTKKTQPAELQYANELGVEDEDIITDEQTTVEQQSVFTAPTGISQPVGKVFVEKSRRFQAADRSELIKTTENIDVSMDVKPSWTTRDVALTVHRAFRMIGLFSHGFLAGCAVWNIVVIYVLAGDQLSNLSNLLQQYKTLAYPFQSLLYLLLALSTISAFDRIDFAKISVAIRNFLALDPTALASFLYFTALILSLSQQMTSDRIHLYTPSSVNGSLWEAGIEEQILQPWIVVNLVVALLVGLSWLFLSYRPGMDLSEELMFSSEVEEYPDKEKEIKASS

SEQ ID NO: 46 - protein, 101 aa, Homo sapiens
  misc_feature: Incyte ID No 4044519CD1
MCFLFFLLFFTMVASTCPSDLRLKDSFLKNMVPALKGCFRTYFICFLLILIFQLNPSSSLPSSLPVYLFSFLSFFFFFFFLEAESCPVTQAEVQWYDHSSL

SEQ ID NO: 47 - protein, 256 aa, Homo sapiens [sequence omitted]

SEQ ID NO: 48 - protein, 104 aa, Homo sapiens
  misc_feature: Incyte ID No 8109363CD1
MPRSSQHSESSPLDTTTQRKGASSLAHQVRVHTLETLLDWPELPQPLLTPPPVIDTAAGSRKRFLNKAQLAQCLAQQTINTCKLNCMILAQVLLMWLTATHLHG

SEQ ID NO: 49 - protein, 855 aa, Homo sapiens
  misc_feature: Incyte ID No 1272746CD1
MSFVRVNRCGPRVGVRKTPKVKKKKTSVKQEWDNTVTDLTVHRATPEDLVRRHEIHKSKNRALVHWELQEKALKRKWRKQKPETLNLEKRRLSIMKEILSDQYQMQDVLEKSDHLIAAAKELFPRRRTGFPNVTVAPDSSQGPIVVNQDPITQSIFNESVIEPQALNDVDGEEEGTVNSQSGESENENELDNSLNSQSNTNTDRFLQQLTEENFELISKLWTDIQQKIATQSQITPPGTPSSALSSGEQRAALNATNAVKRLQTRLQPEESTETLDSSYVVGHVLNSRKQKQLLNKVKRKPNLHALSKPKKNISSGSTTSADLPNRTNSNLDVLKHMIHEVEHEMEEYERWTGREVKGLQSSQGLTGFTLSLVSSLCRLVRYLKESEIQLRKEVETRQQLEQVLGDHRELIDALTAEILRLREENAATQARLQQYMVTTDEQLISLTHAIKNCPVINNRQEIQASESGATGRRVMDSPERPVVNANVSVPLMFREEVAEFPQEELPVKLSQVPDPPDNMNLAKNFPAHIFEPAVLLTPPRQKSNLKFSPLQDVLRRTVQTRPAPRLPPTVEIIEKEQNWEEKTLPIDTDIQNSSEENRLFTQRWRVSHMGEDLENKTQAPFVNLSQPLCNSHSNTQQSRSPTFSEELPVLGDGQQLRTNESLIQRKDIMTRIADLTLQNSAIKAHMNNIIEPRGEQGDGLRELNKQESASDMTSTFPVAQSLTPGSMEERIAELNRQSMEARGKLLQLIEQQKLVGLNLSPPMSPVQLPLRAWTEGAKRTIEVSIPGAEAPESSKCSTVSPVSGINTRRSSGATGNSCSPLNATSGSGRFTPLNPRAKIEKQNEEGWFALSTHVS

SEQ ID NO: 50 - protein, 427 aa, Homo sapiens
  misc_feature: Incyte ID No 1839974CD1
MYVTMMMTDQIPLELPPLLNGEVAMMPHLVNGDAAQQVILVQVNPGETFTIRAEDGTLQCIQGPAEVPMMSPNGSIPPIHVPPGYISQVIEDSTGVRRVVVTPQSPECYPPSYPSAMSPTHHLPPYLTHHPHFIHNSHTAYYPPVTGPGDMPPQFFPQHHLPHTIYGEQEIIPFYGMSTYITREDQYSKPPHKKLKDRQIDRQNRLNSPPSSIYKSSCTTVYNGYGKGHSGGSGGGGSGSGPGIKKTERRARSSPKSNDSDLQEYELEVKRVQDILSGIEKPQVSNIQARAVVLSWAPPVGLSCGPHSGLSFPYSYEVALSDKGRDGKYKIIYSGEELECNLKDLRPATDYHVRVYAMYNSVKGSCSEPVSFTTHSCAPECPFPPKLAHRSKSSLTLQWKAPIDNGSKITNYLLEWDEVSLFSYSPI

SEQ ID NO: 51 - protein, 800 aa, Homo sapiens
  misc_feature: Incyte ID No 1877336CD1
MAANVGDQRSTDWSSQYSMVAGAGRENGMETPMHENPEWEKARQALASISKSGAAGGSAKSSSNGPVASAQYVSQAEASALQQQQYYQWYQQYNYAYPYSYYYPMSMYQSYGSPSQYGMAGSYGSATPQQPSAPQHQGTLNQPPVPGMDESMSYQAPPQQLPSAQPPQPSNPPHGAHTLNSGPQPGTAPATQHSQAGPATGQAYGPHTYTEPAKPKKGQQLWNRMKPAPGTGGLKFNIQKRPFAVTTQSFGSNAEGQHSGFGPQPNPEKVQNHSGSSARGNLSGKPDDWPQDMKEYVERCFTACESEEDKDRTEKLLKEVLQARLQDGSAYTIDWSREPLPGLTREPVAESPKKKRWEAASSLHPPRGAGSATRGGGAPSQRGTPGAGGAGRARGNSFTKFGNRNVFMKDNSSSSSTDSRSRSSSRSPTRHFRRSDSHSDSDSSYSGNECHPVGRRNPPPKGRGGRGAHMDRGRGRAQRGKRHDLAPTKRSRKKMAALECEDPERELKKQKRAARFQHGHSRRLRLEPLVLQMSSLESSGADPDWQELQIVGTCPDITKHYLRLTCAPDPSTVRPVAVLKKSLCMVKCHWKEKQDYAFACEQMKSIRQDLTVQGIRTEFTVEVYETHARIALEKGDHEEFNQCQTQLKSLYAENLPGNVGEFTAYRILYYIFTKNSGDITTELAYLTRELKADPCVAHALALRTAWALGNYHRFFRLYCHAPCMSGYLVDKFADRERKVALKAMIKTFRPALPVSYLQAELAFEGEAACRAFLEPLGLAYTGPDNSSIDCRLSLAQLSAF

SEQ ID NO: 52 - protein, 107 aa, Homo sapiens
  misc_feature: Incyte ID No 2321054CD1
MAGGYGVMGDDGSIDYTVHEAWNEATNVYLIVILVSFGLFMYAKRNKRRIMRIFSVPPTEETLSEPNFYDTISKIRLRQQLEMYSISRKYDYQQPQNQADSVQLSLE

SEQ ID NO: 53 - protein, 522 aa, Homo sapiens
  misc_feature: Incyte ID No 2796034CD1
MTPGKHSGASARAANAGAWGYRDFRGGQKKGWCTTPQLVATMPVSPAGSHKQQNFGLNNATQPKKSISFFATMKATSVKGYTGANQSRMAVSKTVLIPPELKTVEKPNPNIKTTQVFDINGTDVTPRPLYHPDPLTGTAKPSKLLTSQEGSLGSEFISSYSLYQNTINPSTLGQFTRSVLGSSTVSKSSVSASESIAEDLEEPSYKRERLTSFTDLQVIRAAPEKIVTKEDLEKNIEIILTETETLRFFDLPTVMVSVESEEAEKVTQRNKNYEVLCRNRLGNDLYVERMMQTFNGAPKNKDVQCDKIIMEDKGIMSTAWDLYDSYNAMELVSLSVKQSVVESSSKANVLPKDQDQRLPGSTTEKNSETSSLMDIENVILAKIHEDEEDHSDAILKSDKFHQDLFFMERVLMENIFQPKLAAYRQLPVLKEPEPEEPEDVLESAKHEEVEEESKKEEEEEIHAEESTIPANLERLWSFSCDLTKGLNVSSLAWNKTNPDLLAVGYGHFGFKEQKEDWLAAGQ

SEQ ID NO: 54 - protein, 305 aa, Homo sapiens
  misc_feature: Incyte ID No 4413112CD1
MGGTLAWTLLLPLLLRESDSLEPSCTVSSADVDWNAEFSATCLNFSGLSLSLPHNQSLRASNVILLDLSGNGLRELPVTFFAHLQKLEVLNVLRNPLSRVDGALAARCDLDLQADCNCALESWHDIRRDNCSGQKPLLCWDTTSSQHNLSAFLEVSCAPGLASATIGAVVVSGCLLLGLAIAGPVLAWRLWRCRVARSRELNKPWAAQDGPKPGLGLQPRYGSRSAPKPQVAVPSCPSTPDYENMFVGQPAAEHQWDEQGAHPSEDNDFYINYKDIDLASQPVYCNLQSLGQASMDEEEYVIPGH

SEQ ID NO: 55 - protein, 329 aa, Homo sapiens
  misc_feature: Incyte ID No 7654832CD1
MSVLEENRPFAQQLSNVYFTILSLFCFKLFVKISLAILSHFYIVKGNRKEAARIAAEFYGVTQGQGSWADRSPLHEAASQGRLLALRTLLSQGYNVNAVTLDHVTPLHEACLGDHVACARTLLEAGANVNAITIDGVTPLFNACSQGSPSCAELLLEYGAKAQLESCLPSPTHEAASKGHHECLDILISWGIDVDQEIPHLGTPLYVACMSQQFHCIWKLLYAGADVQKGKYWDTPLHAAAQQSSTEIVNLLLEFGADINAKNTELLRPIDVATSSSMVERILLQHEATPSSLYQLCRLCIRSYIGKPRLHLIPQLQLPTLLKNFLQYR

SEQ ID NO: 56 - protein, 236 aa, Homo sapiens
  misc_feature: Incyte ID No 7503849CD1
MARGPGPLGRPRPDTVAMPKRGKRLKFRAHDACSGRVTVADYANSDPAVVRSGRVKKAVANAVQQEVKSLCGLEASQVPAEEALSGAGEPCDIIDSSDEMDAQEESIHERTVSRKKKSKRHKEELDGAGGEEYPMDIWLLLASYIRPEDIVNFSLICKNAWTVTCTAAFWTRLYRRHYTLDASLPLRLRPESMEKLRCLRACVIRSLYHMYEPFAARISKNPAIPESTPSTLKNSK

SEQ ID NO: 57 - DNA, 1485 bp, Homo sapiens
  misc_feature: Incyte ID No 2867236CB1
ctcttcctgagaaacgagcaaacctgaaagctactctctcagcttcagagggaaaaaatggttgtagatttctggacttgggagcagacatttcaagaactaatccaagaggcaaaaccccgggccacatggacgctgaagttggatggcaaccttcagctagactgcctggctcaagggtggaagcaataccaacagagagcatttggctggttccggtgttcctcctgccagcgaagttgggcttccgcccaagtgcagattctgtgccacacgtactgggagcactggacatcccagggtcaggtgcgtatgaggctctttggccaaaggtgccagaagtgctcctggtcccaatatgagatgcctgagttctcctcggatagcaccatgaggattctgagcaacctggtgcagcatatactgaagaaatactatggaaatggcacgaggaagtctccagaaatgccagtaatcctggaagtgtccctggaaggatcccatgacacagccaattgtgaggcatgcactttgggcatctgtggacagggcttaaaaagctacatgacaaagccgtccaaatccctactcccccacctaaagactgggaattcctcacctggaattggtgctgtgtacctcgcaaaccaagccaagaaccagtcagctgaggcaaaagaggctaaggggagtgggtatgagaaattagggcccagtcgagacccagatccactgaacatctgtgtctttattttgctgcttgtatttattgtagtcaaatgctttacatcagaatgatgaaaataggcttgccactttctcttattttaattccatggtagtcaatgaactggctgccactttaatataactgaaaattcattttgagaccaagcaggatcaagtttgtagaataaacactggtttcctagccatcctctgaaaacagtatgaaacatgaccaagtacataatggatttagtaataaatattgtcgaattgctaaaaagtcttcaatcattcattcactaagtcactcagtgatatcaatatacttagctcagaaagtgtgggaggctgaataatggtgtctcccaacatatgcatgacttaatccccagaacctgtaaacatgttactttacatggtagaatggactttgcggatgtaattaaggaccttgaaatggttagattatttcatattgtccgggtggataagaaccaggattttgtaacagggaggcaacaagctcaaaatcagaaaaaagagatttgtcaatggaacaagaggttgaagtgctttgaagttggaggaagaggtcacaggcaaaaaagtacaggcagcctttagaaaaccaaaaggacaaaggaacagattctcccctggagtctgcagaaggaaccagccctgcctgcacatggctttagcccagtgacactgattttggacatctgaccttcagaaatactagctcatacctcg

SEQ ID NO: 58 - DNA, 6176 bp, Homo sapiens
  misc_feature: Incyte ID No 1294096CB1
caccgctcggctccgcgcggctctaggaggtggcggcggtggcggtggcggcggtggcggcggcggcggcggcggggcgcagggctgagcgagcgtccgggttccggggctccggggaaggcggttgcagctcctgagtgcagcgcggcttcctgccactgtcccggcccggccacctctctgtcatggctctggcggacagcacacgtggattacccaacgggggcggcggcgggggcggcagtggctcctcgtcgtcctccgcggagccaccgctcttccccgacatcgtggagctgaacgtggggggccaggtgtacgtgacccggcgctgcacggtggtgtcggtgcccgactcgctgctctggcgcatgttcacgcagcagcagccgcaggagctggcccgggacagcaaaggccgcttctttctggaccgggacggcttcctcttccgctacatcctggattacctgcgggacttgcagctcgtgctgcccgactacttccccgagcgcagccggctgcagcgcgaggccgagtacttcgagctgccagagctcgtgcgccgcctcggggcgccccagcagcccggcccggggccgccgccctcgcggcgcggggtgcacaaggagggctcgctgggtgacgagctgctgccgcttggctactcggagcccgaacagcaggagggcgcctctgccggggcgccgtcgcccacgctggagctggctagccgcagtccgtccgggggcgcggcgggcccgctgctcacgccgtcccagtcgctggacggcagccggcgctcgggctacatcaccatcggctaccgcggctcctacaccatcgggcgggacgcgcaggcggacgccaagttccggcgagtggcgcgcatcaccgtttgcggaaagacgtcgctggccaaggaggtgtttggggacaccctgaacgaaagccgggaccccgaccgtcccccggagcgctacacctcgcgctattacctcaagttcaacttcctggagcaggccttcgacaagctgtccgagtcgggcttccacatggtggcgtgcagctccacgggcacctgcgcctttgccagcagcaccgaccagagcgaggacaagatctggaccagctacaccgagtacgtcttctgcagggagtgagctccccagaccccctcgccactccagcgcccagtccttctcctgcccgagagatgattacagagcctcttgtcccacctttgtcccctggctgctgccctcccattctccccctccagtagtagctgggtgagacctgtccgcccaccttccctccactacagaacctgcagccgcaaatcctctgggctgcttcgtcttctttggacctcctgaaccgagagaacccagaggaacccccaccccacccccacctaccactccatgctttctctactccctgcctcaaaccacccctcccccagatggtacttcagtttggatctattgggggagtgtggccacagaccgggggatgattgaattgttcagaacctgattggaccgtgtccaatgtgcggaagatttccttgaaatcttctcaagctcttatgactcactgggggtttaagagatcaggattggttccactgtctggggttagtgttttacaaggtcattacacagtctttttgacctcttttgaaggtagagttttagaaggctggatggaagattctgagcctggaattaggaccccatggaggcagttcagtaactaaactaataaagttttgaaaagttacacgtaaagtagaagaatctagtgcgtgggacagtaaaggatcctttctcgtacagaataaaaggtctcagcctgtagcttaaacttatagaaagtgatccgcctgcctgcagaggcgcccttttcagctgctgctcgccagaagcccttgattccactggttgacatggcagcagttactggcaagagggagaaaggacgctgccgcctaagagtgcaaggctgctcaggtctccaagcgccgtaggaggtcacctggcagtgactgtagggagctgggtcatagtgcacgtcgtgggtattaggaaagcctgtattctttcaatgaatgtcagtaggaccttcctttagctgtaagacttggtgggcggggtggggtggggagggaggaaagggtaggaagggtgggaagggagaagcagacatagtcatttatgatttgaaagttggaagtttgtaccatctgtttgagtatatgcacatttaaaaaatatcatatagtaaatgcaaacatgccaagtattttataaagattaataacagacctactcttacctggcagtttacttaacttactgttttgagtcctaaacttagagttgttaatgcttatatataatctaaccaaagagttacccagtagggttttagtttttgaacttttattttcttgttgattataaatcctgattttggaatctattgcgcaaaagaagtttcattttggttacttagacctaagatcacttattaaaaatccttattttctccaagcccagcaaacgttgacttctgggcaaacctgaaaacctgaaaatgccactttcatgcagtttgtttgaagttaagtggaatcctttcaaatgacgagctgcagagaactcagcaccaagggctgcctatctgtagatagctgtaaaatggaatatttttaaatgaaggcaaataagtacttaaaagtgagctgagcaataaaatggtccaataataggtaaatgcaacagaaacagaaggagacctggttgccttatgcctttactcttacatggaataaattcccaatgcatatcctatgtaaaccataagtgaagggaaataaacctcgtcatgctccatgctgtgaggtgtcctttggatattctgtgatgacagagaagcctattttgttttgttttcagcatctttctctgatgtacgtttttaaggattttgtaagagctgttttcagtgtttaaattagtgctatttttccttgtttttaaaaatgaatctcgtactgtatcttactatgtccatacagatgttacaaatcgacagttttattcttagactcatgtgatccaagctgtatataccatatataaacattttacatgaatcatttagttttttaattcatttactaatgctataaaatttcctatattaccccagtaatttgcatcagctggtttatatactaaagcaacatgttttgatgagtttcttacatccttatcgaggaattgggttaggaaaaaatacataattgtaaaactgagtttgctgtattatactttttttcttgagtattagttgtattactaatcatatgttgattaactgtctacttaaagtcaaggtacctgtatttttaatccactaatttttttttagttgggaaatagatttcaggtcttttattagactaacattttttgagaagtaaaattgacttcatatacaaagcctgtaattttaggcgaaatggaagcagaaatctaggaagttgtgcttgcttgtatgttgagtttggtctcagactaagtaatgcatcagaattcatctgtttgaagcctgaaataatttaggactctgattcactgaccaaaagtcagtgttgcagagatttctctaccccgtatggtattttgttagattgttcaacaggaagcacatgattgagaacatcttgggacagaccaaaaccactgacagatggcaaggctcggcgattctgatttcccttctcaaatctgctcaactccaagagtcttgagaaactgctaaaattttgcctctgtcactcaagtcttacaaatgttatcttgtaaacctttgaggtgaactattccactgtcttgtacataggcatcttattcactgcaccctgtcacacccagcaccccccgccccgcacattatttgaaagactgggaatttaatggttagggacagtaaatctacttctttttccagggacgactgtcccctctaaagttaaagtcaatacaagaaaactgtctatttttagcctaaagtaaaggctgtgaagaaaattcattttacattgggtagacagtaaaaaacaagtaaaataacttgacatgagcacctttagatcccttcccctccatgggctttgggccacagaatgaacctttgaggcctgtaaagtggattgtaatttcctataagctgtaatagtggaggtattgtgggttcatttgagtaagccctccaaagataccattcaaataacctgggagaatgtcataaattattcagataattaacactgcatgaatctgattcagaggcatgcatttacatatgttgccctaattaccatttgatgatcataaatacaagtgaatgacattggacttttagtaacaaacttaatttttaaaaaggtgtagacaatggtggttaaaaaaaaaaaaaaacaggtaccaggttctgtgtgtttgcaccaagtaattgacatgttttttgtttaatacatgtggaccatgaacagtattcattctcctttttcaaatgatatgctgtagaaaatattccttgaagatgtgagatttaaaaatttttccctttcaatgttgttttaattgtatttcttacttggtttttttgattgatagcacagtgataaatcataatactagacaaaattgtcttctctttcaaaccagagccatatatatgtctgtatatatgggacctactgcttctctgaggaaatgcataatctgttaatatcagacaaaatgagcaattggcagtgctcataatatattccaatttttattggaattttcgatggaatgttatttcaataaagccatgtaaggtgaaactttgataactttttactcttcaagttagggtaaattctgatccaatattcaattcatttgtgtactcccacatgcaaaatgctaaattacaatgcagacattaagaaaaagtattgactggaggggttgaattccttgagaatttattttatagtctaaatcacaaatactttactcaatttagtttttaaaatagtaaactgaatatttttgttgtaagcctatcagagtcaatccttcgtttggaattgttttcctgtttttccttactataaatcatttaaaaactgaattcattttcttagatggcataagtctgtctcttgagaaataagtaaaatactcctattttcagtatctgtagcacctgaaataggtctttgtatagccagaaacaagttatgttgaagttagcttttctttgtcaacagttttggacaataaaaatctgaaagtattaacacttgattttctactggggcccttcaaacttggttggaagaaattcaaccagaatatctacattagagtataatcatgtgtggtaggaagatggactagttaatcaagatttgttgtcacttaaattttttgtgatttttttccaagccagtttttttaaattctaaatgtgttttgaggtatgggtacattaattgtaatgtaaactattatacaactgtttttgcgactttataggcaggtaaattttgctattactattgaatacaaatgacaattcatttatgaccactcaaacagcgttagtaaccatttagtgacaaaggattaaaacatccatctggatgttaattttgaagatgtaaattatatgttgtttaaatttttccaggcatctgaaaaccttatctgctagacaatgtaagattcacacagagttatctgggattctgattttttaaatagtacatatcattaaaccattttctctaaatgtaagaagagcagaaaaaatcttataagattatcagatttttctaatgacacagaaatgtaagaaaaaaatccctttatattgaaaaaagatgcagtcaaagtcttttcagacatgcccaaactttgagaatttcttcaaccatctaatgctataaagatttttgttcttcctgttcacaaccagttgtataacagaaatactagctactgttttccttcctgtgtgtgaagtaatgaatcattgattatgtgacttgttatgtattcaattaaacactaaagaataaaacattcactcctttaattatt

SEQ ID NO: 59 - DNA, 1944 bp, Homo sapiens
  misc_feature: Incyte ID No 7238537CB1
ggggggtgtgggatggccgcggagccgggcggagctggcttgcggctcccggggccggctctccggccggagacatggcccgggggcccggcccgctaggcaggcctcgccccgatacggtcgccatgcccaagagaggaaagcgactcaagttccgggcccacgacgcctgctccggccgagtgaccgtggcggattacgccaactcggatccggcggtcgtgaggtctggacgagtcaagaaagccgtagccaacgctgttcagcaggaagtaaaatctctttgtggcttggaagcctctcaggttcctgcagaggaagctctttctggggctggtgagccctgtgacatcatcgacagcagtgatgagatggatgcccaggaggaaagcatccatgagagaactgtctccagaaaaaagaaaagcaagagacacaaagaagaactggacggggctggaggagaagagtatcccatggatatttggctattgctggcctcctatatccgtcctgaggacattgtgaatttttccctgatttgtaagaatgcctggactgtcacttgcactgctgccttttggaccaggttgtaccgaaggcactacacgctggatgcttccctgcctttgcgtctgcgaccagagtcaatggagaagctgcgctgtctccgggcttgtgtgatccgatctctgtaccatatgtatgagccatttgctgctcgaatctccaagaatccagccattccagaaagcacccccagcacattaaagaattccaaatgcttacttttctggtgcagaaagattgttgggaacagacaggaaccaatgtgggaattcaacttcaagttcaaaaaacagtcccctaggttaaagagcaagtgtacaggaggattgcagcctcccgttcagtacgaagatgttcataccaatccagaccaggactgctgcctactgcaggtcaccaccctcaatttcatctttattccgattgtcatgggaatgatatttactctgtttactatcaatgtgagcacggacatgcggcatcatcgagtgagactggtgttccaagattcccctgtccatggtggtcggaaactgcgcagtgaacagggtgtgcaagtcatcctggacccagtgcacagcgttcggctctttgactggtggcatcctcagtacccattctccctgagagcgtagttactgcttcccatcccttgggggcagcctcgagtgtagtccattagtaatcagattccagtttggacagggtggctggattgtatatctcgttagtaatgtacatgctcttcaggttctagggctcctgttaggggagggagaaatgttgaatcaagagggaaaacaactactatgatttataaacatattttaatgtaaaaatttgcatttaaaaggagtggccctgttttctgtgttaaaaccccatttggtgctattgagtttgttctttattcttttatcccagtgaaaattgttgatcttgctgtagggaaaaattaaactctttgaatctccaaacaaggaagtttcagcattcccttatggatcagaggaaccttagaggcctgaaattgttgcttccagtttagctgcccctcaaattcaagtgaatattttcccttctccctttacccttctccagaaataaagcaggtgacagggttttcagaatcttaccatattgacttgtgtatcttttttaaaaaataatttgtgatatttattgcatacattttcttttggcagttttggattttaggtatttttttgcatgaggctgataatggtgatgatgactttatacccttggtactatactaggaactttacatagtatctctacttctcacagtcttgcaaagtaggtgatatttattcccattttatagtttgagaaaacggaattaggtgactg

SEQ ID NO: 60 - DNA, 1992 bp, Homo sapiens
  misc_feature: Incyte ID No 7494391CB1
gggcggacagcgatgctcagctggctgcggccgagtcatcgcctagcgctggcagggccgctgaccgaccgacggaggcgccgatggccgattgtccactgcgcagaaggagcagctgctccgcgccccgccgcgccgcgctgaggccgaggtccgcagggccgcggggaagccgagggctgccggagaaccctgcaggtgtcactcgggacgcggaagtgcgcttgccgagaatacagctacctacccaggcaatatgaagattttatttgtagaacctgccattttccttagtgcatttgctatgactttgaccggtccactgacaacgcaatatgtttatcggagaatatgggaagaaactggcaactacactttttcatctgatagcaatatttctgagtgtgaaaaaaacaaaagcagcccaatttttgcattccaggaggaagttcagaaaaaagtgtcacgttttaatctgcagatggacataagtggattaattcctggtctagtgtctacattcatacttttgtctattagtgatcactacggacgaaaattccctatgattttgtcttccgttggtgctcttgcaaccagcgtttggctctgtttgctttgctattttgcctttccattccagcttttgattgcatctaccttcattggtgcattttgtggcaattataccacattttggggagcttgctttgcctatatagttgatcagtgtaaagaacacaaacaaaaaacaattcgaatagctatcattgactttctacttggacttgttactggactaacaggactgtcatctggctattttattagagagctaggttttgagtggtcgtttctaattattgctgtgtctcttgctgttaatttgatctatattttattttttctcggagatccagtgaaagagtgttcatctcagaatgttactatgtcatgtagtgaaggcttcaaaaacctattttaccgaacttacatgctttttaagaatgcttctggtaagagacgatttttgctctgtttgttactttttacagtaatcacttatttttttgtggtaattggcattgccccaatttttatcctttatgaattggattcaccactctgctggaatgaagtttttataggttatggatcagctttgggtagtgcctcttttttgactagtttcctaggaatatggcttttttcttattgtatggaagatattcatatggccttcattgggatttttaccacgatgacaggaatggctatgaccgcgtttgccagtacaacactgatgatgtttttagccagggtgccgttccttttcactattgtgccattctctgttctacggtccatgttgtcaaaagtggttcgttcgactgaacaaggtaccctgtttgcttgtattgctttcttagaaacacttggaggagtcactgcagtttctacttttaatggaatttactcagccactgttgcttggtaccctggcttcactttcctgctgtctgctggtctgttactacttccagccatcagtctatgtgttgtcaagtgtaccagctggaatgagggaagctatgaacttcttatacaagaagaatccagtgaagatgcttcagacaggtgactgtgatttaaacaaacaaaaaaaatctatgaatgcacatatcatataccatgacttctgaagactataaatgaattccacaatcagtgcttcactgagaaccaattttacctatcttttcttctaaactgaacagtcagagagacagctcctggctttagcttcttgtggtaccacgcactttgagcactttgtgcgtatcatgcaatatacttgcaatacacagaacaatttcaaatacgcctcacttttagacttagaagagaaacattaaaacttaagggtgtaaggagggatcaagaacttgataaggtcaaacnatatctctccttacagccaggcaaatcatc

SEQ ID NO: 61 - DNA, 3906 bp, Homo sapiens
  misc_feature: Incyte ID No 6451054CB1
aagcccttgcctgccagggaactagtatgtcctgtggggggggagattttccctggtgtctcagggccacagagattgaacaatgggcccgagtcacactgcaagtcagcagcaaaggcagcagtctaaatgagcccccaaaaagaggaggtacccatgttctagaaaggtgagccaaggagccccggggtggcggcagcggagtaggaggcgaggcaagacctgcggctcggcccggccacagccgcggtagtgctaggcaagcccacggagtcacgccggcctcagccagtctgcgaacctctcgcccccgcccacccctgcatccgcccggcccctgcccgcccgagtggtccggggaggcctagtctgcaccacccgccgggctcctggggccgcgcccgcgcggtcctgtcggcgtccgcggtcgcgcccggcccctggcccgctcgcccgccggcaccatgatggaggagatcgaccggttccaggtgcccaccgcgcactcggagatgcagccgctggacccagccgccgcctccatctctgacggagactgtgacgcccgggaggagaagcagcgggagctggcccggaagggctccctgaagaatggcagcatgggtagccctgtcaaccagcaacccaagaagaacaatgtcatggcccgaacaaggctggtcgtccccaataaaggctactcctcacttgaccagagccctgatgagaagccactggtagcccttgacacggacagcgatgatgactttgacatgtctagatactcctcctccggctactcctctgctgagcagatcaaccaagatttgaacatccagctgctgaaggacggctaccggttagatgagatccccgacgacgaggacctagacctcatcccccccaagtccgtgaaccccacgtgcatgtgctgccaggccacgtcctccaccgcctgccacattcagtagcgggcggggccactgcggtcgccggggcggggccgccaagggccgggtaggacgggcagaggccgaccccttcccagctcgtctgcctgcccccggccccgcgcccctacagccgccaacctcgtataacagtcattgcttcctcctatggtaggacgtgtacctctgtgtgttcatggagctggagaaaccaaaactgggtctctctccacccgggagctgaggaggggtgggggccgtttgttcagttacctgggggaacctcacccagcaccctgccccgcttcccagggctggccaagagagaagactggacggcagggaggtcaacattaacgagtggggggatcacaaagccaggggcttggccccaccccagcgaagccatgaactcccaagccccggcctcagcctaaccaggaaaggggctcggagggagaaaggcccagggcagtggggacgggtcccagcacctcggagcccccttcacacgccccctaccggcccatgtttctatttgcatttccccctcctcagatgggaagcctggcaaagctgcccaaccgggtagtgcccctcacccatcggatcactgccttcttcctcctcttgccccattcacccgcttccttgcactctgggagtggggcagggtaaggtgggccttacagacaggctgaggtttgggtggtgtgatctgtcctaattggcccctcaccaatgcatctatctgtctgccaccagcccaccccaccctgcccccaccctcacccacgcttctgctcattggtctcaatcccagcctggaagcagggagtaaggctggactccaatggcccattcccctcaccccagcccaagacgtgggaggctgcttcccagtaagaatccaggagcaagctttggtgaatccagtgggctgagtggctctatggatgtggttgtttggatgtggtagggagtgcttagcagaatgtgtgtgtatctctgaggctgtcagggggtgggtgggtgtgtgtctctgttggacctgtctctcctcagtctattcctgtcatagatgtgggtgtccctggtagggaagtgcctgtgtggtctgtttacagtgcagaaggcggctcagcagtgcaatgggctgacagccttggaccgggagtcagaagcctgggttctggttgtagctctggcccctccctgtgatcttcaacaagccacttgctttctctcgtcttcagtttcctcaactagaaaatgaacagcagctgaaattatgttgaagatccttttatacctgaaagagtttcttcatagaaatttctgtggtggggagggggcaatctctgtgtgtctcagtatacctaaaggttggggtggggcggagttgtgtgtctctgtagaaaggcgaaggggcagttcctatcaggactgtgtatgtctgagcacatgtggctctgtttgggattacgtgtttgtctgtgaatgtgtgtgtgtgttggagggttgtctattgtgtgtggctgtatagggtgtctgtagatcaagatgtgtatacagctgcttctgctattgctggtttgggggaggggactggaaagctgagactgaaaatcaagagggaagaggtgaggaaggcccacccctggggctgggcagtgtagacctgccaccgtgtgaagaaaaatgaaagcacagatagcctaagactgcacaggcctgtgcccacataccttatagcacaggcccaggtggatcatctcatgctaacaccccacatgtgctggtggatccactcccatggatccactcacatgctgcaggcatgcagtagactcacccacagcccactctctggacaccaatcctgggaggtgggagctggaatggggagcaatgagaggagggagtgagactgttggcttaggaagggtggtacctctccctgcatcatacccaggaacttccagctccaaaatattggggtacagctcaccaccccccactccgccaacccattcctacctcctctagtgcagggacccaagttagatgatgagaaaatgccaaacaaagggaggcaaggacagagaaggtacctgcaggacccacaaggcccatactggcccccatctgaacccacctctcccacccagatcaacatccccccaacccctgtgcccccatcatctggaccccagagccagccctcccactaccaccaccaccaccccggggctccttggctcacctttctagaacatgggtacctcctctttttgggggctcatttagactgctgcctttgctgctgacttgcagtgtgactcgggcccattgttcaatctctgtggccctgagacaccagggaaaatctcccccccccacaggacatactagttccctggcaggcaagggcttccaactgaggcagtgcatgtgtggcagagagaggcaggaagctggcagtggcagcttctgtgtctagggaggggtgtggctccctccttccctgtctgggaggttggagggaagaatctaggccttagcttgccctcctgccacccttccccttgtagatactgccttaacactccctcctctctcagctgtggctgccacccagccaggtttctccgtgctcactaatttatttccaggaaggtgtgtggaagacatgagccgtgtataatattttttttaacattttcattgcagtattgaccatcatccttggttgtgtatcgtgtaacacaaataatgatattaaaagcatcaaacaaaaaaaaaataaatggtgctgacgcctctggcttgcgccaggacttggcgtgggcaccgggcgcccccatcccagtgtctgtgtgcgtccagctgtgttgcacaggcctgggctccccactgagtgccaagggtcccctgagcatgcttttctgaagaa

SEQ ID NO: 62 - DNA, 3236 bp, Homo sapiens
  misc_feature: Incyte ID No 7494592CB1
gccggacggctgacagagcggctgacggagccgggctcaccaggtcgctgccgcgagggagttgctgtgctggggcctgggtggcggctggaggcctgagttgggctcgcggcgggggtcgcgcagggggcctgggtggcggaatgatggaggaggaggaactggagttcgtggaggagctggaagccgtgctgcagctcacgcccgaggtgcagctggccatcgagcaggtgtttccaagccaggaccctctagatcgagcagatttcaatgctgttgagtatatcaataccctgttcccaaccgagcaatctctggcgaacatagacgaagtcgtgaacaaaattaggctgaaaataaggagactggatgacaatattcgaactgttgtaagaggtcagacgaacgtggggcaggatggacggcaagcgcttgaagaggctcagaaagctatccaacaactctttggcaaaatcaaagatatcaaagacaaagctgaaaaatcagagcaaatggtgaaagaaatcacccgtgatattaagcaattagatcacgccaaacgccacctgaccacctcaatcaccacactgaaccacctgcacatgctggcaggaggtgtcgactccctcgaagccatgaccaggcgaagacaatacggagaagttgctaatctccttcagggtgtgatgaatgtcctggagcacttccacaagtatatggggattccgcagatccggcagctttccgaaagagtgaaggctgcacagactgagttaggacagcaaatcctggcagattttgaagaagcgtttccttcccagggcaccaagagaccaggaggacccagcaatgttctacgagatgcatgtctggttgctaatattctagatcccaggatcaaacaggaaatcatcaaaaagtttattaaacagcatctgtcagagtatctggtactttttcaagaaaaccaagatgttgcctggctggacaaaatcgacagacgctatgcctggataaaacgccagcttgtggactatgaggagaaatacggccgcatgtttccacgtgagtggtgcatggctgagaggattgcggtggaattttgccatgtgacaagggcagaacttgccaagattatgcgtaccagagcgaaggaaattgaagtgaaattgcttctttttgctattcaaagaacaactaactttgaggggtttcttgcaaaacgcttctccggctgcaccctgaccgatgggaccctgaaaaagcttgagtctccacccccatctaccaatcccttcctggaagatgagccaacaccagagatggaggaactggcaacggagaaaggagatttagatcaaccaaagaagcctaaagccccagacaatccatttcatggcattgtttccaagtgttttgagcctcatctctacgtgtatatcgaatcccaagacaagaacctcggagagctgatagatcggtttgtggctgatttcaaagcccaggggccacctaagcccaacactgatgaagggggtgccgtgctccccagctgcgccgacctctttgtctactacaagaagtgcatggtgcaatgctctcagctcagtactggggagcccatgatcgccctgaccaccattttccagaagtacctccgagaatacgcctggaaaatcctctctggcaacctgcccaaaaccacaaccagcagtggaggactgactatcagcagcctcctcaaggaaaaggagggctcagaagtagccaagttcactctggaggagctctgcctcatctgtaacatcctgagcacggcagagtactgtctggccaccacccagcagctagaagaaaaactcaaagaaaaagtggatgtaagtctgattgaacgaatcaatctgactggagagatggacacgttcagcaccgtcatctccagcagtattcagctgctggttcaggatctggatgctgcctgtgatcctgccctgactgccatgagcaagatgcagtggcagaacgtggagcacgttggtgaccagagcccctacgtcacctctgtcattctgcacatcaagcagaacgtccccatcatccgtgacaacctggcttccacacgcaagtacttcactcagttctgcgttaaatttgcaaactccttcattcccaaattcatcacccacctcttcaagtgcaagccaattagcatggtgggagcagaacagctgctgctggacacccactcgctgaagatggtcctgctcgatctcccctccatcagctcgcaggtggtgaggaaggcacccgccagctacaccaagatcgttgtcaaaggcatgacccgggctgagatgatcctcaaggtagtgatggcccctcatgaaccgttggtggtgtttgttgacaactacatcaaacttctcacagactgcaacacagaaacctttcagaagatactggacatgaaggggctgaagaggagtgagcagagcagcatgctggaactcctgcgccagcggctccccgcaccgccctcgggggcagaaagctccggctcactgtccctgacggcgccaacaccagagcaagagtcgtcacgcatccgcaagctcgagaaactcattaaaaagagactgtagcagcagcaaggggccctttgctcctggctggagaccctcagcgcccgttccccagaagcccccaacctctcctgtgctccccggcactctcacatcgtcggtcttcaaacttcctgggacatgtgggttgttactgagtctctcccatgccctgtcttacttcctgccctaatcggagacgctgagtaagggctgggctctaagagggcgatttaggtgatctctggttcgtgaagcagaggcagcagtggaggagtttgagaccagtctgggcaacctggcaaaaccaaaaatacaaaaaattggctgggtgtggtggctcatgcctgtaatcacagcactttgggaggctgaggcaggtggatcacctgaggtcaggaattcgagaccagcttgcccaacatggcgaaaccctgtctcaactaaaaataaaaaaatttacccggggtcaatgcgaatgcctgtattcccagtactgggaagctaaggaagggattcacttgaacccgggagttgaaggttaattaaccaagtttcaccaatgaattcaacctggtgaaaagcaaacctctntcaaaagtttacggttaggga

SEQ ID NO: 63 - DNA, 1906 bp, Homo sapiens
  misc_feature: Incyte ID No 5202657CB1
cggaagccggggccggggctgcggggcgagttgtcggccctgggccgggagctggagtcccagactcataggtcccggcccagcccccgaagagccgcctcagccggggggagttgctcggactcaaacgtccagtcctcgtgcgaccgcgctgggtcggaagtgagcaggctgaggccaccatggagcagtgtgcgtgcgtggagagagagctggacaaggtcctgcagaagttcctgacctacgggcagcactgtgagcggagcctggaggagctgctgcactacgtgggccagctgcgggctgagctggccagcgcagccctccaggggacccctctctcagccaccctctctctggtgatgtcacagtgctgccggaagatcaaagatacggtgcagaaactggcttcggaccataaggacattcacagcagtgtatcccgagtgggcaaagccattgacaggaacttcgactctgagatctgtggtgttgtgtcagatgcggtgtgggacgcgcgggaacagcagcagcagatcctgcagatggccatcgtggaacacctgtatcagcagggcatgctcagcgtggccgaggagctgtgccaggaatcaacgctgaatgtggacttggatttcaagcagcctttcctagagttgaatcgaatcctggaagccctgcacgaacaagacctgggtcctgcgttggaatgggccgtctcccacaggcagcgcctgctggaactcaacagctccctggagttcaagctgcaccgactgcacttcatccgcctcttggcaggaggccccgcgaagcagctggaggccctcagctatgctcggcacttccagccctttgctcggctgcaccagcgggagatccaggtgatgatgggcagcctggtgtacctgcggctgggcttggagaagtcaccctactgccacctgctggacagcagccactgggcagagatctgtgagacctttacccgggacgcctgttccctgctggggctttctgtggagtccccccttagcgtcagctttgcctctggctgtgtggcgctgcctgtgttgatgaacatcaaggctgtgattgagcagcggcagtgcactggggtctggaatcacaaggacgagttaccgattgagattgaactaggcatgaagtgctggtaccactccgtgttcgcttgccccatcctccgccagcagacgtcagattccaaccctcccatcaagctcatctgtggccatgttatctcccgagatgcactcaataagctcattaatggaggaaagctgaagtgtccctactgtcccatggagcagaacccggcagatgggaaacgcatcatattctgattcctacctggaaggaattttgttgaaaggggttttcacctgtgagccttggtctgtctcggtagggtggtcaacttcagtggactgtggttggtttcagagcgcctggctgaggagttccactgaggggagcactggagcagccctttggcagaggctgaggagggagatggaccagcccacgcctggcacctggctccatggcataaggaaagggagatgctggcctctgtgctcctgctgtcttttcctgtttctgtttgcgtttgacttagtagcaaccgacagagtggcaagggatttggtcttcagcagtagacatccttccacccctgccctcagccaagtctcttgctgccatgccaatgctatgtccacccttgcccctcggcccaagagtgtccagcggtggcccacctcttcctcccactacagcctcaacagtatgtaccatctcccactgtaaatagtcccagttagaacggaatgccgttgttttataactttgaacaaatgtatttactgcccttctcaaaaaaaaa

SEQ ID NO: 64 - DNA, 1347 bp, Homo sapiens
  misc_feature: Incyte ID No 2013529CB1
gcccgggcgcaggcggcggatggagcggaacggctaggggtcttgagaagcaatggccacagaagcccctgtgaatatagcaccacctgagtgtagcactgttgtcagcacagcagttgacagcctcatttggcagccaaactcactaaatatgcacatgataaggcccaagtccgccaagggacggacaagaccgagtctgcagaaatcccagggcgtggaggtgtgcgctcatcatataccatctccgcctccagccattccctatgagttgccaagcagccaaaaaccaggagcctgtgcacccaaatctccaaaccagggagcttctgatgagatccctgagctgcagcagcaagtacccactggggcttcctcttctctcaataagtatccagtccttccttccatcaacagaaagaacctggaggaggaggctgtggaaaccgttgccaaaaaggccagctcactgcaactgagcagtatccgggctctttaccaagacgagacgggcaccatgaagacaagtgaagaagattccagagctcgagcttgtgccgtggagaggaaattcatcgtccgaaccaagaaacagggctcttccagggctggaaatctggaggaaccatcggaccaagaaccaaggttgctgcttgctgttagatcaccaacaggccaaaggtttgtacgccatttccggccaacagatgatttgcaaaccattgttgctgtggccgaacagaaaaacaaaacctcctaccgacactgcagcattgaaacaatggaggtgcccaggaggcgattttctgacctcaccaaatctctgcaagagtgcagaatcccccacaagtctgtgctgggcatctcactggaagatggggaagggtggccctgagtccacagccacccagctgaggtcctgggtctctgagcaaaggagcatgcttgggcgttgtggcctcttaggcagcctgtttcaagtgccatgtggacctggtgcagctgggaagcttgggactctcgtctgcactgcgtgtcctctgaagcagtgaagtctgtgcctatgccgagcgcgctaagaagtctcccttccagctgttccattctctccaccaccagcgtaactggcaagttaccaaggttgttcctgaaacagcagtgatcatgacttctcctttccagagttttgggtccttctgaattaatggtccttttcgaacaccggcttgcctttacagtgaactgtgattctctcgaagccaatgctttcctgtctgtatttgatgcaggattaaacacttcccagagaggattctagtctggtaaataaccaccaaaaaaaaaaaa

SEQ ID NO: 65 - DNA, 1854 bp, Homo sapiens
  misc_feature: Incyte ID No 3841351CB1
gcagagcgagtcgggaaacgattttaaactgaagaggcggcggagggccgaattcccttttctcaacggcttgatttcagagctgggctggtctctgacaggctcagctggagagggacgggttgggacgcactgtccttttgcccttccccctccgcgagcagaagctgactccgcaggagcgagggtcgcagagctgggggatttcagtctccacatagttttggagccggacttttgaagaatgattcgtgaatccggaatgggtgacagcgtcatcacggcattttattgacagaccatggattcttacagtgcaccagagtcaactcctagtgcatcctcaagacctgaagattactttataggtgccactcctctgcagaaacgattagaatcggtcaggaagcagagttcatttatcctgactccacctcgaaggaaaattccccagtgttcgcagttgcaggaagatgttgaccctcaaaaggttgcattccttctgcataaacagtggactttatatagtttaactcccttatataaattctcctatagtaatctcaaagagtattctagacttctcaatgcttttattgttgctgaaaagcaaaaaggacttgctgtggaagtgggagaagacttcaacatcaaagtgattttttctactctcctaggaatgaaaggaacacaaagggacccggaagcatttcttgtccagattgtgtcaaaatctcaattgccatctgagaatagagaaggtaaagtgctgtggactggctggttctgctgtgtatttggagacagtcttctggagactgtttcagaagatttcacctgtctgcccttattccttgcaaatggagcagagtctaacacagcaataattggaacttggtttcagaaaacctttgactgttatttcagtcctttagcaatcaatgcatttaatctttcctggatggctgccatgtggactgcatgcaaaatggaccattatgtggctactactgaatttctttggtctgtaccctgtagccctcaaagtctggacatttctttcgcaatacatccagaggatgcaaaagctctatgggacagtgtccacaaaacacctggggaggttacccaggaagaagttgacctattcatggattgcctttattcacatttccatagacatttcaaaattcatttatcagccacaagattagttcgtgtttcaacatctgtagcttcagcacatactgatggaaaaataaagattctgtgtcataaataccttattggagtgttagcatatttgacagaactggcaatttttcaaattgagtgaagccttatgtggactataagttatagattatatactcttattgataacttgcctaattgctatgctgaaagagactgcaggagaaataggcatctatctctgcatctgttttccccaccatgcctttggagttgccaagatggaagccaagaaggatctagaagaacaaagaatatggtagtagatgagccacagccaggtgcccatgtactaatcatgataacctgacatgccattctcaaaatgctgagttgttaatttcttgtcatctttaaatatatatatataggctgggcttggtggctcacacctgtaattccagcactttgggaggctgaggtgggtggatcatttgaggccaggaattcaagaccagcctggccaacatggtgaaaccccttctctactgaaaatacaataattagctgggcgtggtggcacatgcctatgatcccagctactggggaggctgaggcaggagaatcgttttaacccagaagacaggctgcagtgagcca

SEQ ID NO: 66 - DNA, 1327 bp, Homo sapiens
  misc_feature: Incyte ID No 152116CB1
tgcctcctctggctgctgcctccgcagctccctcctcctaccccacctcctccatctggggagcgtctgcgggggcctgaggggcggcggcggcggcggcggctgcgatatggagccaggggccggcggccgaaatactgctcgtgcgcagagggccgggtccccgaacactccgccgccccgggagcaggagcggaaactggagcaggagaagctctccggtgtggtgaagagcgtccaccggcggctccgcaagaagtaccgagaagtgggagattttgataagatctggcgagaacactgtgaggatgaagaaactctttgtgaatatgccgttgcaatgaaaaatttggcagataatcattgggcaaaaacttgtgagggcgaaggtcgtattgaatggtgttgtagtgtatgcagagaatatttccaaaatggtgggaagagaaaagcacttgagaaagatgaaaaaagagctgtacttgccactaagaccactccagccttaaatatgcatgagtcttctcaacttgaaggtcatttaaccaacctcagctttacaaaccctgaatttataactgagttgctacaagcctcaggaaaaatcagattacttgatgttggcagctgctttaacccatttctgaagtttgaagaatttctaactgttggcatagatattgtacctgctgtagagagtgtctataaatgtgatttcctgaacttacagcttcagcaaccactccagcttgcacaggatgctatagatgcttttttgaagcagctgaaaaaccctattgattctcttcctggagagcttttccatgtggttgttttctctctccttctttcttattttccatctccttaccagcgatggatttgctgcaagaaagcccatgaactgttagtgttaaatggtttattactaatcatcacacctgattcctcccatcagaaccgtcatgctatgatgatgaaaagctggaagattgctatagagtccctgggctttaaacgcttcaagtactcaaaattttcacatatgcatctgatggcatttaggaaaatctctctaaaaaccacaagtgacttggttagtaggaactacccaggaatgttatatattcctcaagatttcaacagtatagaagatgaggaatattctaacccttcctgctatgttcgatcagatatagaagatgaacaactagcatatggtttcacagaactccctgatgcgccatatgactcagattctggagaaagtcaagccagctctattcctttctatgagctagaagaccccatattacttttaagttaa

SEQ ID NO: 67 - DNA, 627 bp, Homo sapiens
  misc_feature: Incyte ID No 2381031CB1
atccgggcctgagagtgcaggcttgagggaagcatggaggtccatggcaagcccaaggctagcccgagttgttcgtcgcccacccgggattcctcaggagtcccagtgtccaaggagctgctgacggcgggaagcgacggccgcggaggtatatgggacaggttgctcatcaactcccaacctaagtccagaaagacctccactcttcaaacagttcggatagagaggagtcccttattggaccaggtacagacatttctcccacagatggcacgggcaaatgaaaagctaagaaaagaaatggcagctgcaccacctggtcgtttcaatattgaaaacattgatgggcctcatagtaaagttatacaaatggatgtggctttgtttgagatgaatcagtcggattcaaaagaagtggacagttcagaagagagttcacaagacagttcagagaacagttcagaatcagaagacgaagatgacagcatcccatctgaagtcaccatagataacattaagcttcccaattctgaaggtggaaaaggcaagattgaagttttggacagtccagcaagtaaaaaaaagaaatagtcaaataaattatctgaaaagaaaaaaaaaaaaaaa

SEQ ID NO: 68 - DNA, 2564 bp, Homo sapiens
  misc_feature: Incyte ID No 2511371CB1
gcacgctgactttgctctcactgcccggccagacgcacttcccgccttcgcctaggtctgcaccgccaccgccgccctctcccgcgcgtccctcacccggtccgccactgccgcgcctcagaatccgaaggctcggttccagcgcagccatccacgccgccgccacgaccgactccggagccacgcgctgcaaccgccaccaacgccttgccgccgccgctcgcccgccgaccaccatggcccagcagcaaacaggtagcaggaaacggaaagcgcccgcggtcgaggcggacgccgagagctcgccgtcgcagggcttggcggcagcggacggtgaggggccgctgctactcaagaggcagaggcggccggcgacgtatcgctcgatggcgcactatctgaaggttcgggaggtaggcgggtggggccccgccaggctccagggcttcgatggcgagctgcggggctacgcggtacagaggctgcccgagctgctgacggagcgccaactggagctgggcacggtcaacaaggtgttcgcgtcacagtggctgaactccaggcaggtggtgtgcggcaccaagtgtaacacgcttttcgtggtggacgtggagtcaggccacatcgcgcgcattcccctcttgcgggacagtgaggccaggctggcccaggaccaacagggctgcggcatccatgccatcgagctgaatccctccaagacgcttctggccaccggcggcgaaaaccccaacagcctggccatctaccagctgccctccctggatcccctgtgcctgggcgaccgccatggccacaaggactggatcttcgccgtcgcctggctgagtgacaccgtagccgtgagcggctcccgcgacggcactgtggcgctgtggcggatggacccggacaagttcgatgacactgttgcctggcatagcgaggtgggtctccccgtatatgcccacatccgtccgagggatgtggaggccatccccagggccatcatcaaccccagtaaccgcaaggtgcgggccctggcctgcggcggcaagaaccaggaactgggagcggtgtccttggacggctacttccacctgtggaaagccgggagcgcactatccaggctgctgtccatcaggctgccctacttccgggataatgtgtgcctgacctactgtgatgatatgtctgtgtacgccgtgggctcccattcccacgtctctttcctggatctgcgccaggaccagcagaacatccggcccctgtgttctcgagagggtggcacaggcgtgcggtcgctgagcttctaccgccacatcatcactgtgggcaccggtcagggctccctgctcttctatgacgtccgggcccagaaattcctggaggaaagagcctccgccaccctggagtcctcttcgggacctgcaaggaggaagctcaggcttgcctgtggcagaggctggctcaaccacaatgatttctgggtgaattactttggtggcatggaagtgtttcccaatgcgctctacacccactgctacaactggcctgagatgaagctctttgtggctggggggcctctccctgcaggcctccatgggaactatgcaggcctctggagctaaggatgaccgccttgtactccaggtggaaccagcccagcttaatttgtactcctccaagcagagagagtgagtggtcctttggtcaatgaaaacttggctttaagactttttgactcctttccaacaccatcttttggaattttcatacaaagaaatttggttaatcgttaatctttagttgcattctctctttgagtgattcatacattctgtttctttaagaagatgcagtcatggtctttcagttatgtatatttgttttccttgtttttcctcgttaccaatattacaacagcattgttttcagttatttttcattgcagatacgaagagaaaattttctagtaagcaaggtgttcctacatcttttctggagagcttaccatagttttatagcattgttcaattggaaaagtacattccaagttctagattacctacgtttggaaacacttaatttacaaattgtgatcttagtgtatatagtttttacgttgttggtgctaaagtgcttttttgaagtttattctctttcaaaataagttatcatagaatcttgtgcttgtatcaaactcctaattggtcggaacactttaggagtgcaggggttctgcttcattgcattttcagaattaacctgaaagttgaaacaaccatttttttttatttcaaaacttatttaagttgttatcacctgtccttaaatagataaaagaatgtgtattttttcacttgttgccttagtactatcaagacttgtgaatatatttgaatgtatgttgtttgaaaaacttgcacagggcattggagccatcatgaaaagcaggaaatgtaaacgtggaatgtatggcaggctgggcagaatgtatactgatccagggtatagactagatatttttgctgttgctgtttttattataaaatgttatatttgag

SEQ ID NO: 69 - DNA, 4134 bp, Homo sapiens
  misc_feature: Incyte ID No 8068623CB1
tgctggtgctgtgccgtcggccctgccggcggcgcagctcgggctgcgttccttctctgcgtgcgcttggcactgcgtcgtcgtctttctggtttgtcctctgtggttctctccccgctcccttgtctgtgtcttcggctgtgctggtcgggttcgttgtcgttgtgtgttcctctgtcgtgtcttgctctgcgtttgctgcttgcgttgtctgtgctcgtcgtgtgctcgctgtgttgtggtgtgtttgtgctctggttggcgtttgcttgtcgtccttgggcgtcggtgcttggttgtgtcggttttctcgtatgactgtcacgttgtttatgttggtctttgtgtgttgtctttgttgtgtttcttttcttgtctcttttctgtttgtgtgatttatctatttagtgtcgttgttgtgctcctctgccttcgtgctgcttctttcctcctgcgtcgtctcgtctgtcggtctacggtgttgcgatatatccgcgatgctgctgtccaatggcggacctaacgcgttgtgttaaccgcctagcgggttctcttcctctagttcaccgtatcggcttggtataagagcgtgactggccctacggtgaacctcgaaattgttaaggggagcactctatacgtagagtctattgaacgttcgcattgtaactgctgtaacgttatagctctggataattcgcgcttcgatgatttgggaaagaaatgggaatatcatcttgttatgacggaagaaagggaaatcgagatcctgaatatacgccatgatgccgctgcttctacgattgacgtggcccacctatttcggcacaactctaaagggggggcaccgggttcaaaccgtgaaacggtgctatcgtaaaaatgggcgcttccccaagggccttcgtgcaagtgcaataactctggcggatcccgttattgaccatggaaacctggcctagaatcctcctaagtatcctcaatgttactgtatgaacaggtagtcttcgtgttggtgggtatttatcgtagatcaaggacagtgagatgaatgcgacaaactgataaggctgatcagacctaggctcgtttgatgcgacgaactacgtctgcatacgagcgccgacacatcttatacccgctatgagatttaacaaggcggcaggacgaagccaaactcctcagagcgctcaccgcgctctttccgcagcctcccccgccgccggccgggcgtagcgcagggtcacgtgccggcgacggggcggggccagcggggcgcctgcgcgcctgcgcgccgctggccgacggaggggagcctgccgatgccgagcgggtgctacgtcccgcggtcggagccgcgtcttctcccggctccgccaccagccggggctcgggtgggggcccggggccccggggcatggccctccggagtgcgcagggcgacggccccacctccggccactgggacggcggcgcggagaaggcagactttaacgccaaaaggaaaaagaaagtggcagagatacaccaggctctgaacagtgatcccactgatgtggctgcccttagacgcatggctatcagtgaaggagggctcctgactgatgagatcagacgaaaagtgtggcccaagctcctcaatgtcaatgccaatgacccacctcctatatcagggaagaacctacggcagatgagcaaggactaccaacaagtgttgctggacgtccggcggtcattgcggcggttccctcctggcatgccagaggaacagagagaagggctccaggaagaactgattgacatcatcctcctcatcttggagcgcaaccctcagctgcactactaccagggctaccatgacattgtggtcacatttctgctggtggtaggcgagaggctggcaacatccctggtagaaaaattatctacccaccacctcagggattttatggatccaacaatggacaacaccaagcatatattaaactatctgatgcccatcattgaccaggtgaatccagagctccatgacttcatgcagagtgctgaggtagggaccatctttgccctcagctggctcatcacctggtttgggcatgtcctgtctgacttcaggcacgtcgtgcggttatatgacttcttcctggcctgccacccactgatgccgatttactttgcagccgtgattgtgttgtatcgcgagcaggaagtcctggactgtgactgtgacatggcctcggtccaccacctgttgtcccagatccctcaggacttgccctatgagacactgatcagcagagcaggagacctttttgttcagtttcccccatccgaacttgctcgggaggccgctgcccaacagcaagctgagaggacggcagcctctactttcaaagactttgagctggcatcagcccagcagaggcctgatatggtgctgcggcagcggtttcggggacttctgcggcctgaagatcgaacaaaagatgtcctgaccaagccaaggaccaaccgctttgtgaaattggcagtgatggggctgacagtggcacttggagcggctgcactggctgtggtgaaaagtgccctggaatgggcccctaagtttcagctgcagctgtttccctgaaagccagagaagaccttcctcttacatcacattaaggtctcacccttccatggaaggattgggaggggtggaatttcctttattaaaagggtttctgtccagagttttttattcctgccaccctgccctgcccgtttgtgtttgcctttacttcagaggccaatagcagagcctacctgacacagaggggacctgggggcctggcacagaatcttatgcctctcccagtgggcttttggatgggctctctgccacctccctggtgttggaggaagtgggtttctgaagcttgcagctgcttttgcaacctgctgggcctgggctgccctagttaccgggaacctcctagtcaccccatagaaggtgaactggaacatgggatgccctcttactgctcctgcttgcctgcatcagatggacacagaaggagggggctgctgctgaaggagtcaggcttgggatggagagcaagaggccaaaggactcgtctgggagcatgatcggcccctcttactagcagctccatcagctgatcagaaatcagagacagaagggaactaggttactcgttaaatcctacctattttcagcctagaaatcacatgggcgtttccagtcaagtgggaaggaagctcatttttagggcctagacctagactagttttgcctgcctttttttcctccctgctttcttctacaagccacacaacattgcttataaaaggtacttttattactatttttagaatatatacaccccatatgtaaattaccctcacttttaaagtaataatagttccttcacggaaaaaaaaatagttctcatcagaaggatgagcttctaagatcctgaattttactttctacttgattaacattttcgctttagccagagttgctatttgcaaacagctttccacttccaaagtgccactttctgaccggtttttcttcctgttttcctcaaatcatcctactcccaaataggcacccactcactaccttggcgtcgttttttgggtctgacttgtctcgtcaactgctggtctctcccacctggttggaaatgtcgttggaaacttgcaaagactctccagaccttagggaacaagaggcatcactcagtccttctgggacagcttccctgtaagcaaagccaggtttgttttagattcactctggcctgaaaaaaaaagtgccttttgctgctttaaagaattggggtatatggtatgaagcagccatgtacttgtattttcctggtctttcctgggcactcttctctcttggcagatgttttcttaaagtgaacacaccagaagcactctaccccaccatatccagtcctctttgcatgcctgttgtgtgcagggtggagggtgtcccacgtgccagagcagctgggagccctgtccgctgagttacactaagcactttatttttatttattttattattattttcttttttctgagatggagcatgggcaaaaagagcgaaagcta

SEQ ID NO: 70 - DNA, 2329 bp, Homo sapiens
  misc_feature: Incyte ID No 677977CB1
atgaaacattgccttccgaaaagcctactacatccaaggacaagttgcaggccatcgagccgcgtcaatgggcggcgagcgcaaggcccagacgtgtgccgcgacattcagcgtgccggcgcgggcatgcgcggctggctcaaggacgatgcccacatgcgctggttcctggagcagctgggcggtgaggtgggcactcagcgcaagaagatgcggctggccaacgaggaggagatcgaccgcgccgtgtacgcctggttcctggcgctgcgccacgacggggtgccgctgtctggcccgctcatccaggcgcagccgaggccttcgcgcgccagatctacgggcccgagtgcaccttcaaggccagccacggctggttctggcgctggcagaagcgccacggcatctccagccagcgcttctacggcgaggcgggcccccagccccgagccccgcgcccggcccgcccgtcaaggaggagcccgcgctgccctccggcgccggccccctgcccgaccgcgccccggccccgccgcccccggccgagggcggctacggggacgagcagatttacagcgccagcgtcaccggcctctactggaagctgcttccggagcaggctgcgcccccgggcgcaggggaccccggggcggggggctgtggccggcgctggcggggcgaccgcgtaacggtgctgctggccgcaaacctgaccggcagccacaagctgaagccgctggtcatcgggcggctgccggacccgcccagcctgcgccaccacaaccaggacaagttcccggcctcctaccgctacagccccgacgcctggctcagccgcccgctgctgcggggctggttctttgaggaatttgtcccaggcgtcaaacgctacctgcgccgaagctgcctgcagcagaaggccgtgctgctggtggcccacccgccctgcccaagcccagctgccagtatgcccgccctggacagcgaggatgcccccgtgcggtgcaggccggagcccctcggtcccccggaggagctgcagacaccggatggcgctgtgcgggtgctgttcctgtccaaaggcagcagccgggcacatatccccgaaccggtggagcagggcgtggtggccgccttcaaacagctgtacaagcgcgagctgctgcgactggctgtgtcctgcgccagcggctccccgctggacttcatgcgcagcttcatgctcaaggacatgctctacctggctggcctctcctgggacctggtgcaggcgggcagcattgagcgctgctggctgctgggcctgcgggctgccttcgagccccggcccggcgaggacagtgctgggcagccggcccaggccgaggaagccgccgagcacagcagggtgctcagcgacctcacccacctggcggctctggcctacaagtgcctggctccggaggaggttgcggagtggctgcacctggacgatgatggggccagtctgccctctgccatggggggcggagaggacgaggaggaggccaccgactatggagggacctccagtctgccctctgccattgggggcggagaggacgaggaggaggccaccgactatggagggacctcagtgccgactgccggggaggccgtgcgggggctagaaacagctctgcggtggctggagaaccaggaccccagagaggtggggccactgaggctggtgcagttgcgctcactcatcagcatggcccggaggctggggggcatcgggcataccccagcaggcccctatgacggtgtgtgaccaggccagcccagtgacctttctcctgctgcacttggagggaggggacatacacacagtctcccatctctcctcccctccccctggggtggcccaccgcatgggtacagggggttccaggaatccaaatccagcatggcttggaggagctctgttggtgagaggtcgccctgcctcactggcaccctgggggcacagctggaagagaggcctggcccatgctcctctcagggcaggcacatgtacggggcatacaaggcacagcgcctgttggaacaggtggctgtgttcctgctctggcccccgtgcggctgggcctccgccccttgcaccagtcacatgcactggacgagggccgaaactcctgtctgctatcgagcctggtgctatgtggccccggagccacagcaaatcatctcgagtggcgaggcacccacttgatctctttttcttaaccctttatctggttcaggttacacaaaaagggggcgcccgattgggccgcccggaaaatccgcgcccggggacgtccttggg

SEQ ID NO: 71 - DNA, 2960 bp, Homo sapiens
  misc_feature: Incyte ID No 1661472CB1
tcaagttctgattctgagctgctccgccactggcctgtgggaagaggcaggaggcttggggtcccccagcctgtgcccagcctcctgtgccttgtagatcttcacatatgagcgcaagtggtactcgcgcaaggacctggcccggcatcgcatgcagggtgaccccgatgacacgtcgcaccgtgggcacccgctctgcaagttctgtgacgagcgctacctggacaatgatgagctgcttaagcacctgcgccgcgaccactacttctgccacttctgcgactcggacggggcccaggactactacagcgactatgcctacctgcgtgagcacttccgggagaagcactttctgtgtgaggaaggccgctgcagcacggagcagttcacccacgccttccgcaccgagatcgacctcaaggcccacaggacggcctgccacagtcgcagccgcgccgaggcacgccagaaccgccacatcgacctgcagttcagctacgcgccacggcactcgcgccggaacgagggggtcgttggtggcgaagactacgaggaggtggacaggtacagccgccagggccgagtggcccgggctggcactcgcggagcccagcagagccgccgaggaagctggaggtacaaaagggaagaagaggaccgagaagtagcagctgctgtccgggcctccgtggccgcacagcagcaggaggaggctcgcaggagtgaggatcaggaggaaggcggtaggcccaagaaggaggaggcagcggcgcggggacctgaggatccccgtggcccccggcgctcaccccggactcagggcgaaggcccaggccccaaggaaacctcgacaaatggtcctgtaagccaagaagccttctcggtgacaggcccagccgccccagggtgtgtgggggtgccaggcgccctcccaccacccagcccgaagctcaaggacgaagacttccccagcctctctgcctccacttcctcctcctgctccactgcagcaaccccgggccctgtggggttggcgctgccgtacgccatccctgccagaggcaggagtgccttccaggaggaggacttccccgccctggtgtcctcggtgcccaagcctggcaccgcccccaccagccttgtctctgcctggaacagcagcagtagcagcaagaaggtagcacagcccccactctcggcgcaggctaccggcagcggccagcccaccaggaaggctgggaaggggagcaggggcggcaggaagggcggcccgcccttcacacaggaggaggaggaggacggcggcccggccctgcaggagcttctgagcacacgccccacgggctccgtctcctccacactggggctggcctccatccaaccctctaaagttgggaagaagaagaaagtgggctcggagaagccaggcaccacattgccacagcccccgcccgctacctgtcccccaggggctttgcaggccccggaagctcctgccagcagagccgaggggccagttgccgtcgtcgttaatggacacacggagggcccggcccctgctcggagtgcccccaaggaaccccctgggctcccaaggcccctggggtccttcccctgccccacgccacaggaggacttcccagcgctcggcggcccctgcccaccccggatgccgccgcccccaggcttcagcgctgtggtgctcctgaagggcacgcctcccccacccccgccgggcctggtgcccccaatcagcaagccgccccctggcttctctggccttctgcctagcccccacccggccagtgtccccagccccgccaccaccaccaccacaaaagcacccaggctgctgcctgccccacgggcctacctagtccccgagaacttccgggagaggaaccttcagctcatccagtccatcagggacttcctgcagagcgacgaggcccgcttcagcgagttcaagagccactcaggggagttcagacagggcctgatctccgcagcccagtattacaagagttgccgggacctgctgggggagaatttccagaaggtctttaatgagctgctggtcctgctgcccgacacggccaagcagcaggagctcctgtctgcacacacggacttctgcaaccgcgagaagcctctgagcaccaagtccaagaagaacaagaagagcgcgtggcaggccaccacccagcaggcgggcctggactgccgtgtgtgccccacctgccagcaggtgctcgcgcatggcgatgccagcagccaccaggcgctgcatgctgcccgggacgacgacttcccctccctgcaagccatcgccaggatcatcacgtagctcccgccagcgtggccagagctgtcgcaccgtgagcgtccttcctccttcctctccgggctgccaggcagccaggtaaggcctggtgaggccacttggcctcttggttggccaggcccaccaggaagtcaccaggacagtccacccgccctgttggcacactcaagcgggagtccacccctgcctcagtggtgggccagtctcggtttgcattcttgtgcttttgggaggtgccaggggagggaagggctgggatgctgggacctgttgttgctggcaaagccagaggtcacagtggcctgatctgggccctcccaaagctgagggctgcagcccgtggggcctcagagctgaaagctgcggcgccactggtgccagagtcagatgtcacagatgtgttgtgtaaacagttggctgtttcgtgcttcaagaatgttcaggattaaaagcagacaagaaattgtgctacttgaagttgaatctttttatgagacaagctgaatctgggatctcaaattgcctctgaccttttataagacagtttatcttcaaataaatttattttgcaataccacgcaaaaaaaaaaaaaaaaaaaaaaaaaaaaaaaaatat

SEQ ID NO: 72 - DNA, 2623 bp, Homo sapiens
  misc_feature: Incyte ID No 1748508CB1
tcagttgatgtgttaggaaaattgattatatgtatatactaaataaaatttgttaaacatacctataacccttcaaaattaaatttttaaaaactttttactgtttcatgtaggtataacttacttccttgtctcttgatagatttcaggcttaaactgaagaagtaattggttcaaaaatcttattactagaaagtaatgacaaatttatagttttcatttctagtgtacattaaccataggtcattgtaacatcccttctccacaataaaaatggaaacttattttgtggaaatcattttatgtaaatatgtatttaatacttatttcatctttcttacctttcagaattaccatgaaattatgactcgtcatcctgagaattatcaatgggaaaactggagtctagaaaatgttgctaccattttagcccaccggttccccaatagttatatttgggtgataaaatgttcccgaatgcatttgcacaaattcagctgctatgacaattttgtgaaaagtaacatgtttggtgccccagaacacaatactgactttggagcttttaagcacctttatatgttattagttaatgcttttaatttaagtcagaatagtttatcaaagaaaagtttgaatgtttggaataaggactccatagcatctaactgtagatccagtccttctcatactacgaatggttgccagggagaaaaagtgaggacctgtgaaaaatctgatgagtctgccatgagtttttatccaccatcactaaatgacgcatcttttactttgattggattcagtaaaggttgtgttgttttgaatcagttgctttttgaattgaaagaagccaagaaagacaagaacatagatgcttttatcaaaagcataagaacaatgtattggctggatggtggtcattctggaggaagcaatacttgggttacttatccagaagtcttgaaagaatttgcacaaacaggaattatcgttcacactcatgtaacaccttaccaagtacgtgatccaatgagatcttggattggaaaggagcacaagaaatttgttcagatacttggggatcttggtatgcaggtgactagccaaattcattttacaaaggaagctccttccatagagaatcacttcagggttcatgaagtattttgagattacaggtatattaatgaacttgttcagtggaagaacataagcacttttgagtgttataaattcagataatgggatgtaattcatagctgcattgtcagttttggggtatggggggaagcacacattcctaaaatgtgagtgtaatgtgcaatagtattttttgcttgtgaatgtgagcagttattaatttggattgagttagaattagttaatttgaaatctaacaaggtggtttgtaataatgctgaggagatataagacccttaaaatgaaagttacaacattgttcttataaaaggtaactaaaattgttactgttggaaataactgattttctgagtaatgttttaaactaatttggtgacattttaacagtaattagctattttgagtggaaatattttcatttctcttcaaacaaaagcaaaggtacgatgctgttttctatcattttggaataactgcaccctgccttttgtgtttttgtaaactccttgactcattctttcatgtgtcaccaagtacttttctcatgagagtcaacatatatttgtttccaaatgtccacaagtgtacaatagtgtaaaggtggtttttaaaaacatagccaggtgtggtggcacgtgcctttagttccagctactcaggaggctaaggcaggaggattgcttgagcccaggctgtgtggttcaccataattgtgtttgtgactagctactgcactccaacctgggcaacatagtgggacttcatctctaaaacaaaacaaaacaaaattacacttaagcactattgtttaatttttaattgtcagtttatcattattttgggtaagacattctggggtttcttgaatcttgtccaaaaaccagttgttttggaaaattgctttaaattgagcatatttatgtatattggataaaaatgtactacagagcaaatttcaaatttttcattatatcagtctttttgaaaggatcaacttggataaaataaatatataatgctctatttgttagagctctattaaaaaggaaacagattccatagatctaagtcaatgtttctccagaagcatgattttgtctgccaaaagaaaatagctctctttggccaaaatgcaaaattacattgctataagaaaagttacaagggaaagtttgaagacacaaatgatttaattttggctcaaaaactgaatttgcttaacactgctacataatttgggtgaagtttccttctgcccgtttttcttgacctagataaatacactttgagaaatccagatctaataaatgtcaaccaacattgacattgtaattgggtgattacaataaaaggtgagcagtttgttgtttattaataattagcttttgcaggtaatgaaatagcagggaagtaacatgctgctttaggactaaaaaaaaaaaaaaaaaaaaactcggtc

SEQ ID NO: 73 - DNA, 4518 bp, Homo sapiens
  misc_feature: Incyte ID No 2159545CB1
gtggctgcaccttagcgacatcaggaagaacagggtcgaggatcgagtcacagcaaactgtacaatggcagttgagtgggttttgaaaaacgtctgaattgtagtgtctacatctaccttctgccttctctctaccctttgaaaaaaaataaatggcatgtttgagaagagagctctattaaaagaggaatattgcaaatggcttcagattctatgagtagtaaacaagctaggaatcacattacaaaggggaaaaggcaacaacagcaccagcaaataaagaacagatcctcaattagtgatggtgatggagaagattcctttatttttgaagcaaatgaagcttggaaagattttcatggttctcttcttcgattttatgaaaatggagaactctgtgatgtcacactcaaggttggctcaaagctaatctcttgtcacaagctggtattggcttgtgttattccctactttagagccatgtttctttctgaaatggctgaagccaagcaaacgctgattgagattagagattttgatggtgatgcaatagaagacttggtaaagtttgtctattcttcacggctcactttgactgttgacaatgtccagcctctcttatatgcagcctgtattctgcaggttgaactggtggctagagcttgttgtgaatacatgaagttacattttcatccctccaattgcctggcagtaagagcctttgcagaaagtcacaatcgaatagacttaatggacatggcggatcagtatgcctgtgaccattttactgaagtagtggagtgtgaagactttgtaagtgtatcaccgcagcacctccataagcttttgtcctccagtgatctaaatattgaaaatgaaaagcaggtctataatgctgccatcaagtggcttcttgccaatcctcagcatcattccaaatggttggatgaaacacttgcacaggttcgcctgccattgttgccggttgattttcttatgggtgttgtggcaaaagaacagattgtcaagcaaaatctaaaatgtagagatttactggatgaagcaagaaattaccaccttcacttgagtagcagagcagtacctgactttgaatactccattcggactaccccaaggaagcatactgctggtgtgctgttttgtgtaggtggtcgaggtggatctggtgacccctttcgcagtattgaatgctattctatcaacaaaaacagttggttctttggaccagaaatgaatagtcgaaggcgacatgtgggtgtaatctctgtggaaggtaaagtgtatgcagtaggtggacatgatggaaatgaacatttagggagtatggagatgtttgatcctctcactaataaatggatgatgaaggcatcaatgaacacaaagaggcgaggaattgccttggcttccttaggaggcccaatttatgcaattggagggttagatgacaatacttgcttcaatgatgtggagagatatgacatagaatctgatcagtggagtacagtggcaccaatgaatactccccgtggaggagttggctctgttgctctagtaaaccatgtttatgcagtaggtggcaatgatggaatggcttctttatctagcgtggagagatatgatccacatctggataagtggatagaagttaaagaaatgggtcagcgaagagcaggcaatggagttagcaagcttcatggttgcttatacgtagttggtggttttgatgataattctcctctgagttcagttgagcggtatgacccccgaagcaacaagtgggattatgtggcagcacttactactcccagaggtggagtgggaatcgcaacagtgatgggcaaaatctttgcagttggtggtcataatggcaatgcatacttaaatacagtagaagcgtttgatccagtgctgaataggtgggagcttgttggatctgtgtctcactgcagagctggagcaggagtagctgtgtgttcctgtttaactagccaaattcgagatgtaggtcatggatccaataatgtggttgactgtatgtgatttgagttctggccaccaacaatttagtcatatctgataggtacaaaagaaaaccaagattttgatatgaccacctttcaacactttttactgcaactagagtcttatttaaatgttaactgttgtattgtgactaagtacaacttttggaatgatacctgaagaattattagtaatagagttaaatttgatacttcctgcttttgcaaataatggagtagggaagaagatagtcgaacttaaaatcatacctaccctgaaaaatgcttaaagtagtgccaaaaatcttgaaatctctctctctctctctatttggatgaatcaaaatgtaatttgttgagttttgccttttcttttgcctaaagatgttaacgtcaggcagtgggtttttcctagaggagagattagtacctattcattaacacaaggatacataagagacttgccccttttgaagatcatttctacttggtgctgatgaatttatgcctagctttttttttctaggtggtgatttttcaatttcttttatatttacggaagtagaaaaaatgttctgtctttttgaatgccacttagctgcattttcagaagcttataggctataagaagcctttaatttcagtttgatttgctaggacaccaaaagacctagctgggctaactccatccaaatgattttacagttttattttttcatcttttgcaaaaagtatttgttacataatcaaaaggtgaaccagaggaaatattgaacaaggttttagatctgatgttattctggtgcccaattgagagaccttgagaaagaatgatgcagattacatgactctaatgtgggcagtctgaaaatatctttaactgtcttctttgatcatttgattttggatccaagatttatcatctctctattttgctatttgccaccatcctggtttttctgactgtacagaatcaatctgcatttatttattatcagaaatgaatgcatttttacaaaaacctttttgtagactgtagtttattgattagttcaagcccatcacgaactaattaaacatattgttggtaaaaacagagattgaaattactgtttttatcatatttaaatccctctggtgtatttattgaaatttaattaaaaatatttctacatattgatttgaagccaggatattgatggacctacagatttaacagctcttcaaagttttaactagcattataaaatggcctattcattatatgtatatttttttataaatatttatcaagaacatgaaaatattttattttgtatttgcagtgcagtttggcacctctcttccaggccttcctaagtttttgttagttgtgtcttcgaatttattgtagttcaccgctggtcagtaatctggttcataaggaagctggggcagtgaaaaacacttcgttgatccatccagccctaaagttcattgactaatagctcatgcatcttattaaatgggattttggacttctacaatagtgtggaaagccacatcacaaaaaaagccacatcacaaaaaaaagccagtgcctgttgagtatatcaaaccaaaaaagaaaaaaaaaagttttaagaggggtcaggctggcagataaaagtctcttatgaagcagatataaatccagtaagctatttttttctacctatgggcaagttcataagtcattgctggatctgaaccagtgttcttttgtgtaagtacaaccatatttcagtcagtccagaggcagttaaccctgtagcacttactagactggtgacaactggctggatgtggagaataagtgtcaaaacctggcttcaatgtctatttcttatgtctaatggagttcttatattacagtgtttgtttagctatggttactaatatgtttgttatatttattttcggggagaggatcttgttattgtttcctgagattatttaaaatgttttttctttgatgtatttctgtctttcttatatgtaaggagttataagggttattatattttcatttctggtttttactatacaatggttatctaacatactaagttggtaccctacctacccgtaaaatatgaaatactcagaagtaaagtgtgtgggcaatcagaagcatagccttgaaggacctgacagcagtatgaaaagataaataggccaggcatggtggctcacgcctgtgatcccagcactttggggggccgaggtgggtggaccacctgagatcaggtgttcaagaccagcctgaccaacatggtgaaaccctgtctctactaaaagtacaaaaattagccaggcgtggtggcgcatacctgtaattccagctactcaggaggctgaggcaggagaattgcttgaacccaggaggtggaggttgcaatgagccgaggttgagccatcgcactccagcggtgaaacaagagtgaaactccatctc

SEQ ID NO: 74 - DNA, 1238 bp, Homo sapiens
  misc_feature: Incyte ID No 8560269CB1
ccgcccccgcgtcgcgggctctttaaggccggcggttttcggcagccagcccggggcggggaaagcggagcgcgcgctccacgcgggaccgcctcccgggccgtctgagcagagggcggggtgcaggcggaatggccctcgtgccctatgaggagaccacggaatttgggttgcagaaattccacaagcctcttgcaactttttcctttgcaaaccacacgatccagatccggcaggactggagacacctgggagtcgcagcggtggtttgggatgcggccatcgttctttccacatacctggagatgggagctgtggagctcaggggccgctctgccgtggagctgggtgctggcacggggctggtgggcatagtggctgccctgctgggtgctcatgtgactatcacggatcgaaaagtagcattagaatttcttaaatcaaacgttcaagccaacttacctcctcatatccaaactaaaactgttgttaaggagctgacttggggacaaaatttggggagtttttctcctggagaatttgacctgatacttggtgctgatatcatatatttagaagaaacattcacagatcttcttcaaacactggaacatctctgtagcaatcactctgtgattcttttagcatgccgaattcgctatgaacgggataacaacttcttagcaatgctggagaggcaatttattgtgagaaaggttcactacgatcctgaaaaagatgtacatatttacgaagcacagaagagaaaccagaaggaggacttataattggctataatttataagaatgttgtcattgagtgtgtcacttaaggtcttagactgcaaatctaaccatatttaatgaaatgtcttactgtacaaaaagtctaagccaaaggttctcaggggagaaagcacatgtgcagttttaaaacaaagcagtgctttgtcccattgctgtgatttttagtcagactttactcagtctgaaatgcaattaacattaaaggattaagtgtgagatttcgatttatgctatttgtgtatcccatactcctcccttttaataaacagtttccactgatgatatgaagggccggtataaagaagtctttaaatgagtaagctttcttggtaagattaaatcttacaaattatttttaaaaccttgtgatatatacaatgttagctgagttttctaattttctggatgtaaaacaaaaggtttaacctata

SEQ ID NO: 75 - DNA, 1771 bp, Homo sapiens
  misc_feature: Incyte ID No 8710302CB1
gcgatacgccgagcgctcaacatccgaggactttggcccagagtaaccccgctctcgtgacctttcccctccattccgcacctccgagtgctggccgggcgagaggctggcggctgggctctcgcgcccctccctgcagggctcaggctctccccctcctgtcttctccgcgctgttcctcgtcatggcggccctcagcaagtccatccctcataactgctatgagatcggccacacttggcacccttcctgccgggtctccttcctgcagatcaccgggggcgccctggaggagtccctgaagatctatgctcctctgtacttgattgcagcaattctccggaaacggaaattagactattatttacacaaactactccctgagatcctacaatccgcttcatttctaactgctaatggggccttgtatatggctttcttttgcattttaaggagagggctgctcacaatttatatggccaacttggccacagaaacactattcagaatgggtgtagcaagaggaaccatcacaacattaagaaatggagaagtccttttgttttgcatcacagctgccatgtacatgttctttttcaggtgcaaggatggcttgaaaggatttacattttctgcacttaggttcattgtagggaaggaagaaattcccacacattctttttcaccagaggcagcatatgcaaaagtggaacaaaagagagagcaacatgaggaaaaacccagaagaatgaatatgattggtctagtcaggaaatttgtggattcaatatgcaaacatggaccaaggcatagatgttgcaaacattatgaagataattgcatctcttattgcattaaaggtttcatcagaatgtttagcgtggggtacttgatccagtgctgcctccgaatcccttctgcatttaggcatctgtttacacagccatctcggctactttctctcttctacaataaagaaaacttccagcttggagcttttcttggctcttttgttagtatatacaagggtactagttgcttcctgcgctggatcagaaacttagatgatgaactacatgctattatagctggatttttggcaggtatatcaatgatgttttataaaagcacaacaatttccatgtatttagcgtccaaattggtagagacaatgtatttcaaaggcattgaagcagggaaggttccctattttcctcatgcagatactatcatctattccatctctacagcaatttgcttccaggcagctgtcatggaagttcagactttgagaccatcttactggaagttccttttaagactcaccaagggcaaatttgctgtcatgaaccgaaaagtccttgatgtttttggtactggtgcatctaaacactttcaggatttcatccccaggttggatccaagatacacaactgtaacaccagagttgcccacagagttttcctgaagatgactgtaacttattaatgtgactaaatgtttcatcttgaagagttaattatgttgaacacaaaggagggggcccaagctcgaacttcagtgttatttcagttagagatactcttttcatttgttttgtttttcttatgaatcagaaattcagaagctttttaggaagatgttgcttaataattaagcttcctccatagccagaataagattctggatcactgtagtgactgacattatatattattgatcaaattatgtccacaagcaatattatataatctacgtagaagtgtaataa

SEQ ID NO: 76 - DNA, 2909 bp, Homo sapiens
  misc_feature: Incyte ID No 6778214CB1
tttccgtggcccttcctgccctttgtttctctcttgatgtcacccgcgggacaactctacgtgcaagtcgctgaaatcgattttctgcttcttatagtaagcggcgggctcgccagcctcgagcgaaagtgtgactgcgaacgggcaggcgcgcgcggggctcggcggaggcgcgcttgggctcccggcggcgacgactacgaccactaggagagcggacggaggcggcgcctgaagcggcggcggagcccatgccccgggacggcgggcggacccggagagacaaatccggggcccggggcatgtccccggggcccccgtgaggaggcggcggcggctatggagatcgcgccgcaggaggcgccgcccgtgccgggcgcggacggcgacattgaagaggccccagctgaggccgggtctcccagccccgcgtcgccccccgccgatgggcgcctcaaggctgcagccaagcgcgtcacattcccgtccgacgaggatatcgtgtctggagcagtggagcccaaagacccctggagacatgcccagaatgtgaccgtggacgaggtcatcggcgcctacaagcaggcctgccagaagctgaactgcaggcagatccccaagctcctcaggcagctgcaggaattcacagacctcgggcaccgcctcgactgtctggacctgaaaggtgagaagcttgactacaagacctgtgaggccctggaagaggtcttcaagaggctgcagttcaaggtcgtggacctggagcagacaaacctggatgaagatggtgcctcggccctcttcgacatgatcgagtactacgagtcggccacccacctcaacatctccttcaacaagcacatcggcacccggggctggcaggcggccgcccacatgatgcgcaagacgagctgcctgcagtatctggacgcccgcaacacgcccctgctggaccactcggcgcccttcgtggcccgtgccctgcgcatccgcagcagcctggcagtgctgcacttggagaacgccagcctgtcggggcggcccctcatgctgctcgccacggccctgaagatgaacatgaacctgcgggagctgtacctggcggacaacaagctcaacggcctgcaggactcggcccagctgggtaacctgctcaagttcaactgctccctgcagatcctggacctccggaacaaccacgtgctagactcgggtctggcctacatctgcgagggcctcaaggagcagaggaaggggctggtgaccctggtgctgtggaacaaccagctcacgcacacaggcatggccttcctgggcatgacactgccgcacactcagagcctggagacgctgaacctgggccacaaccccatcgggaacgagggtgtgcggcacctcaagaacgggctcatcagcaaccgcagcgtgctgcgcctcgggctggcctccaccaagctcacgtgcgagggcgcggtggcggtggcggagttcatcgctgagagcccccgcctcctgagactggaccttcgggagaacgagatcaagacaggcgggctcatggcactgtcgttggccctcaaggtgaaccactcactgctgcgcctggacctcgaccgtgaacccaagaaagaggcggtgaagagcttcatcgagacgcagaaggcgctgctggccgagatccagaacggctgcaagcgcaacttggtgctggcgcgggagagggaggagaaggagcagccgccacagctgtcggcctccatgcctgagaccaccgccaccgagccccagcccgacgacgagcccgccgctggggtgcagaacggggcccccagccccgcacccagcccggactcagactcagactcggactcggatggggaggaagaggaggaagaggaaggggagagggacgagaccccctccggggccattgacacccgggacacagggtcctctgagcctcagccaccaccggagccgcctcggtcagggccaccactgcccaacggcctgaagcccgagttcgccctggcactgccccctgagccgcccccggggcctgaggtcaaggggggcagctgcggcctggagcacgaactgagctgctccaagaacgagaaggagctcgaggagctgcttctggaagccagtcaggaatccgggcaggagacactgtgacactttagttccctttttccggtcggtctgcgatgagctgaggccagagccatgagaatctgctcaccttccccccagccttcctgaggcccaggatgccaggggtgggggccattctggggcccccctccccccacagcaacactacaaggggtgcaggagctacagggagtggccctccgcgcgtgactcaagcacttctatttatgagcccagcactggaagactctgggggtgaatgggaggagggggagcaggaggaggaggaggtctccaagtacatcaggcgcctgttctggaggggccaggcttgccctgcggagggcaggcgtcctgggtggtggtgggatggtcccctgtggccccgggcacagggccgggcaggcagcctggtgccggagaggcggtgcgtgctggtggtggttgagatgcgcagaacagccccagacagcgcaggccgggcagggtggggggatgggagcagaggatcagagctttctttttctcaagtgcaataaatctatcagggagctggggcgggagcagccggcattccgggaccctgttgtccaggccactggaggctgcgccctgagaggcactacagccctttgggggcgagtggcatgggtgtgggtgagggtgggcagagggctggggcgactcctgtcggtgcaactctgttcacaccttttagaataaactggcatggtcggtaaaagacaaatagc

SEQ ID NO: 77 - DNA, 2216 bp, Homo sapiens
  misc_feature: Incyte ID No 258383CB1
gcgatacgccgagcgctcaacatccgaggactttggcccagagtaaccccgctctcgtgacctttcccctccattccgcacctccgagtgctggccgggcgagaggctggcggctgggctctcccgcccctccctgcagggctcaggctctccccctcctgtcttctccgcgctgttcctcgtcatggcggccctcagcaagtccatccctcataactgctatgagatcggccacacttggcacccttcctgccgggtctccttcctgcagatcaccgggggcgccctggaggagtccctgaagatctatgctcctctgtacttgattgcagcaattctccggaaacggaaattagactattatttacacaaactactccctgagatcctacaatccgcttcatttctaactgctaatggggccttgtatatggctttcttttgcattttaaggaagatacttggaaaattctactcatggactcctggctttggtgccgctctgccagcatcttatgtggccattctcattgaaagaaaaagcaggagagggctgctcacaatttatatggccaacttggccacagaaacactattcagaatgggtgtagcaagaggaaccatcacaacattaagaaatggagaagtccttttgttttgcatcacagctgccatgtacatgttctttttcaggtgcaaggatggcttgaaaggatttacattttctgcacttaggttcattgtagggaaggaagaaattcccacacattctttttcaccagaggcagcatatgcaaaagtggaacaaaagagagagcaacatgaggaaaaacccagaagaatgaatatgattggtctagtcaggaaatttgtggattcaatatgcaaacatggaccaaggcatagatgttgcaaacattatgaagataattgcatctcttattgcattaaaggtttcatcagaatgtttagcgtggggtacttgatccagtgctgcctccgaatcccttctgcatttaggcatctgtttacacagccatctcggctactttctctcttctacaataaagaaaacttccagcttggagcttttcttggctcttttgttagtatatacaagggtactagttgcttcctgcgctggatcagaaacttagatgatgaactacatgctattatagctggatttttggcaggtatatcaatgatgttttataaaagcacaacaatttccatgtatttagcgtccaaattggtagagacaatgtatttcaaaggcattgaagcagggaaggttccctattttcctcatgcagatactatcatctattccatctctacagcaatttgcttccaggcagctgtcatggaagttcagactttgagaccatcttactggaagttccttttaagactcaccaagggcaaatttgctgtcatgaaccgaaaagtccttgatgtttttggtactggtgcatctaaacactttcaggatttcatccccaggttggatccaagatacacaactgtaacaccagagttgcccacagagttttcctgaagatgactgtaacttattaatgtgactaaatgtttcatcttgaagagttaattatgttgaacacaaaggagggggcccaagctcgaacttcagtgttatttcagttagagatactcttttcatttgttttgtttttcttatgaatcagaaattcagaagctttttaggaagatgttgcttaataattaagcttcctccatagccagaataagattctggatcactgtagtgactgacattatatattattgatcaaattatgtccacaagcaatattatataatctacgtagaagtgtaataacaaacaagagtacacttaaaattactttaaaagatgtctttagttcattccaatataattcttgattaaaattaggattatttctacattttaggatttacaaaggatcacgggtacatggatttggtctatatatttttttaaagttttgaattggtatctgtagtagtggaatgttatagatttgaagtaactctccacggacagtgctgctttcgtgtagagcaatttaattggagaagtggccattcttacttcagggatgcaaagatgggtctcataccatttggataaatgtcgtggtatccatgctttttttcaactaataacatcatctctcttcatgaccagt

SEQ ID NO: 78 - DNA, 5320 bp, Homo sapiens
  misc_feature: Incyte ID No 2804937CB1
tcgccgtgaacgcagcacatttaggcgccaggcagtacggcgccggcacaatgcagggagtaaccctacccctcctacattgctcatcggatcacccctaagccttcaagatggtcagcaaggccagcagtccacagcccaggtcaaagtccagtcccgccccccttcccaggctgcagtgctcagtgctagtgcctccttgctggtgagaaatgggagtgtccacttagaagcatcacatgacaatgcatctgctgtaggcggtagcagtttgcacgatgaacttggtaagttctcttctacgctgtatgagactggtggctgtgatatgtcacttgtgaattttgaaccagcagcaagaagagcatccaatatctgtgacacagattctcatgtatccagttctacctcagttcgattttatccacatgatgtgctctctctcccacagattcgattgaatagactattgaccattgatacagatttgttggagcaacaggacattgatctaagccctgacttggcagctacttacggcccaacagaagaagctgcccaaaaggttaaacactattatcgcttttggatcctaccccagctgtggattggcattaactttgacagactcacacttttggccctgtttgatagaaatcgtgagatcctggaaaatgtgttagctgtcatcctggctattctcgtggcctttttgggatctattcttctcatacaaggattcttcagagatatctgggtcttccagttctgcctcgtcatagccagctgtcaatactcactgcttaagagtgttcaaccagattcttcttctcccagacatggtcataatcgtatcattgcctacagtagaccagtttatttctgcatatgttgcggtcttatttggctcttggattatggtagcagaaacctgactgcaaccaagttcaaattatatggaataactttcaccaatccactggtgtttatatcagccagggatttagttatagtgtttacactctgtttcccaatagtgtttttcattggtctcctgcctcaggtgaatacatttgtaatgtacctttgtgaacaattggatattcatatttttggtggtaatgccactacaagcctgcttgcagcactttacagttttatctgtagcattgttgcagtagccttattgtatggattatgttatggggctttaaaggattcttgggatggccagcatattccagtacttttctccattttttgtggtttattagtggcagtgtcttaccatctcagccgacaaagcagtgatccatctgtacttttctctttagtgcaatccaagatttttccaaaaacggaagagaaaaatccagaagaccctctatctgaagtaaaagatccactgcctgaaaaacttagaaattctgttagtgagcgattacagtctgacctggtagtatgcattgtaattggtgtgctgtattttgctattcatgtaagcacagtcttcacagtattgcagcctgccctcaagtatgtgttgtatacattggttggctttgtgggttttgtaacccattatgtgctgcctcaagttagaaaacagctaccatggcactgtttctctcatcctctgctaaagacactagagtataatcagtatgaagttcgaaatgcagccactatgatgtggtttgagaaacttcatgtgtggcttctttttgtggagaagaatataatctatccattgattgttctcaatgaactgagcagcagtgcagagacaattgctagtccaaagaaactgaatacagaattaggtgctttaatgatcactgttgctggtttgaagttgctacgatcctcttttagcagccctacatatcagtatgttacagtcatctttactgtgctgtttttcaaatttgactatgaagctttttcagagaccatgctgttggatctcttctttatgtccatactcttcaacaagctttgggaactactttataaattgcagtttgtgtatacctatattgccccatggcagatcacatggggttctgctttccatgcttttgctcagccttttgcagtgcctcattcagccatgctgtttattcaggctgctgtctcggccttcttctctactccactgaacccctttctgggaagtgcaatattcatcacttcatatgtccgacctgtgaaattctgggagagagactataacacaaaacgagtggatcattcaaataccagattggcttcccagcttgatagaaatccaggatcagatgacaacaatctgaattccatcttttatgagcatttaactagatccctacagcacagcctctgtggtgatttgctactaggacggtggggaaactacagtacaggggactgtttcatccttgcctctgactatctcaatgcattagtacaccttatagagataggcaatggtctggtcacttttcagctgcggggacttgaattcagaggtacctactgtcaacaacgggaagtggaggccattactgaaggtgtagaggaagatgaaggattttgctgttgtgaacctggccatattcctcacatgctttcatttaatgctgcatttagccagcgatggctagcttgggaagtgatagtcacaaagtacattctggagggttatagcatcactgataacagtgctgcttctatgcttcaagtctttgatcttcggaaagtactcaccacttactatgtcaagggtatcatttattatgttacgacctcgtctaagctagaggagtggctagctaatgagacaatgcaggaaggacttcgtctgtgtgctgatcgcaattatgtcgatgtggacccgacctttaatccaaacattgatgaagactatgaccaccgactggcaggcatatctagggagagtttctgtgtgatttacctcaactggatagagtactgctcttcccgaagagcaaagcctgtggatgtggacaaagattcatccctagtgactctctgttatggactctgtgttctgggacggagagctttggggactgcatcccatcatatgtccagtaatttagagtcattcctctatggattgcatgccctatttaaaggagatttccgtatttcttcaattcgagatgaatggatctttgctgacatggaattgctaagaaaagtagtagtccctgggatccgtatgtccattaaacttcatcaggatcattttacttctccagatgaatatgatgaccctactgtgctctatgaagccatagtatctcatgagaagaacctcgtaatagcccatgaaggggaccctgcatggcggagtgcagtacttgccaactctccctccttgcttgctctgcggcatgtcatggatgatggcaccaatgaatataaaatcatcatgctcaacagacgctacctgagcttcagggtcattaaagtgaataaggaatgtgtccgaggtctttgggcagggcaacagcaggagcttgtttttctacgtaaccgtaacccagagagaggtagcatccaaaatgcaaagcaagccctgagaaacatgataaactcatcttgtgatcaacctattggctacccaatctttgtctcacccctgacaacttcttactctgacagccacgaacagcttaaagacattcttgggggtcctatcagcttgggaaatatcaggaacttcatagtgtcaacctggcacaggcttaggaaaggttgcggagctggatgtaacagtggtggcaatattgaagattctgatactggaggtgggacttcctgcactggtaacaatgcaacaactgccaacaatccccacagcaacgtgacccagggaagcattggaaatcctgggcagggatcaggaactggactccacccacctgtcacatcttatcctccaacactaggcactagccacagctctcactctgtgcagtcgggcctggtcagacagtctcctgcccgggcctcagtagccagccagtcttcctactgctatagcagccggcattcatccctccggatgtccaccactgggtttgtgccttgtcggcgctcttctactagtcagatatcgcttcgaaacttgccatcatccatccaatcccgactgtcgatggtgaaccaaatggaaccctcaggtcagagcggcctggcctgtgtgcagcacggcctgccttcctccagcagctccagccaaagcatcccagcctgcaaacatcacactctcgtgggctttcttgcgacagagggaggtcagagcagtgccactgatgcacagccaggcaacaccttaagtcctgccaacaattcacactccagaaaggcagaagtgatttacagagtccaaattgtggatcccagtcaaattctggaagggatcaacctgtctaaaaggaaagagctacagtggcctgatgaaggaatccggttaaaagctgggagaaatagctggaaagactggagtccgcaggagggcatggaaggccatgtgattcaccgatgggtgccttgcagcagagatccaggtaccagatcccacatcgacaaggcagtgcttctggtccagattgatgataaatatgtgactgtaattgaaactggggtactagaacttggggctgaagtgtgagccagtgtttattataaagacatttctttttccctctcaattccaaggcattggaaaaagagaggaacaagcagaagatgcctgcaggtatcactttgatcctatgtgggagcgactgaaaatagaatgagcttggttaagcacctctcctttgcccttcaccctgactcctgtcactgtctccatccccaaataaagctgaaatatttttttaagttagctgccgagaaaacattttgcatgaaggataaagttctgttaaaatacatccttaaaaaaagtttttcctatgcattgcctatgctttaaatcaacaaactcttcatttctaaactatgatctcatatttttctaatttctttgccaaaaataattgccatgttttgtcatagaacatgaaattcatttaccttgatttttaagaacagaccagtgtagaaatgttccttttgacttatagcagtataaaagtttaatgtacagtgaaagagactatgaacagacatagatttatcttattccttgagcgctaaaaactttaataaaaaaaactgcactaattttttacagcaatgcactaaagtctgagatttctcagaacatttatttatatataaaaataaaataccattattt

SEQ ID NO: 79 - DNA, 653 bp, Homo sapiens
  misc_feature: Incyte ID No 7494915CB1
tgcctttcatgtgaagaaccaggggctactccaggctagatttccccatgatgccaggagagaaaaaacagtcaggctcccagcaacagaacaatgggcaggcaagcaaaaaccgcacacagaaagaaatagtaacccagaaaagaccaataacaagcaatgagattgaattagtagttaaaaaaaaattgccaagagaaaaaggtccaggaccagatggattcatagctgaattcttccggacagtcaaagaagaattggaaccaaccctactgaaactattccaaaagattgagagagaaagaatcctccctaacacattttatggagtcagtatcacccttatgccaaagccagaaaaggacacaacagcaacaacaacaacaacaacaacaaactacagaccaacatccctgatgaatgtagattcaaaaatcctcaacaaaatactagctaaccaaatccaacctcacatcaaaaagataatacaccataatcaaaagctctttagtttaattagatcccatttgtcaattttggcttttgttgccattgcttttggtgttttagacgtgaagcctttgcccatacctatgcactgaatggtaatgcctaggttttcttctagggtttttatggttttaggtcttaca

SEQ ID NO: 80 - DNA, 1794 bp, Homo sapiens
  misc_feature: Incyte ID No 2073751CB1
aattcggctcgagaggagtggtccggggcaccggtttcaaagtcgcagggcgggccgagtggacttccgctgctggcctggggcttcccagccgtcttggcgttgtcctctccaacccccgccgctccgcgtagaacgccgctctcaggctgccgtcaagctcccgcggcactctcctaggtggcccgacgagacccagagtgacccgcgggacgcctgtatcgaccgcgtcctcttcccaccagcgtgggattcggttgaacgtggagtccccagcaatcttcagtctctcaccagggccagggactcgtctggggcgcgggggaaagaagcgtggcggggctgtagatgccgcgtgagtaggatgcagattgcaccgctggagcgcttgacaaccaaccgagcgttggcttagttttgttttcccgcacagcaagctctctgtctttcagaggaaggtaaaggtggtgaaagctctaaactaaaattgtatcgaaatggctgcagaaatcgactttctgagagagcaaaatcgaagactaaatgaagattttaggcggtatcaaatggaaagtttttccaaatattcatctgtacagaaagctgtctgccaaggagaaggagacgacacatttgaaaacctagtatttgaccaaagctttttagctcctcttgttactgagtatgataaacacctaggagaactaaatgggcagctgaaatattaccagaaacaggtgggtgagatgaaattacaatttgaaaatgtcatcaaggaaaatgaaaggttgcacagtgaattaaaagatgctgttgaaaaaaaattggaggcctttcccctgggcacagaggtaggaactgacatatatgcagatgatgaaacagtcagaaaccttcaagaacaattgcagctagccaatcaagaaaaaactcaggctgtggaactctggcagactgtttctcaggagttggacagactacacaagctttaccaggaacatatgactgaggcccagattcatgtatttgaaagtcaaaaacaaaaggatcagctatttgattttcaacaactgaccaaacaacttcatgttactaatgagaacatggaagtgactaaccaacagtttctgaaaacagtaactgaacaaagtgtgataatcgaacaactccgaaaaaaacttaggcaagccaaattagagctgagagttgctgtagcaaaagtggaagagttaactaatgtgactgaagatctgcagggacagatgaaaaagaaggagaaggatgtggtgtctgcccatggaagagaggaagcatcagataggcgtttacagcagttacagtctagtataaaacaattagaaataagattatgtgtgacaatccaagaagccaaccaattaagaaccgaaaatacacatctggaaaaacagaccagagagctacaagcaaagtgcaatgaattagaaaatgagagatatgaggctattgtaagagccagaaatagcatgcaactcttagaagaagctaaccttcaaaaaagtcaggctctacttgaggagaagcaaaaagaagaagacatagagaaaatgaaagagacagtttctcggtttgtacaagatgctaccataagaaccaagaaagaagttgcaaacaccaaaaaacaatgtaatatacaaatttctcgattaacagaagaactttcagcccttcaaatggagtgagctgagtcgacagaaacttcatacccaagagctgctttctcagctggaaatggcaaatgaaaaggtagctgag

SEQ ID NO: 81 - DNA, 2691 bp, Homo sapiens
  misc_feature: Incyte ID No 3178841CB1
gcgaccagcggaaggagggagggggccgcgctcggcgccccggccgggccactgggccacaggccacgcggccacgcagtccgagcgggagccgagccgggcggggcgagggcagctccgggaaggaacgtcccagggatggaagtgcttggatgcggtgctgctggctgcggatgtgcgcaaggagatgggatggagagcctgagttggcattcgtataaatgacctgcctggctcccaccatgagtgctgagcttaacgtgcctatcgacccctctgctcctgcctgccctgagcccggccataagggcatggattaccgggactgggtccgccgcagctacctggaactggtcacctctaaccaccactcggtacaggccctgtcgtggcggaagctctacctgagcagggccaagctgaaggcctccagcaggacctccgccctcctctccggctttgccatggtggccatggtggaggtgcagctggagacgcagtaccagtacccgcggccgctgctgattgccttcagcgcctgcaccacggtgctggtggccgtgcacctgttcgccctcctcatcagcacctgcatcctgcccaatgtggaggccgtgagcaacatccacaacctgaactccatcagcgagtccccgcatgagcgcatgcacccctacatcgagctggcctggggcttctccaccgtgcttggcatcctactcttcctggccgaggtggtgctgctctgctggatcaagttcctccccgtggatgcccggcgccagcctggccccccacctggccctgggagtcacacgggctggcaggccgccctggtgtccaccatcatcatggtgcccgtgggcctcatcttcgtggtcttcaccatccacttctaccgctccctggtgcgccacaaaacggagcgccacaaccgcgagatcgaggagctccacaagctcaaggtccagctggacgggcatgagcgcagcctgcaggtcttgtgaggggccgagggccggggctgggagcggccctgtgcccgggagtccgcagaggcggggatttgtcagatgcagacattttgcaaggctgccgggtagttcaagaccaaagttttcctcttgtcttaataccataaggactggatgacttctcctgagatagaaccgtttggttcaatgagggactgtgttgctaagagcgttgggggcaaagccaggctggttccttggcctcggggtttcctgggtcggggacacggtgaagaggctccagcgggacctgcccatcagtcctgggccaggaggggctccaagcagcacccagcggtccgggggagtctcagacccggcatgcgtggctggcagacctgggagagccagggcagggttttgcgttcagagaaggattgccccagagacccgtggtggacttcatgggtgctgagtggcccgtgtgacagtgatgacacgaaggcttcggcgtttgagtgggtgcaggtgcacgccagggcttggtgcttccctgcctggccctggagggagctgggtggcctggcttcaggggaagacaggagccaggacacacgtcagcccagcaggtgtggggggtgctgcagccctcggcagtggggtcaggccctgggggatgtttccaatggtgggcagcctggccaggccggagaagacatgttcacgggcatctatcagatgcccccttgaggaggctgagttatttgagggctgctgcaaagtacgctaggctcaaattctcttttcccagccagagccctggccacacggactcagaggggccaccggggtggggaaaggacccctccccgaccccccgcagccactggcctccagctctcggccacagaatggcctctaaggctgactcagccgctcccttgggctgtggcagcaggaggcgggggctctggctcaggccccggagcctgtgcagcttgcccatggccctaggcagcgaggggacagcctgggggacttcctgcctaggcaaggtcattggccgggcctggcctgtggatagtggggccaggggccggcccaggccaaatgagtgccctccttgttatgacaccaagtgactacaagggaggcaagacccctccaggcctctcagccgacactgggtcccaccacacacagtgactgtgccgtgcagtgcaggttctggccttttccttgaaggcatctggtagacccgaagccacgctctcgggccgcacatgcacgccgcagcaccagctgccctgagctgcttgtacaaccaaacacctttcccctcttctccagctgtaacctggagagtcagccatgccttgtcttttgttctcataaatagtcactggggccgggcgcagtgactcacgcctgtaatcccagcactttgggaggcctaggtgggcggatcacttgaggtcaggagttcgagaccagcctggccaacatggtgaaaccctgtctctactaaaaaaatacagaaaattagctgggcgtggtggcgggcgcctgtagccccagctacttgggaggctgaggcgggagaatggcaatggcgtgaacccgggaggcagagcttgcagtgagctgagatggcgccactgcactccagcctgggcgacagagccagactcaatctcaaaaaaaaaaaaaaaaaaaa

SEQ ID NO: 82 - DNA, 2056 bp, Homo sapiens
  misc_feature: Incyte ID No 3674807CB1
gcagctgatagaaccatggcgaccattgctgctgctgcgtttgaggccctcatggatggagtgacatgctgggatgtccccagaggccccatccccagtgaactccttcttattggagaagccgccttccccgtgatggtgaatgacaagggccaggtgctcattgctgcctcctcctacggccgaggccgcctcgtggttgtgtcccatgagggctacctgtcgcatgctggcttggctccatttctcctcaatgcagtgagctggctctgtccctgtcctggggctcccgtgggagtgcatccatccctggcacctctagtaaacatcctacaggatgctgggcttgaggcacaggtcaagccagaaccaggagagcccctaggggtttactgtatcaatgcctacaatgacaccttgactgcaacgctgatccagtttgtgaaacatggagggggcttgttaatcgggggccaggcctggtactgggccagccagcacggccctgacaaggtgctctccaggttccctgggaacaaggtgacaagtgtagccggagtgtacttcactgacacctatggggacagagaccggttcaaggtctctaagaaggtgcccaagatcccactccatgtcaggaggtagagcccggtactcacccagcaatcattgttgcctgacaaaagtcacagagaaggctcagttcttccttcctatgtgagtatatctccacttggggcctcccagcccctcccagtggtacagcttctcccctgtgtatctcaagtcatccatacttcttagactcatcccctctaaaccttaagcagtgtcccaatccccatcttcttgaaagtacctgatttttgtatttttagtagagacggggtttcgccgtgctggccgggatggtctggatctcttgaccttgtgatccgccagcctcagcctcccaaagtgctgggactacaggtgtaagccaccatgcccggcctggtctttcttagtgatacataaaataatggcatttctcacaatcaatttgtcatagatttgaggaaacgctatagaggtagtgcccttacttgcatatcttaaaatccacattttctaaatgaacacctccaggaattcctcccatttagtcaactatgtgttgaatattttggcttcagtttcttgacttttaaaataattaaacatataaacaagttagaaaaactagtttattatgcccagagaaagtagtactgcttatgaatttctcgatctaactttctgcaaaggaaggctgtaaaaacaccaaatctggactctccgtagggtagttctgacctaggaacacatcacaattaaattgtttaacactacgcttgaagatttatacaaacacacactgtttttaaaacttcacttagatctggttagagtattcacttcttcttaaggaatggtcaaaatgtaatgaatcaagaaaaggctttgtatttcaagaatttttttaaaacaatttgtatatactgtaaatgtcaagcaataggaggttggttaaataaattatggttaacaatagattgcaaaccatatagtgattaaaataattattaggtaacttgaaaaatgcttaatggaaaaagttttaaaattagattccaggataccatgctctgaataagccaaattttggggtaaaaaaaataaaaaaccatgaaaccagacatagaagcagaacctaaagcatatttatcaaaatatctttgtttaaaaaaaatgtcttcagtggtcgtcttggctggtgggattttaggtcattttaattatctttataaaactttattctctcgaattttaaaattaacatctatttctcttataattaaaaattaaatgttatattttttaaaaagtatagtttatttattccagtagtaaaacattgttgaaaaatgtaactcttgtgcagattctctttaatttcttttcagcatgaaaatgaggagatggcatgtcttagttttgtcatcccattttcagactcacatggcaaatgtcatag

SEQ ID NO: 83 - DNA, 2275 bp, Homo sapiens
  misc_feature: Incyte ID No 1794922CB1
ctttcatatccttagactgtatctgaaattttcatagctttgcccaccttttagggttcagagttactacccattcagtgtcggtggcctttagctgttggcaagcttccaagtaaatagctcttttgcagagatctctacccattctattcttccactcctgggtagtggagaaccaaagttcacctgagtaatgcccgtgcccagcacccagtttcctgcaggggttagcacggatattggaggctcccccgatcccacagatactcaggatgggtattggagggggcttggtttttaagaggacatgtgaaagtaattaacccgatttacatttgtataatgagtccaaacaccaccttgcaaaagaactgagagtttagcctgcttaatggagtttctgcttttcacggtccttggccttaccgacggtttacagtccagccagatttagttcacaaaaaaccaaaaccaggtcgacctcactgcagtatatgatggcagcgagcgttttaaagcgcaggacctttagtctcctcaatagttttgtgtaaagttgtccaaaagtgtttttttttaaagtgagatgatccagtcggtaaacgctttgaaaaagaatagctgtcccctgagcgggcattagtcgcgtgtgaggtcagggcccatcattgcgcgtgttgggagcgccgcgctggtctatgagcgagcgcctcggcttgtggctgggccgggccggggcggggctgtcttcccgcggagcgtgctgggggcgggtcgcgccgggccgggccgctagtgcgcatgggcgggcgtcctcggctctaactgccgccactttccacacgctgggagggccgttacctcagagatacccgtggccggcatgttggttgaaaaagcttcccggaagggagacgaagagaaaggagaggagcagctcgtgatcatccccggtagcgagtacgcggcgaagtaggcggcggcggagggagcgctgatgaagatggatgtgtcagtgagggccgcgggctgctccgacgacctcagctctggggaggccgacgtagacccaaagctcctggagctcaccgctgacgaggagaagtgccgcagcatccgcaggcagtaccggcagctcatgtactgcgtgcggcagaaccgggaggacatcgtgagctcggcgaacaactccttaaccgaggctctggaggaagccaacgtcctctttgatggcgtgagccgaaccagagaagcagccctcgacgcccggtttcttgttatggcttctgatttgggtaaagaaaaggcaaagcagttaaactcagatatgaacttctttaatcagttagcattttgtgactttctgtttctgttcgtgggtctgaattggatggaaggcgatcctgacaagttgagtgattgtgatgatagcatagctctttccttctggaaggcaatagaaaaggaagcaacatcctggatggtaaaagctgagacattccattttgtttttggttcattcaagctagaacgttctgcaccaaagccccgacttgaacaccagaaaaaagttcgcaagatggaagaaaatggcaacatgcctacaaagttgcagaagttggacctgagtagttatccagaagcgacagaaaaaaacgtagaaaggattttgggattgttgcaaacctactttcgaaagtatcctgatactcctgtgtcctattttgagtttgtgattgatccaaactctttttctcgtactgtggagaatatattttatgtttcttttattgtaagagatggttttgcaagaataaggcttgatgaagacaggctgccaatattagagccgatgaatgttaaccaaatgggtgagggaaatgattccagttgccatggcaggaaacagggagttatatctttgactttacaggagtggaaaaacattgtggcagcttttgaaatttctgaggctatgattacatactcctcatactaaagatttcttagtatagcatcctttttgtgttttttttctgaagttagatggagagtaaaatgtaaactgaagcacatattgtatctcttgtaaagtgaaaaagtattttcaagaacatcagacattgttttactgtgcagcatatttttcttagtaatttataaggtcatgatcttctgttattaaaacaaattcactggcatatttatgggaacgttttattgaatgccctttaagactattaataaaaacaagttttggataccaaaaaaaaaa

SEQ ID NO: 84 - DNA, 1219 bp, Homo sapiens
  misc_feature: Incyte ID No 1795509CB1
gcttgttgcagctctagcccaggtcctgccctcttcccgccccgcccctagggtccagctcccttcacctaggagctgccaaacatctggatcaacctgggcactacgaggggttgaatttctaccattatcgcgccttttgatatttttttccagacctcctgctcacatccgtaaagcccactgattcttttactacactttttatgagaacaagacattttctaggaagatggtggcagaaaaagagaccctgagcttaaacaaatgcccagacaagatgccgaagaggaccaagctgctggcacaacagccgctcccggtgcaccagcctcactctctggtttctgagggtttcacagtcaaagccatgatgaaaaactcagtcgtgagaggccctccagctgcaggggcatttaaagaaagaccaaccaagcccacagcatttcgaaaattctatgagcgaggtgacttcccaattgcccttgagcatgattcgaaaggaaacaaaatcgcctggaaggtagaaattgagaagctggattaccatcattatctgcctctgttttttgatgggctttgtgaaatgacatttccctatgagttttttgctcggcaaggaatccacgacatgctggaacacggtgggaacaagatcctacctgtccttccacagctcattatcccgataaaaaatgccttgaacctccgaaaccgacaggtcatctgtgtcactctcaaggtcctccagcatctggttgtgtcagctgagatggtgggcaaggccttggtgccttattaccgtcaaatcctccctgtcctgaacatctttaagaatatgaatgtgaactccggagacggcattgactacagccagcagaagagggagaacattggggacttgatccaggagacactggaggccttcgagcgctacggaggagaaaatgcctttatcaacattaagtacgtggtcccaacctacgagtcttgcttgctaaactaacagtggcagcagctgggacttgaaacctcccgttggtgttgggatcatctgtctctgttgcttttagcatctcattccttgtgacttccacagctttcttttctacagctgctaaaatagtggcttatgggccattggactgttagccctattgagagcaaggctttccaatacataaatagtgcctgtttcttagattaaaaaaaaaaaaaa

SEQ ID NO: 85 - DNA, 1015 bp, Homo sapiens
  misc_feature: Incyte ID No 2017180CB1
ggaagcgggggtgcagcgcggcagaatgagggttgattcctcggctgaccccacaatgtcgcaggagcaagggccggggtcctccacgcctcccagttctccgacacttcttgacgctctgctccagaacctttacgactttggaggtacagaaggtgaaacagaacagaagaagatcataaagaaaagggaaaacaagaagagagatgtgatggcttcagcggccttggcagcagagccatctcccctacctggttctctcataagaggccagaggaagagcgcttcgagcttcttcaaggaacttagagaagagcggcattgtgctccttctgggacccccacaggaccagagatccttgctgctgcagttcctccctcttccctaaagaacaatagggaacaagtagaagtggtagaatttcacagcaataaaaaaagaaaattgacgccagatcataacaagaacacaaagcaggctaatcctagtgttttggagagagatgtggatacacaagaatttaacctagaaaaagctcgtttagaagtgcaccggtttggtatcacgggttatggaaaaggaaaggagagaatcctggaacaggaacgtgccattatgctgggcgctaagcctcctaaaaagagttatgtgaattacaaggttttacaggagcaaattaaagaaaaaaaggcagcaaaggaagaagaaaagagactggcccaagaaacagatattttcaagaaaaagaagaggaaaggacaggaggacaggaaatccaaaaagaagtccgctcccagtattttgtcaaatggacggattggacaggttggaaaattcaaaaatggaacactgattctgagcccagttgatatcaagaaaataaattcttccagagtggccaaatgaagtactttgtcaaataaaacgaggatgggaacaactgagtcaactgcagcggccctggaccctgtgcgactctagatagatcgttcttaacacgcatttcacagactg

SEQ ID NO: 86 - DNA, 2392 bp, Homo sapiens
  misc_feature: Incyte ID No 219442CB1
ggcggagtgaccacatagagtaaaaagaatgtaaccctctttggttttatgtgttttggctcaggttggacatacttatataaatgtgattgacattgaagctaatgatcttctacaggaattacctgtgagagaagagccttcaaatgataatgttatcaaacagcaaagcgatcatctagcagttccatcgtctgcagagttacattatatggcagcttcagttactaatgctgttcccccacataattttaagagtcaagaagtaactccagcttgtctggatggaaaaagcttgagagcaggcattacagaagtgaaggagcctagtgtcacctcacctacaccatcagacatacagcagaacaaaggtctgccaaaaccagagttccgattcaaaggacagagcacaaagtcagactctgcagaagattatctattgtggaaacggctgcaaggtgtctctgcagcttgccctgcaccaagctctgcagctcaccaactagagcatctcagtgctaagcttcagaaaattgacgagcagttgctagcaatacagaacattgctgaaaacatagaacaggatttccccaagcctgaaatgctagatctacattgtgataagattggaccagtggatcacattgaattctcttctggccctgaattcaaaaaaacattagcttcaaaaaccattagcatttctgaagaagtgcgttttttgacccatatggatgaagaagatcaaagtgacaaaaaggagacttcagaacctgaattttcaataacagaaaattattctggtcagaaaacctgtgtgtttcctactgccgattcagctgtcagcctttccagttccagtgatcagaatactacttctcctggtatgaatagcagtgatgaattgtgtgagagtgtttcagtacatccgctccagatgactggattgactgatattgcagacattattgatgaccttataattaaagacggagtttccagtgaagaacttggcttaacagaacaagctatgggcacctccagaattcagcattattctggcagacattcacaaagaactgacaaggaaagaagagagattcaagcctggatgaaaagaaaacgaaaagaaagaatggcaaagtacttaaatgagctggcagaaaagagagggcaagaacatgatcctttctgtcccagaagcaatccactttacatgacttcaagggaaataaggctgagacaaaagatgaagcatgaaaaagacagattgctgctctctgaacactatagtcgtcgaatctcacaagcgtacggtctgatgaatgaactgttatctgagtcagtacagctaccaactctaccacagaaaccattgcctaacaaacccagccctactcagtcttccagttgtcaacactgcccttctccaagaggagagaatcaacatggtcacagttttctaataaatcgacctggaaaagtcaaatatatgtccaaaccgagttatatccataagaggaagtcttttgggcaacctcaaggctcaccttggccacatggaactgccactttcaccatacagaaaaaagctggtggagccaaagcagcagtaagaaaggctacgcagtctccagttaccttccaaaaaggctctaatgctccgtgtcatagtctgcagcatacaaaaaaacatggaagtgctgggcttgcacctcaaaccaagcaggtgtgtgtagagtatgaaagagaggagactgtggtgagtccctggacgataccttcagaaatccataagattcttcatgagagtcacaattcccttctacaagacttgtctccaactgaagaggaagagccagagcatccttttggggtgggcggtgtggacagcgtgtctgagagcactggcagcatcctcagcaagctggactggaatgccatcgaagacatggtggccagcgtggaggaccagggcctgtctgtccactgggccctggacctgtaagacctggatatcattgggtttccatgcacaggccagcacctcagtaatgtggttctgaaagattaacaggtttaagggacagaagcaatgaaagaagcaatgtgaattttccatttgctttcatattattacctggattagccattaccagaggaaaaataaacatttctcagtaactttgcctttatggggaaagggttgactattgatgtattatatgtttttgtatttgatgcatcattaggcataatttttaaaatgataagtacctttcaagccaagtttgcataacctactttcaataaaaaccctctatcttgcctcctcctttattaccctctgagttttgagaaacaaccatatacagatgaatctaataggaaa

SEQ ID NO: 87 - DNA, 1799 bp, Homo sapiens
  misc_feature: Incyte ID No 2597459CB1
gcagagcgaagctactgcgggttctgttaacctcagcatcgtggggcgaagcagagccattgtgcatcaaggagaggccggtgcctgcgctgccgtctctggcacctaacccagcagaccgctcaccccatcggctggaatgcttgctgattcctctgcacacttctaaggctgagaacctgaggaacccagctggaaaatgccgtctgaacgctgcctcagtattcaagaaatgctgacaggccagaggctctgccactccgaatctcacaatgacagtgtcctggcagcgctgaatcagcagaggagtgatggcatcctctgcgacatcaccctgattgctgaggaacagaaattccatgctcacaaggcagtcctagcagcatgcagtgactatttccgggcaatgttcagtctttgtatggtggaaagtggagctgatgaggttaatttgcacggtgtgaccagccttggcttaaagcaggctctggagtttgcatacacaggacagattttgctggagccaggtgtgatccaggatgtgctagcagcgggcagtcacctacagctgttggagcttctcaatttatgctcccactatctcatccaggaattaaatagctttaattacttggatctgtacagacttgctgacctctttaacctcactttgttggagaaggcagtgatcgatttcttagtgaaacatctctctgaactcctgaagagccgcccagaagaagttctaacgcttccctattgcctgcttcaggaggtgctgaagagcgaccgcctgacctccctgagtgaagagcagatctggcagctagctgtgaggtggttggaacacaactgccactaccagtacatggacgagctcctgcaatacatccgctttggcctaatggatgtggatactctccatacagttgccctgtcccacccccttgtccaagcaagtgagactgcaacagcccttgtcaacgaggccctggaataccaccagagcatctatgcacagcctgtctggcagactcgcaggaccaaaccacgattccagtcagacactctgtatatcattggtgggaaaaagcgcgaggtctgcaaggtcaaggaacttcggtacttcaatcctgttgatcaggagaatgctctcatagctgccattgccaactggagtgagctggctcccatgcctgtgggaaggagccaccattgtgtggcagtcatgggggacttcctgtttgtggcaggaggggaagttgagcatgccagtggccggacgtgtgctgtgaggactgcctgtcgctatgacccccgcagtaattcctgggcagagatagcacccatgaaaaactgccgggagcattttgtgctgggtgccatggaggaatacctctatgcagttgggggcagaaatgaactgcgccaggttctgcctacagttgagcgatattgccccaagaagaacaaatggacttttgttcagtcctttgacagatccctttcatgccatgctggatatgtggctgatggtcttctttggatatcaggtagaacatacctcatgttggatttatcaaaacacactttcattgtggtatatatttaaaagtcaagctttaatactgtggccatgtgtaattgaaagattgaaccaagcatgccattttagctaattaacattcatttattgagcacctactctagagagcaatgtaattggaaataatatgcctttctgtgactttctgagtgactaatggaataaaaattcaaggtggaaaaaaaaaaaaaaa

SEQ ID NO: 88 - DNA, 3718 bp, Homo sapiens
  misc_feature: Incyte ID No 2783863CB1
ggcgctggcggcagtggccggtgtgagcgcagagccgggccctgggcagcagcggcagcgcggtaggacctcgcgcagcgtccgcgggctccggggcgggggcgccagcggcgaagccccctccccggggaggcgggacctgggggagctcccgagccggggagcggcggcggccgggaacgatgcatcagaagctgctgaagagcgcgcattacatcgagctgggcagctaccagtactggccggtcctggtgccccgtggcatccgcctgtacacctacgagcagatccccgggtccctcaaggacaacccgtacatcaccgacggctaccgggcctacctgccgtccaggctgtgtatcaaaagtttgtttattttatctaatgagacagtaaacatctggagtcatttgctgggtttctttctcttcttcaccctgggaatatatgacatgacatctgtgttaccttcagcaagtgcgtccagagaagattttgtaatttgttctatttgtcttttctgcttccaggtctgtatgctttgctctgtgggctatcatcttttttcctgccatcggtcagaaaaaacatgtcgaagatggatggcattagattatgcaggaatttctattggaatactgggctgctatgtctcaggagtattttacgcattttattgtaataactactggcgtcaggtgtacttgatcacagtgcttgctatgatcctggcagtgttctttgcgcagattcatcccaattacctcacgcagcaatggcaaaggctccgttctatcatcttttgttctgtttcgggatatggagtgattcctactcttcactgggtttggctcaatggaggaattggtgctcctattgtacaggactttgcaccccgtgtaattgtgatgtatatgattgctcttcttgctttcctattctacatttccaaagtcccagagcggtactttccaggacaactaaactacctcggatcaagccaccaaatatggcatatccttgcagtagtgatgttatattggtggcatcagtcaacagtgtatgtcatgcagtacagacatagcaagccttgtcctgactatgtttcacatttgtgaattaggtatggccacctggtgaattcagttgttaagcaatatataatggggaattgtataccccactatttctaagattcccattagttttccctttttcctttttaatatgagtaatgctttataaaaatgggaaaaaaagtatacttaaggatctgtagtaataactgctttacaaaatccttaaaactactaatttgctgcttgtacagaaagtgaaaattagttggcaatcataagaaacatctgaataacaacgatgaatgggaaactagtgttgaaataggattcattttacttagcaccagcttaatttccttaggaagggctcatctccattagaaaatggagtcatcttatgtgcttaattattttcagttaattgtcaagtttaagtgcctaatcaaggcaagtgttgtttcagcctatgcttaatgcaagctaggatagtgattttaaataatcactaaaatcactagatttaaataatcactaaaatgatttgtgagaaactggcacttcagatattatatcctttagctataggttcttctctccctaagaacattagatattttagttttccagaacaaaagctttaaacttctgcagtaagttgagagaagggttgagaagaggaaaagaacttctcattttctatcagataagaatcacattagaaactaagtacaagattagacaacaaattatgtggtcaaataatatagtcattagccacctaaacattttaattccagatattatttaattccatataataactgaattcttgtgagtggattacaggtttttgatcccaaaattccagagctttcaactctctgaatttgtagtcctgaatatcccagtggtgggggttcccagcattgtgggtgctacttgcaaggccatagaatctagatggccctgtcttgaccctgaaatgaaccttaagccttagaacaaagtcatgcagatgccccatttgataataatcttattcacctgtgctctggtcctcggtttctgcatgtgttagcattgcattgataactcagaatcttgataaacacttaatatttgggcctgaagcattaaactttctttttattgtatatacttaaaaaatagaactcactgccctatcatacattgtagccctcttattctttggtctttcatatgcattagttaaatcccttaaagtagacattcataaaaacttacattgtttattggagtataaaatattacccaagtttcttcatgagttgacatgagctgttttaaatactggtgtattttcagaacagtaaaattactgaatatcagaaaaaatgttaattgatgatgaagcttattcccaaaatgccttttgtgcatatgatacttggaaagtcactaatgtgcctcagttaatacatcagtaaaatgttgtgtttcttttccagtgtagtgtttttggaatataaattccccatgctagtatagtatctcagcaaagagaatttccccccaggaggctcagtaaaggaataccgtgtcttacccatcgttatgatggaaggctgctttgaaaatggctgttttaccttataaggttaaaattttgatccatatgttaagtgatagaagattttggtgcaacagtagtaggatatatttctcctagaacatcccttattggcttacatgattttattgccttttaatagatattttgtcattttggccaaacaaaagacactgagtagttacacttaagttaaaaatgaggggaaaatcattattttaggtgtggagccatttttattataaaactttctcaaaataaaaaaacattgaatcatttcaatttttgcagtccctgtattagtatatgaatacatacttgccatttgaattaataacatgaaaagagtatactgtgtttttaaatccgtgtttctttgaatttaaagggtgtacaggtctttctgtagggaaaattattccatgtaaacatttcaactctgtatgaaaatgttaaatattgtaagaaagttatcctctcattttttcactgctatgatatatttattataaaatagggaatgaatgaatgaatatggattgctgttaactagaaacacttctgtatgtcagtcagcatttaatgaccacctactgtgtgcacagcactactggtaaaattttgaagacattgttaacattaaaaaatattttaaagttgtctacaaatctgagccttgtaatgatgtatatttaagttatttttgtttttatagattaaagtaagattatactatccagttttattactaaaaaagactggttttaattttaccaatgtgtgaactataaaagctttttgcctacagattttacattttaaaattatctatggctgttttaaattgtctagcaatttatatggttgtggttaactcatttaagaaacaattatctttctatattaagccattttcaaatagcaagacagtgcttgtctttttttgttattacactaactgcaattcagtaagctgcatgacaaaatatgtattatgtaaataaactgggtttactaaatactttct

SEQ ID NO: 89 - DNA, 3250 bp, Homo sapiens
  misc_feature: Incyte ID No 2902971CB1
atggcaacaagtatggcggctgctagtggtagatttgaaagtgcgaagagtatcgaagagcggaaagaacagacccggaatgccagggccgaggtgttgcgccaggctaaagccaattttgaaaaagaagaaaggcgtaaagaacttaagcgacttcggggtgaggatacatggatgctacctgatgtgaatgagagaattgaacagttctcacaggaacactctgtgaagaaaaagaagaaaaaagacaagcattcaaaaaaagcaaagaaagaaaagaaaaaaaagagcaagaaacagaaatatgaaaaaaacaatgagtcatctgatagctcatcaagctctgaagatgagtgggttgaggctgttccatcccagactcctgacaaggaaaaagcctggaaagtgaaagatgaaaagtcaggaaaagatgacacccaaattatcaagagggatgagtggatgactgttgattttatgtctgttaaaactgtgtcatcatcatcactcaaagctgaaaaggaaactatgaggaaaatagagcaagagaaaaaccaagcgcttgaacagtccaaactgatggaaagagaattgaatccgtactggaaggatggtgggacaggtcttccacctgaagactgtagtgtgtcatcgattactaaagtttcagtggtagaagatggtggattaagctggctaaggaaatcttatctaagaatgaaggaacaagctgagaaacaaagtagaaactttgaggacattgtagccgaaagatatgggtcaatggaaatatttcagtcaaaattagaagatgctgaaaaagctgcatccacgaaagaagattatagacgggaacggtggaggaaacccacatattcagataaagcacaaaattgtcaagaaagtagagaatcagacttagtaaaatatggtaacagttcaagggatagatatgctacaacagatactgcaaaaaatagcaataatgaaaaatttattggtgatgaaaaagataagagacctgggtctttagaaacgtgtagaagagaatctaacccaaggcaaaatcaagagttttcttttggcaatttgagagctaaattcttgagaccctctgatgatgaagaactgtcatttcacagcaagggcagaaaatttgaaccacttagttcatcttcagcattggtagctcagggctctttgtgtagtggttttagaaaacccaccaagaacagtgaagaaagattaacatcatggagtcgctctgatgggagaggagacaagaaacattcaaatcaaaagccatcggaaaccagtactgatgaataccaacatgttccagaagacccaagagaaaaatcacaagatgaagtcttgagagatgaccctccaaaaaaagaacatctacgggatacaaagtctacatttgctggcagtccagagcgtgagtccattcacatcctgagtgttgatgagaagaacaagttgggagccaagattatcaaagcagagatgatggggaatatggaattagctgaacaacttaaagttcaacttgaaaaggcaaataaattcaaagaaactataacacagataccaaaaaaatctggggtagagaatgaagaccagcaagaagtaatccttgtcagaacagatcagtctggaagagtatggcctgtgaacacacccggaaaatctctggaatcacaaggaggaagaagaaagagacagatggtttcaacccatgaggaaagagaaagggtcagatactttcatgatgatgataatctaagcctaaatgatttagtcaaaaatgaaaagatgggaacagcagaaaatcaaaacaagctctttatgagaatggcatctaagtttatgggaaaaacagatggagactattacaccctggatgacatgtttgtctccaaagcagctgagagagaacgtcttggtgaagaggaagagaaccaaaggaaaaaagctattgctgagcatcggagtcttgctgcacaaatggaaaaatgtctgtattgttttgacagctctcaatttcccaagcatcttattgttgcaataggtgttaaggtttatttatgtttacccaacgtacggtctcttactgaggggcactgcctgatagtccctttgcagcaccatagagcagctactttgttggatgaagacatctgggaggagatccagatgttcagaaaatcattggtaaagatgtttgaagataaaggattagactgcatttttttggaaactaatatgagcatgaagaaacagtatcacatggtttatgaatgtattcctcttcccaaggaagtgggtgacatggctcccatctattttaagaaggccataatggaatctgatgaagagtggtccatgaacaagaagttgatggatctctcttcaaaagatatcagaaagtctgtacccagagggttaccttacttctctgtggattttggccttcacggagggtttgcccatgtcattgaagatcagcacaaattccctcattactttggaaaggaaatcataggtgggatgctggatatagaaccaagactttggaggaaaggcatccgagaaagctttgaggatcagaggaaaaaagcactgcagtttgctcagtggtggaaaccatatgacttcaccaaaagtaaaaactattgaggtgtaccttccattttaaaatttttcttcagatcccgttcagttttatttccattgcatctaatgaagcaactgaccctcaggtcacaggcagagagagtcacagcagcagactgctctgggtcagtgacaactgtacatcaggagattgtgttgttttcttcttcacctgctcctatctatggacttcattttagtcaccatggagtaaaaacatatattcgatgatctgctcacattcctgctctctacctacttcttggtattccgaattgttcagttaatttagagaaggccttttctgaagggatatctagaaagcataggaaatccttggttcagagggaagtgggtctgcttcatggttgttaagtctactgtctggcttcttttgcatttttaaaaataagacgatttaattttaatttcataggattttatagaaaagctcttaattttctactagcagtatatagtatcacttctattctttttgttaaaggaaaaggaaatgtccccaaagtattaaagaattaaatgatactgaaaattaaaaaaaaaaaaa

SEQ ID NO: 90 - DNA, 2295 bp, Homo sapiens
  misc_feature: Incyte ID No 368660CB1
gtaccacccggctcaagtagcggacacggaacagggaactatcagcccgtcggcctccgggccctgcattctctagccatggaccgggaccttttgcggcagtcgctaaattgccacgggtcgtctttgctctctctacttcggagcgaacagcaggacaatccacacttccgtagcctcctggggtcggccgccgagccagcccggggcccgccgccccagcacccgttgcagggcagaaaagagaagagagttgacaacatcgagatacagaaattcatctccaaaaaagcggatctgctttttgcactttcctggaaatcagatgcacctgcaacttctgaaattaatgaagacagtgaagatcattatgcaatcatgccacctttagagcaattcatggagatacctagtatggatcggagagagctgtttttccgagatattgagcgtggtgatatagtgattggaagaattagttctattcgggaattcggttttttcatggtgttgatctgtttaggaagtggtatcatgagagatatagcccacttagaaatcacagctctttgtcccttaagagatgtgccttctcacagtaaccatggggatcctttatcatattaccaaactggtgacatcattcgagctggaatcaaggatattgacagataccatgaaaagctagcagtatctctgtatagctcttctcttccaccacacctatctggtattaaattaggtgtaattagctctgaagagcttcctttatactacaggagaagtgttgagctaaatagcaattctttggagtcctatgaaaatgtcatgcagagttccttgggatttgttaatccaggagtagttgaattccttctagaaaaactaggaatagatgaatctaatccaccatctttaatgagaggcctacaaagcaaaaatttctctgaagatgattttgcttctgcattgagaaaaaaacaatccgcatcttgggctttaaaatgtgtgaagatcggagttgactattttaaagttggacgccatgtggatgctatgaatgaatacaataaagctttggaaatagacaaacaaaacgtggaagctttggtagctcgtggagcattatatgcgacaaaaggaagtttgaacaaagcaatagaagattttgagcttgcattagaaaactgtccaactcacagaaatgcaagaaaatacctctgccagacacttgtagagagaggaggacagttagaagaagaagaaaagtttttaaatgctgaaagttactataagaaagctttggctttggatgagacttttaaagatgcagaggatgctttgcagaaacttcataaatatatgcagaaatctttggaattaagagaaaaacaagctgaaaaggaagaaaagcagaaaacaaagaaaatagaaacaagtgcagaaaagttgcgtaacgtcttaaaagaagagaagaggctaaagaagaaaagaagaaaatcaacttcttcttcaagtgtttcttctgctgatgaatcagtgtcttcatcatcatcctcttcctcttctggtcacaaaaggcataagaaacataagaggaaccgttcagagtcttctcgcagttccagaaggcattcatctagggcatcctcaaatcagatagatcagaataggaaagatgagtgctacccagttccagctaatacttcagcatcttttcttaaccataaacaagaagtggagaaactactggggaagcaggataggttacagtatgaaaagacacagataaaagagaaagatagatgccctctctcttcatcttcacttgaaataccggatgattttggagtgtactcctatttatttaaaaagttaactataaaacagcctcaggcaggtccttcaggagatattccagaagagggcattgttatcatagatgacagctccattcatgttactgaccctgaagaccttcaagtgggacaagatatggaggtggaagacagtggtattgatgatcctgaccacgggtaggcttaggtttatgtgtgtgtatgtgtcttagtttttaacaaaaaaattaaaaagtaaaaaaactaaaaatagaaaaatgcttagagaataaggatataaagaatatttttgtgcagttgaacaatgagtgcttaagctaaatgtcatcacaaaagagtaaaaaaattttacaaaattaaaaatgtttaaagttaaaaagctctaggaagctaaggtcaatttattattggagaaataaaattatttttatgaatt

SEQ ID NO: 91 - DNA, 1777 bp, Homo sapiens
  misc_feature: Incyte ID No 2804990CB1
actaaatgtgccagtacctgagattccaggaaaaactaaaaatttgtttttgcaattagccaaacatgtagcccagtctctacaagtcgcctatgtcatgtttgtggaggaaccgtaacaggagatcaatggccatgggaagcccgagaattggttcctatagacccggttcctgatgaattcccagcccaaaagaaccaccctgacaaattttgggttctgaaagtcttattattggacagtattgcatagctagagaaggaaaaggatttactcatcctgtagggcagcttagttgtcttgggcaaaagatgtataatggtaccacaaaaacagttacatggtggagttccaattacacagaaaaaaatccattcagtaaatttccaaagttgcagactgtttgggcccacccagaattgcactgggactggacggcccccactgggttatactggatatgtggacacagagcttatgctaagctgcctgatcagtggacaggtagctgtgtaattggcaccattaagccatctttcttcttagtgcccataaaaacaggtaaacttctgggcttcccagtctgtgcttcctgtgaaaaatgaagcatagccataggtgattggaaagacgatgaatggccccctgaaaaaatcttacaatactatggacctgccacttgggcacaagatggctcatggggatatcgaacccccatctacatgctcaaccgaatcatatggttacaagctgttttagaaattattactaataaaaccactcaagccttgactgttcttgcctggcaagagactctgatgagaaatgctatctatcaaaatagactagctcttgattacttgctagcagctgaaggaggagtttgtgaaaaatttgaccttactaattattgtctacacatagatgatcaggggcaagtagttgaggatatagttaaagatataacaaaactggcacatgcacctgtgcaagtgtggcacggactcaatctgggagccatgtttggaaattggttcccagcaataggtggatttaaaactcttataataagagtaataatagtaataggaacctgcttactgctcccttgtctgatacctgtatttctccaaatgataaaaaacttcgtcgcttaaccttagttcaccaaaatgcttcagcacaagcatactatataaatcactatcaatctattgcacgaaaatacataagtagcaaaaataagagtgagaactcccactgataaaaagtgagagtctcaaagaggggaaatgagggaagagagagaccctctcatattgttttatattgttttatactcagtacctcttttaagaaaaaaacaacaaggaagtagaaccaaagacaagcagcccagcgccaggcccaaaaccaggcctgggcctgcctggcctaaacccagtagttaaaaatcaactcataacttagaaactgatgttattcatagattccagacattgtatagaagaacattgtgaaactccctgccctgttctgtttctctctgaccaccggtgcatgcagcccctgtcacataccgcctgcttgctcaaatcaatcatgaccctttcatgtgaaatctttagtattgtgagcccttaaaagggacggaaattgtgcattcggggagctcggattttaaggcagtagcctgctgatgctcccagctgaataaagcccttccttctacaaaaaaaaaaaaaaaaaaaaaaaaaaaaaaaaaaaaaaaaaaaag

SEQ ID NO: 92 - DNA, 3181 bp, Homo sapiens
  misc_feature: Incyte ID No 168571CB1
gactccaccagccacccctgagctccaaccttggggggatgcagagatcggggaggcccaaggccacctcctcaggcccaaagggaggccactgcaggcaagatgaggtggcccagaggagccacatggggcctggaggccatctgggccgaggcagagccaccgtcacacgtggatctcagagacccacaggaagaggatggtggctgggagggctgaggcagtcccccagagaactgtcctgccaggccacaaggtcagtgagagggagcccggctcccaccccagggaccaggtgtcctttttcaggtccaggtggcagtgtccatcactccaggggcccccactggggaacttctgagaagccagggcatggagcaagagggtgatacccacaaactctttcctcatccttccctgatttgaaatggagggtgggatgcagaggagagaggtggaaagacaggtgcacagccccaggcctctcccccacccctgcctcgcccaccatgtggctggaacggtgaccacccagcaagaacccatctcccaccctcaccagtgatcccggaaaaccagtcactggggtcccgcggggcatccgaaggcccaagggcaagtcacctttcagaatgtcccctctgtgcagcctttgccaggaaggaagctggaccgggccagctgcatggatgccaggacctctaggccctgagcatcagggggtccagcccaggacaccccaagcctgggcccctctccctgctgaagggctctggggagcgcggggagaagccagcaggcatggaggctgcccatcccccagccatggcctggggccccacgcagctctctgcctcccccaggagaacccccgcctcacagaagacttcgtgtcccacctggagaccgagctggagcagtcgaggctgcgggagacggagacactgggggcccttcgggagatgcaggacaaggttctcgacatggaaaagaggaacagctcgctgcccgacgagaacaatgtggcgcagctgcaggaggagctgaaggcgctcaaggtgcgggaaggccaggcggtggcctcgacgcgagagcttaaactgcagctgcaggagctctcggacacctggcaggcccatctggcccgcggcggccgctggaaggagtccccacggaagctggtcgtgggcgagctgcaggacgagctgatgagcgtgcgtctgcgcgaggcccaggccctggccgagggccgcgagctgcggcagcgcgtggtggaacttgagacgcaggaccacatccaccgcaaccttctgaaccgcgtggaggcggagcgcgcggcgctgcaggagaagctgcagtacctggctgcacagaacaaggggctgcagacgcagctcagcgaaagccgccgcaagcaggccgaggccgagtgcaagagcaaggaggaggtgatggctgtgcgactgcgggaggcggacagcatggctgcggtggccgagatgcggcagcgcattgccgagctggagatccagagggaggaaggccgcatccagggccagctgaaccactcggactcatcgcagtacatccgcgagctcaaggaccagatcgaggagctgaaggccgaggtgcggctgctgaagggcccgccgcccttcgaggacccgctggctttcgatgggctgagcctggcgcggcacttggacgaggactcgctgccgtcgtcggacgaggagctacttggcgtaggcgtgggcgctgccctgcaggacgcattgtaccctctgtccccgcgcgatgcgcgcttcttccgccgtctggagcggccggccaaggacagcgagggcagctcagacagcgacgccgatgagctggccgcgccctacagccagggtctggacaactgaggccatgcccagcgcgcccggagtcaggaggccgcagccgcggggggcgcccgggcagtccgcgttctgctccccacctgccgcacttgacaaactacgcgccctctgtggctcggccacccctaaagcgaggcccggcgaggcagcgcagagggtagggtccgacctgggctcctcagggccccggggcaggctctctctccccagcagtgtttacccatcttggtctgtacccctccgggccctctggcgttccaggggtgcctggaggggctgactgctctcttaacaggagggcagagggcaggggacagacgacccagaggtcccagcactgaatgagcaggcagctcccacctcctggcaggcttccttctgggacaggggcgacattgctgggaagtgctcaggaggtagccgaggcctgaggaaggagagcgccagctctgggctggacatcagcaccccacaacactcctcgggatgaagtgacccttgactagcccctggccatctctaggggagtcaggccgctggggacagatggccaggccggcctctcctgcctggcgcaggcaccgtggcccctgcagcggaaaccaaagtccccctactatgtgcggggcgcctggggactgagtggctaacgggaagcctccctgcttctctggggccagagcagcttccaggaagtgaagagacccctctccccagtgccccacatctttctctgggagacactcgcgccccctatcctggccagtgatggaggggtatctctacaggggtcctcggtctccatctacttccctttcttcatgtgtttccagccccaccctcagccagagccaggcccccaggcaggagcttcccagcgagcggcctcccctcacttcctcctggtggtcccgttgtctctgctgaatcagagctgagaacggtgccaaaatccaacgatgccccccaggccccttcctcccttcccccggccccccgggcctcatgaccctgggggctgcccccctcggtgctcccggagcctccagtagagtagcgtcacaagcaatctccctggcgcttcctgggtggggacccctgctccgtccccgcttcctagctgcccacttttcagtgttacgaagcctggggaccggggcaggcacccacggggctctccacacgccccctacactgcccgccaccattttgcacactgcctgttcacatgtcgcccaggcgggaaaaatggaaaataaagtgtatttacacagtca

SEQ ID NO: 93 - DNA, 5987 bp, Homo sapiens
  misc_feature: Incyte ID No 1286391CB1
ccagtgctgccaacagggcaagggaagcccgtggctcaaggggggctgacagatcagtcagtacctggagctcaaacagtgcctgaaactctcaaagtgcccatggctgcagcagtgcccaaagctgaaaatccctccagaactcaagtgccatctgcagctcccaaactgcctacatctcgaatgatgctggcagtgcacacagagcctgcagctcccgaagtgcctttggctccaacaaagccaacagctcaactgatggccacagctcaaaaaacagttgtgaatcaaccagtgttggtagctcaagtggaacccacaactccaaaaactccccaggctcagaagatgcctgtagcaaaaacatcacctgcaggtcccaaaacacccaaagctcaagccgggcctgcagctacagtttccaaagcacctgcagcttccaaagcacctgcagctcccaaagtacctgtgacccccagagtctccagagctcccaaaacacctgcagctcagaaggtgcccacggatgcagggccaaccttggatgtagccagacttctgagtgaggtccagcctacatcaagggctagtgtctccttactgaagggccaggggcaggctggaaggcagggtccccagtccagtggcaccttggccctcagcagtaagcaccagtttcagatggaggggctcctgggggcttgggagggggccccaaggcagccacctcgccacctgcaagcgaacagcacagtgaccagcttccagaggtaccacgaggccctgaatacacccttcgagctgaacctgtcaggggaacctggaaaccaggggttgcggcgagtggtcatcgatggcagcagtgtggccatggtgcatggcctgcagcacttcttctcctgccgaggaattgccatggcagtgcagtttttctggaaccggggacaccgagaggtcactgtgtttgtacccacctggcagctgaagaagaaccggagggtgagagagagccactttctgacgaagctacactcgctcaagatgctttcaatcacaccctcccagcttgagaatggcaagaagatcaccacctacgattataggttcatggtaaagctggcagaggagacagatggcatcattgtcaccaatgagcagattcacatcctgatgaatagttccaagaaactgatggtcaaagatcgcttgctgcctttcacctttgcggggaatctcttcatggtgcctgatgaccccctgggccgtgatggccccaccttggatgagtttctgaagaagccaaacaggttggacactgacattggcaacttcctgaaggtgtggaagacccttcctcccagctcagccagtgtcactgagctgagtgatgacgctgactctgggcccctggagagtctgccgaatatggaagaagtcagggaagagaaggaggagaggcaggatgaggagcagagacaggggcagggcacacagaaggcggctgaggaggacgaccttgactcttcgctggcgtcagtgttcagggtggagtgcccgtccctttcggaggagatcctgcggtgcctcagcctccatgatccccctgatggggccctggacatcgacctcctgccaggggcagcttctccctacctgggcatcccctgggatggaaaggctccctgccagcaggttcttgcccacctggcccagctcaccatccccagcaacttcaccgcactctccttcttcatgggcttcatggactcccacagggatgccatccctgactatgaagccctagtgggccccctgcacagcctcctcaagcagaagcctgactggcagtgggaccaggagcatgaggaggccttcctggccctgaagcgagccctggtgtctgccctctgcctgatggcccccaactcccagctgcccttccgcctggaggtgaccgtgagccacgtggccctgacggccatcctccatcaggagcactcagggaggaagcaccccatagcctatacctcaaaacccctcctccctgatgaggagagccagggcccccagtcagggggtgacagcccctatgctgtggcctgggccctcaagcatttttcccgctgcattggagacaccccggtggtcctggacctttcctatgcctcccggaccactgcggaccctgaggtgcgggagggccgcagggtttccaaagcttggttgatccgatggtccctcttggttcaggacaaaggcaagagggccctggaattggccctcctccagggcctgctgggggagaaccgcctgctcacccccgcggcctccatgcctcgcttcttccaggttctgccgcctttctctgacctgtccacgttcgtctgcatccacatgtcgggctactgcttctaccgtgaggatgagtggtgtgctggctttggtctctatgttctatcgcccaccagcccccctgtctccctttccttctcctgctccccttacacgccaacctatgcccacctggcagccgtggcctgcggcctggagcgctttggccagtccccactcccagtggttttcctcactcactgcaactggatcttcagcctcctgtgggagctcctgcccctctggagggctcggggcttcctctcctctgatggggctccactccctcacccaagcctgctctcctacattatatccctcacctctggcctctcatcccttccgtttatctaccgaacctcctaccggggctctctgtttgctgtgacagtggacaccctggccaagcagggtgcccaggggggtgggcagtggtggagtttgccaaaggatgtgccagcccctacagtgagtccccatgccatgggcaagaggcccaatttgctggcattacagctgagtgacagcaccctggccgacatcattgccaggctgcaggctgggcagaaactgtctggctcctcaccgtttagttctgcctttaactcactcagcctcgacaaggagagtggcctgcttatgttcaagggagataagaagcccagggtctgggtagtcccgacgcaactccggagggatctgattttctctgtgcatgacattcccttgggggcccaccagaggcccgaagagacctacaagaagttgcgtttgctggggtggtggcctgggatgcaggagcatgtgaaagattactgcaggagctgcttgttctgcatcccccgaaatctcataggcagcgagttgaaggttattgagtccccatggcccctcaggtcgaccgccccctggtcgaacctgcagatcgaggtggtgggcccggtcaccataagtgaggagggccataagcatgtacttattgtggctgacccaaacaccaggtgggtggaggcattccccctgaagccctacacacacacggctgtggcccaggtgctgcttcagcatgtgtttgcaaggtggggtgttcctgtgaggctggaggcagcccaggggccccagtttgcccggcacgtccttgtgagctgtgggctggccctgggagcccaggtggcctccctgagtcgggacctccagttcccctgcctgacgagctcaggggcctactgggaattcaagagggccctcaaggagttcatcttcctgcatgggaagaagtgggcggcctccctgcctttgctgcacctggccttcagggcctcctccactgatgccacaccgttcaaggtcctgaccgggggtgagtcaaggctcacggagcccctgtggtgggagatgagcagcgcaaacattgaagggctcaagatggacgtcttcctgctacagctggtgggggagctgctggagctccactggagggtggctgacaaggcgagtgaaaaggccgagaacaggcgtttcaagcgggagagccaggagaaggagtggaatgtgggtgaccaggtccttttgctgtccctccccaggaatggcagcagtgccaaatgggtgggtcccttctatatcggggaccggctgagcctgtcactctataggatatggggcttcccaaccccagagaagctggggtgcatctatcccagcagtctgatgaaggcctttgccaagagtggcaccccgctgtccttcaaggtcttggagcagtgagcgggagcagcgggggtgccccctgccccagggccgtgggtttctgctgctaggcctccccctgtcccagcagtgctctcagtccactgggggccctcagttgtgccttttgtagagaacttgcttcataaagctttgctgaattgccttgaactagggaccagcatccccatggaaacatccccagtttggggtacttggagaatttgccaaaggctgtcagatatgggtccctggcagttttacactgggaaatagagtgctcctctaggctataccaggctcagtgtcttctccccaagtatcctctttccccttcacatgtaggtgtgtggtggtgtgcacacacactcacgaaagaatctatcttggccatgaaactgtggctgttgaccttggaattggaaccacagtcctttcccatacagaaaccccaaatgtgggctcctccctccaacctgttcttttgggggaccctttgcccttagagctgtcacagatgacataagcctgggcaggctgtggggaggccgctgcctcctagcctcacggtcagctttctcaagccaggtgtggcctgcacagctgtcagggcagggctggccatcctcccaggcctcaagggcagtgtcttggagtgggaggatggccagccacaagccaccagcttgtcagcatgggaagggcaagggggaaatgggttggcctacctcatttgactccagccaatggagacaattcctgacccctgctttgatggtgttgaccccacaggaatcaaggatcttgatgccaagtgtggcatctctacctgaagagctgcttctgttagacccaggggccccggcctctgttttaagggggcagggcgtctgcaacaggagtggcacacggtgaagtgctggcatggctctacctcccaacccctcccaaccccatcccaaagcctacagtcctctgctccttctacctccactgtatcctgcatcccagaccctacagattgtgtgattgcttcatctgtatcaccccccgagtcctgtggacctgccttctgtgtagagcaaaacccaggctccctcacagccattctttgcagagatcacccttgcagtgagtgtgagttccttccaggtgtgtgcgcgtacactcaccctctgaattgctgccgctgccaaggaagtgggctccaggtgaaggccagtgccacgtcactctggctggccttcttagtagtttcatttctccggaagctgagccagtctcctggtctagcccaggttgccagaacgcttggcattgcagagtgctagagccagtggagaacttgccaacttgattgttttacagcagaggaaagaggatcacagagggaaagtgattcacccaaagtcacacagcaagttcatggctgagctgagaccaggattaagcttcctgactcccagttcgccatgaaaagggttctggcaacaggttcaagctggagaatccttcaaaatgctacacccacattctctccaactcttcatctccctgatcttccagacaaactacctggatgttgcccttaaaccatttctagctgttaaccctgtccagaaaaatgattgagtgatagctgagaagtggaaagtgtgggatttttggcaggtgctctctttcctccgccccccgcgccattctttctcttcctcctctctgtaatggtatgtccagcctcactctccctccctggtgctgtatgcgttccccctgttagctacatttgtgatcacatacccttcttttaagtgaatttttttcatttgatttgtcaataaacgaat

SEQ ID NO: 94 - DNA, 765 bp, Homo sapiens
  misc_feature: Incyte ID No 2007684CB1
gtcggggctctgaggctccggcctgacctctcctcggggtcgacgggaaggtctccggatgccaggactcgcaaagggccgaccagaatgaggaaatcccaggcggagtccgggggaggcagcacggcatcccagcctcagggctgcccggacggtgttggttggggtaagattggggaagatgccaggtgatagaatccaccctgcctctgacagcatccaatctggagcaaaggcttctttcaaccttcccccaagctcccaatacaagcccaaattctggaacaagtcctttccaacacctccttcagagaagctccctggctccaggcagtgcttgctcccctcactgcagcaggaagcctaacttgcgaactgcaggtgtgctccaggtggtcactggatgcaagaaatggtgcgagaactttggatgtggaacgtggaggaggaggaacacgaagtggggatctgcacttggggtggtcagcactgcggatgcccagcaaagtcactgcctggtccacatcccggaggggtctctgcgcctcagtcggcatcgcagctgatggtgaaacttttggtgtggcagaagagtgtccacaaacttcggaagttgctagagaaaactgaaaactactagttttgaaagcctcacttctgccaatggaagcacattccagcatgtcgccaacgcaatccactttccaccactttcacaaaaaacgttactgcacaattatactatcctacccttaaaaaaaaaaa

SEQ ID NO: 95 - DNA, 2674 bp, Homo sapiens
  misc_feature: Incyte ID No 2227040CB1
gcgacctcctaaatcaaagttgggcggcgaggactgggcggaggggccgccgggtgccgggctggggccggcgggggaggggaggggatggggggcgggcgcagcggacggcggagccggctggacacggacagctccttggctccctccctcggctcatccccgcggccccacttccctcctccccgcgctgcgagcagcatcctctgcagaccctcggtcctcgcgcccggggtcgtcccgctcctgcggctcagcgtggtggcctcccctcgcccgccaccccggcaactttctctgcccgctcccgcgggttgggggctgccgtgccgggggctaactgggggcagcctctggagaacggcttcagagtcccggagacgcccgccacccgcagcctgcccgcggtgggcctgccgtcggatctcggcaccctctcctcccctctccccgaaccatgaccgagatgagcgagaaggagaacgaaccggatgacgcggccacccacagccccccagggaccgtctccgccctccaggaaaccaagctccagcgattcaagcgctccctctccctcaagaccatcctccgaagtaagagcttggagaacttcttccttcgctcgggctctgagctcaagtgccccaccgaggtgctgctgacgcccccaaccccactgccccctccctccccaccacccacagcctcggacaggggcctggctaccccatccccctccccatgcccagtcccacgccccctggcagcgctcaaaccagtgacgctgcacagcttccaggaacatgtcttcaagcgagctagcccttgtgagctgtgccaccagctcatcgtaggaaactccaaacagggcttgcgatgtaagatgtgcaaagtcagcgtccacctctggtgctctgaggagatctcccaccagcaatgcccaggcaagacgtccacctccttccgccgcaacttcagttcccctctcctggtgcatgagccgccaccagtctgtgccacaagcaaagagtccccacccactggggacagtgggaaggtggaccctgtctacgagaccctgcgctatggcacctccctggcactgatgaaccgctccagtttcagcagcacctctgagtccccgacaaggagcctgagtgagcgggatgagctgaccgaggatggggaaggcagcatccgcagctctgaggaggggcctggtgacagtgcatctccagtattcacagccccagcagagagtgaagggccaggaccagaggagaagagtcctggacagcagctccccaaagccaccctgcggaaggatgtggggcccatgtactcctacgttgcactctacaagtttctgccccaggagaacaatgatctggctctgcagcctggagatcggatcatgctggtggatgactctaacgaggactggtggaagggcaagatcggcgaccgggttggcttcttcccagctaattttgtgcaacgggtgaggccaggcgagaatgtttggcgctgctgccaacccttctccgggaacaaggaacagggttacatgagcctcaaggagaaccagatctgcgtgggcgtgggcagaagcaaggatgctgacggcttcatccgcgtcagcagtggcaagaagcggggcctggtgccagtcgacgccctgactgagatctgagaggagccaagggaacccagatgacacccttgcccatgcctggaccttgcttctggccagggaggggatcaggcccccttgtccactccatacccttcctccctctgtccctctcctaggtgccacttaccgtggcttaggagccttttgtactggggaagattttatttcttgggtggggtgccctgagtgggttgatcctctgggacttggcggggatggggtggggtgggagggaatgaggaagctacaggtaggtccacccaacgcccaggcaccccagtctacttgctgggctgagtccagccctggggaagattcctggggaatttatttgctgcttctcccagccttcccctgcccacaccacctgcgcatgcctggcacaaccctctccccacagagcctctgcttgggtctatgtgtgtgtgaccgtgcatctgctcctttgcaagtggggtcttgggagcaggcctttttgtgtcctcggccctccacccttggtggggaggggacccaggaccataggaaggtcctgagtccacctcctggtctccacctctgtcattccgtcaattctcaggaaagttttgcaggaatctctcccgttacttgaaacctgggcggacagatgaggggaaggctgaggtccctggaaggtgtaacaaatcagagactatctgttcaagtagaaaacccaagagctggccaggtgtggtggctcatgcctgtaatcccagcactttgggaggctgaggtgggcagatcacctgaggtcaggagttcaagaccagcctggccaacatggtgaaaccccctctctactaaaagtacgaaacttagccaggcatgatgggggtctgcctgtaatgccagctactccagaggctgaggcaagagaatcacttgaacccgggaggcggaggtttcagtgagccgagattgcaccattgcaccccagcctgggcaacagagccgaga

SEQ ID NO: 96 - DNA, 5920 bp, Homo sapiens
  misc_feature: Incyte ID No 4346130CB1
cgccaggcagcagaggcgactgcggacagccggcggggaccgggaaagaggaggcctggcgcctccggcagaggggcgaggaccgagcgcccaaggctgggagagacgccggcgctgagcttcccagcgccgcctcacctcgcttttcgaactccgcgacccgagcgcaaggcttaagcaggtaaagcagtcactgtgaagaaaataacattttaagaaacttttctgtcacaattgatagaggaaaagccagtagagaagtacatcttcttgtgtgtgtgtctcatcgtgccactttttgtaactcagcttagtaatgaagccaccattatgtcctcagtcagtgaagtaaatgtggatataaaagatttcctaatgagcattaatttggagcagtatctcttacatttccatgagtctggttttactactgtgaaggactgtgcagcaataaatgacagcctgctgcagaaaattggaatatcacctacaggtcaccgtaggaggatacttaaacagttacagataatcttgtcaaaaatgcaagatattccaatatatgcaaatgttcataaaactaagaagaatgatgacccttcaaaggattaccatgttccatcttctgatcagaatatctgcatagaactttccaattctggtagtgttcagacatctagcccaccgcagttggagactgttagaaaaaatcttgaagacagtgatgcaagtgtggaaagaagccagtatcctcaatcagatgataagctgtctcctcctaaacgcgacttccccactgcagaggaaccacacctgaatttgggttctttgaatgattctttatttggtagtgacaatattaaaatagaatcattgattacaaagaagactgtggatcacacagttgaagaacaacaaacagaaaaagttaaattgatcacagaaaatctcagtaagctccctaatgcagactctgaatgcctttcttttgttggctgttcaacatcaggaacaaattctggaaatggaacaaatggtttattagaaggatcaccaccatccccattctttaagtttcaaggagaaatgattgtaaatgacttgtatgttccatcatcaccaatcctagcacctgtgagaagtcgtagcaagttggtttcaagaccatctcgatcttttctgctaagacatcgacctgtaccagagattccagggtcaacaaaaggagtttctgggagctatttccgtgaaagaagaaatgttgctacctcaactgaaaaatctgtggcatggcaaaattcaaatgaggagaattcatcttccatctttccttatggagagacctttctcttccagagactagaaaattccaagaagcgatctataaagaatgaatttttgacccagggagaagcactcaaaggggaagcagctactgcaacaaactcttttatcatcaaatcaagcatatacgataacagaaaggagaaaataagcgaggacaaggtggaagatatttggatacctcgagaggacaaaaacaattttttgatagacactgcttctgaatcagaatactcaacagtagaagaatgctttcagagtttaagaagaaaaaattcaaaggcatctaaatctaggactcaaaaagccttgattttggactccgttaataggcacagttatccgttaagctcaacaagtggaaatgctgattcatcagccgtttcttcacaggcaatatctccctatgcctgcttttatggagcatctgcaaagaaggttaaatcaggatggctggataaactctctcctcaaggaaaacgcatgtttcaaaagagatgggtgaaatttgatggccttagcatttcttactacaataatgagaaggagatgtattcgaaaggaataattcccctttctgctatatcaacagtacgagttcaaggagacaacaaatttgaagttgttacaacacaaagaacttttgtttttagagtagaaaaagaagaggagagaaatgactggatcagcatactattaaatgcactgaaatcacaatcccttacctcgcagtctcaagctgttgttacacctgagaaatgtggatatcttgaattgagaggctataaggcaaaaatttttactgtgttaagtggaaacagtgtgtggctttgcaaaaacgaacaggattttaagagtggacttggtattaccataattcctatgaatgtagcaaatgtaaagcaagtggaccgaactgtgaaacaatcttttgaaataatcactccctacaggagtttcagctttacagccgagactgaaaaggagaaacaagactggattgaagctgtgcagcaatcaatagcagaaactctctctgattatgaagtagctgagaagatttggttcaatgaatccaacaggagctgtgcagattgtaaagccccagatcctgactgggcatccatcaatctctgtgttgtcatctgtaagaagtgtgcaggacagcatagatctttaggaccaaaagattccaaggttagaagtctaaaaatggatgctagcatttggagcaatgaactcatcgagctttttattgtcattggaaacaaaagagcaaatgacttttgggctggtaatcttcaaaaggatgaagaattacatatggactcaccagtagaaaagagaaaaaactttattactcagaaatataaagaaggaaaattcagaaaaactcttttggcatctctcaccaaagaagaattaaataaggctctatgtgctgctgtagtgaaaccggatgttctagaaacaatggctttgctgttcagtggagcagatgtcatgtgtgccaccggagaccccgtgcatagcaccccctatctgctagccaagaaagctgggcaaagtctgcaaatggaatttctctaccataacaaattctcagatttccctcaacatgatattcattccgagggtgtattaagtcaagagtcttcccagtccacattcctctgtgactttttatatcaagctccttctgctgcttctaaactctcttcagagaaaaaactgcttgaagagacaaataaaaaatggtgtgttttggaaggaggcttcttgagttactatgaaaatgataagtctaccacacctaatggcaccattaatatcaatgaagttatctgcctggctatacacaaagaggacttctatttaaatactgggcccatctttatctttgagatctacttaccctccgaacgtgtgtttttatttggagctgaaacatctcaagctcaaagaaaatggacagaggcaatagccaagcattttgttcccttatttgctgaaaacttaacagaagctgactatgatttgattggtcaactcttctacaaagactgccatgccctggatcagtggagaaaaggctggtttgctatggacaaatccagcttgcatttttgccttcaaatgcaagaagttcagggagatagaatgcacttaagaagactgcaagagctaacaatcagcacaatggttcaaaatggggaaaaactggatgttttactcttggtagaaaaagggagaacattatacatccatgggcataccaagttggatttcacagtctggcatactgcaattgaaaaagcagcaggtacagatggtaatgctttacaagatcagcagctcagcaaaaatgacgttcccattatagtgaacagctgtatagcatttgttacacagtatggtttaggatgcaaatatatctatcaaaagaatggtgatcctttgcatataagtgaactcctggagagtttcaaaaaggatgcaagaagctttaaattgagggctggaaaacatcagcttgaagatgtgacggctgtgttgaaaagttttctctctgacattgatgatgcactgcttactaaggagctctacccatattggatctctgctttagatacgcaagatgacaaggaaagaattaaaaaatatggagcatttatacgttctcttccaggggtcaaccgagcaacactagcagctatcattgaacacctgtatagggttcagaaatgctcagaaatcaatcacatgaatgcccataatttggccttggtcttttcatcctgtttgtttcaaacgaagggacaaactagtgaagaagtgaatgtaattgaggacctaattaataattatgtagaaatatttgaggttaaagaagatcaagtcaaacaaatggacatagaaaatagctttattaccaagtggaaagacacccaagtttcccaggctggagatttgttaattgaagtatatgtagaaaggaaggaacccgactgtagtattataattcggatatctcctgtgatggaagcagaagaattaactaatgatatattagcgataaaaaatattattcctacaaaaggtgatatttgggccacatttgaagtcattgaaaatgaagagctagagcgtcctcttcactacaaggaaaatgtactggagcaggtgcttcggtggagttcattagctgaacctggctctgcttacctggtggtgaagagattcttaaccgctgacacaattaaacactgcagtgaccggagtacactgggaagcatcaaagaaggaatcttgaaaatcaaagaagaaccatccaaaatactatctggaaataagtttcaagaccggtattttgttttacgagatgggtttctctttctttacaaggatgtgaagagtagtaaacatgacaagatgttttctctcagttccatgaagttttatcgtggagtgaaaaagaaaatgaagcctccaacaagctggggattgaccgcatattctgagaaacatcactggcacctgtgttgtgatagttcacaaactcagacggagtggatgaccagtatctttattgcccagcatgaatatgatatatggccaccagctggaaaggaacgaaaacgttcaataaccaaaaatcccaaaattggaggtttgcctctgattcctatacagcatgaggggaatgcaaccttggcccggaaaaatattgagagtgcaagagcagaacttgaaaggctgcggctcagtgaaaagtgtgataaagagtccgtggactctagcttaaaggagagagcttccatggtggcccactgcctggagcacaaggacgataaacttcgaaatcgaccccgaaaacatcggagtttcaactgcctggaggacacagagcctgaagccccacttgggcaaccaaaaggccataaaggcctaaagacattgaggaagactgaggacaggaatagcaaagctactttggactctgatcataagttaccatcaagggtaattgaagaacttaatgtggttctacaacggtcaagaacccttccaaaagaattacaggatgagcagattttgaagtaggaaataaaatgaattgctccccagattatttttttaatattgtatgcaatctttatgtgtaactccaaaactgaactataagataattcataagaatttgcctattgataggcaaatttgtctatgcttaggcatttaaggagcatttaaaaatatgtatatatgtgactgaatgtaagattaactttattttggtctgaacaaaccttgtaaagttccattgtattaacgtgtgtaagaagacattgggttacttaggtggtcagtccttttaaagtagttgtctgttaagtggtataatttatgagcagaaatctcaaactgtactgtattgtataaaatgctgtgtttaaatgaagcctaagaaactatctgtaacatattttgcactttgagaaaccttgtatattaaattaccatatgtttttaggtttacagaagttctaccttagggaaaagaaagggaagtaaattg

SEQ ID NO: 97 - DNA, 1689 bp, Homo sapiens
  misc_feature: Incyte ID No 55117040CB1
ctggtttctcctggctgttaaaagcagatggtggctgaggttgattcaatgccggctgcctcttctgtgaagaagccatttggtctcaggagcaagatgggcaagtggtgctgccactgctttccctgctgcagggggagcggcaagagcaacgtgggcacttctggagaccacaacgactcctctgtgaagacgcttgggagcaagaggtgcaagtggtgctgccactgcttcccctgctgcagggggagcggcaagagcaacgtggtcgcttggggagactacgatgacagcgccttcatggatcccaggtaccacgtccatggagaagatctggacaagctccacagagctgcctggtggggtaaagtccccagaaaggatctcatcgtcatgctcagggacacggatgtgaacaagagggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaagcactgctcttatacggtgctgatatcgaatcaaaaaacagcatggcctcacaccactgctacttggtatacatgagcaaaacagcaagtggtgaaattttaatcaagaaaaaagcgaatttaaatgcgctggatagatatggaagaactgctctcatacttgctgtatgttgtggatcagcaagtatagtcagccctctacttgagcaaaatgttgatgtatcttctcaagatctggaaagacggccagagagtatgctgtttctagtcatcatcatgtaatttgccagttactttctgactacaaagaaaaacagatgttaaaaatctcttctgaaaacagcaatccagaacaagacttaaagctgacatcagaggaagagtcacaaaggcttaaaggaagtgaaaacagccagccagaggcatggaaacttttaaatttaaacttttggtttaatgtttttttttttggccttaataatattagatagtccaaatgaaattacctatgagactaggcttggagaatcaatagattctttttttaagaatctttgggctaggagcggtgtctcacgcctgtaattccagcaccttgagaggctgaggtgggcagatcacgagatcaggagatcgagaccatcctggctaacacggtgaaaccccatctctactaaaaatacaaaaacttagctgggtgtggtggcgggtgcctgtagtcccagctactcaggaggctgaggcaggagaatggcatgaacccgggaggtggaggtttgcagtgagccgagatccgcaatacactccagccctgggtgacagagcaagactcttgtctcacaaaaaacataaaacacaagggggggccgtcaacatgcttttaaagggcccaatttcgccttatagggagtcggttttacacaaattcaggggcccggcgttttaacaacgtccggaattgggaaaccccttgggtttaccccaatttaaatcgccttggaagatc

SEQ ID NO: 98 - DNA, 1314 bp, Homo sapiens
  misc_feature: Incyte ID No 7472392CB1
gcgagctgcgcgtggcctgggctccttccgaatggtggcgcctctgagcgccagttcgaggaggagtcggaggaggagcctgaatgtttggagatagacttcaagtcccggaccttatccgtgcgccgcttcggtttgcagggactgtaccagctcgccatggatatcatcataatgatccgagtgtgtaaaatgttccgccaaggcctcaggggattccgggaatatcaaatcattgagactgctcactggaagcaccctatcttctccttctgggataaaaagatgcaaagccgagtcacatttgataccatggacttcattgcagaggagggtcactttcctccaaaggccattcagatcatgcagaagaagccttcctggagaacagaggatgagatccaggccgtctgtaacatcttgcaggttctggatagctatcggaactacgcagagcccctgcagctgctcctggccaaagtcatgcgctttgaacggaaatggacgttctgcatgcttcagtgaggaggtccaccatcgtctgtatggaagaaacggagttcctggttgttgaccgggaggacttctttgctaataagctggaccaggaagttcagaaggatgctcagtatcggtttgaattttttaggaagatggagctgtttgcatcatggtctgatgagaagctctggcagctggtagccatggcgaagatagagaggttctcgtatgggcagctgatctcaaaagattttggagagtcacccttcatcatgtttatcagcaagggcagctgtgaagtcctgcggctgttggaccttggggcctccccttcctaccgtagatggatctggcagcacctggagctgatagatggcagacctctgaagacccacctgagtgaatactctcctatggaaagatttaaggaattccagatcaaatcatatcctctgcaagactttagctccttgaaacttccacatctcaaaaaagcctgggggctacaggggacaagcttcagcaggaagatcagaacctcaggagacactctccccaagatgctgggcccgaagatccaatccaggcctgctcagtcgatcaaatgtgccatgatcaatatcaagcctggtgagctccccaaggaggctgcagtgggggcctacgtgaaggtgcacactgtggagcagggagaaattttggtgagtgtgccaagagctctgttcaccatggagtacgtaacatgaatgggctggaatgttaagtaaatgacagtaaaggttttacaaacaaaaaaaaaaaaaaggggcggcg

SEQ ID NO: 99 - DNA, 3322 bp, Homo sapiens
  misc_feature: Incyte ID No 4028960CB1
ggagcccccacccaggcgcatggctccatgaagcaagaggaggaggcagagcgcgagaggtcccgtccgccccagccgcgtctgggccaggagaaaaggtacaggaagaaggaatggaatcggaaggaagaaaattcattccagagacaaaatagctcccctcctctgttgtagctgttcctctggcaatacaaactttctgctttcaggttctgcagcattccagaccttgagcactgaatcaggatgggaaaaagacgttgtgttcctccactcgagcccaagttggcagcaggctgttgtggggtcaagaagcccaaattatctggaagtggaacgcacagtcacgggaatcagtccacaactgtccccggctctagttcaggacctcttcaaaaccaccagcatgtggacagcagcagtggacgggagaatgtgtcagacttaactctgggacctggaaactctcccatcacacgaatgaatcccgcatcgggagcgctgagccctcttccccggcctaatggaactgccaacaccactaagaatctggtggtgactgcagagatgtgctgctactgcttcgacgtactctactgtcacctctatggcttcccacagccacgacttcctagattcaccaatgacccctatccgctctttgtgacgtggaagacagggcgggacaagcggcttcgtggctgcattgggaccttctcagccatgaatcttcattcaggactcagggaatacacgttaaccagtgcacttaaggacagccgatttccccccctgacccgagaggagctgcctaaacttttctgctctgtctccctccttactaactttgaggatgccagtgattacctggactgggaggtaggggtccatgggattcgaattgaattcattaatgaaaaaggtgtcaaacgcacagccacatatttacctgaggttgctaaggaacaagactgggatcagatccagacaatagactccttgctcaggaaaggtggctttaaagctccaattaccagtgaattcagaaaaacgatcaaactcaccaggtaccgaagtgagaaggtgacaatcagttacgcagagtatattgcttcccgacagcactgtttccagaacggcactcttcatgccccgcccctctacaatcattactcctgacacacggctgcatgaccagtcccacccccccgtggccaccaatggctatgacatcattggagcagatgcctcctcttcctggtccagttacttctattactgcaccattttatgatgctagcttccgttgccaagtctgcctcccgctgactgagggagggtagggttaccttgaacgaaacagaacttgaggggcccaagccttatctcagcctttcctcaatatggggttccgttggattggggctcctccatgactagtgggaattactgtgggttcagaagacccttgtctggtatttgccacatggggtattggccacacgctggaagctgaaattgatgatccctgaaggttgaacccacacacacccctgcagcctccccagatgaagtaggtgtattcccctggcagtctgggcaacggagaccaagaaacatttttaggttgttttaaattcctttttttaaacttccagtttattgcgtaccaagagttgattacaacctccatgcttcataagcggacgccacgttagggttggacgtgggcaccacgagtcctttgaggctcctggacagagacccacatcaagatcggaagccctttgggtggcgttgcagatctcattgctcagtaggccgtgaagattttcatcctcatcccactctcagttggattttctggcactcttcctgcattgagtctcctgattactgaacagagctccgtcatgtagcctgctgaggaatggaatggaatggagatgcccacaggaggtcctgatgtcatcactgcacgcaggtgtgagaggagagacctcttctgcaccgcctggctacctcactcctctgctggtagcagtgcctatagctggacctaagttctcagaagcgtagatgtgcaaacaagcgattgagttgggctttaggaggacacatcataggagagaatccagggtctgtaagctggttttcttttcaggtgacatcctgaggggcctgtaagcaggggagctcctttttctagtttgcctgtagaggtgggaagactgttggtgtttctgtcctttacaggacattaggaaacagttgtgtaattacacaaggtggacctttatcttgcctgacatgctgggaatcttcaccccaccagggcaaatttccaaatagctcattttattctaggtctttcaaactttcatgtgacatatttccctttcccattgttgctgatttccaaatcgctgtcagcaattttttcctctctccttgcctattcttcactcatttggtggcaaagttcatagaactaggggacttggaagatgctttgaaaatattgttacaaaggcactgctaaaatgattcacagggagagtggccagttggaagaaggatcctaaggatgtgacactggttttcaacaacatgcttagagaactcatgaagtggattgggtgtcaacccagtgaacatgtttttatttaatttattttttgaagtttatgtggtgatggtgtggctttccgaaatgggcaaatattcagaagatcttttgcattttcttctgccaggaatggggaaggggagtgggggcacaatctgagaaaggacacctgtgctgttctaggcatcgctggcaagtttgtgggaagggatgggcaagggtgagtgggtttgctccacaccgtcctgtgctgctcgagaggacctgggacgtgcgagggaaacgtgggtgacggtgcctaggctgcggcccttcactgctgtgctgggttcctgcagcctgctacgtttcccttggcaatgtaaatgaagatggaggggtcgtttcgtgatttcctgctgctgagaataaatgtcttgttaaaaacgtggcaacggttactcttaggtgtcatggatcgatgtcagggtggtcagctctggactaagccacccacctccaatttgtacaacagtattgatacatagggctacactcattactgttcaagtgttctatgttaagagttgtgtttaatttctaaagattaaaaaaagcaaaaaaattggtgctaaaccttcacccctgagcacgctcagtgagactggtcatgcaagcatttacagtgccatgctcctcaagaccattggcagg

SEQ ID NO: 100 - DNA, 3621 bp, Homo sapiens
  misc_feature: Incyte ID No 8227004CB1
cggtgagcgcgtttcccgcctgagcgcaactagcggcgggtcgtgggcacctccagaccagcatgttttggaaatttgatcttcactcatcatcccacatagacacacttctagaaagagaagatgtaacactgaaggagttaatggatgaggaagatgttttacaggaatgtaaagctcagaaccgcaaacttatagagtttctgttaaaagcagaatgtctcgaagatttagtctcattcattatagaagaaccacctcaagacatggatgaaaagatcagatacaagtatccaaatatatcttgtgagttgctcacttctgatgtctcccagatgaatgatagactgggagaagatgaatccttgctaatgaaattatatagcttcctcctaaacgattcccctttgaatccactacttgccagtttcttcagcaaggtgctaagtattcttatcagcagaaaaccagaacagattgtggatttcttaaagaagaagcatgattttgtagaccttattataaagcacataggaacttctgctatcatggatttgttgctcaggctcctgacgtgtatcgaacctccacagcccaggcaagatgtgctgaattggttaaatgaggagaaaattatccagaggcttgtggaaatagttcatccatcgcaagaagaagatcgacattcaaatgcatcacaatcactttgtgaaattgttcgcctgagcagagaccagatgttacaaattcagaacagtacagagcccgaccccctgcttgccactctagaaaagcaagaaattatagagcagcttctatcaaatattttccacaaggagaaaaatgagtcagccatagtcagtgcaatccagatattgctgactttacttgagacacgacgaccaacatttgaaggccatatagagatctgcccaccaggcatgagccattcagcttgttcagtaaacaagagtgttctagaagccatcagaggaagacttggatcttttcatgaactcctgctggagccacccaagaaaagtgtgatgaagaccacatggggtgtgctggatcctcctgtggggaatacccggttgaatgtcattaggttgatatccagcctgcttcaaaccaataccagcagtataaatggggaccttatggagctgaatagcattggagtcatattgaacatgttcttcaagtatacatggaataactttttgcatacacaagtggaaatttgtattgcactgattcttgcaagtccttttgaaaacacagaaaatgccacaattaccgatcaagactccactggtgataatttgttattaaaacatcttttccaaaaatgtcaattaatagaacgaatacttgaagcctgggaaatgaatgagaagaaacaggctgagggaggaagacggcatggttacatgggacacctaacgaggatagctaactgtatcgtgcacagcactgacaagggccccaacagtgcattagtgcagcagcttatcaaagatcttcccgacgaagtcagggaacgatgggagacgttctgcacaagctccttaggagaaactaacaagaggaacacggtagatctaatgcaacaaatgacgtccaattttattgaccagtttggcttcaacgatgagaagtttgcagatcaagatgacattggcaatgtttcttttgatcgagtatcagacatcaactttactctcaatacaaatgaaagtggaaatattgccttgtttgaagcatgttgtaaggaaagaatacaacagtttgatgatggtggctctgatgaggaagatatatgggaggaaaagcacatcgcattcacaccagaatcccaaagacgatccagctcggggagtacagacagtgaggaaagtacagactctgaagaagaagatggagcaaagcaagacttgtttgaacccagcagtgccaacacggaggataaaatggaggtggacctgagtgaaccacccaactggtcagctaactttgatgtcccaatggaaacaacccacggtgctccattggattctgtgggatctgatgtctggagcacagaggagccgatgccaactaaagagacgggctgggcttctttttcagagttcacgtcttccctgagcacaaaagattctttaaggagtaattctccagtggaaatggaaaccagcactgaacccatggaccctctgactcccagtgcggctgccctggcagtgcagccagaagcggcaggcagtgtggccatggaagccagctctgacggagaggaggatgcagaaagtacagacaaggtaactgagacagtgatgaatggcggcatgaaggaaacgctcagcctcactgtagatgccaagacagagactgcggtcttcaaaagtgaggaagggaaactgtctacctctcaagatgctgcttgtaaagacgcagaggagtgtcccgagactgcagaggcgaagtgcgcggcgcccaggcctcccagcagcagtcccgagcagaggactggccaaccaagcgcaccaggtgacacttcagtgaatggccctgtatgacgggtgacgtctgctgctgctgactgaggactgcagaccgccaccactcaggggctctggaggggtcagctggagcccaccaagctgtcactgctgcactcactctgcaagggatcaggaccagcaacctttatattctagattctaagacattgtacagagaaattcagaagtgtaaaaatattgcacattgacaaataccaagaatttttgcgtatgtttatattgtattgttctaaataatgggtagcctgtgaaataagatcttgccacccatgtaataatagtagtaatactatagttaaaatggctgtaagaatagttttataaaagtgaatacacagatctattgtatttgaaacataactttgacaattattagtgtgaccaaagtattaggcggttttcatacatttttcaccttgtacaaaattatgaattcatttttcctccaggccgacaaggagttgtagaatgaaaatgccctctaagtgttattttggttgttctaacttacaaaagtgattttgaataagaaatatttggtgttctttttataaccagtttttgattggtaattgttttctgtattgtttaaaacggatcaaaaatgtaagtctattggtagagattaagtatttattgctacatcatagttgataaattgatgttatcgtaaagccatatgttctgttcaagtcttgtttgcttgaaatgattattcctacaagtgaaacactagactatttggagtgtatatggcttgtgttttgggatttttttttttttttttggcttttgtttttgtttgtatttttggttcgtttggtagttcatctgccttttaacccattcaccaaaatttaccttgttaacaagcatcaccaatgaacatttcagagcaatctgcatatttaacagacctaaaataaatcctattaagcaagtcagttgaaaatgctcgtgctgctaatgggaataaagtgcgttccatttacagggcaagtatttttaaaataggaatcaaaatctgggcaccgaa

SEQ ID NO: 101 - DNA, 2704 bp, Homo sapiens
  misc_feature: Incyte ID No 3044763CB1
ctggctcagggggcggctgcctcgccaggcgccacgaagacccggcgtcccacaggtcccggaggccgctgcgtcgcggggagctgggctcgggccgccggcggttgcgccggggcagccccgcagggagagcaccatgaggactgactcgggggctcggctggaggagggccacctgcgtcctccacgagctcttccacctgtgccaagtcaagatgatattccacttagtcgtcctaagaaaaagaagcccagaacaaaaaacacaccagcaagtgcttctttggaaggccttgctcagactgctggtcgaaggccctctgagggcaatgagccatcaactaaagaactcaaagagcacccagaggctcctgttcaaagaagacagaaaaagacaaggctacctcttgaattggagacttcctccacccaaaagaagtcatctagttcatctttattacgaaatgaaaatggtattgatgcggagccagctgaggaggcagttattcaaaaacctcggaggaagacaaagaaaacccagccagcagaattacagtatgccaatgagctaggagtagaagatgaagacataatcactgatgagcaaactactgtggaacagcagtctgtattcactgcacccactggcattagccagcctgtaggcaaagtatttgtggaaaaaagccggagattccaggctgctgatcgttcagagttgataaagaccacagaaaacatagatgtgtcaatggacgtgaagccttcctggaccaccagagatgtggcacttacagtgcaccgggctttcaggatgattggtctcttttctcatggattcttggctggctgtgctgtgtggaatattgttgtgatatatgttctagcaggagatcagctatccaacctctcaaaccttctgcaacaatacaagaccctagcgtatccattccagagtcttctgtacttgcttttggctctgagtacaatttcagcttttgacaggattgactttgctaaaatatcagtagcaatccgaaattttttggccctggatcctacagctttagcatctttcttgtactttactgctcttatactatctctgagtcagcaaatgacaagtgacagaatccacctttacacaccttcttctgttaatggtagcctctgggaagcaggaattgaggaacagattctccagccatggattgtggtgaatctcgtggtggctcttctggttggattatcttggctatttttgtcttataggccaggcatggatcttagtgaagagttaatgttctcctcagaggtggaagaatatcctgataaagagaaagaaatcaaagcctcttcataataccagctcacctttggaggaataatgacccagtatttgtcccatttttgtttttaaatgtatttttataagatgtatacatgtgtatttgtaatagattttttgattatataatactgaaacatctctcaatattatgaaaaatgttaaaattgtgtttgctgtttatgtctaaacattaatttgtctagcattatcatcttaatgacaaagggaataatgaactagaaaccagcaagtgaaagtgtttatttcctattttctcaaaacagttgtatttataactattaccttaaaaagcactggtttagaaaaagccataacttaaatagtgttataaaatatatatcaggtttaaacataaatttagcgaatatggtagaagggaaaaaagccttcatttttgacctccccttactgaataaattgaaatatgaagtttgtcttttctgaaactggcttagtgattgagtatcatgtaataattataatataatttagcttgaaagatgctctacattatgaccaataaaaagggaatgtatgttttgtttgaaaaattatataacctaaatttcttacctagaagtaactaaaaagttgcttctcattataatctgtactagtggttctcatatctggttgtacatcaggatcatagagggagattttaaaaatctatgtagcaaaagaggctgagcagggcaaggctcaggggaataaaggagagacctgtgagcttgtgggctcccagttgacatctgcagtaccctttcctctgtcccttttttctgagtgacaagaaataggagtagaaattcaccatctctgttctccagctctttgtcagagaggtttctggttttccaggaaatccccctcttgagatgggtcctgcactgggaaagatctcttcagaactatatccaatggtacctcagctgttgtatatgagaccctaaagctcaagttgaggagaagactagctatgggaaaaaatgtttcaaaggctgttgagcacatcaataaaactattgagcctgccctgattagcaagcacctgaatgttatagaacagaagaggattgacaaattgatgatagagacagttgaccctgacaataggtctaaatttggagtgaacattatactgggaatctcttttgctgtttgtaaggctggagctgccgaaaagggattctccctgctgtcacagaattgtgaatttgctggcaattctgaaggcatcctgctagttccagctttcactgtgaccagcaatggttctcaatctggcaataagctggcagtataggagttcataatcttccccgtcagcaa

SEQ ID NO: 102 - DNA, 1345 bp, Homo sapiens
  misc_feature: Incyte ID No 4044519CB1
cagaattcaaattcaatcttgtcacatccctgtttaaaactttttggttactacttactgcttttaggagaaagaacaaaatcctcaggtggctgacagatctgccatgatctggttcccacctaccccttggctgggttccacaccatttcttgtctctctcagtgacaggacagttctgctaattttctttgagttaactaaatgcctaattccacccactcccagtagcccttcatttcaaaggattcacatgggctgctccttctgcttgaatgtgcttccttttttttcttttgttcttcactatggtagcttctacctgcccttcagatctcaggctcaaagattccttcctcaagaacatggtccctgcattgaaaggctgtttcaggacttattttatatgcttccttctcattctaattttccaattgaacccaagttccagtttaccatccagtttaccagtatatcttttttcttttctttcttttttttttttctttttttttcttgaggcagagtcctgccctgtcacccaggccgaagtgcagtggtatgatcatagctcactgtagtctcaaactcctgggcttaagtgatccttcagcctcagcctcccaagaatctggaactacaggtgtgcaccaccatacctggctagcttatctaatttttttgtatagccagagcctcagtatgttgcccatgttggtctcaaacttctggactcaagtgatagtccctccttggcctctcaaagtgctaggattacagacctgagctactgcacctgactactgttttccctttttaaatctatttattaatgcctatctctcctatgagactatcagcgcacacatacatcttttctatattcttcataactttgttcttataacttcctaccacatgacctgtcacacaatagccattcaaaaaacttgttcaatgaatgcacacatagattatttgcttctccgaatatgccacaaatgcatgttgatataatcttctgcaaattgagttccgtgttcgtatataaagtgtatgtactttctacatatgttgaatatatttagattatcttcctcattcacaaatgaagaaactaagatctaagggaggtaagtgacttttccagggtcactcagggaatttgtgaacagatcgtaaccaaaattcaggccagtaaactgacagtccagacagtttagtaaaattgaaactgcaaatggcgattgtcttcaactgacttgaatccaaatcctgaagttatttcttgttaagtctattactttgggaaatgttccctactgaagaaaaaaaaaaaaaaagg

SEQ ID NO: 103 - DNA, 1607 bp, Homo sapiens
  misc_feature: Incyte ID No 71351918CB1
ttgggcggaagaggtgggctggtggaggcggggtcgagatggcggcgcctttgaggattcagagcgactgggcgcaagccctcaggaaggatgaaggggaggcctggctgagctgtcatcccccagggaaaccatctttgtatggcagcctgacttgtcaaggaattggcctagatggcatcccagaggttacagcttcagaaggatttactgtgaatgaaataaacaagaaaagcattcatatttcatgtccaaaggaaaatgcatcttctaagtttttggcaccatatactactttttccagaattcatacaaagagtataacatgcctggacatttccagcagaggaggtcttggtgtgtcttctagtactgacgggaccatgaaaatctggcaggcttccaatggagaactcagggtttttcccatcaggccttgtggtcctgagtgggggaatggatgcccagctgaagatatggtcagctgaagatgctagctgcgtggtgaccttcaaaggtcacaaaggaggtatcctggatacagccatcgttgatcgggggaggaatgtggtgtctgcttctcgagatgggacagcacgactttgggattgtgggcgctcaggctgcttgggagtccttgcagattgtggttcttctatcaatggagtggcggtgggtgctgctgacaactccataaaccttggctcccctgagcagatgcccagtgaacgggaggttggaacagaggccaaaatgctgctcttggcccgggaagataagaaacttcagtgcttgggactacagagcaggcagctggtgttcctctttattggctcagacgctttcaactgctgtacttttctctctggcttcttgctattggctgggactcaagatggaaacatttatcagctggatgtgaggagtccaagggctccggtacaagtcatccacagatcaggagcaccagttctatccctgctaagtgtcagagatggattcattgctagccaaggtgatggaagctgttttattgtccagcaagacttagactatgtcactgagctcactggggctgactgtgaccctgtgtacaaggtagccacatgggagaagcagatctacacatgctgtcgagacggtcttgtacgacgctaccagctttctgacctctgacttcttggaaagagcagtcccggttagtgaaaaggtttgaccctgatcaacaatgagcagaaacatcatcagtccttcccaaggaccatggcgtttaatgtcttgggcaccccttggaaatcacagaaagtcagctgtactggccgtgtggaactctcatcccaagacctactttgaactgagtaagaaggtcattgtgcccactgcatttgttccaacttctccttgtataaactcaccccagcaacacagggcaaggatatagatgcttttagtttgttcttaaaccagttttgttaaatgtttacaaggacctcagtactaaagcctgttctctggaggaaataaagaaaatatgtttggaggtgcctgaatatgaagaactttgttaaatactcttactccagcaaaaagttggtcaggacgatgga

SEQ ID NO: 104 - DNA, 2622 bp, Homo sapiens
  misc_feature: Incyte ID No 8109363CB1
atcaattccctggtcccaggagaagttttattgtgagtttgttaatttaagaactcactcagcagaaggacatgtaatgtctgagtaatcttgatggaagaaaggaaatagatctgtgattaaaacacacacacacacacacacacacacaccctacttcggagagagaatgtagaatggaggaggtgaggaatttaactaatagagatgctttttctaacaggcagaaattagagaaatggatcaaaagatttaaacattgattacatataaacgagtttcctttttttctacatagcacacaggtgctcaacatttaatgaataatataacagatactgtcttaaaagtttaaaaacaggatttgtacttaatcctaaattcctcttatcacacaccagaaaataatttggattttatttacaaactacatccaaatactcttaaggaaagagtgtcatttagcttggagtagggactgactggggtctaagtgttggaaattaaattctgccattcagtccaacctcaaaggtcttccaggcaaggagttttgcaaggactagaaaccctttcacacttgaagtgcaggtgttggctgaaagaatagagggacatttcacaaacacagaacacggctcccctacaggaaagggatggagacagctacctttgctcttgctctcacattctctgtcctcatggactttcttctctgtttccctctgcctatctatctgcctggttctcttgcttctcctggccaaccagctttctcattagtgcatatgtagctgaaaaatccacccctcaagaacccactccacacaacctttccttttcagtacccaccacgctctgactagcatctctgtgatgcctaaggcaaatttttgaggatctgattggtttagttcatctgcctatgggtgagccaccctggggtcaggtgcctgtccaatcacctgttaccaaggaggcagggccgtgtcatttaagcatggccccatggactgaccctttcagcagggacttttattgggggctggtacccagaagaaggagcatgagtgggcaagaccttcaaaaagaacctactacacactggaactgactagtactcctgggatgccatgccacgcagcagccagcactcggagagcagtcccctggatactaccacacagagaaaaggggcatcttcacttgcacatcaggtgagggtgcacacacttgaaactctgctagactggccagagctgccccagccacttctcaccccaccaccagttattgatacagccgctgggtctaggaagcgatttctgaacaaggcccagttagcacaatgtcttgctcagcagacaataaatacttgtaaattgaattgcatgatcttagctcaggtgctactaatgtggctcacggccacacacttgcatggataagaaaaccaatgagcccctcaacagagtcttttgaaacaaaattggtttcaagtgaaacttacattattttttggctgtgaccttctgaatataataacttagtgtcctgagaattgaaaggcctcttgatttcttaggtgaactgttacagaatcctagcctgtggtaaatgctaaaatgtaaggcgtttctgagaaggaatatttaggaaatataattgtgccacacacacattactttagaagatatgcagttgctctgattctgcaggtttaaaaaaaatgggaagtttgtgatatggcagaatctgtctccattggtcaaaccatacttcagtgagagccattataagtgaaatctaaaaattgatcgttttcattctctgactttggtcagacttttgcaaattatggagagaggccatcaacatattttacataaattactgtggccccatcttatttctcctcatttcttccttatgtttaaacacgcccccatctcacacacaaactgagaaaagatttgtatcaaacagaatcagaggcatatgaatggactggaaattgtgaaaaaggtataaatatcctagaccttgatcaactcactttaaactggtctctgattcctcatctgtcaaatgagtataagaccctctgtacttcccatggtgattttgaggagcacatgcataatgtatctgaaaatggcttgacaagcacttgaagccattcttactaaattattagttttgtaattcggttttatccaggaaaatattttagctcctcctatctgctgaattaatttatacagtcacatgttccatgacaccatgttttgccaaggaaaaataaagaaattcctaaatttgaaattgcccacaagatctagcctcatctggagttgctgttattgtccttgttgtctatgatttggaggatatgtaagtacttgtacatgtgctttaactctgggtgaaaaaccccaaccggtgagtctttaaaaccatattagacacattgggcaaagtactgagcagaagctttgaatgtagctgttgttattttaatgttcgctttctaatgaactttgtacgtccttagcctctgccgggaacagagtgttcatatgctgttagaattttttattgtaaaataaaatgacaaaggct

SEQ ID NO: 105 - DNA, 3489 bp, Homo sapiens
  misc_feature: Incyte ID No 1272746CB1
cggcagtcgggaccgactgcaaggcaggttgagatgatggatctgaggcttgcagctcgcagtcccaacgagccttagggaccagggctaccaaaagggtggggagaacggtggtggcatctgtctgttgcctgggatccaggcgcccggttggggaatccttacttcaggaagcgcagccgcggctctgtgttttgagagtgcaaatgtcatttgtcagagtgaaccgctgtggtccccgagttggtgtaagaaagacaccgaaagtaaagaagaagaaaacttcagtgaaacaagaatgggataataccgtgactgatctaaccgttcatcgggcaactcccgaagatctggtacgccgtcatgaaatacacaaatcgaagaatagagcattagtacactgggaactccaagaaaaagctttgaagagaaaatggaggaagcagaaaccagaaactttaaatcttgagaaaagaagattgtctatcatgaaggagattctttctgatcaataccagatgcaagatgtgttggagaaatctgatcatctaatagctgcagcaaaagagctgtttcctcgtaggcgcacagggtttccaaatgtaacagtggctcctgattcctctcagggtcccattgtggtaaatcaagaccctatcacccaatctatctttaatgagtctgtcatagaacctcaggctcttaatgatgtagatggtgaagaagaaggaactgttaatagccagtcaggagaaagtgagaatgagaatgagttggataactctctaaactctcagtctaacacgaatacagacaggtttctccaacaactaacagaagagaattttgagttaattagtaagttgtggactgacattcagcagaaaatagcaacccagtcacaaataactcctccaggaacgccatcatctgctctttcatcaggggagcaaagagctgctctgaatgctaccaatgctgtcaagagactccaaaccaggcttcagcctgaagaatctactgagactctagactcaagctacgttgtgggacacgtgctgaactcaaggaagcaaaaacagctgttaaataaagtgaaaaggaaaccgaatttgcatgctctttccaagccgaagaaaaacatatcatcaggtagcacaacctctgcagacttaccaaataggactaattccaacctggatgtcctcaaacacatgatacatgaagtggaacatgaaatggaagaatatgagcggtggacaggtcgcgaggtcaagggtctgcagagcagtcagggtcttacaggcttcactttgtcgctggtgagctccctctgtcgcctggttcggtaccttaaagagagtgagatccagctacgtaaagaagtagagacaaggcaacaactggaacaagtattaggtgatcatcgagagctcattgatgctctgacagctgaaattcttcgtcttagagaagaaaacgctgctacacaggcaagacttcagcagtacatggtcacaacagatgagcaactgatatcactcacacatgctattaagaactgtcctgtgataaataacagacaagaaattcaggcatcagaaagcggagccacaggtagaagagttatggacagtccagagcgtccagttgtaaatgccaatgtctcagtgccattgatgttcagagaggaagtggctgaattcccacaggaagagttgcccgttaaactgtctcaggtgccagaccctccagataacatgaatctggccaagaattttccagcacatatttttgagccagctgtgttgttaacaccacccaggcagaagagcaacttaaaattctctcctcttcaggacgtattgagaaggactgttcaaactcgtcctgctccacgacttcctccaactgtggaaataattgagaaggaacaaaattgggaagagaagaccttacctattgatacagacattcagaattcaagtgaagagaatcgtctcttcactcagagatggagagtctctcacatgggagaagatttggagaacaaaactcaggctccttttgttaacctctcacagcccctctgcaattcccattccaacactcaacagtcaagaagccccacattctcagaagagctcccagtactgggagatgggcagcagctgagaacaaatgagtcattaatacaaagaaaggacataatgacacgaattgctgatttgacattgcagaattcagctatcaaggcacatatgaataatattattgagcccagaggagagcaaggggatggactccgggagttgaacaaacaagaaagtgcaagtgacatgacttctacttttccagtagcacagtctctaacaccaggtagtatggaggaacggattgcagaattgaatcgacaaagtatggaggctcgtggaaaactactgcagttgatagagcagcagaaacttgttggtttgaatctttctccaccaatgtcacctgttcagttacctctcagagcatggactgaaggagcaaagaggacaattgaggtatctattccaggagcagaagccccagaaagctcaaaatgtagtactgtctctcccgtcagcgggataaatacaagaagatcttccggggctactggtaattcttgttctccactaaatgccacctcaggaagtgggagattcacacctcttaatccaagagcaaagattgagaaacagaatgaagaaggctggtttgctctttctacccatgtatcataagtgaagtcaagtctcactgagtttgttcttaataatttattacttccctttccctgctctgacttttaagtttttatatccttctttcagataaaacctacaaagatcctgtgaattagtactaatgaaacagagaaataaagcaattattttttgactttctcaaataagttttcaacaaccaactgacctacagctccctgtgaatgaaactttgactgtagagaagttaaagttttgtattttaatactttcttaagtattttaagggtttttttttattaatacatttttacctttatctttattcagggttttttgaggtttagtatatcttagttgatccatttatttatttattttcctgaagaactaatttgctttgcatgtaacttacaataaaattcctctgtgtgattagagtctggctttcaggaatatgtaacacccacttttctttcttttttcttgggaagtcattgctgttcatcactttttccatcaagtgaaatatacagccttaaaaacaataggcctgggagtgtttgtttatatttgcttaagcaggtaagagtggttgcatttattataaaatcttatgtagtttttaaatgtgaaaatgtcatcaataatggtaatgcaaatcaaataaatatgattctagagctcaaaaaaaaaaaaaggggggggcgccgaataagtgggttcgtgaacg

SEQ ID NO: 106 - DNA, 2269 bp, Homo sapiens
  misc_feature: Incyte ID No 1839974CB1
ggctggaggaggagagcggcggcggcgggagcagcgaagggggcggcaggatcctccaggctgccggctgggaaggcgtgggcgacccggtgtgtggcgcgcccagagccccgcgtttcagccctagggaaggaagccagttgagggaagttctccatgaatgtacgtcacaatgatgatgaccgaccaaatccctctggaactgccaccattgctgaacggagaggtagccatgatgccccacttggtgaatggagatgcagctcagcaggttattctcgttcaagttaatccaggtgagactttcacaataagagcagaggatggaacacttcagtgcattcaaggacctgctgaagttcccatgatgtcacccaatggatccattcctcccattcatgtgcctccaggttatatctcacaggtgattgaagatagtactggagtccgccgggtggtggtcacaccccagtctcctgagtgttatcccccaagctacccctcagccatgtctccaacccatcatctccctccctatctgactcaccatccacattttattcataactcacacacggcttactacccacctgttaccggacctggagatatgccgcctcagttttttccccagcatcatcttccccacacaatatatggtgagcaagaaattataccattttatggaatgtcaacctacatcacccgagaagaccagtacagcaagcctccgcacaaaaaactgaaagaccgccagatcgatcgccagaaccgcctcaacagccctccttcttctatctacaaaagcagctgcacaacagtatacaatggctatgggaagggccatagtggtggaagtggcggaggcggcagcggtagtggtcccggaattaagaaaacagagcgacgagcaagaagcagcccaaagtcgaatgattcagacttgcaagaatatgagttggaagtaaagagggtgcaagacattctttcgggaatagagaaaccacaggtttctaatattcaggcaagagcagttgtgttgtcctgggctccccctgttggactttcctgtggaccccacagtggtctttccttcccctacagttacgaggtggccttatcagacaaaggacgagatggaaaatacaagataatttacagtggagaagaattagaatgtaacctgaaagatcttagaccagcaacagattatcatgtgagggtgtatgccatgtacaattccgtaaagggatcctgctccgagcctgttagcttcaccacccacagctgtgcacccgagtgtcctttcccccctaagctggcacataggagcaaaagttcactaaccctgcagtggaaggcaccaattgacaacggttcaaaaatcaccaactaccttttagagtgggatgaggtaagcttattttcatattcacccatctaaaacaccatttcaaaaacaaaattaactgaacaaatgccatcttattcttttataagacatgctaaacacagaaaaaaggaatgctagtagagaaatttaaatcaatagttattgactatggaaaatgaagttagattgaagtagtattatgagttgtgtattgaaatatatgtacctggcttatttgcaagcactactgctgaaagaaaaaaagtggggtcttatctattttggatctccagtgtctagccagccaataatttgtattaaaaattgactgctttgtcatcttgactgctcagatgattttgataaggtaaaacacactgccgcttggaaagcataatttgcttttcctttttgaatgccatgtatgtactttggtgttacactgttgagtggcctctgtgagagcagagcagtacatagaagtcacattctacccatgtgcgagtgccctgtgggttagaatgtcctgtatcggatgccccccttgctgttcatgtcaccagaggcccttggaaggtatctacggtggagtgtccttattctcaaagccactgtgtgccaactggtggacccagtgcatttctggcctagctaactctacacacagaatctcaggcaggaaacacttgacctagaagcctttcatttctaaaaagcaactgtaggtcgggcgcgatgactcacgcctgtaatcccagcactttgggaggccaaggcaggtggaacacgaggtcaggagatcaagaccatcctggctaacacgatgaaacaccgtctctactaaaaatacaaaaaaaaaaaaaaaaaaagatcttt

SEQ ID NO: 107 - DNA, 3075 bp, Homo sapiens
  misc_feature: Incyte ID No 1877336CB1
aagaagaccagagccagccgggtggcacagcggtgtcgtggccgtgttgctgatcgcctgggtggttgttggcgtgtccctgcagcgaaggatcctggttgacagtgaaaaagcagtctggctcccgaggtccaccccttataccccaaggtccagatggcggccaacgtgggtgatcaacgtagcacagattggtcttctcagtacagcatggtggctggggcaggccgagagaatggcatggagacgccgatgcacgagaacccggagtgggagaaggcccgtcaggccctggccagcatcagcaagtcaggagctgccggcggctctgccaagtccagcagcaatgggcctgtggccagtgcacagtacgtgtcccaggcagaagcctcagctttgcagcagcagcagtactaccagtggtaccagcagtacaactatgcctacccctacagctactactatcccatgagcatgtaccagagctatggctccccttcccagtatgggatggccggctcctatggctcagccacaccccagcagccatccgcaccccaacaccaagggactctgaaccagcccccagtccccggcatggatgagagcatgtcctaccaggctccccctcagcagctgccgtcggctcagccccctcagccctcaaatcccccacatggggctcacacgctgaacagtggccctcagcctgggacagctccagccacacagcacagccaggcggggcccgccacgggccaggcctatgggccacacacctacaccgaacctgccaagcccaagaagggccaacagctgtggaaccgcatgaaacccgcccctgggactggaggtctcaagttcaacatccagaagcgaccctttgctgttaccacccagagctttggctccaacgcagagggccagcacagtggttttggcccccagcccaaccctgagaaagttcagaaccacagcgggtcctctgcccgggggaacctgtctgggaagccggatgactggccccaggacatgaaagagtatgtggagcgctgcttcaccgcctgtgagtcggaggaggacaaggaccgcacggaaaagctgctcaaggaggtgctgcaggcgcggctgcaggacggctcggcctataccattgactggagccgggagcccttgccggggctgacccgggagcctgtggctgagagccctaagaagaagcggtgggaggccgctagcagccttcaccctcctagaggggcaggctcggcgacaaggggcgggggtgccccgtcccagcgagggacgcccggggctgggggtgccggtcgagcccggggcaacagcttcaccaagtttggcaaccgcaacgtcttcatgaaggacaacagctcttcttccagcacagactcccgctcccgctcctcctccaggtccccgacgcgccacttccgcagaagtgactcccactcagactccgacagctcctactcagggaatgagtgtcaccctgtgggccgcaggaacccgccccctaagggccggggcggtcgaggggcccatatggatcggggccgaggcagggcgcagcgtgggaagaggcacgatctggcgcccaccaagcgcagtcgaaagaagatggcggcgctggagtgtgaggacccggagcgagagctgaagaagcagaagcgggcagcccgcttccagcacggacactcccgccgcctgcgcctcgagcccctggtgctgcagatgagcagcctggagagcagtggggctgaccctgactggcaggagctgcagatcgtgggcacctgccctgacatcaccaagcactacctgcgcctcacctgtgcccccgacccgtccaccgtgcgccctgtggcagttttgaaaaagtcgctgtgcatggtcaagtgccactggaaagagaagcaggactacgcgtttgcctgcgagcagatgaagtcgatccggcaggatctgacggtgcagggcatccgcaccgagttcacggtggaggtgtacgagacccatgcccggatcgccttggagaagggtgaccatgaagagtttaaccagtgccagacgcagctcaagtcgctgtacgccgagaacttgcctggcaatgtgggcgagtttactgcctaccgaatcctctactacatcttcaccaagaactcgggagacatcaccacggagctggcatacctcacacgagaactgaaggcagatccttgcgtggcccacgccttggcattaaggacagcctgggccctgggcaactaccaccgctttttccggctctactgccatgcaccctgcatgtctggctacctcgtggacaagtttgcagatcgggagcgcaaggtcgccctcaaggccatgatcaaaaccttccgccctgcgctgccagtctcctacctgcaggccgagctggccttcgagggcgaggccgcctgccgggccttcctagagcccctgggcctggcctacacgggcccggacaactccagcatcgactgccgcctcagcctggcgcagctgtcagccttctgagcacccagcgaggaggggcgggggcaggggctgcagcccccagcgctgcctttgcggattctgtttttgagccgtggacttgggttgtaaatttatttgtggggagtgcgctccaggaagagccaccatccctgcccccgttttcccttggttcagcacaggtaaaacgggttcccctccctccctgccttcatggatcaccagctcacgtcatgttgccttctcttttctttgtgtgtgtgtttatttaagttatttttcttcctcctctcccttttctttttggccctccctccctccctcttctgccatgtaactggaggatgtgctatgagtttgcaaacagctggactgtcaggctgctttttttccagatgttcctcctctgcctccccttcccctcctctcccctccttttccttccttccttcctttccttggagcactgagcaccatttggaagcttgagagaaaccaaaattaaagagagaaagagagagcgtgcaaaaaaaaaaa

SEQ ID NO: 108 - DNA, 849 bp, Homo sapiens
  misc_feature: Incyte ID No 2321054CB1
cgtccctagagcctcgccgccctgggacccgggctgcttcctgggagaggattacattttctccctgctccacttgactttcttccggaaggagcaggtctcgcaggatcggggccaagactggaagagttcagccccatggctgggggttatggagtgatgggtgacgatggttctattgattatactgttcacgaagcctggaatgaagccaccaatgtttacttgatagttatccttgttagcttcggtctcttcatgtatgccaaaaggaacaaaaggagaattatgaggatattcagtgtgccacctacagaggaaactttgtcagagcccaacttttatgacacgataagcaagattcgtttaagacaacaactggaaatgtattccatttcaagaaagtacgactatcagcagccacaaaaccaagctgacagtgtgcaactctcattggaatgaaacctcagaaaaagagcaacagaagtaattgtttcaagctcctgattctttctactaaatcatgaacagctttaaaaacatttctgtctgcataaaattattttacttgtaacttttccccaattgttctgtgcattgttttgcctttttaaattacatctccaagtggctcaaaaggccttgacacagggaacctgcacatatccaggatatgtgtaaccagcgatggtgacttgaccttgccaagacctgtgattccttcaggatacaatcagtgagaaataaaaacacatcttgggaagtgggaatcctggagtttatgccatttgcaatattaaaaaataaaaatgcaagttattatttcaataataaaaaaaaaaaaaaaaa

SEQ ID NO: 109 - DNA, 2659 bp, Homo sapiens
  misc_feature: Incyte ID No 2796034CB1
atatactttttcatttctttgtatctacaatggccaactggaatttctgcttgaaacatggagcccaccataatctcctttttccagtcttctgatggaatataatcttcatcttcttcagattcttcttctacttcactatttgtatcaaaatatttacatcgacgtggcggattatttcctgggcatcttgagaagcaacagattgtgatggatcatcattggaagactgagtttcatcctcctgacctgatgaatcctttatattctcctctttattttccccactacagccactgttgtcatcattatcagcatcttcatcatcttcaccttcttcttcctcttcttcctcttcctcatcttcttcaggtagtcgaacagtactaccataaccataaaggctgagaagttcatgaattggcatgtcgccttcccttgcaagatcttctatttcagagctgaagtttgtttctccttccatcatttcttcctcttctaatgttcgttcatcatcaaaatcatgaaccagcatgtcagctgatggatcaaattcatggtcatctgatgttgctgaacctcctggacttgaagattcaacagatggcagcatcacatccgtcttgtctgcattgaacctcaaccagttggttcttaaccacgtccgaaactcagccaggcactgggagaatcgagcgaccacaccatagccgctgccgctgctgccgccgccaccttcgctgctgccgccaccgcctgaagaagccccatccatccgcctgcagccgggagactcagagcctgggaggggcctgaggcccgggccgcccgcctccttcttccagtcgtcgtccggagcttcctccagctccaccaaccctctgccctcccctccgcctccgccctcctcctcggcctcgtgccgccagccaaccgcgcggccgcttgttgagccaggctccaccgtcgccatgacgcccggcaaacattccggagcctcggcccgagccgctaacgcaggagcttgggggtacagggacttcagaggcggccaaaaaaaggggtggtgcaccactccccagctggtcgccaccatgccagtctctccggcaggcagtcacaagcagcagaacttcgggttgaacaatgccacacaaccaaagaagtctattagcttttttgctacaatgaaagcaacttcagtgaaaggatatactggtgcaaatcaaagcagaatggctgtgtccaaaaccgtgcttattccacctgaactgaaaactgtagaaaaaccaaatcccaatataaagacaacacaggtatttgacataaatggaactgatgttactccccgacctctttaccatccagatccacttactggtacagcaaaaccaagtaaactcttgacatcacaagaaggatcacttggatcagaatttatatcttcctatagcctttatcagaatacaataaatcctagtacgttagggcagtttacaaggtcagttttaggaagcagtacagtttctaagtcaagtgtatcagcaagtgaatcaatagcagaagacctggaagaaccatcctataaacgggaaagattgactagtttcacagatttgcaagttataagggcagcacctgaaaaaattgtaacaaaagaagacctggagaagaatatagagataatactcacagagacagaaacactgagattttttgacttgcccacagtcatggtctctgtagaatctgaagaagctgagaaagtaactcagagaaacaagaattatgaagtcctttgtagaaacagattaggcaatgacctatatgttgaaaggatgatgcagactttcaatggagcaccaaagaataaagatgttcaatgtgataaaatcataatggaagataaaggcataatgtccactgcctgggatttgtatgattcttacaatgctatggaacttgtatctttatcagtaaaacaatctgtggttgagtcaagtagtaaagcaaatgtacttcctaaagatcaggaccaaagattgccagggagcactacagaaaaaaatagtgaaactagttctctaatggacatagaaaatgtaattctggcaaaaatccatgaagatgaggaagaccactcagatgcaatattaaaatctgacaaatttcatcaggacttattttttatggaacgggttctgatggaaaatatatttcagcccaaacttgcagcttatcgtcagcttcctgttttaaaagaacctgaacctgaagagcctgaagatgttttagaaagtgcaaaacatgaagaggtagaggaagaatctaagaaggaggaggaagaagaaatacatgcagaagaatcaacaatacccgccaacttggaacgactttggtctttttcctgtgacttaaccaaaggcctcaatgtgagcagccttgcctggaataaaacaaatccagatcttttggctgttggctatgggcactttggatttaaagagcaaaaagaggactggcttgctgctggtcaataaagaatcccatggtaacccaaacaagaataagaagcatttttatctggctgttgacaaaattctcataaaatatataattacatgtaataattaaataaagtctagaaaattttatcaaa

SEQ ID NO: 110 - DNA, 2205 bp, Homo sapiens
  misc_feature: Incyte ID No 4413112CB1
ggaacgccagcgaccccagcagcgctgcggacggtgctggccgtggccgctgcggcccccgtgtccaggtgggccaggacgcagcctctgggcgccgtcgcttttccagcatcgcagaggcaaaagcgtggcagtgggacccaaaaggtaggactgaggctctagaacttgcacctgtgcagggactgcaaaccagacctgggaggaccctttcagcagcccccactccaccctatcccaggacttcccagcgacccgccgttctgggagataccgggagcgtgatcagggggcggggccgtttccaaggcaaccgcttatttgcatagggtcccgtcctggccaacgagggcgccccaaatgttcaggacatagaagaaggggttaactggcccggatctcctcctcgccttccaagcccgctaagcactggggttatctacccattccccagaaggggagactgaggcagcccaccagccaaaggaggcgaccagactggggctgcgttttaccatttcagaagcggcttgagctggtctgagctataataataaacactggcggtggaggcgagggcgaccacagggctgaggtcagggctaggattccggtgtctctacgtaggttgcttgaatggggggcaccctggcatggacgctgctgttgccgctgctgctgcgggagtcagacagcctagaaccgtcgtgcaccgtgtcctccgcggatgtggactggaacgcggagttcagtgccacgtgcctgaatttcagtggcctcagcctgagcctgcctcacaaccagtctctgcgggccagcaacgtgattctccttgacctgtctgggaacggcctgcgagagcttccagtgaccttctttgcccacctgcagaagcttgaggtcctgaacgtgctacgcaaccccttgtctcgtgtggatggggcgctggccgcccgctgtgaccttgacctgcaggccgactgcaactgtgccctggagtcctggcacgacatccgccgagacaactgctctggccagaagcctctgctctgctgggacacaaccagctcccagcacaacctctctgccttcctggaggtcagctgcgcccctggcctggcctctgcaactatcggggcagtggtggtcagcgggtgcctgcttcttggacttgccatcgctggccctgtgctggcctggagactctggcgatgccgagtggccagaagccgggagctgaacaaaccctgggctgctcaggatgggcccaagcccggtttaggcttgcagccacggtacggcagccggagcgcccccaagccccaagtggccgtgccatcctgcccctccactcccgactatgagaacatgtttgtgggccagccagcagccgagcaccagtgggatgaacaaggggctcacccttcagaggacaatgacttttacatcaactacaaggacatcgacctggcttcccagcctgtctactgtaacctgcagtcactgggccaggcctcaatggatgaagaggagtacgtgatccccgggcactgagcctaagatgtcctaacctccacccagaaccccttcagtccctgctgggtgactcagggcgtcctaactcctccatggcctcagtttccccatctgaagaatggggacaggaaaggattgtccttgaggccccaggaagctctgccgccccctccctgtccctcatgccgctcctcagctccctcagctcctagagggggaagaggagagacccccaacaaggggacaggagggtcactgtgccaatcctgtcatcaccctcctgtggatgtacaggcagtgctcaataaatgcttcgaggctgatgaggctgctggctcagggtgcgtgggttcctcaaggtggggatttctgagttctaagaccaagtctccatctgagactcccaaattgctccccacctcccatccctgtttttttttgttgttgttgtttgtttgtttgtttttgaaactgagtctcactctgtcacccaggctggagtgcaatgctgcgatctcagctcactgcaacctccgcctcctgggttcaagtgattctcctgcctcagcctcctgagtagctgggattacagcacccaccaccatgccgagctaatttttgtatttataatagagatggggtttcgccatgttggccaggctggtctcg

SEQ ID NO: 111 - DNA, 3042 bp, Homo sapiens
  misc_feature: Incyte ID No 7654832CB1
accagctgatattttctttcctcaggaccagctgttcaggagcatcccggacgagagccaggcacatttgagacttggatccaactaaagaccgccgcagattcttctgcagcaatgtcggtgttagaagaaaatcggccgtttgctcaacaattatccaatgtctactttacaatactttcgctgttctgttttaagctttttgtgaaaatcagccttgccatcctcagtcatttctacatagtgaaaggcaaccgcaaggaagcggcaaggatagcagctgaattttatggagtaacccaaggacaaggttcctgggcagatcgatcaccactacatgaagcagcaagtcaaggtcgccttcttgctctgagaacattattatcacagggttataatgtaaatgcagtaaccttagaccatgtcaccccattgcacgaagcctgccttggagatcacgtggcatgtgccagaactctgctggaagcaggagctaatgtaaatgcaatcacgatagatggcgtgactccgttattcaacgcatgctcccaaggcagtccaagctgtgcagagctgcttctggagtatggtgccaaagcccagctggagtcatgtcttccatccccaacgcatgaggccgccagtaaaggtcaccatgaatgtcttgacatcctgatatcctggggcatagatgttgaccaagaaattcctcatttgggaactcctctctatgtagcttgtatgtcacagcaattccattgcatctggaagcttctttatgctggtgctgacgtacagaaaggcaaatattgggatactccattacatgctgctgctcaacaatccagcacagaaattgtaaacttactgctagaatttggagcagatatcaatgccaaaaatacagagcttctgcgacctatagatgtagctacgtctagcagtatggtggaaaggatattgcttcaacatgaagctaccccaagctctctttaccaactttgccgactctgtatccgaagctacataggaaaaccaagattgcaccttatcccacaactccagctgccaacgttactgaagaatttcttacagtatcgataaaacagtaaagtaattctaaataccttgaaaatcaaaatttctatttcttttgcttaaggaatagttcatataaaaatatgctaaagataggataaaagtgagtgtgagatcacccagggaagcagtaaaatatcaattttcattttaagtgtattagtactattgttttatcatttatgctgtctttggattatagcatatttataatatctattttttgttatttcaatataccctcttgtttattcaggaaaaaataataaagtcactatcttatgttttcttttggattggaaagaaataaagctcctagtaaagttattcatattatatttaaaaaggtctatgttcttcaactaatgggttaactttttaattttggctattgtaattggctaacttgttactgatattgacataaaatatgaatatggcttagcaaagagcaactaatatgttttctgaaaccaggatgatgctcattcaggaaatccactatgttgaatgtccactggtcattagaatgaacacttcataaagataaattgcccccctcccccccacttacccctggcaaaggactcacaagtcacttctcttccccatagtacatggacttgttacagaaaagtgataggatgaaaggtcaatactgccctctcaccctgagatatttagaacattacacaccttctaaggctatgttaagactgcctccttctcaggagtagcccctgatgatctcttatcaaggcggttatatgcatattactccagccttcttccatttcagaaattgtcaatagctagtgatacttggacttaagaaaatgaactatatatctaattaacaaacaggccctggatcatattccggggatcatcacatcagtccaaggatataaaaaaatggaagattaaaaactcaaattgtggagtttttaaagagtgggtgccacttaacacattggagaccttaaaaaatgtatatagagaacttaatcgcactgtttctaattctttttacacaaaacaagggcttagagtatactgtatttgcaagtaatagcaagagagtattgtataaaagaagatattactaagctagtcctctgcaagggtaaatactatgtttattctcttcagctttaactatacttgagtgcaataaaaaacttaataaaattttatgcctgttttgtaattcagggcctattatgattttagaaataagataatcaacgtacaactctttgtaaaacaaaagcaactttgggtgagttttatagatttattaaattttgataaagtaaatattttgaccactattttcaacaacccagggcttattttcttctgacttcttggctaacttccagactatgggaaaataaattatgattgtatgtgacccccagatcactaagcttgcattttggcagaaagaaaaaaaatcagttgtgatttaaaacgtttgttgagattttcaaacatagaaataaagctgtagcatattcactaaacaatgttttatatgaggatagggacctttaaatatgtgttttatagaatatctcacacatttgtaatggataacgtgcattaaaatggagttcttattatagaataacctggttaaacagatattaaagctgcttttgggtaaaacaggtttccaccagtattttccctgagctagaggagaagtcttagattttaagagtaatacaacccatctatcttattagtcttgtgccaataagagaagtaaatatcaaacattaaaatggtcactttacaatttcagtatgtctgtaccttgtatcttagcttgagtatgattgtcttatcaaagtagtgttctaactttaatgtattgtatgtttgttattatattaaagtgtgattattattcattggaataaaaaaaaaaaaaaa

SEQ ID NO: 112 - DNA, 2112 bp, Homo sapiens
  misc_feature: Incyte ID No 7503849CB1
ggcctccggtgtgggatggccgcggagccgggcggagctggcttgcggctcccggggccggctctccggccggagacatggcccgggggcccggcccgctaggcaggcctcgccccgatacggtcgccatgcccaagagaggaaagcgactcaagttccgggcccacgacgcctgctccggccgagtgaccgtggcggattacgccaactcggatccggcggtcgtgaggtctggacgagtcaagaaagccgtagccaacgctgttcagcaggaagtaaaatctctttgtggcttggaagcctctcaggttcctgcagaggaagctctttctggggctggtgagccctgtgacatcatcgacagcagtgatgagatggatgcccaggaggaaagcatccatgagagaactgtctccagaaaaaagaaaagcaagagacacaaagaagaactggacggggctggaggagaagagtatcccatggatatttggctattgctggcctcctatatccgtcctgaggacattgtgaatttttccctgatttgtaagaatgcctggactgtcacttgcactgctgccttttggaccaggttgtaccgaaggcactacacgctggatgcttccctgcctttgcgtctgcgaccagagtcaatggagaagctgcgctgtctccgggcttgtgtgatccgatctctgtaccatatgtatgagccatttgctgctcgaatctccaagaatccagccattccagaaagcacccccagcacattaaagaattccaaatagcatagcttgtgtgggacactggagccgttgtgatggcagcagaagtgttttccccttaaagccaagcccattaattttgatggaacagcaggacgtacagggcatgtctgaagggcaggacagctggcacggcggacgacccaccccttatcccctgggagtgcttacttttctggtgcagaaagattgttgggaacagacaggaaccaatgtgggaattcaacttcaagttcaaaaaacagtcccctaggttaaagagcaagtgtacaggaggattgcagcctcccgttcagtacgaagatgttcataccaatccagaccaggactgctgcctactgcaggtcaccaccctcaatttcatctttattccgattgtcatgggaatgatatttactctgtttactatcaatgtgagcacggacatgcggcatcatcgagtgagactggtgttccaagattcccctgtccatggtggtcggaaactgcgcagtgaacagggtgtgcaagtcatcctggacccagtgcatcagcgttcggctctttgactggtggcatcctcagtacccattctccctgagagcgtagttactgcttcccatcccttgggggcagcctcgagtgtagtccattagtaatcagattccagtttggacagggtggctggattgtatatctcgttagtaatgtacatgctcttcaggttctagggctcctgttaggggagggagaaatgttgaatcaagagggaaaacaactactatgatttataaacatattttaatgtaaaaatttgcatttaaaaggagtggccctgttttctgtgttaaaaccccatttggtgctattgagtttgttctttattcttttatcccagtgaaaattgttgatcttgctgtagggaaaaattaaactctttgaatctccaaacaaggaagtttcagcattcccttatggatcagaggaaccttagaggcctgaaattgttgcttccagtttagctgcccctcaaattcaagtgaatattttcccttctccctttacccttctccagaaataaagcaggtgacagggttttcagaatcttaccatattgacttgtgtatcttttttaaaaaataatttgtgatatttattgcatacattttcttttggcagttttggattttaggtatttttttgcatgaggctgataatggtgatgatgactttatacccttggtactatactaggaactttacatagtatctctacttctcacagtcttgcaaagtaggtgatatttattcccattttatagtttgagaaaacggaattaggtgactg

(19) United States
(12) Patent Application Publication
(10) Pub. No.: US 2004/0249128 A1
(43) Pub. Date: Dec. 9, 2004

(54) Molecules for disease detection and treatment

(76) Inventors: Michael B Thornton, Oakland, CA (US); Janice K Au-Young, Brisbane, CA (US); Yalda Azimzai, Oakland, CA (US); Olga Bandman, Mountain View, CA (US); Ines Barroso, Cambridge, CA (US); Mariah R Baughn, Los Angeles, CA (US); Shanya D Becha, San Francisco, CA (US); Mark L Borowsky, Needham, MA (US); Li Ding, Creve Coeur, MO (US); Brendan M Duggan, Sunnyvale, CA (US); Vicki S Elliott, San Jose, CA (US); Brooke M Emerling, Chicago, IL (US); Ian J Forsythe, Edmonton (CA); Ameena R Gandhi, San Francisco, CA (US); Kimberly J Gietzen, San Jose, CA (US); Ann E Gorvad, Bellingham, WA (US); Jennifer A Griffin, Fremont, CA (US); Rajagopal Gururajan, San Jose, CA (US); April J A Hafalia, Daly City, CA (US); Huijun Z Ring, Foster City, CA (US); Craig H Ison, San Jose, CA (US); Karen Anne Jones, Bollington (GB); Preeti G Lal, Santa Clara, CA (US); Ernestine A Lee, Kensington, CA (US); Sally Lee, San Jose, CA (US); Joana X Li, Millbrae, CA (US); Dyung Aina M Lu, San Jose, CA (US); Joseph P. Marquis, San Jose, CA (US); Patricia M. Lehr-Mason, Morgan Hill, CA (US); Narinder K. Chawla, Union City, CA (US); Chandra S. Arvizu, San Diego, CA (US); Bharati Sanjanwala, Los Altos, CA (US); Thierry R. Sornasse, Mountain View, CA (US); Anita Swarnakar, San Francisco, CA (US); Y Tom Tang, San Jose, CA (US); Kavitha Thangavelu, Sunnyvale, CA (US); Bao Tran, Cupertino, CA (US); Uyen K. Tran, San Jose, CA (US); Bridget A. Warren, San Marcos, CA (US); Yuming Xu, Mountain View, CA (US); Monique G. Yao, Mountain View, CA (US); Henry Yue, Sunnyvale, CA (US); Huibin Yue, Cupertino, CA (US); Yeganeh Zebarjadian, San Francisco, CA (US); Hsin-Ru Chang, bELMONT, CA (US)

Correspondence Address: INCYTE CORPORATION, EXPERIMENTAL STATION, ROUTE 141 & HENRY CLAY ROAD, BLDG. E336, WILMINGTON, DE, 19880, US

(21) Appl. No.: 10/483,512

(22) PCT Filed: Jul. 9, 2002
(86) PCT No.: PCT/US02/21767

Related U.S. Application Data
(60) Provisional application No. 60/304,298, filed on Jul. 9, 2001.
(60) Provisional application No. 60/305,324, filed on Jul. 13, 2001.
(60) Provisional application No. 60/307,003, filed on Jul. 19, 2001.
(60) Provisional application No. 60/308,185, filed on Jul. 27, 2001.
(60) Provisional application No. 60/310,096, filed on Aug. 3, 2001.
(60) Provisional application No. 60/311,551, filed on Aug. 10, 2001.
(60) Provisional application No. 60/363,649, filed on Mar. 8, 2002.

(51) Int. Cl. C07H 21/04; C07K 14/705; C12N 5/06

(52) U.S. Cl. 530/350; 435/69.1; 435/320.1; 435/325; 536/23.5

(57) ABSTRACT

Various embodiments of the invention provide human molecules for disease detection and treatment (MDDT) and polynucleotides which identify and encode MDDT. Embodiments of the invention also provide expression vectors, host cells, anti-bodies, agonists, and antagonists. Other embodiments provide methods for diagnosing, treating, or preventing disorders associated with aberrant expression of MDDT.

### TECHNICAL FIELD

The invention relates to novel nucleic acids, molecules for disease detection and treatment encoded by these nucleic acids, and to the use of these nucleic acids and proteins in the diagnosis, treatment, and prevention of cell proliferative, autoimmune/inflammatory, developmental, and neurological disorders, diseases treated with steroids and disorders caused by the metabolic response to treatment with steroids. The invention also relates to the assessment of the effects of exogenous compounds on the expression of nucleic acids and molecules for disease detection and treatment.

### BACKGROUND OF THE INVENTION

It is estimated that only 2% of mammalian DNA encodes proteins, and only a small fraction of the genes that encode proteins are actually expressed in a particular cell at any time. The various types of cells in a multicellular organism differ dramatically both in structure and function, and the identity of a particular cell is conferred by its unique pattern of gene expression. In addition, different cell types express overlapping but distinctive sets of genes throughout development. Cell growth and proliferation, cell differentiation, the immune response, apoptosis, and other processes that contribute to organismal development and survival are governed by regulation of gene expression. An example of a mammalian apoptosis-associated protein is Diablo, which can bind to apoptosis inhibition proteins and antagonize their antiapoptotic effect, a function analogous to that of the proapoptotic Drosophila molecules, Grim, Reaper, and EID (Ekert, P. G. et al. (2001) J. Cell Biol. 152:483-90). Appropriate gene regulation also ensures that cells function efficiently by expressing only those genes whose functions are required at a given time. Factors that influence gene expression include extracellular signals that mediate cell-cell communication and coordinate the activities of different cell types. Gene expression is regulated at the level of DNA and RNA transcription, and at the level of mRNA translation.

Aberrant expression or mutations in genes and their products may cause, or increase susceptibility to, a variety of human diseases such as cancer and other cell proliferative disorders. The identification of these genes and their products is the basis of an ever-expanding effort to find markers for early detection of diseases and targets for their prevention and treatment. For example, cancer represents a type of cell proliferative disorder that affects nearly every tissue in the body. The development of cancer, or oncogenesis, is often correlated with the conversion of a normal gene into a cancer-causing gene, or oncogene, through abnormal expression or mutation. Oncoproteins, the products of oncogenes, include a variety of molecules that influence cell proliferation, such as growth factors, growth factor receptors, intracellular signal transducers, nuclear transcription factors, and cell-cycle control proteins. In contrast, tumor-suppressor genes are involved in inhibiting cell proliferation. Mutations which reduce or abrogate the function of tumor-suppressor genes result in aberrant cell proliferation and cancer. Thus a wide variety of genes and their products have been found that are associated with cell proliferative disorders such as cancer, but many more may exist that are yet to be discovered.

DNA-based arrays can provide an efficient, high-throughput method to examine gene expression and genetic variability. For example, SNPs, or single nucleotide polymorphisms, are the most common type of human genetic variation. DNA-based arrays can dramatically accelerate the discovery of SNPs in hundreds and even thousands of genes. Likewise, such arrays can be used for SNP genotyping in which DNA samples from individuals or populations are assayed for the presence of selected SNPs. These approaches will ultimately lead to the systematic identification of all genetic variations in the human genome and the correlation of certain genetic variations with disease susceptibility, responsiveness to drug treatments, and other medically relevant information. (See, for example, Wang, D. G. et al. (1998) Science 280:1077-1082.)

DNA-based arrays can also provide a simple way to explore the expression of a single polymorphic gene. When the expression of a single gene is explored, DNA-based arrays are employed to detect the expression of specific gene variants. For example, a p53 tumor suppressor gene array is used to determine whether individuals are carrying mutations that predispose them to cancer. A cytochrome p450 gene array is useful to determine whether individuals have one of a number of specific mutations that could result in increased drug metabolism, drug resistance or drug toxicity.

DNA-based array technology is especially important for the rapid analysis of global gene expression patterns. There is a growing awareness that gene expression is affected in a global fashion. In some cases the interactions may be expected, such as when the genes are part of the same signaling pathway. In other cases, such as when the genes participate in separate signaling pathways, the interactions may be totally unexpected. Therefore, DNA-based arrays can be used to investigate how genetic predisposition, disease, or therapeutic treatment affects the expression of a large number of genes. In this case, it is useful to develop a profile, or transcript image, of all the genes that are expressed and the levels at which they are expressed in that particular tissue. A profile generated from an individual or population affected with a certain disease or undergoing a particular therapy may be compared with a profile generated from a control individual or population. Such analysis does not require knowledge of gene function, as the expression profiles can be subjected to mathematical analyses which simply treat each gene as a marker. Furthermore, gene expression profiles may help dissect biological pathways by identifying all the genes expressed, for example, at a certain developmental stage, in a particular tissue, or in response to disease or treatment (See, for example, Lander, E. S. et al. (1996) Science 274:536-539.)

Certain genes are known to be associated with diseases because of their chromosomal location, such as the genes in the myotonic dystrophy (DM) regions of mouse and human. The mutation underlying DM has been localized to a gene encoding the DM-kinase protein, but another active gene, DMR-N9, is in close proximity to the DM-kinase gene (Jansen, G. et al. (1992) Nat. Genet. 1:261-266). DMR-N9 encodes a 650 amino acid protein that contains WD repeats, motifs found in cell signaling proteins. DMR-N9 is expressed in all neural tissues and in the testis, suggesting a role for DMR-N9 in the manifestation of mental and testicular symptoms in severe cases of DM (Jansen, G. et al. (1995) Hum. Mol. Genet. 4:843-852).

Other genes are identified based upon their expression patterns or association with disease syndromes. For example, autoantibodies to subcellular organelles are found in patients with systemic rheumatic diseases. A recently identified protein, golgin-67, belongs to a family of Golgi autoantigens having alpha-helical coiled-coil domains (Eystathioy, T. et al. (2000) J. Autoimmun. 14:179-187). The Stac gene was identified as a brain specific, developmentally regulated gene. The Stac protein contains an SH3 domain, and is thought to be involved in neuron-specific signal transduction (Suzuki, H. et al. (1996) Biochem. Biophys. Res. Commun. 229:902-909).

Evi-5 is a site of retroviral integration in AKXD T-cell lymphoma cells. Tumors with Evi-5 integrations have also been shown to possess other integration sites associated with T-cell disease. Retroviral disease induction occurs as a result of insertional mutagenesis of cellular proto-oncogenes or tumor suppressor genes. The AKXD recombinant inbred murine model is useful in the study of retrovirally-induced myeloid tumors, as well as T- and B-cell leukemias (Liao, X. et al. (1997) Oncogene 14:1023-1029). Lymphomas with integrations in Evi-5 may also possess integrations in Myc, in sites located near and activating Myc, or that synergize with Myc. This suggests a possible cooperation between Evi-5 with Myc in tumor induction, consistent with other observations showing that Myc is a frequent target of retroviral integration in mouse and rat T-cell lymphomas.

The contiguous gene deletion syndrome AMME is characterized by Alport syndrome, midface hypoplasia, mental retardation and elliptocytosis and is caused by a deletion in Xq22.3, comprising several genes including COL4A5, FACL4 and AMMECR1. AMMECR1, found in eukaryotic and prokaryotic cells, contains six exons and codes for a protein with a molecular mass of 35.5 kDa. Evidence suggests that this protein is a regulatory factor potentially involved in the development of AMME contiguous gene deletion syndrome. The mouse ortholog has 95.2% identity at the amino acid level with human AMMECR1 and maps to chromosome MmuXF1—F3 (Vitelli, F. et al. (1999) Genomics 55:335-340; Vitelli, F. et al. (2000) Cytogenet. Cell Genet. 88:259-263).

Sporulation-induced transcript 4 (SIT4) gene is a type 2A-related serine/threonine protein phosphatase which when overexpressed confers lithium tolerance in galactose medium to the budding yeast, Saccharomyces cerevisiae. It is a regulator of the cell cycle and is involved in nitrogen sensing, normal g1 cyclin expression, and bud initiation (Masuda, C. A. et al. (2000) J. Biol. Chem 275:30957-30961). The SIT4-associated proteins (SAPs), SAP155, SAP185, SAP190, and probably SAP4, associate with SIT4 in separate complexes. The SAPs are not functional in the absence of SIT4 and likewise, SIT4 is not functional in the absence of the SAPs. However, SAPs and SIt4 have distinct functions (Luke, M. M. et al. (1996) Mol. Cell. Biol. 16:2744-2755). C11 or f23 is a human ortholog of the yeast SAP family. C11 or f23 has been mapped to the 400-kb region of the IDDM4 locus of chromosome 11q13, a region involved in type 1 diabetes (Twells, R. C. et al. (2001) Genomics 72:231-242).

Dendritic cells are antigen-presenting cells that play a major role in the initial phases of the immune response. Dendritic cells located in peripheral tissues are generally immature and exhibit a strong capacity to capture surrounding antigens whereas they exhibit limited T cell activation capacity. Reciprocally, mature dendritic cells found in lymphoid organs exhibit a strong capacity to activate T cells but have lost most of their ability to pick up new antigens. Dendritic cells migrating out of transplanted organ play a major role in the induction of graft rejection. Therefore, genes that are modulated during the maturation of dendritic cells represent potential targets for drugs aimed at limiting the rejection of transplanted organs.

Rho-family GTPases are critical mediators of dendritic growth and remodeling. Three of these Rho GTPases, RhoA, Rac1 and Cdc42 (cell division cycle 42), regulate distinct aspects of dendritic development, such as dendrite initiation, dendrite growth, dendrite branching, and spine formation. In cortical neurons, Rho-family GTPases play a central role in determining the number of primary dendrites in both pyramidal and non-pyramidal neurons. Research suggests that Rac1 is an important effector of dendrite initiation and that a common effector of Rac 1 and Cdc42 mediates dendrite initiation. Suggested effectors include the p21-activated kinase (PAK) family of serine threonine kinases and LIM-domain-containing protein kinase, which can modulate actin dynamics by phosphorylation of cofilin.

Rho-family GTPases also can influence large-scale dendritic remodeling. Many neurons in the cortex initially acquire a pyramidal morphology and undergo a developmentally-regulated transformation into non-pyramidal neurons. This transformation involves the withdrawal of the apical dendrite and the extension of new primary dendrites, and is inhibited by expression of dominant-negative Cdc42 and, to a lesser extent, by dominant-negative Rac1. This inhibition suggests that the acquisition of cell-type-specific dendritic morphologies is under the control of Rac1 and Cdc42 signaling Redmond, L. and Ghosh, A. (2001) Curr. Opin. Neurobiol. 11:111-117).

ADP-ribosylation factors (ARFs) are small guanine-nucleotide-binding proteins that regulate membrane traffic and organelle structure in eukaryotic cells. In general, the inactive GDP-bound form of ARF is soluble, although it can associate weakly with membranes, whereas the active GTP-bound form binds tightly to the membrane. ARFs function on membrane surfaces where they encounter their effectors and regulators, the guanine nucleotide exchange factors (GEFs) and GTPase-activating proteins (GAPs). ARF effectors include lipid-modifying enzymes and cytosolic coat complexes (such as COPI) that are recruited onto membranes by ARF-GTP. Hence, ARF activation leads to changes in both the lipid and protein composition of the membrane on which it is localized; changes which in turn result in modulation of membrane structure and function.

ARF proteins are highly conserved and have been found in all eukaryotic organisms examined. Mammalian ARF proteins are divided into three classes: Class I (ARF1-ARF3), Class II (ARF4 and ARF5) and Class m (ARF6). Class I ARFs are involved in trafficking in the ER-Golgi and endosomal systems, and their functions have been extensively studied. ARF1 binding to endosomal membranes is regulated by endosomal pH, which explains the pH dependence of COPI binding to endosomes. The Class m ARF, ARF6, functions exclusively in the endosomal-plasma membrane systerm. ARF6 is involved in endosomal recycling to the plasma membrane (PM), in regulated secretion, and in coordinating actin cytoskeleton changes at the PM. ARF6 is present at the apical surface of Madin Darby Canine Kidney (MDCK) epithelial cells, where it plays a role in modulating clathrin endocytosis. ARF6 has also been implicated in Fc-mediated phagocytosis in macrophages and in insulin stimulation of adipsin secretion and Glut4 translocation. By contrast, virtually nothing is known about the functions of the class II ARFs (Donaldson, J. D. and Jackson, C. L. (2000) Curr. Opin. Cell Biol. 12:475-482).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

The potential application of gene expression profiling is particularly relevant to improving diagnosis, prognosis, and treatment of disease. For example, both the levels and sequences expressed in tissues from subjects with a cardiovascular disorder may be compared with the levels and sequences expressed in normal tissue.

Atherosclerosis and the associated coronary artery disease and cerebral stroke represent the most common cause of death in industrialized nations. Although certain key risk factors have been identified, a full molecular characterization that elucidates the causes and provides care for this complex disease has not been achieved. Molecular characterization of growth and regression of atherosclerotic vascular lesions requires identification of the genes that contribute to features of the lesion including growth, stability, dissolution, rupture and, most lethally, induction of occlusive vessel thrombus. Vascular lesions principally involve the vascular endothelium and the surrounding smooth muscle tissue.

Development of atherosclerosis is understood to be induced by the presence of circulating lipoprotein. Lipoproteins, such as the cholesterol-rich low-density lipoprotein (LDL), accumulate in the extracellular space of the vascular intima, and undergo modification. Oxidation of LDL (Ox-LDL) occurs most avidly in the sub-endothelial space where circulating antioxidant defenses are less effective. Mononuclear phagocytes enter the intima, differentiate into macrophages, and ingest modified lipids including Ox-LDL. During Ox-LDL uptake, macrophages produce cytokines (e.g. tumor necrosis factor α (TNF-α) and interleukin-1 (IL-1)) and growth factors (e.g. M-CSF, VEGF, and PDGF-BB) that elicit further cellular events that modulate atherogenesis such as smooth muscle cell proliferation and production of extracellular matrix by vascular endothelium Additionally, these macrophages may activate genes in endothelium and smooth muscle tissue involved in inflammation and tissue differentiation, including superoxide dismutatse (SOD), IL-8, and ICAM-1.

The vascular endothelium influences not only the three classically interacting components of hemostasis: the vessel, the blood platelets and the clotting and fibrinolytic systems of plasma, but also the natural sequelae: inflammation and tissue repair. Two principal modes of endothelial behavior may be differentiated, best defined as an anti- and a prothrombotic state. Under physiological conditions endothelium mediates vascular dilatation (formation of nitric oxide (NO), PGI₂, adenosine, hyperpolarising factor), prevents platelet adhesion and activation (production of adenosine, NO and PGI₂, removal of ADP), blocks thrombin formation (tissue factor pathway inhibitor, activation of protein C via thrombomodulin, activation of antithrombin III) and mitigates fibrin deposition (t- and scuplasminogen activator production). Adhesion and transmigration of inflammatory leukocytes are attenuated, e.g. by NO and IL-10, and oxygen radicals are efficiently scavenged (urate, NO, glutathione, SOD).

When the endothelium is physically disrupted or functionally perturbed by postischemic reperfusion, acute and chronic inflammation, atherosclerosis, diabetes and chronic arterial hypertension, then completely opposing actions pertain. This prothrombotic, proinflammatory state is characterised by vaso-constriction, platelet and leukocyte activation and adhesion (externalization, expression and upregulation of, for example, von Willebrand factor, platelet activating factor, P-selectin, ICAM-1, IL-8, MCP-1, and TNF-α), promotion of thrombin formation, coagulation and fibrin deposition at the vascular wall (expression of tissue factor, PAI-1, and phosphatidyl serine) and, in platelet-leukocyte coaggregates, additional inflammatory interactions via attachment of platelet CD40-ligand to endothelial, monocyte and B-cell CD40. Since thrombin formation and inflammatory stimulation set the stage for later tissue repair, complete abolition of such endothelial responses cannot be the goal of clinical interventions aimed at limiting procoagulatory, prothrombotic actions of a dysfunctional vascular endothelium (See, e.g., Becker et al. (2000) Z Kardiol 89:160-167.)

Tumor necrosis factor α is a pleiotropic cytokine that a mediates immune regulation and inflammatory responses. TNF-α related cytokines generate partially overlapping cellular responses, including differentiation, proliferation, nuclear factor-κb (NF-KB) activation, and cell death, by triggering the aggregation of receptor monomers (Smith, C. A. et al. (1994) Cell 76:959-962). The cellular responses triggered by TNF-α are initiated through its interaction with distinct cell surface receptors (TNFRs). NF-κB is a transcription factor with a pivotal role in inducing genes involved in physiological processes as well as in the response to injury and infection. Activation of NF-κB involves the phosphorylation and subsequent degradation of an inhibitory protein, IKB, and many of the proximal kinases and adaptor molecules involved in this process have been elucidated. Additionally, the NF-κB activation pathway from cell membrane to nucleus for IL-1 and TNF-α is now understood (Bowie and O'Neill (2000) Biochem Pharmacol 59:13-23).

Monocyte chemoattractant protein-1 (MCP-1) is known to play an important role in the pathogenesis of atherosclerosis by inducing monocyte migration. TNF-α treatment of human umbilical vein endothelial cells (HVECs) increased the cellular secretions of MCP-1 119-fold compared with untreated cells. Troglitazone, an insulin-sensitizing drug, significantly inhibited this TNF-α-induced increase in MCP-1 secretions and decreased mRNA levels (Ohta et al. (2000) Diabetes Res Clin Pract 48:171-176).

Treatment of confluent cultures of vascular smooth muscle cells (SMCs) with TNF-α suppresses the incorporation of [³H]proline into both collagenase-digestible proteins (CDP) and noncollagenous proteins (NCP). Such suppression by TNF-α is not observed in confluent bovine aortic endothelial cells and human fibroblastic IMR-90 cells. TNF-α decreases the relative proportion of collagen types IV and V suggesting that TNF-α modulates collagen synthesis by SMCs depending on their cell density and therefore may modify formation of atherosclerotic lesions (Hiraga et al. (2000) Life Sci 66:235-244).

Human coronary artery smooth muscle cells (CASMC) are primary cells isolated from the tunica media (an intermediate muscular layer) of a human coronary artery. Vascular smooth muscle cells are a model of increasing significance in vascular biology. It is now well known that besides their obvious role in the regulation of vascular tone and, consequently, oxygen supply to various tissues, their behavior under inflammatory conditions is an important factor in the development of atherosclerosis and restenosis.

Human aortic endothelial cells (HAECs) are primary cells derived from the endothelium of a human aorta. HAECs have been used as an experimental model for investigating in vitro the role of the endothelium in human vascular biology. Activation of the vascular endothelium is considered to be a central event in a wide range of both physiological and pathophysiological processes, such as vascular tone regulation, coagulation and thrombosis, atherosclerosis, and inflammation.

Thus, vascular tissue genes differentially expressed during treatment of CASMC and HAEC cell cultures with TNFa may reasonably be expected to be markers of the atherosclerotic process.

The potential application of gene expression profiling is particularly relevant to improving diagnosis, prognosis, and treatment of disease. For example, both the levels and sequences expressed in tissues from subjects with ovarian cancer may be compared with the levels and sequences expressed in normal tissue. Ovarian cancer is the leading cause of death from a gynecologic cancer. The majority of ovarian cancers are derived from epithelial cells, and 70% of patients with epithelial ovarian cancers present with late-stage disease. As a result, the long-term survival rate for individuals with this disease is very low. Identification of early-stage markers for ovarian cancer would significantly increase the survival rate. The molecular events that lead to ovarian cancer are poorly understood. Some of the known aberrations include mutation of p53 and microsatellite instability. Since gene expression patterns likely vary when normal ovary is compared to ovarian tumors, examination of gene expression in these tissues can identify possible markers for ovarian cancer.

Steroids are a class of lipid-soluble molecules, including cholesterol, bile acids, vitamin D, and hormones, that share a common four-ring structure based on cyclopentanoperhydrophenanthrene and that carry out a wide variety of functions. Cholesterol, for example, is a component of cell membranes that controls membrane fluidity. It is also a precursor for bile acids which solubilize lipids and facilitate absorption in the small intestine during digestion. Vitamin D regulates the absorption of calcium in the small intestine and controls the concentration of calcium in plasma. Steroid hormones, produced by the adrenal cortex, ovaries, and testes, include glucocorticoids, mineralocorticoids, androgens, and estrogens. They control various biological processes by binding to intracellular receptors that regulate transcription of specific genes in the nucleus. Glucocorticoids, for example, increase blood glucose concentrations by regulation of gluconeogenesis in the liver, increase blood concentrations of fatty acids by promoting lipolysis in adipose tissues, modulate sensitivity to catcholamines in the central nervous system, and reduce inflammation. The principal mineralocorticoid, aldosterone, is produced by the adrenal cortex and acts on cells of the distal tubules of the kidney to enhance sodium ion reabsorption. Androgens, produced by the interstitial cells of Leydig in the testis, include the male sex hormone testosterone, which triggers changes at puberty, the production of sperm and maintenance of secondary sexual characteristics. Female sex hormones, estrogen and progesterone, are produced by the ovaries and also by the placenta and adrenal cortex of the fetus during pregnancy. Estrogen regulates female reproductive processes and secondary sexual characteristics. Progesterone regulates changes in the endometrium during the menstrual cycle and pregnancy.

Steroid hormones are widely used for fertility control and in anti-inflammatory treatments for physical injuries and diseases such as arthritis, asthma, and auto-immune disorders. Progesterone, a naturally occurring progestin, is primarily used to treat amenorrhea, abnormal uterine bleeding, or as a contraceptive. Endogenous progesterone is responsible for inducing secretory activity in the endometrium of the estrogen-primed uterus in preparation for the implantation of a fertilized egg and for the maintenance of pregnancy. It is secreted from the corpus luteum in response to luteinizing hormone (LH). The primary contraceptive effect of exogenous progestins involves the suppression of the midcycle surge of LH. At the cellular level, progestins diffuse freely into target cells and bind to the progesterone receptor. Target cells include the female reproductive tract, the mammary gland, the hypothalamus, and the pituitary. Once bound to the receptor, progestins slow the frequency of release of gonadotropin releasing hormone from the hypothalamus and blunt the pre-ovulatory LH surge, thereby preventing follicular maturation and ovulation. Progesterone has minimal estrogenic and androgenic activity. Progesterone is metabolized hepatically to pregnanediol and conjugated with glucuronic acid.

Medroxyprogesterone (MAH), also known as 6α-methyl-17-hydroxyprogesterone, is a synthetic progestin with a pharmacological activity about 15 times greater than progesterone. MAH is used for the treatment of renal and endometrial carcinomas, amenorrhea, abnormal uterine bleeding, and endometriosis associated with hormonal imbalance. MAH has a stimulatory effect on respiratory centers and has been used in cases of low blood oxygenation caused by sleep apnea, chronic obstructive pulmonary disease, or hypercapnia.

Danazol is a synthetic steroid derived from ethinyl testosterone. Danazol indirectly reduces estrogen production by lowering pituitary synthesis of follicle-stimulating hormone and LH. Danazol also binds to sex hormone receptors in target tissues, thereby exhibiting anabolic, antiestrognic, and weakly androgenic activity. Danazol does not possess any progestogenic activity, and does not suppress normal pituitary release of corticotropin or release of cortisol by the adrenal glands. Danazol is used in the treatment of endometriosis to relieve pain and inhibit endometrial cell growth. It is also used to treat fibrocystic breast disease and hereditary angioedema.

Corticosteroids are used to relieve inflammation and to suppress the immune response. They inhibit eosinophil, basophil, and airway epithelial cell function by regulation of cytokines that mediate the inflammatory response. They inhibit leukocyte infiltration at the site of inflammation, interfere in the function of mediators of the inflammatory response, and suppress the humoral immune response. Corticosteroids are used to treat allergies, asthma, arthritis, and skin conditions. Beclomethasone is a synthetic glucocorticoid that is used to treat steroid-dependent asthma, to relieve symptoms associated with allergic or nonallergic (vasomotor) rhinitis, or to prevent recurrent nasal polyps following surgical removal. The anti-inflammatory and vasoconstrictive effects of intranasal beclomethasone are 5000 times greater than those produced by hydrocortisone.

The anti-inflammatory actions of corticosteroids are thought to involve phospholipase A₂ inhibitory proteins, collectively called lipocortins. Lipocortins, in turn, control the biosynthesis of potent mediators of inflammation such as prostaglandins and leukotrienes by inhibiting the release of the precursor molecule arachidonic acid. Proposed mechanisms of action include decreased IgE synthesis, increased number of β-adrenergic receptors on leukocytes, and decreased arachidonic acid metabolism. During an immediate allergic reaction, such as in chronic bronchial asthma, allergens bridge the IgE antibodies on the surface of mast cells, which triggers these cells to release chemotactic substances. Mast cell influx and activation, therefore, is partially responsible for the inflammation and hyperirritability of the oral mucosa in asthmatic patients. This inflammation can be retarded by administration of corticosteroids. ENDFIELD

The potential application of gene expression profiling is particularly relevant to measuring the toxic response to potential therapeutic compounds and the metabolic response to therapeutic agents. Diseases treated with steroids and disorders caused by the metabolic response to treatment with steroids include adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease. Response may be measured by comparing both the levels and sequences expressed in tissues from subjects exposed to or treated with steroid compounds such as medroxyprogesterone (MAH) or budesonide (bude) with the levels and sequences expressed in normal untreated tissue.

The effects upon liver metabolism and hormone clearance mechanisms are important to understand the pharmacodynamics of a drug. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein iii) conversion of ammonia to urea and glutamine; iv) metabolism of aromatic amino acids; and v) proliferation in glucose-free and insulin-free medium. The C3A cell line is now well established as an in vitro model of the mature human liver (Mickelson et al. (1995) Hepatology 22:866-875; Nagendra et al. (1997) Am J Physiol 272:G408-G416).

There is a need in the art for new compositions, including nucleic acids and proteins, for the diagnosis, prevention, and treatment of cell proliferative, autoimmune/inflammatory, developmental, and neurological disorders, diseases treated with steroids and disorders caused by the metabolic response to treatment with steroids.

### SUMMARY OF THE INVENTION

Various embodiments of the invention provide purified polypeptides, molecules for disease detection and treatment, referred to collectively as “MDDT” and individually as “MDDT-1,” “MDDT-2,” “MDDT-3,” “MDDT-4,” “MDDT-5,” “MDDT-6,” “MDDT-7,” “MDDT-8,” “MDDT-9,” “MDDT-10,” “MDDT-11,” “MDDT-12,” “MDDT-13,” “MDDT-14,” “MDDT-15,” “MDDT-16,” “MDDT-17,” “MDDT-18,” “MDDT-19,” “MDDT-20,” “MDDT-21,” “MDDT-22,” “MDDT-23,” “MDDT-24,” “MDDT-25,” “MDDT-26,” “MDDT-27,” “MDDT-28,” “MDDT-29,” “MDDT-30,” “MDDT-31,” “MDDT-32,” “MDDT-33,” “MDDT-34,” “MDDT-35,” “MDDT-36,” “MDDT-37,” “MDDT-38,” “MDDT-39,” “MDDT-40,” “MDDT-1,” “MDDT-42,” “MDDT43,” “MDDT-44,” “MDDT-45,” “MDDT-46,” “MDDT-47,” “MDDT-48,” “MDDT-49,” “MDDT-50,” “MDT-51,” “MDDT-52,” “MDDT-53,” “MDDT-54,” “MDDT-55,” and “MDDT-56” and methods for using these proteins and their encoding polynucleotides for the detection, diagnosis, and treatment of diseases and medical conditions. Embodiments also provide methods for utilizing the purified molecules for disease detection and treatment and/or their encoding polynucleotides for facilitating the drug discovery process, including determination of efficacy, dosage, toxicity, and pharmacology. Related embodiments provide methods for utilizing the purified molecules for disease detection and treatment and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56. Another embodiment provides an isolated polypeptide comprising an amino acid sequence of SEQ ID NO:1-56.

Still another embodiment provides an isolated polynucleotide encoding a polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56. In another embodiment, the polynucleotide encodes a polypeptide selected from the group consisting of SEQ ID NO:1-56. In an alternative embodiment, the polynucleotide is selected from the group consisting of SEQ ID NO:57-112.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56.

Still yet another embodiment provides an isolated polynucleotide selected from the group consisting of a) a polynucleotide comprising a polynucleotide sequence selected from the group consisting of SEQ ID NO:57-112, b) a polynucleotide comprising a naturally occurring polynucleotide sequence at least 90% identical or at least about 90% identical to a polynucleotide sequence selected from the group consisting of SEQ ID NO:57-112, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides. Yet another embodiment provides a method for detecting a target polynucleotide in a sample, said target polynucleotide being selected from the group consisting of a) a polynucleotide comprising a polynucleotide sequence selected from the group consisting of SEQ ID NO:57-112, b) a polynucleotide comprising a naturally occurring polynucleotide sequence at least 90% identical or at least about 90% identical to a polynucleotide sequence selected from the group consisting of SEQ ID NO:57-112, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:57-112, b) a polynucleotide comprising a naturally occurring polynucleotide sequence at least 90% identical or at least about 90% identical to a polynucleotide sequence selected from the group consisting of SEQ ID NO:57-112, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56, and a pharmaceutically acceptable excipient. In one embodiment, the composition can comprise an amino acid sequence selected from the group consisting of SEQ ID NO:1-56. Other embodiments provide a method of treating a disease or condition associated with decreased or abnormal expression of functional MDDT, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional MDDT, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional MDDT, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-56, b) a polypeptide comprising a naturally occurring amino acid sequence at least 90% identical or at least about 90% identical to an amino acid sequence selected from the group consisting of SEQ ID NO:1-56, c) a biologically active fragment of a polypeptide having an amino acid sequence selected from the group consisting of SEQ ID NO:1-56, and d) an immunogenic fragment of a polypeptide having an amino acid sequence selected from the group consisting of SEQ ID NO:1-56. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:57-112, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:57-112, ii) a polynucleotide comprising a naturally occurring polynucleotide sequence at least 90% identical or at least about 90% identical to a polynucleotide sequence selected from the group consisting of SEQ ID NO:57-112, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:57-112, ii) a polynucleotide comprising a naturally occurring polynucleotide sequence at least 90% identical or at least about 90% identical to a polynucleotide sequence selected from the group consisting of SEQ ID NO:57-112, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide embodiments, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“MDDT” refers to the amino acid sequences of substantially purified MDDT obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of MDDT. Agonists may include proteins, nucleic acids, carbohydrates, small molecules, or any other compound or composition which modulates the activity of MDDT either by directly interacting with MDDT or by acting on components of the biological pathway in which MDDT participates.

An “allelic variant” is an alternative form of the gene encoding MDDT. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding MDDT include those sequences with deletions, insertions, or substitutions of different nucleotides, resulting in a polypeptide the same as MDDT or a polypeptide with at least one functional characteristic of MDDT. Included within this definition are polymorphisms which may or may not be readily detectable using a particular oligonucleotide probe of the polynucleotide encoding MDDT, and improper or unexpected hybridization to allelic variants, with a locus other than the normal chromosomal locus for the polynucleotide encoding MDDT. The encoded protein may also be “altered,” and may contain deletions, insertions, or substitutions of amino acid residues which produce a silent change and result in a functionally equivalent MDDT. Deliberate amino acid substitutions may be made on the basis of one or more similarities in polarity, charge, solubility, hydrophobicity, hydrophilicity, and/or the amphipathic nature of the residues, as long as the biological or immunological activity of MDDT is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of MDDT. Antagonists may include proteins such as antibodies, anticalins, nucleic acids, carbohydrates, small molecules, or any other compound or composition which modulates the activity of MDDT either by directly interacting with MDDT or by acting on components of the biological pathway in which MDDT participates.

The term “antibody” refers to intact immunoglobulin molecules as well as to fragments thereof, such as Fab, F(ab′)₂, and Fv fragments, which are capable of binding an epitopic determinant. Antibodies that bind MDDT polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic MDDT, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding MDDT or fragments of MDDT may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of MDDT or a polynucleotide encoding MDDT which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:57-112 can comprise a region of unique polynucleotide sequence that specifically identifies SEQ ID NO:57-112, for example, as distinct from any other sequence in the genome from which the fragment was obtained. A fragment of SEQ ID NO:57-112 can be employed in one or more embodiments of methods of the invention, for example, in hybridization and amplification technologies and in analogous methods that distinguish SEQ ID NO:57-112 from related polynucleotides. The precise length of a fragment of SEQ ID NO:57-112 and the region of SEQ ID NO:57-112 to which the fragment corresponds are routinely determinable by one of ordinary skill in the art based on the intended purpose for the fragment.

A fragment of SEQ ID NO:1-56 is encoded by a fragment of SEQ ID NO:57-112. A fragment of SEQ ID NO:1-56 can comprise a region of unique amino acid sequence that specifically identifies SEQ ID NO:1-56. For example, a fragment of SEQ ID NO:1-56 can be used as an immunogenic peptide for the development of antibodies that specifically recognize SEQ ID NO:1-56. The precise length of a fragment of SEQ ID NO:1-56 and the region of SEQ ID NO:1-56 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlmnih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (April-21-2000) set at default parameters. Such default parameters may be, for example:

Matrix: BLOSUM62

Rewardfor match: 1

Penalty for mismatch: −2

Open Gap: 5 and Extension Gap: 2 penalties

Gap×drop-off 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (April-21-2000) with blastp set at default parameters. Such default parameters may be, for example:

Matrix: BLOSUM62

Open Gap: 11 and Extension Gap: 1 penalties

Gap×drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., Cot or Rot analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytolines, chemoldnes, and other signaling molecules, which may affect cellular and systemic defense systems.

An “immunogenic fragment” is a polypeptide or oligopeptide fragment of MDDT which is capable of eliciting an immune response when introduced into a living organism, for example, a mammal. The term “immunogenic fragment” also includes any polypeptide or oligopeptide fragment of MDDT which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of MDDT. For example, modulation may cause an increase or a decrease in protein activity, binding characteristics, or any other biological, functional, or immunological properties of MDDT.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an MDDT may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of MDDT.

“Probe” refers to nucleic acids encoding MDDT, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989; Molecular Cloning: A Laboratory Manual, 2^{nd} ed., vol. 1-3, Cold Spring Harbor Press, Plainview N.Y.), Ausubel, F. M. et al. (1999) Short Protocols in Molecular Biology, 4^{th} ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 pmmer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing MDDT, nucleic acids encoding MDDT, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

The Invention

Various embodiments of the invention include new human molecules for disease detection and treatment (MDDT), the polynucleotides encoding MDDT, and the use of these compositions for the diagnosis, treatment, or prevention of cell proliferative, autoimmune/inflammatory, developmental, and neurological disorders, diseases treated with steroids and disorders caused by the metabolic response to treatment with steroids.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to polypeptide and polynucleotide embodiments. The full length clones encode polypeptides which have at least 95% sequence identity to the polypeptides shown in column 3.

Table 2 shows sequences with homology to the polypeptides of the invention as identified by BLAST analysis against the GenBank protein (genpept) database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog. Column 4 shows the probability scores for the matches between each polypeptide and its homolog(s). Column 5 shows the annotation of the GenBank homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are molecules for disease detection and treatment. For example, SEQ ID NO:2 contains a potassium channel tetramerisation domain domain as determined by searching for statistically significant matches in the hidden Markov model (HMM)-based PFAM database of conserved protein family domains. (See Table 3.)

In another example, SEQ ID NO:22 is 93% identical, from residue M1 to residue V1451, to mouse pecanex 1, which is the mouse homolog of Drosophila pecanex, a matemal-effect neurogenic protein (GenBank ID g665⁰377) as determined by the Basic Local Alignment Search Tool (BLAST). (See Table 2.) The BLAST probability score is 0.0, which indicates the probability of obtaining the observed polypeptide sequence alignment by chance. Data from further BLAST analyses provide corroborative evidence that SEQ ID NO:22 is a pecanex 1 protein.

In another example, SEQ ID NO:31 is 33% identical, from residue R17 to residue G452, to Drosophila melanogaster Diablo (GenBank ID g7243777) as determined by the Basic Local Alignment Search-Tool (BLAST). (See Table 2.) The BLAST probability score is 2.9e-50, which indicates the probability of obtaining the observed polypeptide sequence alignment by chance. SEQ ID NO:31 also contains a BTB-POZ protein interaction domain as determined by searching for statistically significant matches in the hidden Markov model (HMM)-based PFAM database of conserved protein family domains. (See Table 3.) Data from BLIMPS and additional BLAST analyses provide further corroborative evidence that SEQ ID NO:31 is an apoptosis-associated protein.

In another example, SEQ ID NO:36 is 62% identical, from residue E84 to residue L370, to a human EVI-5 protein (GenBank ID g3093476) as determined by the Basic Local Alignment Search Tool (BLAST). (See Table 2.) The BLAST probability score is 6.9e-90, which indicates the probability of obtaining the observed polypeptide sequence alignment by chance. Data from MOTIFS analysis provides further corroborative evidence that SEQ ID NO:36 is a protein with potential utility for disease detection or treatment.

In another example, SEQ ID NO:44 is 78% identical, from residue D224 to residue V838, and 98% identical, from residue M1 to residue W333, to human sporulation-induced transcript 4 (SIT4)-associated protein SAPLa (GenBank ID g11527201) as determined by the Basic Local Alignment Search Tool (BLAST). (See Table 2.) The BLAST probability score is 6.7e-250 for the first homologous section and 1.8e-171 for the second, which indicates the probability of obtaining the observed polypeptide sequence alignment by chance. Data from other BLAST analyses provide further corroborative evidence that SEQ ID NO:44 is a cycle cell phosphorylation sit4-associating protein (a protein which associates with the sit4 phosphatase in a cell cycle-dependent manner).

In another example, SEQ ID NO:47 is 52% identical, from residue F6 to residue L256, to a WD-40-containing Xenopus laevis protein that is upregulated by thyroid hormone (GenBank ID g1314316) as determined by the Basic Local Alignment Search Tool (BLAST). The BLAST probability score is 6.3e-73, which indicates the probability of obtaining the observed polypeptide sequence alignment by chance. SEQ ID NO:47 also contains a WD, G-beta repeat domain as determined by searching for statistically significant matches in the hidden Markov model (HMM)-based PFAM database of conserved protein family domains. (See Table 3.) Data from MOTIFS and PROFILESCAN analyses provide further corroborative evidence that SEQ ID NO:47 is a full-length human molecule for disease detection and treatment. SEQ ID NO:1, SEQ ID NO:3-21, SEQ ID NO:23-30, SEQ ID NO:32-35, SEQ ID NO:37-43, SEQ ID NO:45-46 and SEQ ID NO:48-56 were analyzed and annotated in a similar manner. The algorithms and parameters for the analysis of SEQ ID NO:1-56 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:57-112 or that distinguish between SEQ ID NO:57-112 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (ie., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3} . . . , if present, represent specific exons that may have been manually edited during analysis (See Example V). Alternatively, the polynucleotide fragments in colum 2 may refer to assemblages of exons brought together by an “exon-stretching” algorithm. For example, a polynucleotide sequence identified as FLXXXXXX_gAAAAA_gBBBB_{—1}_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (ie., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for |
| GFG, | example, GENSCAN (Stanford University, CA, USA) |
| ENST | or FGENES (Computer Genomics Group, The Sanger |
| | Centre, Cambridge, UK). |
| GBI | Hand-edited analysis of genomic sequences. |
| FL | Stitched or stretched genomic sequences (see |
| | Example V). |
| INCY | Full length transcript and exon prediction from |
| | mapping of EST sequences to the genome. Genomic |
| | location and EST composition data are combined |
| | to predict the exons and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide embodiments, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP(SNP ID). Column 5 shows the position within the EST sequence at which the SNP is located (EST SNP), and column 6 shows the position of the SNP within the full-length polynucleotide sequence (CB 1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses MDDT variants. A preferred MDDT variant is one which has at least about 80%, or alternatively at least about 90%, or even at least about 95% amino acid sequence identity to the MDDT amino acid sequence, and which contains at least one functional or structural characteristic of MDDT.

Various embodiments also encompass polynucleotides which encode MDDT. In a particular embodiment, the invention encompasses a polynucleotide sequence comprising a sequence selected from the group consisting of SEQ ID NO:57-112, which encodes MDDT. The polynucleotide sequences of SEQ ID NO:57-112, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding MDDT. In particular, such a variant polynucleotide will have at least about 70%, or alternatively at least about 85%, or even at least about 95% polynucleotide sequence identity to a polynucleotide encoding MDDT. A particular aspect of the invention encompasses a variant of a polynucleotide comprising a sequence selected from the group consisting of SEQ ID NO:57-112 which has at least about 70%, or alternatively at least about 85%, or even at least about 95% polynucleotide sequence identity to a nucleic acid sequence selected from the group consisting of SEQ ID NO:57-112. Any one of the polynucleotide variants described above can encode a polypeptide which contains at least one functional or structural characteristic of MDDT.

In addition, or in the alternative, a polynucleotide variant of the invention is a splice variant of a polynucleotide encoding MDDT. A splice variant may have portions which have significant sequence identity to a polynucleotide encoding MDDT, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding MDDT over its entire length; however, portions of the splice variant will have at least about 70%, or alternatively at least about 85%, or alternatively at least about 95%, or alternatively 100% polynucleotide sequence identity to portions of the polynucleotide encoding MDDT. For example, a polynucleotide comprising a sequence of SEQ ID NO:112 and a polynucleotide comprising a sequence of SEQ ID NO:59 are splice variants of each other. Any one of the splice variants described above can encode a polypeptide which contains at least one functional or structural characteristic of MDDT.

It will be appreciated by those skilled in the art that as a result of the degeneracy of the genetic code, a multitude of polynucleotide sequences encoding MDDT, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring MDDT, and all such variations are to be considered as being specifically disclosed.

Although polynucleotides which encode MDDT and its variants are generally capable of hybridizing to polynucleotides encoding naturally occurring MDDT under appropriately selected conditions of stringency, it may be advantageous to produce polynucleotides encoding MDDT or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding MDDT and its derivatives without altering the encoded amino acid sequences include the production of RNA transcripts having more desirable properties, such as a greater half-life, than transcripts produced from the naturally occurring sequence.

The invention also encompasses production of polynucleotides which encode MDDT and MDDT derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding MDDT or any fragment thereof.

Embodiments of the invention can also include polynucleotides that are capable of hybridizing to the claimed polynucleotides, and, in particular, to those having the sequences shown in SEQ ID NO:57-112 and fragments thereof, under various conditions of stringency (Wahl, G. M. and S. L. Berger (1987) Methods Enzymol. 152:399407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase L SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding MDDT may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode MDDT may be cloned in recombinant DNA molecules that direct expression of MDDT, or fragments or functional equivalents thereof, in appropriate host cells. Due to the inherent degeneracy of the genetic code, other polynucleotides which encode substantially the same or a functionally equivalent polypeptides may be produced and used to express MDDT.

The polynucleotides of the invention can be engineered using methods generally known in the art in order to alter MDDT-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected t6 DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of MDDT, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding MDDT may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, MDDT itself or a fragment thereof may be synthesized using chemical methods known in the art. For example, peptide synthesis can be performed using various solution-phase or solid-phase techniques (Creighton, T. (1984) Proteins. Structures and Molecular Properties, WH Freeman, New York N.Y., pp. 5560; Roberge, J. Y. et al. (1995) Science 269:202-204). Automated synthesis may be achieved using the ABI 431A peptide synthesizer (Applied Biosystems). Additionally, the amino acid sequence of MDDT, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing. (Creighton, supra, pp. 28-53).

In order to express a biologically active MDDT, the polynucleotides encoding MDDT or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding MDDT. Such elements may vary in their strength and specificity. Specific initiation signals may also be used to achieve more efficient translation of polynucleotides encoding MDDT. Such signals include the ATG initiation codon and adjacent sequences, e.g. the Kozak sequence. In cases where a polynucleotide sequence encoding MDDT and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding MDDT and appropriate transcriptional and translational control elements. These methods include in vitro recombinant DNA techniques, synthetic techniques, and in vivo genetic recombination (Sambrook, J. et al. (1989) Molecular Cloning. A Laboratory Manual, Cold Spring Harbor Press, Plainview N.Y., ch. 4, 8, and 16-17; Ausubel et al., supra, ch. 1, 3, and 15).

A variety of expression vector/host systems may be utilized to contain and express polynucleotides encoding MDDT. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Imimunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding MDDT. For example, routine cloning, subcloning, and propagation of polynucleotides encoding MDDT can be achieved using a multifunctional E. coli vector such as PBLUESCRIPT (Stratagene, La Jolla Calif.) or PSPORT1 plasmid (Invitrogen). Ligation of polynucleotides encoding MDDT into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of MDDT are needed, e.g. for the production of antibodies, vectors which direct high level expression of MDDT may be used. For example, vectors containing the strong, inducible SP6 or T7 bacteriophage promoter may be used.

Yeast expression systems may be used for production of MDDT. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of MDDT. Transcription of polynucleotides encoding MDDT may be driven by viral promoters, e.g., the ³⁵S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding MDDT may be ligated into an adenovirus transcription/translation complex consisting of the late promoter and tripartite leader sequence. Insertion in a non-essential E1 or E3 region of the viral genome may be used to obtain infective virus which expresses MDDT in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of MDDT in cell lines is preferred. For example, polynucleotides encoding MDDT can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G418; and als and pat confer resistance to chlorsulfuron and phosphinotricin acetyltransferase, respectively (Wigler, M. et al. (1980) Proc. Natl. Acad. Sci. USA 77:3567-3570; Colbere arapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), P-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding MDDT is inserted within a marker gene sequence, transformed cells containing polynucleotides encoding MDDT can be identified by the absence of marker gene function. Alternatively, a marker gene can be placed in tandem with a sequence encoding MDDT under the control of a single promoter. Expression of the marker gene in response to induction or selection usually indicates expression of the tandem gene as well.

In general, host cells that contain the polynucleotide encoding MDDT and that express MDDT may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of MDDT using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on MDDT is preferred, but a competitive binding assay may be employed. These and other assays are well known in the art (Hampton, R. et al. (1990) Serological Methods, a Laboratory Manual, APS Press, St. Paul Minn., Sect IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding MDDT include oligolabeling, nick translation, end-labeling, or PCR amplification using a labeled nucleotide. Alternatively, polynucleotides encoding MDDT, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding MDDT may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode MDDT may be designed to contain signal sequences which direct secretion of MDDT through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HBEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding MDDT may be ligated to a heterologous sequence resulting in translation of a fusion protein in any of the aforementioned host systems. For example, a chimeric MDDT protein containing a heterologous moiety that can be recognized by a commercially available antibody may facilitate the screening of peptide libraries for inhibitors of MDDT activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the MDDT encoding sequence and the heterologous protein sequence, so that MDDT may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled MDDT may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

MDDT, fragments of MDDT, or variants of MDDT may be used to screen for compounds that specifically bind to MDDT. One or more test compounds may be screened for specific binding to MDDT. In various embodiments, 1, 2, 3, 4, 5, 10, 20, 50, 100, or 200 test compounds can be screened for specific binding to MDDT. Examples of test compounds can include antibodies, anticalins, oligonucleotides, proteins (e.g., ligands or receptors), or small molecules.

In related embodiments, variants of MDDT can be used to screen for binding of test compounds, such as antibodies, to MDDT, a variant of MDDT, or a combination of MDDT and/or one or more variants MDDT. In an embodiment, a variant of MDDT can be used to screen for compounds that bind to a variant of MDDT, but not to MDDT having the exact sequence of a sequence of SEQ ID NO:1-56. MDDT variants used to perform such screening can have a range of about 50% to about 99% sequence identity to MDDT, with various embodiments having 60%, 70%, 75%, 80%, 85%, 90%, and 95% sequence identity.

In an embodiment, a compound identified in a screen for specific binding to MDDT can be closely related to the natural ligand of MDDT, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor MDDT (Howard, A. D. et al. (2001) Trends Pharmacol. Sci. 22: 132-140; Wise, A. et al. (2002) Drug Discovery Today 7:235-246).

In other embodiments, a compound identified in a screen for specific binding to MDDT can be closely related to the natural receptor to which MDDT binds, at least a fragment of the receptor, or a fragment of the receptor including all or a portion of the ligand binding site or binding pocket. For example, the compound may be a receptor for MDDT which is capable of propagating a signal, or a decoy receptor for MDDT which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Immunex Corp., Seattle Wash.), which is efficacious for treating rheumatoid arthritis in humans. Etanercept is an engineered p75 tumor necrosis factor (TNF) receptor dimer linked to the Fc portion of human IgG1 (Taylor, P. C. et al. (2001) Curr. Opin. Immunol. 13:611-616).

In one embodiment, two or more antibodies having similar or, alternatively, different specificities can be screened for specific binding to MDDT, fragments of MDDT, or variants of MDDT. The binding specificity of the antibodies thus screened can thereby be selected to identify particular fragments or variants of MDDT. In one embodiment, an antibody can be selected such that its binding specificity allows for preferential identification of specific fragments or variants of MDDT. In another embodiment, an antibody can be selected such that its binding specificity allows for preferential diagnosis of a specific disease or condition having increased, decreased, or otherwise abnormal production of MDDT.

In an embodiment, anticalins can be screened for specific binding to MDDT, fragments of MDDT, or variants of MDDT. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit MDDT involves producing appropriate cells which express MDDT, either as a secreted protein or on the cell membrane. Preferred cells include cells from mammals, yeast, Drosophila, or E. coli. Cells expressing MDDT or cell membrane fractions which contain MDDT are then contacted with a test compound and binding, stimulation, or inhibition of activity of either MDDT or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with MDDT, either in solution or affixed to a solid support, and detecting the binding of MDDT to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

MDDT, fragments of MDDT, or variants of MDDT may be used to screen for compounds that modulate the activity of MDDT. Such compounds may include agonists, antagonists, or partial or inverse agonists. In one embodiment, an assay is performed under conditions permissive for MDDT activity, wherein MDDT is combined with at least one test compound, and the activity of MDDT in the presence of a test compound is compared with the activity of MDDT in the absence of the test compound. A change in the activity of MDDT in the presence of the test compound is indicative of a compound that modulates the activity of MDDT. Alternatively, a test compound is combined with an in vitro or cell-free system comprising MDDT under conditions suitable for MDDT activity, and the assay is performed. In either of these assays, a test compound which modulates the activity of MDDT may do so indirectly and need not come in direct contact with the test compound. At least one and up to a plurality of test compounds may be screened.

In another embodiment, polynucleotides encoding MDDT or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding MDDT may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding MDDT can also be used to create “knockin” humanized animals (pigs) or transgenic animals (mice or rats) to model human disease. With knockin technology, a region of a polynucleotide encoding MDDT is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress MDDT, e.g., by secreting MDDT in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of MDDT and molecules for disease detection and treatment. In particular, SEQ ID NO:110 shows co-expression with osteoporosis-relevant genes. In addition, examples of tissues expressing MDDT can be found in Table 6 and can also be found in Example XI. Therefore, MDDT appears to play a role in cell proliferative, autoimmune/inflammatory, developmental, and neurological disorders, diseases treated with steroids and disorders caused by the metabolic response to treatment with steroids. In the treatment of disorders associated with increased MDDT expression or activity, it is desirable to decrease the expression or activity of MDDT. In the treatment of disorders associated with decreased MDDT expression or activity, it is desirable to increase the expression or activity of MDDT.

Therefore, in one embodiment, MDDT or a fragment or derivative thereof may be administered to a subject to treat or prevent a disorder associated with decreased expression or activity of MDDT. Examples of such disorders include, but are not limited to, a cell proliferative disorder such as actinic keratosis, arteriosclerosis, atherosclerosis, bursitis, cirrhosis, hepatitis, mixed connective tissue disease (MCID), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helninthic infections, and trauma; a disease treated with a steroid and a disorder caused by the metabolic response to treatment with steroids, such as adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; and a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia. SEQ ID NO:2 can be used in the diagnosis and treatment of Tangier disease and SEQ ID NO: 5 can be used in the diagnosis and treatment of type II diabetes.

In another embodiment, a vector capable of expressing MDDT or a fragment or derivative thereof may be administered to a subject to treat or prevent a disorder associated with decreased expression or activity of MDDT including, but not limited to, those described above.

In a further embodiment, a composition comprising a substantially purified MDDT in conjunction with a suitable pharmaceutical carrier may be administered to a subject to treat or prevent a disorder associated with decreased expression or activity of MDDT including, but not limited to, those provided above.

In still another embodiment, an agonist which modulates the activity of MDDT may be administered to a subject to treat or prevent a disorder associated with decreased expression or activity of MDDT including, but not limited to, those listed above.

In a further embodiment, an antagonist of MDDT may be administered to a subject to treat or prevent a disorder associated with increased expression or activity of MDDT. Examples of such disorders include, but are not limited to, those cell proliferative, autoimmune/inflammatory, developmental, and neurological disorders, diseases treated with steroids and disorders caused by the metabolic response to treatment with steroids described above. In one aspect, an antibody which specifically binds MDDT may be used directly as an antagonist or indirectly as a targeting or delivery mechanism for bringing a pharmaceutical agent to cells or tissues which express MDDT.

In an additional embodiment, a vector expressing the complement of the polynucleotide encoding MDDT may be administered to a subject to treat or prevent a disorder associated with increased expression or activity of MDDT including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of MDDT may be produced using methods which are generally known in the art. In particular, purified MDDT may be used to produce antibodies or to screen libraries of pharmaceutical agents to identify those which specifically bind MDDT. Antibodies to MDDT may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with MDDT or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to MDDT have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of MDDT amino acids may be fused with those of another protein, such as KLH, and antibodies to the chimeric molecule may be produced.

Monoclonal antibodies to MDDT may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce MDDT-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for MDDT may also be generated. For example, such fragments include, but are not limited to, F(ab)₂ fragments produced by pepsin digestion of the antibody molecule and Fab fragments generated by reducing the disulfide bridges of the F(ab)₂ fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between MDDT and its specific antibody. A two-site, monoclonal-based immunoassay utilizing monoclonal antibodies reactive to two non-interfering MDDT epitopes is generally used, but a competitive binding assay may also be employed (Pound, supra).

Various methods such as Scatchard analysis in conjunction with radioimmunoassay techniques may be used to assess the affinity of antibodies for MDDT. Affinity is expressed as an association constant, Kₐ, which is defined as the molar concentration of MDDT-antibody complex divided by the molar concentrations of free antigen and free antibody under equilibrium conditions. The Kₐ determined for a preparation of polyclonal antibodies, which are heterogeneous in their affinities for multiple MDDT epitopes, represents the average affinity, or avidity, of the antibodies for MDDT. The Kₐ determined for a preparation of monoclonal antibodies, which are monospecific for a particular MDDT epitope, represents a true measure of affinity. High-affinity antibody preparations with Kₐ ranging from about 10⁹ to 10¹² L/mole are preferred for use in immunoassays in which the MDDT-antibody complex must withstand rigorous manipulations. Low-affinity antibody preparations with Kₐ ranging from about 10⁶ to 10⁷ L/mole are preferred for use in immunopurification and similar procedures which ultimately require dissociation of MDDT, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of MDDT-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding MDDT, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding MDDT. Such technology is well known in the art, and antisense oligonucleotides or larger fragments can be designed from various locations along the coding or control regions of sequences encoding MDDT (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding MDDT may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, ILG. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparuin and Trypanosoma cruzz). In the case where a genetic deficiency in MDDT expression or regulation causes disease, the expression of MDDT from an appropriate population of transduced cells may alleviate the clinical manifestations caused by the genetic deficiency.

In a further embodiment of the invention, diseases or disorders caused by deficiencies in MDDT are treated by constructing mammalian expression vectors encoding MDDT and introducing these vectors by mechanical means into MDDT-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Recipon (1998) Curr. Opin. Biotechnol. 9:445-450).

Expression vectors that may be effective for the expression of MDDT include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). MDDT may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451456), commercially available in the T-REX plasmid (Invitrogen)); the ecdysone-inducible promoter (available in the plasmids PVGRXR and PIND; Invitrogen); the FK506/rapamycin inducible promoter, or the RU486/mifepristone inducible promoter (Rossi, F. M. V. and H. M. Blau, supra)), or (iii) a tissue-specific promoter or the native promoter of the endogenous gene encoding MDDT from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to MDDT expression are treated by constructing a retrovirus vector consisting of (i) the polynucleotide encoding MDDT under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding MDDT to cells which have one or more genetic abnormalities with respect to the expression of MDDT. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding MDDT to target cells which have one or more genetic abnormalities with respect to the expression of MDDT. The use of herpes simplex virus (HSV)-based vectors may be especially valuable for introducing MDDT to cells of the central nervous system, for which HSV has a tropism The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding MDDT to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for MDDT into the alphavirus genome in place of the capsid-coding region results in the production of a large number of MDDT-coding RNAs and the synthesis of high levels of MDDT in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of MDDT into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. L Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding MDDT.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding MDDT. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding MDDT. Compounds which may be effective in altering expression of a specific polynucleotide may include, but are not limited to, oligonucleotides, antisense oligonucleotides, triple helix-forming oligonucleotides, tmmscription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased MDDT expression or activity, a compound which specifically inhibits expression of the polynucleotide encoding MDDT may be therapeutically useful, and in the treatment of disorders associated with decreased MDDT expression or activity, a compound which specifically promotes expression of the polynucleotide encoding MDDT may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding MDDT is exposed to at least one test compound thus obtained. The sample may comprise, for example, an intact or permeabilized cell, or an in vitro cell-free or reconstituted biochemical system. Alterations in the expression of a polynucleotide encoding MDDT are assayed by any method commonly known in the art. Typically, the expression of a specific nucleotide is detected by hybridization with a probe having a nucleotide sequence complementary to the sequence of the polynucleotide encoding MDDT. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of MDDT, antibodies to MDDT, and mimetics, agonists, antagonists, or inhibitors of MDDT.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising MDDT or fragments thereof. For example, liposome preparations containing a cell-impermeable macromolecule may promote cell fusion and intracellular delivery of the macromolecule. Alternatively, MDDT or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example MDDT or fragments thereof, antibodies of MDDT, and agonists, antagonists or inhibitors of MDDT, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind MDDT may be used for the diagnosis of disorders characterized by expression of MDDT, or in assays to monitor patients being treated with MDDT or agonists, antagonists, or inhibitors of MDDT. Antibodies useful for diagnostic purposes may be prepared in the same manner as described above for therapeutics. Diagnostic assays for MDDT include methods which utilize the antibody and a label to detect MDDT in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring MDDT, including ELISAs, RIAs, and FACS, are known in the art and provide a basis for diagnosing altered or abnormal levels of MDDT expression. Normal or standard values for MDDT expression are established by combining body fluids or cell extracts taken from normal mammalian subjects, for example, human subjects, with antibodies to MDDT under conditions suitable for complex formation. The amount of standard complex formation may be quantitated by various methods, such as photometric means. Quantities of MDDT expressed in subject, control, and disease samples from biopsied tissues are compared with the standard values. Deviation between standard and subject values establishes the parameters for diagnosing disease.

In another embodiment of the invention, polynucleotides encoding MDDT may be used for diagnostic purposes. The polynucleotides which may be used include oligonucleotides, complementary RNA and DNA molecules, and PNAs. The polynucleotides may be used to detect and quantify gene expression in biopsied tissues in which expression of MDDT may be correlated with disease. The diagnostic assay may be used to determine absence, presence, and excess expression of MDDT, and to monitor regulation of MDDT levels during therapeutic intervention.

In one aspect, hybridization with PCR probes which are capable of detecting polynucleotides, including genomic sequences, encoding MDDT or closely related molecules may be used to identify nucleic acid sequences which encode MDDT. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding MDDT, allelic variants, or related sequences.

Probes may also be used for the detection of related sequences, and may have at least 50% sequence identity to any of the MDDT encoding sequences. The hybridization probes of the subject invention may be DNA or RNA and may be derived from the sequence of SEQ ID NO:57-112 or from genomic sequences including promoters, enhancers, and introns of the MDDT gene.

Means for producing specific hybridization probes for polynucleotides encoding MDDT include the cloning of polynucleotides encoding MDDT or MDDT derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding MDDT may be used for the diagnosis of disorders associated with expression of MDDT. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and heliminthic infections, and trauma; a disease treated with a steroid and a disorder caused by the metabolic response to treatment with steroids, such as adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; and a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmn-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia. SEQ ID NO:58, encoding SEQ ID NO:2, and SEQ ID NO:2 can be used in the diagnosis and treatment of Tangier disease and SEQ ID NO:61, encoding SEQ ID NO:5, and SEQ ID NO:5 can be used in the diagnosis and treatment of type II diabetes. Polynucleotides encoding MDDT may be used in Southern or northern analysis, dot blot, or other membrane-based technologies; in PCR technologies; in dipstick, pin, and multiformat ELISA-like assays; and in microarrays utilizing fluids or tissues from patients to detect altered MDDT expression. Such qualitative or quantitative methods are well known in the art.

In a particular aspect, polynucleotides encoding MDDT may be used in assays that detect the presence of associated disorders, particularly those mentioned above. Polynucleotides complementary to sequences encoding MDDT may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding MDDT in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of MDDT, a normal or standard profile for expression is established. This may be accomplished by combining body fluids or cell extracts taken from normal subjects, either animal or human, with a sequence, or a fragment thereof, encoding MDDT, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding MDDT may involve the use of PCR. These oligomers may be chemically synthesized, generated enzymatically, or produced in vitro. Oligomers will preferably contain a fragment of a polynucleotide encoding MDDT, or a fragment of a polynucleotide complementary to the polynucleotide encoding MDDT, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding MDDT may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding MDDT are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In FSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of MDDT include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem 212:229-236). The speed of quantitation of multiple samples may be accelerated by running the assay in a high-throughput format where the oligomer or polynucleotide of interest is presented in various dilutions and a spectrophotometric or calorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, MDDT, fragments of MDDT, or antibodies specific for MDDT may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurning environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467471). Ifa test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for MDDT to quantify the levels of MDDT expression. In one embodiment, the antibodies are used as elements on a microarray, and protein expression levels are quantified by exposing the microarray to the sample and detecting the levels of protein bound to each array element (Lueling, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding MDDT may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding MDDT on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, MDDT, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between MDDT and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with MDDT, or fragments thereof, and washed. Bound MDDT is then detected by methods well known in the art. Purified MDDT can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding MDDT specifically compete with a test compound for binding MDDT. In this manner, antibodies can be used to detect the presence of any peptide which shares one or more antigenic determinants with MDDT.

In additional embodiments, the nucleotide sequences which encode MDDT may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications and publications, mentioned above and below, in particular U.S. Ser. No. 60/304,298, U.S. Ser. No. 60/305,324, U.S. Ser. No. 60/307,003, U.S. Ser. No. 60/308,185, U.S. Ser. No. 60/310,096, U.S. Ser. No. 60/311,551 and U.S. Ser. No. 60/363,649, are expressly incorporated by reference herein.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOIR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch.

7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:4143); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of fill length polynucleotide and polypeptide sequences were also used to identify polynucleotide sequence fragments from SEQ ID NO:57-112. Fragments from about 20 to about 4000 nucleotides which are useful in hybridization and amplification technologies are described in Table 4, column 2.

IV. Identification and Editing of Coding Sequences from Genomic DNA

Putative molecules for disease detection and treatment were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode molecules for disease detection and treatment, the encoded polypeptides were analyzed by querying against PFAM models for molecules for disease detection and treatment. Potential molecules for disease detection and treatment were also identified by homology to Incyte cDNA sequences that had been annotated as molecules for disease detection and treatment. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example m. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example m were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example m were queried against public databases such as the GenBank primate, rodent, mammalian, vertebrate, and eukaryote databases using the BLAST program The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of MDDT Encoding Polynucleotides

The sequences which were used to assemble SEQ ID NO:57-112 were compared with sequences from the Incyte LIFESEQ database and public domain databases using BLAST and other implementations of the Smith-Waterman algorithm. Sequences from these databases that matched SEQ ID NO:57-112 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook, supra, ch. 7; Ausubel et al., supra, ch. 4).

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and 4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding MDDT are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding MDDT. cDNA sequences and cDNA library/tissue information are found in the LIFESEQ GOLD database (Incyte Genomics, Palo Alto Calif.).

VIII. Extension of MDDT Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2×carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosysterns).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in MDDT Encoding Polynucleotides

Common DNA sequence variants known as single nucleotide polymorphisms (SNPs) were identified in SEQ ID NO:57-112 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:57-112 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 μmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1×saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porqus surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 μg/μl oligo-(dT) primer (21 mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHI kits (Incyte). Specific control poly(A)+ RNAs are synthesized by in vitro transcription from non-coding yeast genomic DNA. After incubation at 37° C. for 2 hr, each reaction sample (one with Cy3 and another with CyS labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5× SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and CyS labeled cDNA synthesis products in SX SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20×microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for CyS. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibited at least about a two-fold change in expression, a signal-to-background ratio of at least 2.5, and an element spot size of at least 40% were identified as differentially expressed using the GEMTOOLS program (Incyte Genomics).

Expression

For example, SEQ ID NO:58 was downregulated by at least two-fold in five out of the eight endothelial cell lines treated with 10 ng/ml TNF-α within four hours. SEQ ID NO:58 was also down regulated in Tangier disease fibroblasts as compared to normal controls suggesting that SEQ ID NO:58, encoding SEQ ID NO:2, can be used for the diagnosis, prognosis or treatment of a cardiovascular disorder such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, coronary artery bypass, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheurnatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and Tangier disease.

For example, SEQ ID NO:90 showed differential expression in treated versus non-treated C3A cells as determined by microarray analysis. The expression of SEQ ID NO:90 was decreased by at least two fold in C3A cells treated with 10 μM MAH for one to six hours and with 100 μM for one or six hours versus untreated C3A cells. SEQ ID NO:90 expression was also decreased by at least two-fold when C3A cells were treated for one to three hours with either 1 μM, 10 μM, or 100 μM bude when compared with untreated C3A cells. These experiments indicate that SEQ ID NO:90 was significantly under-expressed in C3A cells when tested with two steroid compounds, further establishing the utility of SEQ ID NO:90 as a diagnostic marker or as a potential therapeutic target for liver disorders associated with steroid therapy such as adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease.

Human CD34 positive precursor cells were isolated by positive immunomagnetic selection from the leukapheresis of normal volunteer donors who had undergone G-CSF-induced stem cell mobilization. The purified CD34+ cells were cultured in vitro for 10 days in the presence of recombinant GM-CSF, Stem Cell Factor, TNF-alpha, TGF-beta1, and Flt3-Ligand. The resulting expanded cell population was enriched for cell cluster-forming immature dendritic cells (Lci) by sedimentation over a 7.5% BSA column at 1 g for 30 min. Immature dendritic cells were cultured for two additional days in the presence of the same combination of cytokines supplemented with LPS, IL-1beta, TNF-alpha, or double strand RNA. In addition, cluster-forming immature dendritic cells were disrupted by vigorous pipetting and cultured for two additional days in the presence of the same combination of cytokines without addition of any additional factor. The partially mature dendritic cells derived by mechanical disruption of cell clusters are characterized by the presence of intracellular rod-shaped structures called Birbeck's Granules. The dendritic cell population produced using this method was called Birbeck's Granule-positive dendritic cells, or BG.

CD34+ precursor cells were compared to immature dendritic cells (Lci); Lci were compared to mature dendritic cells derived in the presence of LPS, IL-1b, TNF-alpha, or double strand RNA; and undisturbed Lci (Clusters) were compared to BG. Array elements that exhibited about at least a two-fold change in expression and a signal intensity over 250 units, a signal-to-background ratio of at least 2.5, and an element spot size of at least 40% were identified as differentially expressed using the GEMTOOLS program (Incyte Genomics). SEQ ID NO:96, which contains a GTPase activating protein motif for Arf and a Rho GAP domain, showed at least a two-fold increased expression during the differentiation of dendritic cells when induced by simple mechanical disaggregation and a greater than two-fold expression during maturation of these cells when induced by TNF-alpha. TNF-alpha is a factor produced by many cell types in response to stress. In addition, mechanical disruption is a significant stress factor during organ transplantation. Further, these experiments indicate that SEQ ID NO:96 was significantly over-expressed during differentiation and maturation of human dendritic cells, further establishing the utility of SEQ ID NO:96 as a diagnostic marker or as a potential therapeutic target for organ transplant disorders.

For example, SEQ ID NO:110 showed differential expression in diseased versus normal tissue as determined by microarray analysis. Matched normal ovary and ovarian tumor tissue samples are provided by the Huntsman Cancer Institute, (Salt Lake City, Utah). The expression of MDDT was decreased in ovarian tumor cells relative to non-tumorous ovarian cells. Therefore, SEQ ID NO:110 is useful in diagnostic assays for ovarian cancer.

XII. Complementary Polynucleotides

Sequences complementary to the MDDT-encoding sequences, or any parts thereof, are used to detect, decrease, or inhibit expression of naturally occurring MDDT. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of MDDT. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the MDDT-encoding transcript.

XIII. Expression of MDDT

Expression and purification of MDDT is achieved using bacterial or virus-based expression systems. For expression of MDDT in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express MDDT upon induction with isopropyl beta-D-thiogalactopyranoside (IPTG). Expression of MDDT in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding MDDT by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, MDDT is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from MDDT at specifically engineered sites. FLAG, an 8-amino acid peptide, enables immunoaffiity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified MDDT obtained by these methods can be used directly in the assays shown in Examples XVI, and XVIII where applicable.

XIV. Functional Assays

MDDT function is assessed by expressing the sequences encoding MDDT at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of MDDT on gene expression can be assessed using highly purified populations of cells transfected with sequences encoding MDDT and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding MDDT and other genes of interest can be analyzed by northern analysis or microarray techniques.

XV. Production of MDDT Specific Antibodies

MDDT substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the MDDT amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-MDDT activity by, for example, binding the peptide or MDDT to a substrate, blocking with 1% BSA, reacting with rabbit antisera, washing, and reacting with radio-iodinated goat anti-rabbit IgG.

XVI. Purification of Naturally Occurring MDDT Using Specific Antibodies

Naturally occurring or recombinant MDDT is substantially purified by immunoaffinity chromatography using antibodies specific for MDDT. An immunoaffinity column is constructed by covalently coupling anti-MDDT antibody to an activated chromatographic resin, such as CNBr-activated SEPHAROSE (Amersham Biosciences). After the coupling, the resin is blocked and washed according to the manufacturer's instructions.

Media containing MDDT are passed over the immunoaffinity column, and the column is washed under conditions that allow the preferential absorbance of MDDT (e.g., high ionic strength buffers in the presence of detergent). The column is eluted under conditions that disrupt antibody/MDDT binding (e.g., a buffer of pH 2 to pH 3, or a high concentration of a chaotrope, such as urea or thiocyanate ion), and MDDT is collected.

XVII. Identification of Molecules Which Interact with MDDT

MDDT, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled MDDT, washed, and any wells with labeled MDDT complex are assayed. Data obtained using different concentrations of MDDT are used to calculate values for the number, affinity, and association of MDDT with the candidate molecules.

Alternatively, molecules interacting with MDDT are analyzed using the yeast two-hybrid system as described in Fields, S. and 0. Song (1989; Nature 340:245-246), or using commercially available kits based on the two-hybrid system, such as the MATCHMAKER system (Clontech).

MDDT may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of MDDT Activity

Phorbol ester binding activity of MDDT is measured using an assay based on the fluorescent phorbol ester sapinotoxin-D (SAPD). Binding of SAPD to MDDT is quantified by measuring the resonance energy transfer from MDDT tryptophans to the 2-(N-methylamino)benzoyl fluorophore of the phorbol ester, as described by Slater et al. ((1996) J. Biol. Chem. 271:4627-4631).

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | | | Incyte | |
|---|---|---|---|---|---|
| Incyte | Polypeptide | Incyte | Polynucleotide | Polynucleotide | Incyte Full Length |
| Project ID | SEQ ID NO: | Polypeptide ID | SEQ ID NO: | ID | Clones |
| 2867236 | 1 | 2867236CD1 | 57 | 2867236CB1 | 1536179CA2 |
| 1294096 | 2 | 1294096CD1 | 58 | 1294096CB1 | |
| 7238537 | 3 | 7238537CD1 | 59 | 7238537CB1 | 90171762CA2, |
| | | | | | 90171778CA2, |
| | | | | | 90171786CA2, |
| | | | | | 90171870CA2, |
| | | | | | 90171886CA2, |
| | | | | | 90171894CA2, |
| | | | | | 90190048CA2, |
| | | | | | 90190116CA2, |
| | | | | | 90190124CA2, |
| | | | | | 90190132CA2 |
| 7494391 | 4 | 7494391CD1 | 60 | 7494391CB1 | 90140260CA2 |
| 6451054 | 5 | 6451054CD1 | 61 | 6451054CB1 | |
| 7494592 | 6 | 7494592CD1 | 62 | 7494592CB1 | |
| 5202657 | 7 | 5202657CD1 | 63 | 5202657CB1 | 3343965CA2 |
| 2013529 | 8 | 2013529CD1 | 64 | 2013529CB1 | 4010537CA2, |
| | | | | | 6300041CA2 |
| 3841351 | 9 | 3841351CD1 | 65 | 3841351CB1 | |
| 152116 | 10 | 152116CD1 | 66 | 152116CB1 | |
| 2381031 | 11 | 2381031CD1 | 67 | 2381031CB1 | 2381031CA2 |
| 2511371 | 12 | 2511371CD1 | 68 | 2511371CB1 | |
| 8068623 | 13 | 8068623CD1 | 69 | 8068623CB1 | |
| 677977 | 14 | 677977CD1 | 70 | 677977CB1 | |
| 1661472 | 15 | 1661472CD1 | 71 | 1661472CB1 | |
| 1748508 | 16 | 1748508CD1 | 72 | 1748508CB1 | 90132493CA2 |
| 2159545 | 17 | 2159545CD1 | 73 | 2159545CB1 | |
| 8560269 | 18 | 8560269CD1 | 74 | 8560269CB1 | |
| 8710302 | 19 | 8710302CD1 | 75 | 8710302CB1 | |
| 6778214 | 20 | 6778214CD1 | 76 | 6778214CB1 | |
| 258383 | 21 | 258383CD1 | 77 | 258383CB1 | 90140053CA2, |
| | | | | | 90140161CA2 |
| 2804937 | 22 | 2804937CD1 | 78 | 2804937CB1 | |
| 7494915 | 23 | 7494915CD1 | 79 | 7494915CB1 | |
| 2073751 | 24 | 2073751CD1 | 80 | 2073751CB1 | |
| 3178841 | 25 | 3178841CD1 | 81 | 3178841CB1 | |
| 3674807 | 26 | 3674807CD1 | 82 | 3674807CB1 | 3674807CA2 |
| 1794922 | 27 | 1794922CD1 | 83 | 1794922CB1 | 90144984CA2 |
| 1795509 | 28 | 1795509CD1 | 84 | 1795509CB1 | 690351CA2, |
| | | | | | 90131912CA2, |
| | | | | | 90131949CA2, |
| | | | | | 90131952CA2, |
| | | | | | 90131960CA2, |
| | | | | | 90131976CA2, |
| | | | | | 90131992CA2, |
| | | | | | 90132060CA2, |
| | | | | | 90132084CA2, |
| | | | | | 90132092CA2 |
| 2017180 | 29 | 2017180CD1 | 85 | 2017180CB1 | 2807727CA2 |
| 219442 | 30 | 219442CD1 | 86 | 219442CB1 | |
| 2597459 | 31 | 2597459CD1 | 87 | 2597459CB1 | 90140160CA2 |
| 2783863 | 32 | 2783863CD1 | 88 | 2783863CB1 | |
| 2902971 | 33 | 2902971CD1 | 89 | 2902971CB1 | |
| 368660 | 34 | 368660CD1 | 90 | 368660CB1 | 90130001CA2 |
| 2804990 | 35 | 2804990CD1 | 91 | 2804990CB1 | 7616219CA2 |
| 168571 | 36 | 168571CD1 | 92 | 168571CB1 | |
| 1286391 | 37 | 1286391CD1 | 93 | 1286391CB1 | |
| 2007684 | 38 | 2007684CD1 | 94 | 2007684CB1 | |
| 2227040 | 39 | 2227040CD1 | 95 | 2227040CB1 | |
| 4346130 | 40 | 4346130CD1 | 96 | 4346130CB1 | |
| 55117040 | 41 | 55117040CD1 | 97 | 55117040CB1 | 55117036CA2 |
| 7472392 | 42 | 7472392CD1 | 98 | 7472392CB1 | 6622373CA2 |
| 4028960 | 43 | 4028960CD1 | 99 | 4028960CB1 | |
| 8227004 | 44 | 8227004CD1 | 100 | 8227004CB1 | 3279686CA2 |
| 3044763 | 45 | 3044763CD1 | 101 | 3044763CB1 | 90126287CA2 |
| 4044519 | 46 | 4044519CD1 | 102 | 4044519CB1 | 4044519CA2, |
| | | | | | 90106511CA2, |
| | | | | | 90106619CA2, |
| | | | | | 90106627CA2, |
| | | | | | 90106659CA2 |
| 71351918 | 47 | 71351918CD1 | 103 | 71351918CB1 | |
| 8109363 | 48 | 8109363CD1 | 104 | 8109363CB1 | 3853651CA2, |
| | | | | | 6859649CA2 |
| 1272746 | 49 | 1272746CD1 | 105 | 1272746CB1 | |
| 1839974 | 50 | 1839974CD1 | 106 | 1839974CB1 | 90120531CA2 |
| 1877336 | 51 | 1877336CD1 | 107 | 1877336CB1 | |
| 2321054 | 52 | 2321054CD1 | 108 | 2321054CB1 | 1236972CA2, |
| | | | | | 1398127CA2, |
| | | | | | 2245649CA2, |
| | | | | | 2321054CA2, |
| | | | | | 90106305CA2, |
| | | | | | 90106337CA2, |
| | | | | | 90106353CA2, |
| | | | | | 90106361CA2, |
| | | | | | 90106369CA2, |
| | | | | | 90106377CA2, |
| | | | | | 90106385CA2, |
| | | | | | 90106393CA2, |
| | | | | | 90106405CA2, |
| | | | | | 90106429CA2, |
| | | | | | 90106437CA2, |
| | | | | | 90106469CA2, |
| | | | | | 90106493CA2 |
| 2796034 | 53 | 2796034CD1 | 109 | 2796034CB1 | |
| 4413112 | 54 | 4413112CD1 | 110 | 4413112CB1 | 90108176CA2, |
| | | | | | 90172662CA2 |
| 7654832 | 55 | 7654832CD1 | 111 | 7654832CB1 | 90110764CA2, |
| | | | | | 90110788CA2, |
| | | | | | 90110880CA2 |
| 7503849 | 56 | 7503849CD1 | 112 | 7503849CB1 | |

TABLE 2

| | | GenBank ID NO: | | |
|---|---|---|---|---|
| Polypeptide SEQ | Incyte | or PROTEOME | Probability | |
| ID NO: | Polypeptide ID | ID NO: | Score | Annotation |
| 1 | 2867236CD1 | g11065721 | 3.50E−134 | [Homo sapiens] 28 kD interferon responsive protein |
| 4 | 7494391CD1 | g13506808 | 5.70E−42 | [Mus musculus] thymic stromal co-transporter |
| | | | | Chen, C., et al., Biochim. Biophys. Acta 1493: 159-169 (2000) |
| 7 | 5202657CD1 | g3006139 | 7.90E−42 | [Schizosaccharomyces pombe] hypothetical zf-C3HC4 zinc finger protein |
| 15 | 1661472CD1 | g6899934 | 1.90E−24 | [Arabidopsis thaliana] putative zinc-finger protein |
| 17 | 2159545CD1 | g4650844 | 3.40E−120 | [Homo sapiens] Kelch motif containing protein |
| 18 | 8560269CD1 | g18252514 | 1.00E−123 | [Homo sapiens] hepatocellular carcinoma-associated antigen HCA557b |
| 20 | 6778214CD1 | g57671 | 8.90E−15 | [Rattus norvegicus] ribonuclease inhibitor |
| | | | | Kawanomoto, M., et al., Biochim. Biophys. Acta 1129: 335-338 (1992) |
| 22 | 2804937CD1 | g6650377 | 0 | [Mus musculus] pecanex 1 |
| 23 | 7494915CD1 | g2072953 | 1.60E−34 | [Homo sapiens] putative p150 |
| | | | | Sassaman, D. M. et al. (1997) Nature Genet. 16: 37-43 |
| 24 | 2073751CD1 | g18652658 | 4.00E−17 | [Schmidtea mediterranea] myosin heavy chain A |
| 31 | 2597459CD1 | g7243777 | 2.90E−50 | [Drosophila melanogaster] Diablo |
| 35 | 2804990CD1 | g1196425 | 4.60E−28 | [Homo sapiens] envelope protein |
| | | | | Cohen, M. et al. (1985) Virology 147: 449-458 |
| 36 | 168571CD1 | g3093476 | 6.90E−90 | [Homo sapiens] EVI-5 homolog |
| | | | | Liao, X. et al. (1997) Oncogene 14: 1023-1029 |
| 39 | 2227040CD1 | g1799568 | 1.50E−80 | [Homo sapiens] stac |
| | | | | Suzuki H., et al. (1996) Biochem. Biophys. Res. Commun. 229: 902-909 |
| 40 | 4346130CD1 | g15625572 | 0 | [Homo sapiens] centaurin delta1 |
| 42 | 7472392CD1 | g12853030 | 3.00E−81 | [Mus musculus] Cyclic nucleotide-binding domain containing protein˜data |
| | | | | source: Pfam, source key: PF00027, evidence: ISS˜putative |
| 43 | 4028960CD1 | g6063688 | 4.50E−102 | [Homo sapiens] AMMECR1 |
| | | | | Vitelli, F., et al., Genomics 55: 335-340 (1999) |
| 44 | 8227004CD1 | g11527201 | 6.70E−250 | [Homo sapiens] sporulation-induced transcript 4-associated protein SAPLa |
| | | | | Twells, R. C. J., et al., Genomics 72: 231-242 (2001) |
| 47 | 71351918CD1 | g1314316 | 6.30E−73 | [Xenopus laevis] WD-40 motifs; up-regulated by thyroid hormone in tadpoles |
| | | | | Brown, D. D., et al., Proc. Natl. Acad. Sci. U.S.A. 9: 1924-1929 (1996) |
| 52 | 2321054CD1 | g15278367 | 4.00E−52 | [Homo sapiens] Similar to fasciculation and elongation protein zeta 2 (zygin II) |
| 55 | 7654832CD1 | g15420869 | 0 | [Mus musculus] ankyrin repeat-containing SOCS box protein 5 |
| | | | | Kile, B. T., et al., Mol. Cell. Biol. 21: 6189-6197 (2001) |

TABLE 3

| | | Amino | | | | Analytical |
|---|---|---|---|---|---|---|
| SEQ | Incyte | Acid | Potential | Potential | | Methods |
| ID | Polypeptide | Resi- | Phosphorylation | Glycosylation | | and |
| NO: | ID | dues | Sites | Sites | Signature Sequences, Domains and Motifs | Databases |
| 1 | 2867236CD1 | 246 | S99 S212 T11 T26 | N129 N201 | | |
| | | | T73 T111 T131 | | | |
| 2 | 1294096CD1 | 325 | S6 S25 S54 S142 | N254 | K+ channel tetramerisation domain: D34-Q134 | HMMER_PFAM |
| | | | S151 S162 S204 | | | |
| | | | S304 S316 T47 T51 | | | |
| | | | T252 T267 Y119 | | | |
| 3 | 7238537CD1 | 376 | S34 S52 S96 S106 | N152 N319 | Cytosolic domain: S321-A376 | TMHMMER |
| | | | S113 S118 S264 | | Transmembrane domain: T298-V320 | |
| | | | S359 S373 T38 | | Non-cytosolic domain: M1-V297 | |
| | | | T231 T286 | | | |
| 4 | 7494391CD1 | 461 | S48 S70 S93 S233 | N38 N46 N53 | Sugar transport proteins signature 1: L91-S107 | MOTIFS |
| | | | S253 S440 S454 | N143 N229 | | |
| | | | S459 T166 Y39 | N251 | | |
| | | | | | Cytosolic domain: M1-V6, D96-K101, D157-R168, | TMHMMER |
| | | | | | G219-E258, S319-D324, K374-L384, K437-R461 | |
| | | | | | Transmembrane domain: E7-Y29, I78-S95, F102- | |
| | | | | | F124, S134-V156, I169-I191, F196-L218, L259- | |
| | | | | | F281, F296-F318, I325-T347, M351-S373, F385- | |
| | | | | | Y407, F417-V436 | |
| | | | | | Non-cytosolic domain: R30-D77, A125-A133, R192- | |
| | | | | | G195, I282-V295, T348-M350, S408-G416 | |
| 5 | 6451054CD1 | 168 | S26 S28 S47 S80 | N50 | | |
| | | | S85 S98 S113 T96 | | | |
| 6 | 7494592CD1 | 832 | S201 S363 S470 | N586 | | |
| | | | S489 S557 S580 | | | |
| | | | S658 S702 S785 | | | |
| | | | S799 S818 T166 | | | |
| | | | T228 T324 T352 | | | |
| | | | T366 T371 T399 | | | |
| | | | T545 T659 T734 | | | |
| | | | Y430 | | | |
| 7 | 5202657CD1 | 393 | S28 S135 S180 | N189 | PROTEIN C29A3.03C CHROMOSOME II | BLAST_{—} |
| | | | S190 Y35 Y218 | | ZINCFINGER NUCLEAR DNABINDING CODED | PRODOM |
| | | | Y252 | | FOR BY | |
| | | | | | PD024560: E158-F393 | |
| 8 | 2013529CD1 | 280 | S41 S77 S138 S144 | N232 | | |
| | | | S159 S183 S193 | | | |
| | | | S235 S252 S257 | | | |
| | | | S272 T20 T155 | | | |
| | | | T158 T178 | | | |
| 9 | 3841351CD1 | 344 | S5 S14 S15 S33 | N217 | | |
| | | | S38 S169 T130 | | | |
| | | | T173 T271 T276 | | | |
| 10 | 152116CD1 | 405 | S139 S163 S292 | N147 | | |
| | | | S322 S346 S361 | | | |
| | | | T10 T70 T151 | | | |
| | | | T325 | | | |
| 11 | 2381031CD1 | 185 | S52 S56 S130 S134 | N121 N141 | | |
| | | | S143 S145 S181 | | | |
| | | | T60 | | | |
| 12 | 2511371CD1 | 463 | S8 S261 S300 S330 | | Trp-Asp (WD) repeat protein BL00678: S210-W220 | BLIMPS_{—} |
| | | | T47 T88 T166 | | | BLOCKS |
| | | | T313 | | | |
| | | | | | WD domain, G-beta repeat: C186-R221, P343-D376, | HMMER_PFAM |
| | | | | | P254-K290 | |
| 13 | 8068623CD1 | 403 | S85 S106 S164 | | TBC domain: E57-C268 | HMMER_PFAM |
| | | | S389 T324 T364 | | | |
| | | | | | Cytosolic domain: R261-T364 | TMHMMER |
| | | | | | Transmembrane domain: L238-Y260, N365-V387 | |
| | | | | | Non-cytosolic domain: M1-R237, K388-P403 | |
| 14 | 677977CD1 | 574 | S45 S57 S65 S79 | N214 | signal_cleavage: M1-A22 | SPSCAN |
| | | | S80 S218 S234 | | | |
| | | | S246 S340 S376 | | | |
| | | | T53 T58 T519 | | | |
| | | | | | Cell attachment sequence: R203-D205 | MOTIFS |
| 15 | 1661472CD1 | 731 | S9 S100 S121 S156 | N335 | PROTEIN ZINC FINGER CONSERVED | BLAST_{—} |
| | | | S160 S219 S264 | | CHROMOSOME IV COSMID CODED FOR BY C | PRODOM |
| | | | S339 S340 S393 | | PD043678: D4-R101 | |
| | | | S422 S450 S590 | | | |
| | | | S625 S669 S672 | | | |
| | | | T85 T222 T359 | | | |
| | | | T377 T491 T561 | | | |
| | | | T648 Y23 | | | |
| | | | | | PROLINE-RICH PROTEIN DM03894\|P05142\|1- | BLAST_DOMO |
| | | | | | 134: V460-P550 | |
| | | | | | Zinc finger, C2H2 type, domain: C16-H37 | MOTIFS |
| 16 | 1748508CD1 | 299 | S75 S117 S166 | N42 N110 | | |
| | | | S206 T226 | | | |
| 17 | 2159545CD1 | 620 | S7 S32 S34 S127 | N29 | BTB/POZ domain: K51-F164 | HMMER_PFAM |
| | | | S218 S297 S307 | | | |
| | | | S354 S475 S522 | | | |
| | | | S531 T70 T106 | | | |
| | | | T311 T316 T390 | | | |
| | | | T401 T448 T542 | | | |
| | | | Y306 | | | |
| | | | | | Kelch motif: R355-N400, R496-T541, K402-N447, | HMMER_PFAM |
| | | | | | P449-G494, P543-S588, S307-N353 | |
| | | | | | PROTEIN REPEAT MATRIX RING CANAL | BLAST_{—} |
| | | | | | KELCH R12E2.1 C47D12.7 KIAA0132 KIAA0469 | PRODOM |
| | | | | | PD001473: P166-L294 | |
| | | | | | POZ DOMAIN | BLAST_DOMO |
| | | | | | DM00509\|Q04652\|131-335: E65-L238 | |
| | | | | | DM00509\|A45773\|130-334: E65-L238 | |
| | | | | | DM00509\|P21073\|1-198: F60-Q243 | |
| 18 | 8560269CD1 | 218 | S68 S133 T55 T91 | N28 N165 | Leucine zipper pattern: L141-L162 | MOTIFS |
| | | | T93 T151 | | | |
| 19 | 8710302CD1 | 427 | S22 S38 T118 T368 | | Cytosolic domain: R90-T115 | TMHMMER |
| | | | Y331 | | Transmembrane domain: E67-L89, I116-F138 | |
| | | | | | Non-cytosolic domain: M1-P66, R139-S427 | |
| 20 | 6778214CD1 | 612 | S50 S191 S232 | N70 N168 N228 | | |
| | | | S359 S370 S388 | N275 N360 | | |
| | | | S435 S473 S505 | N416 | | |
| | | | S507 S509 S511 | | | |
| | | | S590 S607 T47 T72 | | | |
| | | | T144 T439 T478 | | | |
| 21 | 258383CD1 | 458 | S22 S38 S120 T149 | | Cytosolic domain: R90-K95, R170-Q299, S355-S458 | TMHMMER |
| | | | T399 Y362 | | Transmembrane domain: E67-L89, F96-L115, I147- | |
| | | | | | F169, L300-R322, I332-A354 | |
| | | | | | Non-cytosolic domain: M1-P66, I116-T146, N323- | |
| | | | | | I331 | |
| 22 | 2804937CD1 | 1451 | S14 S28 S168 S222 | N201 N265 | Cytosolic domain: M1-N116, R200-V219, G293- | TMHMMER |
| | | | S367 S369 S483 | N689 N830 | H303, Q399-Y404 | |
| | | | S490 S649 S691 | N1092 N1172 | Transmembrane domain: V117-F139, I177-S199, | |
| | | | S768 S787 S821 | N1182 N1344 | F220-L242, L270-Y292, I304-S323, L376-L398, | |
| | | | S822 S884 S885 | N1370 | V405-P422 | |
| | | | S923 S944 S968 | | Non-cytosolic domain: R140-I176, L243-S269, R324- | |
| | | | S978 S1045 S1093 | | D375, Q423-V1451 | |
| | | | S1111 S1242 S1247 | | | |
| | | | S1262 S1267 S1330 | | | |
| | | | S1348 S1372 S1392 | | | |
| | | | S1397 S1421 T637 | | | |
| | | | T832 T977 T1438 | | | |
| | | | Y446 Y562 Y635 | | | |
| | | | Y845 Y1355 | | | |
| | | | | | PROTEIN COSMID 30B8 KIAA0435 B0511.12 | BLAST_{—} |
| | | | | | PECANEX DEVELOPMENTAL NEUROGENESIS | PRODOM |
| | | | | | TRANSMEMBRANE GLYCOPROTEIN | |
| | | | | | PD014553: N636-G1198 | |
| | | | | | PROTEIN B0511.12 COSMID 30B8 PECANEX | BLAST_{—} |
| | | | | | DEVELOPMENTAL NEUROGENESIS | PRODOM |
| | | | | | TRANSMEMBRANE GLYCOPROTEIN REPEAT | |
| | | | | | PD018553: L66-L287, S354-I471, S280-P359, | |
| | | | | | G1148-N1178 | |
| | | | | | PROTEIN COSMID 30B8 PECANEX | BLAST_{—} |
| | | | | | DEVELOPMENTAL NEUROGENESIS | PRODOM |
| | | | | | TRANSMEMBRANE GLYCOPROTEIN REPEAT | |
| | | | | | B0511.12 PD025614: W1379-V1444, P1179-P1207 | |
| 23 | 7494915CD1 | 184 | S9 T24 T30 T36 | N22 | Reverse transcriptase (RNA-dependent DNA | HMMER_PFAM |
| | | | T66 | | polymerase): R86-S159 | |
| | | | | | Cytosolic domain: M1-L153 | TMHMMER |
| | | | | | Transmembrane domain: F154-V176 | |
| | | | | | Non-cytosolic domain: K177-H184 | |
| | | | | | DNA RNADIRECTED POLYMERASE PUTATIVE | BLAST_{—} |
| | | | | | P150 TRANSCRIPTASE REVERSE PROTEIN L1 | PRODOM |
| | | | | | SEQUENCE PD002004: Q25-L88 | |
| | | | | | TRANSCRIPTASE; REVERSE; ORF2; ENCODE; | BLAST_DOMO |
| | | | | | DM01377\|P08548\|132-516: T24-M100 | |
| | | | | | DM01377\|P08547\|132-516: Q25-M100 | |
| | | | | | DM01377\|I38588\|130-517: Q25-M100 | |
| | | | | | DM01377\|S16783\|1-259: Q18-M100 | |
| 24 | 2073751CD1 | 407 | S179 S273 S283 | N247 | PROTEIN COILED COIL CHAIN MYOSIN | BLAST_{—} |
| | | | T61 T129 T213 | | REPEAT HEAVY ATPBINDING FILAMENT | PRODOM |
| | | | T294 T306 T376 | | HEPTAD PD000002: E128-E366 (Pvalue = 2.3e−10) | |
| | | | T379 T386 Y22 | | | |
| | | | | | Leucine zipper pattern: L301-L322 | MOTIFS |
| 25 | 3178841CD1 | 261 | S40 S56 S70 S140 | | HYPOTHETICAL 32.0 KD PROTEIN C09F5.2 IN | BLAST_{—} |
| | | | S144 T234 | | CHROMOSOME III TRANSMEMBRANE | PRODOM |
| | | | | | PD128096: V45-V183 | |
| | | | | | Cytosolic domains: 1-72, 123-155, 227-261 | TMHMMER |
| | | | | | Transmembrane domains: 79-90, 101-122, 156-178, | |
| | | | | | 204-226 | |
| | | | | | Non-cytosolic domains: 91-99, 179-203 | |
| 26 | 3674807CD1 | 209 | S197 T17 T187 | N132 | Signal peptide: M41-G90 | SPSCAN |
| 27 | 1794922CD1 | 333 | S6 S17 S39 S65 | N63 N295 | | MOTIFS |
| | | | S140 S174 S212 | | | |
| | | | S241 T31 T217 | | | |
| | | | T310 Y261 | | | |
| 28 | 1795509CD1 | 257 | T43 T168 Y105 | | COSMID E04F6 PD132304: F72-C254 | BLAST_{—} |
| | | | | | | PRODOM |
| | | | | | Cell attachment sequence: R78-D80 | MOTIFS |
| 29 | 2017180CD1 | 293 | S5 S120 S135 S155 | N273 N287 | | MOTIFS |
| | | | S251 S255 S279 | | | |
| | | | S288 T27 T42 | | | |
| | | | T107 T142 | | | |
| 30 | 219442CD1 | 598 | S28 S66 S71 S102 | N195 N220 | | MOTIFS |
| | | | S149 S163 S180 | N227 | | |
| | | | S186 S191 S215 | | | |
| | | | S228 S263 S283 | | | |
| | | | S287 S361 S365 | | | |
| | | | S406 S439 S543 | | | |
| | | | S561 S571 S584 | | | |
| | | | T45 T67 T172 | | | |
| | | | T185 T276 T290 | | | |
| | | | T337 T472 T492 | | | |
| | | | T545 | | | |
| 31 | 2597459CD1 | 470 | S3 S8 S23 S78 | N25 N155 N325 | BTB/POZ domain: D26-L139 | HMMER_PFAM |
| | | | S177 S196 S201 | | | |
| | | | S359 S377 S451 | | | |
| | | | T157 T279 Y101 | | | |
| | | | Y292 Y309 | | | |
| | | | | | Kelch motif: G335-K385, C387-D433 | HMMER_PFAM |
| | | | | | BTB Domain PF00651: A55-F67 | BLIMPS_{—} |
| | | | | | | PFAM |
| | | | | | PROTEIN DNA-BINDING ZINC FINGER METAL | BLAST_{—} |
| | | | | | BINDING NUCLEAR ZINC FINGER | PRODOM |
| | | | | | TRANSCRIPTION REGULATION | |
| | | | | | CHROMOSOME PD000632: Q16-L139 | |
| | | | | | POZ DOMAIN DM00509 | BLAST_DOMO |
| | | | | | Q04652\|131-335: S21-N218 | |
| | | | | | A45773\|130-334: S21-N218 | |
| | | | | | P21073\|1-198: S23-E216 | |
| | | | | | S59069\|1-171: H24-L135 | |
| 32 | 2783863CD1 | 311 | S39 S101 S133 | N68 | PROTEIN CHROMOSOME READING FRAME | BLAST_{—} |
| | | | T136 | | ORF TRANSMEMBRANE COSMID D8035.34P | PRODOM |
| | | | | | XV YOL002C PD005362: N68-S301 | |
| | | | | | Cytosolic domains: 1-73, 129-140, 196-201, 257-275 | TMHMMR |
| | | | | | Transmembrane domains: 74-96, 106-128, 141-163, | |
| | | | | | 173-195, 202-224, 239-256, 276-298 | |
| | | | | | Non-cytosolic domains: 97-105, 164-172, 225-238, | |
| | | | | | 299-311 | |
| | | | | | MEMBRANE; YOL002C; CHROMOSOME; | BLAST_DOMO |
| | | | | | C30D11.11; DM02642 | |
| | | | | | Q09749\|49-323: T32-K302 | |
| | | | | | Q09910\|169-441: T32-V283 | |
| | | | | | S62569\|169-441: T32-V283 | |
| | | | | | S61982\|50-325: Y31-V283 | |
| 33 | 2902971CD1 | 894 | S9 S14 S17 S75 | N106 N312 | PROTEIN CHROMOSOME C30D11.09 I B0361.1 | BLAST_{—} |
| | | | S86 S97 S113 S114 | N596 N757 | III PD033465: E673-K786 | PRODOM |
| | | | S115 S141 S161 | | | |
| | | | S169 S223 S267 | | | |
| | | | S277 S293 S313 | | | |
| | | | S327 S351 S372 | | | |
| | | | S420 S431 S439 | | | |
| | | | S454 S489 S536 | | | |
| | | | S554 S580 S598 | | | |
| | | | S705 S759 S792 | | | |
| | | | S806 S868 T176 | | | |
| | | | T278 T291 T323 | | | |
| | | | T346 T409 T438 | | | |
| | | | T581 T629 T635 | | | |
| | | | T723 Y310 | | | |
| | | | | | SPAC30D11.09; DM04663 | BLAST_DOMO |
| | | | | | Q10945\|1-144: I662-K786 | |
| | | | | | Q09909\|388-532: M672-W796 | |
| | | | | | S62567\|388-532: M672-W796 | |
| | | | | | Cell attachment sequence: R425-D427 | MOTIFS |
| | | | | | ATP/GTP-binding site motif A (P-loop): A622-T629 | MOTIFS |
| 34 | 368660CD1 | 653 | S37 S69 S79 S135 | N288 N508 N542 | TPR Domain: V341-H374, A307-N340, C382-F415 | HMMER_PFAM |
| | | | S225 S290 S303 | | | |
| | | | S475 S482 S483 | | | |
| | | | S512 S515 S516 | | | |
| | | | S520 S582 S645 | | | |
| | | | T373 T384 T414 | | | |
| | | | T448 T453 T476 | | | |
| | | | T601 T629 | | | |
| | | | | | Cell attachment sequence: R125-D127 | MOTIFS |
| 35 | 2804990CD1 | 144 | T16 | N17 | Cytosolic domain: 137-144 | TMHMMR |
| | | | | | Transmembrane domain: 114-136 | |
| | | | | | Non-cytosolic domain: 1-113 | |
| 36 | 168571CD1 | 424 | S88 S123 S124 | N122 N311 | signal_cleavage: M1-L24 | SPSCAN |
| | | | S151 S179 S194 | | | |
| | | | S258 S269 S364 | | | |
| | | | S365 S386 S407 | | | |
| | | | S409 T92 | | | |
| | | | | | EVI5 HOMOLOG TRUNCATED EVI5 | BLAST_{—} |
| | | | | | ECOTROPIC VIRAL INTEGRATION SITE | PRODOM |
| | | | | | COSMID F01G12 PD075221: E84-P180 | |
| | | | | | Protein kinases ATP-binding region signature: L370- | MOTIFS |
| | | | | | K401 | |
| 37 | 1286391CD1 | 1351 | S187 S266 S311 | N215 N235 | Integrase core domain: R1062-L1130 | HMMER_PFAM |
| | | | S320 S359 S360 | N358 N543 | | |
| | | | S423 S486 S496 | N1293 | | |
| | | | S556 S633 S657 | | | |
| | | | S704 S884 S1000 | | | |
| | | | S1204 S1262 S1274 | | | |
| | | | S1296 S1345 T25 | | | |
| | | | T74 T95 T124 | | | |
| | | | T159 T230 T329 | | | |
| | | | T392 T463 T642 | | | |
| | | | T691 T940 T1015 | | | |
| | | | T1079 T1215 | | | |
| | | | | | POLPOLYPROTEIN | BLAST_DOMO |
| | | | | | DM00159\|S08405\|760-943: H1009-A1139 | |
| | | | | | DM00140\|S52564\|1-364: Y517-K670 | |
| | | | | | Leucine zipper pattern: L837-L858 | MOTIFS |
| 38 | 2007684CD1 | 78 | | | | |
| 39 | 2227040CD1 | 411 | S5 S39 S56 S84 | N172 N218 | Phorbol esters/diacylglycerol binding domain: H111- | HMMER_PFAM |
| | | | S112 S122 S168 | | C161 | |
| | | | S198 S224 S232 | | | |
| | | | S234 S246 S249 | | | |
| | | | S325 S372 S396 | | | |
| | | | T82 T188 T207 | | | |
| | | | T286 | | | |
| | | | | | SH3 domain: Y295-V349 | HMMER_PFAM |
| | | | | | Phorbol esters/diacylglycerol binding domain | BLIMPS_{—} |
| | | | | | proteins BL00479: H111-G133, Q137-C152 | BLOCKS |
| | | | | | Phorbol esters/diacylglycerol binding domain: R120- | PROFILE- |
| | | | | | S213 | SCAN |
| | | | | | Diacylglycerol/phorbol-ester binding signature | BLMPS_{—} |
| | | | | | PR00008: H158-R170, V108-S122, C124-G133, | PRINTS |
| | | | | | Q137-V148 | |
| | | | | | STAC | BLAST_{—} |
| | | | | | PD027304: Q347-I411 | PRODOM |
| | | | | | PD032205: G163-G245 | |
| | | | | | SRC HOMOLOGY 3 (SH3) DOMAIN | BLAST_DOMO |
| | | | | | DM00025\|S61138\|55-108: Y297-Q347 | |
| | | | | | Cytochrome c family heme-binding site signature: | MOTIFS |
| | | | | | C124-Q129 | |
| | | | | | Phorbol esters/diacylglycerol binding domain: H111- | MOTIFS |
| | | | | | C161 | |
| 40 | 4346130CD1 | 1704 | S3 S165 S295 S323 | N42 N167 N205 | Putative GTP-ase activating protein for Arf: Y685- | HMMER_PFAM |
| | | | S346 S350 S378 | N231 N327 | L807 | |
| | | | S416 S424 S480 | N694 N697 | | |
| | | | S677 S683 S696 | N943 N1009 | | |
| | | | S699 S739 S781 | N1572 | | |
| | | | S834 S911 S932 | | | |
| | | | S977 S1124 S1165 | | | |
| | | | S1172 S1194 S1211 | | | |
| | | | S1404 S1428 S1435 | | | |
| | | | S1476 S1477 S1487 | | | |
| | | | S1510 S1582 S1593 | | | |
| | | | S1604 T34 T83 | | | |
| | | | T122 T155 T183 | | | |
| | | | T190 T196 T312 | | | |
| | | | T314 T357 T409 | | | |
| | | | T411 T417 T437 | | | |
| | | | T534 T548 T643 | | | |
| | | | T663 T681 T791 | | | |
| | | | T808 T919 T1011 | | | |
| | | | T1096 T1105 | | | |
| | | | T1215 T1279 | | | |
| | | | T1369 T1423 | | | |
| | | | T1524 T1639 | | | |
| | | | T1656 | | | |
| | | | | | PH domain: S1435-H1537, K483-K574, P588-A679, | HMMER_PFAM |
| | | | | | I879-V1003 | |
| | | | | | RhoGAP domain: P1129-E1282 | HMMER_PFAM |
| | | | | | SAM domain (Sterile alpha motif): V4-L68 | HMMER_PFAM |
| | | | | | HIV Rev interacting protein signature PR00405: | BLIMPS_{—} |
| | | | | | N697-C716, C716-K733, V484-V505 | PRINTS |
| | | | | | PROTEIN GTPASE DOMAIN AC PD00930: P1129- | BLIMPS_{—} |
| | | | | | G1154, L1232-L1272 | PRODOM |
| | | | | | PROTEIN ZINC FINGER NUCLEAR DNA | BLAST_{—} |
| | | | | | BINDING PUTATIVE GTPASE ACTIVATING | PRODOM |
| | | | | | FACTOR CHROMOSOME REPEAT PD002425: | |
| | | | | | N694-E775 | |
| | | | | | PROTEIN GTPASE DOMAIN SH2 ACTIVATION | BLAST_{—} |
| | | | | | ZINC 3 KINASE SH3 PHOSPHATIDYLINOSITOL | PRODOM |
| | | | | | REGULATORY PD000780: V1128-E1282 | |
| | | | | | PH DOMAIN DM00470 | BLAST_DOMO |
| | | | | | \|S54307\|1621-1845: K1125-E1301 | |
| | | | | | \|P34588\|1-285: K1125-N1291 | |
| | | | | | \|A49307\|566-842: T1096-Q1274 | |
| | | | | | \|P15882\|109-331: I1107-T1275 | |
| 41 | 55117040CD1 | 243 | S13 S38 S53 S59 | N50 N203 | Ankyrin repeat: Q135-N167, K168-E200, Y201-Q230 | HMMER_PFAM |
| | | | S75 T45 T171 | | | |
| | | | | | Aldehyde ferredoxin oxid PF01314: A110-V122, R73- | BLIMPS_{—} |
| | | | | | L103, A82-K114 | PFAM |
| 42 | 7472392CD1 | 248 | S7 S57 S112 S137 | | | |
| | | | S157 S175 S200 | | | |
| | | | T12 T180 | | | |
| 43 | 4028960CD1 | 310 | S59 S277 S283 T98 | N36 N63 N93 | | |
| | | | T141 T144 T184 | N97 N297 | | |
| | | | T199 T269 | | | |
| 44 | 8227004CD1 | 838 | S11 S133 S202 | N79 N210 N231 | PROTEIN SIT4-ASSOCIATING | BLAST_{—} |
| | | | S214 S304 S315 | N261 N302 | PHOSPHORYLATION CELL CYCLE SAP155 | PRODOM |
| | | | S402 S417 S503 | N358 N409 | KIAA0685 SAP185 SAP190 SAP4 PD014556: D68- | |
| | | | S582 S599 S604 | N542 N552 | E284 | |
| | | | S616 S673 S687 | N558 N647 | | |
| | | | S696 S700 S705 | | | |
| | | | S742 S817 T15 T23 | | | |
| | | | T171 T233 T274 | | | |
| | | | T278 T282 T390 | | | |
| | | | T411 T413 T472 | | | |
| | | | T507 T512 T608 | | | |
| | | | T614 T711 T751 | | | |
| | | | T789 | | | |
| | | | | | SIT4-ASSOCIATING PROTEIN SAP190 | BLAST_DOMO |
| | | | | | DM03002\|P40856\|222-821: M92-N363 | |
| | | | | | SIT4-ASSOCIAT1NG PROTEIN SAP185 | BLAST_DOMO |
| | | | | | DM03002\|P36123\|229-825: E98-N363 | |
| 45 | 3044763CD1 | 408 | S25 S62 S68 S103 | N257 N342 | Leucine zipper pattern: L261-L282 | MOTIFS |
| | | | S184 S286 S330 | | | |
| | | | S344 S377 S392 | | | |
| | | | T98 T131 T158 | | | |
| | | | T213 | | | |
| | | | | | Cytosolic domains: M1-G229, R290-R301, S377- | TMHMMER |
| | | | | | S408 | |
| | | | | | Transmembrane domains: L230-G252, T267-D289, | |
| | | | | | N302-L324, I354-L376 | |
| | | | | | Non-cytosolic domains: D253-K266, S325-Q353 | |
| 46 | 4044519CD1 | 101 | T89 | | Signal_cleavage: M1-S15 | SPSCAN |
| | | | | | Signal Peptide: M1-S19, M1-T16, M31-S59 | HMMER |
| | | | | | Cytosolic domains: M1-M31, E82-L101 | TMHMMER |
| | | | | | Transmembrane domains: V32-L54, L64-L81 | |
| | | | | | Non-cytosolic domain: N55-S63 | |
| 47 | 71351918CD1 | 256 | S26 S107 S174 | | WD domain, G-beta repeat: C32-D68, Q136-D171, | HMMER_PFAM |
| | | | S194 S202 T35 T63 | | V179-Q212 | |
| | | | T222 | | | |
| | | | | | Non-cytosolic domain: M1-L256 | TMHMMER |
| | | | | | Trp-Asp (WD-40) repeats signature: L44-A92 | PROFILE- |
| | | | | | | SCAN |
| | | | | | Trp-Asp containing, G-protein WD-40 repeats GENE | BLAST_{—} |
| | | | | | 16 PD106308: R71-L256 | PRODOM |
| | | | | | Trp-Asp (WD) repeats signature: V55-C69 | MOTIFS |
| 48 | 8109363CD1 | 104 | | | Non-cytosolic domain: M1-G104 | TMHMMER |
| 49 | 1272746CD1 | 855 | S27 S93 S159 S184 | N132 N157 | Non-cytosolic domain: M1-S855 | TMHMMER |
| | | | S245 S287 S329 | N254 N312 | | |
| | | | S593 S708 S726 | N325 N486 | | |
| | | | S793 S811 S827 | N592 N615 | | |
| | | | T18 T26 T35 T45 | N623 N659 | | |
| | | | T128 T200 T202 | N822 | | |
| | | | T319 T352 T438 | | | |
| | | | T470 T601 T642 | | | |
| | | | T807 | | | |
| 50 | 1839974CD1 | 427 | S254 S277 S321 | N73 N258 N405 | Fibronectin type III domain: L276-G364 | HMMER_PFAM |
| | | | S334 S361 S365 | | | |
| | | | T50T182T247 | | | |
| | | | Y329 | | | |
| | | | | | Non-cytosolic domain: M1-I427 | TMHMMER |
| | | | | | Fibronectin type III repeat signature PR00014: S393- | BLIMPS_{—} |
| | | | | | P402, G406-W416 | PRINTS |
| 51 | 1877336CD1 | 800 | S58 S74 S252 S277 | N272 N281 | Non-cytosolic domain: M1-F800 | TMHMMER |
| | | | S283 S306 S351 | N411 N785 | | |
| | | | S380 S415 S424 | | | |
| | | | S437 S439 S446 | | | |
| | | | S491 S521 S534 | | | |
| | | | S539 S605 S786 | | | |
| | | | T208 T302 T313 | | | |
| | | | T488 T553 T572 | | | |
| | | | T747 T781 Y331 | | | |
| | | | | | FIBRILLAR COLLAGEN CARBOXYL- | BLAST_DOMO |
| | | | | | TERMINAL DM00019\|S42886\|221-377: G112-N237 | |
| 52 | 2321054CD1 | 107 | S87 T17 T62 | | Signal_cleavage: M1-A43 | SPSCAN |
| | | | | | Non-cytosolic domain: M1-E24 | TMHMMER |
| | | | | | Transmembrane domain: A25-A43 | |
| | | | | | Cytosolic domain: K44-E107 | |
| 53 | 2796034CD1 | 522 | S10 S49 S77 S191 | N59 N85 N120 | Non-cytosolic domain: M1-Q522 | TMHMMER |
| | | | S195 S204 S212 | N487 N494 | | |
| | | | S335 S339 S344 | | | |
| | | | S361 S371 S397 | | | |
| | | | S443 S453 T72 | | | |
| | | | T125 T138 T146 | | | |
| | | | T228 T241 T245 | | | |
| | | | T267 T318 T363 | | | |
| | | | T496 | | | |
| 54 | 4413112CD1 | 305 | S28 S57 S98 S122 | N44 N55 N130 | Signal_cleavage: M1-S20 | SPSCAN |
| | | | S238 S294 | N148 | | |
| | | | | | Signal Peptide: M1-S20 | HMMER |
| | | | | | Non-cytosolic domain: M1-T165 | TMHMMER |
| | | | | | Transmembrane domain: I166-W188 | |
| | | | | | Cytosolic domain: R189-H305 | |
| | | | | | Leucine-rich repeat signature PR00019: L84-L97, | BLIMPS_{—} |
| | | | | | V63-L76 | PRINTS |
| 55 | 7654832CD1 | 329 | S2 S67 S150 S244 | | Signal_cleavage: M1-G46 | SPSCAN |
| | | | S277 T121 | | | |
| | | | | | Cytosolic domain: M1-T20 | TMHMMER |
| | | | | | Transmembrane domain: I21-I43 | |
| | | | | | Non-cytosolic domain: V44-R329 | |
| | | | | | Ank repeat: Y232-T264, D102-I134, A69-L101, | HMMER_PFAM |
| | | | | | D135-C167, S170-P199, H200-K231 | |
| 56 | 7503849CD1 | 236 | S34 S52 S96 S106 | N152 | Signal peptide: M18-A80 | SPSCAN |
| | | | S113 S118 T38 | | | |
| | | | T231 | | | |

TABLE 4

| Polynucleotide | |
|---|---|
| SEQ ID NO: / | |
| Incyte ID/Sequence | |
| Length | Sequence Fragments |
| 57/2867236CB1/ | 1-222, 1-461, 1-464, 1-572, 4-516, 7-279, 12-266, 16-285, 23-278, 24-281, 24-522, 30-320, 31-309, 31-316, 34-172, |
| 1485 | 34-327, 34-639, 42-282, 46-218, 46-335, 51-290, 51-342, 141-404, 141-405, 143-579, 182-471, 208-654, 208-684, |
| | 211-695, 217-453, 321-836, 335-910, 381-933, 390-996, 475-747, 475-762, 482-1009, 537-1153, 591-970, 717-993, |
| | 796-1090, 797-1382, 808-1485, 963-1280 |
| 58/1294096CB1/ | 1-210, 1-383, 1-416, 1-508, 1-516, 1-6176, 33-443, 33-570, 228-695, 228-1034, 365-486, 495-1170, 511-962, 574- |
| 6176 | 961, 647-975, 794-1062, 959-1554, 1000-1160, 1009-1246, 1009-1497, 1034-1327, 1075-1207, 1111-1292, 1170- |
| | 1258, 1250-1842, 1260-1540, 1261-1835, 1288-1927, 1292-1877, 1352-1518, 1417-1679, 1438-1085, 1446-1787, |
| | 1542-1841, 1697-1891, 1860-2146, 1872-1935, 1971-2491, 2021-2670, 2028-2623, 2108-2393, 2173-2698, 2174- |
| | 2729, 2179-2427, 2192-2528, 2194-2469, 2196-2775, 2198-2432, 2205-2471, 2206-2456, 2208-2458, 2211-2451, |
| | 2212-2465, 2226-2443, 2230-2787, 2242-2498, 2270-2481, 2281-2922, 2296-2933, 2310-2564, 2320-2575, 2327- |
| | 2614, 2329-2701, 2337-2663, 2350-2472, 2384-2671, 2384-3130, 2397-2558, 2397-2976, 2418-2677, 2450-2878, |
| | 2455-2608, 2470-2665, 2479-2725, 2480-2676, 2484-2768, 2496-3042, 2506-2905, 2509-2739, 2509-3040, 2512- |
| | 2772, 2563-3211, 2603-2902, 2665-2928, 2667-3092, 2673-3232, 2706-3159, 2711-3168, 2719-2972, 2726-3166, |
| | 2729-2927, 2738-3165, 2743-2840, 2744-3166, 2750-2997, 2763-3037, 2779-3309, 2780-3064, 2797-3413, 2822- |
| | 3091, 2829-3021, 2836-3285, 2843-3169, 2848-3438, 2852-3032, 2852-3209, 2856-3109, 2861-3546, 2881-3106, |
| | 2881-3375, 2890-3411, 2907-3364, 2910-3158, 2964-3435, 2993-3483, 2993-3597, 3009-3277, 3051-3494, 3056- |
| | 3286, 3069-3279, 3069-3583, 3071-3355, 3071-3660, 3098-3530, 3103-3348, 3126-3582, 3134-3337, 3140-3709, |
| | 3145-3396, 3174-3319, 3199-3388, 3199-3854, 3201-3770, 3204-3779, 3222-3627, 3250-3667, 3284-3920, 3308- |
| | 3625, 3336-3570, 3336-3594, 3336-3598, 3336-3601, 3336-3615, 3336-3617, 3336-3629, 3336-3631, 3336-3636, |
| | 3336-3669, 3342-3867, 3342-3957, 3342-3961, 3361-3965, 3370-3645, 3381-3575, 3381-3580, 3420-3832, 3435- |
| | 4105, 3460-3723, 3513-4121, 3519-3784, 3521-3985, 3542-3680, 3544-3775, 3559-4065, 3569-4170, 3576-3824, |
| | 3601-3810, 3625-3753, 3633-3852, 3639-4169, 3644-3940, 3651-4246, 3673-4260, 3682-3853, 3716-4031, 3720- |
| | 4094, 3720-4186, 3720-4194, 3721-4281, 3728-4206, 3728-4340, 3728-4427, 3749-4132, 3750-4001, 3750-4297, |
| | 3751-3880, 3759-4040, 3768-4266, 3778-4243, 3781-4065, 3783-4453, 3789-4049, 3796-4095, 3803-4392, 3820- |
| | 4049, 3820-4052, 3820-4106, 3835-4309, 3841-4163, 3841-4185, 3852-4098, 3853-4437, 3865-4082, 3865-4389, |
| | 3868-4165, 3892-4082, 3898-4136, 3899-4211, 3937-4467, 3940-4545, 3945-4098, 3954-4496, 3963-4183, |
| | 3973-4467, 3978-4383, 3987-4288, 3987-4535, 3991-4468, 3993-4595, 4004-4256, 4009-4482, 4011-4475, 4013- |
| | 4496, 4014-4292, 4017-4496, 4018-4481, 4020-4496, 4027-4496, 4028-4496, 4033-4480, 4037-4496, 4049-4496, |
| | 4050-4479, 4050-4496, 4054-4481, 4055-4481, 4057-4496, 4063-4481, 4067-4496, 4071-4480, 4077-4496, 4079- |
| | 4475, 4080-4480, 4088-4355, 4096-4480, 4099-4544, 4099-4580, 4107-4481, 4125-4421, 4130-4448, 4131-4486, |
| | 4136-4236, 4144-4481, 4145-4780, 4152-4421, 4162-4479, 4169-4480, 4169-4486, 4178-4464, 4180-4484, 4185- |
| | 4451, 4185-4544, 4185-4769, 4187-4402, 4192-4361, 4198-4476, 4224-4405, 4224-4445, 4237-4483, 4241-4496, |
| | 4249-4481, 4278-4715, 4283-4397, 4327-4587, 4370-4630, 4373-4627, 4402-4496, 4409-4717, 4449-4731, 4451- |
| | 4750, 4467-4618, 4468-4820, 4478-4720, 4478-5043, 4490-4908, 4492-4738, 4493-4740, 4497-5079, 4499-4646, |
| | 4503-4776, 4506-4737, 4525-4787, 4527-4755, 4552-5003, 4556-4852, 4561-4822, 4562-5252, 4564-5277, 4572- |
| | 4875, 4574-4841, 4591-5200, 4603-4856, 4604-4867, 4605-4849, 4605-4988, 4606-4864, 4610-4892, 4614-4818, |
| | 4614-5195, 4625-4875, 4625-4881, 4644-5326, 4649-4940, 4655-4824, 4657-5067, 4672-4965, 4677-5283, |
| | 4690-4934, 4694-4916, 4698-4976, 4700-4949, 4707-5179, 4715-4975, 4717-5277, 4727-4987, 4732-5006, 4732- |
| | 5009, 4743-4962, 4744-5327, 4749-5009, 4770-5078, 4780-5025, 4789-4995, 4798-5040, 4804-5290, 4823-4912, |
| | 4823-5407, 4824-4943, 4825-5077, 4829-5103, 4833-5086, 4855-5387, 4874-5558, 4912-5366, 4916-5718, 4919- |
| | 5475, 4987-5562, 4991-5497, 5003-5554, 5009-5551, 5015-5373, 5059-5359, 5087-5585, 5100-5412, 5117-5368, |
| | 5118-5354, 5123-5387, 5132-5378, 5132-5443, 5135-5600, 5136-5360, 5140-5388, 5159-5473, 5165-5390, 5166- |
| | 5424, 5169-5429, 5184-5409, 5189-5356, 5190-5680, 5205-5467, 5205-5505, 5206-5507, 5206-5759, 5213-5491, |
| | 5223-5507, 5227-5503, 5233-5953, 5236-5506, 5236-5734, 5238-5493, 5238-5507, 5248-5499, 5249-5575, 5255- |
| | 5376, 5267-5525, 5278-5587, 5279-5566, 5282-5555, 5286-5424, 5289-5901, 5290-5505, 5298-5566, 5301-5559, |
| | 5353-5640, 5354-5549, 5354-5597, 5354-5601, 5389-5912, 5412-5957, 5479-6039, 5544-5877, 5547-6168, 5574- |
| | 6129, 5591-5846, 5591-5990, 5591-6127, 5594-6062, 5599-6176, 5601-5849, 5603-6069, 5609-5864, 5609-5868, |
| | 5609-5925, 5609-5979, 5609-6133, 5611-5894, 5617-5868, 5619-5882, 5623-5862, 5632-5890, 5638-6176, |
| | 5645-5866, 5655-6169, 5659-5853, 5659-6082, 5664-5866, 5664-5911, 5671-5929, 5671-6063, 5671-6068, 5671- |
| | 6169, 5672-5929, 5673-5914, 5674-6169, 5682-6154, 5688-6169, 5689-5913, 5689-5961, 5689-5973, 5691-6095, |
| | 5692-6145, 5696-5948, 5697-6149, 5697-6169, 5699-5987, 5699-5993, 5703-6169, 5704-5982, 5704-6151, 5704- |
| | 6169, 5709-6166, 5710-5872, 5710-6170, 5712-5984, 5712-5995, 5712-6169, 5713-5872, 5716-6153, 5720-6176, |
| | 5729-6169, 5735-6145, 5745-6170, 5746-6169, 5749-5942, 5752-5994, 5752-6169, 5753-6022, 5753-6171, 5754- |
| | 6170, 5756-5999, 5756-6169, 5756-6174, 5757-6169, 5759-6169, 5759-6176, 5761-6169, 5762-6149, 5763-5996, |
| | 5764-6169, 5771-6035, 5771-6174, 5772-5994, 5775-5959, 5776-6016, 5780-6169, 5783-6061, 5783-6169, 5785- |
| | 6095, 5786-5991, 5786-6113, 5788-6169, 5789-6073, 5803-6071, 5805-6076, 5811-6173, 5812-6169, 5812-6176, |
| | 5814-6075, 5814-6142, 5814-6176, 5815-6174, 5816-6169, 5816-6171, 5817-6153, 5818-6169, 5819-6145, 5820- |
| | 6160, 5820-6173, 5820-6176, 5822-6176, 5824-6031, 5824-6169, 5824-6171, 5827-6149, 5830-6168, 5830-6169, |
| | 5832-6017, 5844-6170, 5846-6083, 5846-6095, 5846-6107, 5846-6170, 5850-6169, 5852-6152, 5852-6170, |
| | 5854-6095, 5861-6173, 5868-6169, 5874-6133, 5874-6169, 5881-6122, 5883-6136, 5887-6173, 5928-6162, 5928- |
| | 6176, 5931-6171, 5932-6133, 5935-6151, 5935-6162, 5935-6163, 5935-6174, 5937-6169, 5941-6166, 5941-6176, |
| | 5945-6166, 5957-6150, 5958-6119, 5958-6145, 5958-6154, 5962-6163, 5962-6166, 5963-6139, 5971-6147, 5971- |
| | 6176, 5973-6145, 5979-6175, 5989-6164, 5996-6137, 6005-6171, 6006-6151, 6006-6176, 6020-6169, 6022-6145, |
| | 6027-6142, 6051-6169, 6063-6169, 6087-6166, 6101-6169 |
| 59/7238537CB1/ | 1-496, 13-568, 15-704, 16-417, 16-532, 17-371, 18-272, 18-325, 19-309, 20-571, 21-215, 25-288, 25-322, 27-288, |
| 1944 | 32-623, 34-671, 35-287, 40-682, 41-638, 44-642, 54-372, 138-407, 147-380, 161-429, 204-838, 233-497, 233-767, |
| | 268-897, 297-829, 305-729, 310-664, 336-894, 338-586, 346-615, 352-938, 354-702, 369-771, 380-1045, 388-837, |
| | 394-1041, 428-710, 428-1061, 428-1206, 462-892, 472-896, 494-1232, 496-1077, 511-1063, 519-777, 527-797, 530- |
| | 772, 531-770, 548-1220, 558-1229, 559-1143, 561-1116, 561-1220, 578-1194, 584-1232, 586-864, 595-861, 603- |
| | 870, 634-1312, 662-1147, 681-1042, 681-1388, 686-1169, 703-1209, 710-1189, 723-1149, 731-1284, 741-1209, 744- |
| | 1175, 760-1324, 766-1079, 774-1188, 792-1051, 802-1160, 807-1052, 813-1120, 813-1386, 834-1191, 840-1129, |
| | 861-1318, 861-1439, 861-1470, 866-1128, 868-1403, 881-1117, 883-1122, 889-1516, 891-1156, 891-1168, 893- |
| | 1131, 894-1158, 896-1601, 904-1387, 908-1634, 915-1398, 924-1163, 924-1500, 924-1544, 930-1600, 931-1187, |
| | 931-1489, 934-1658, 938-1535, 942-1456, 947-1210, 951-1250, 957-1214, 959-1422, 967-1585, 970-1422, 976- |
| | 1226, 981-1419, 985-1418, 987-1704, 992-1520, 995-1259, 1003-1269, 1003-1567, 1005-1703, 1006-1585, |
| | 1010-1204, 1010-1263, 1010-1623, 1013-1303, 1014-1632, 1019-1354, 1024-1483, 1025-1545, 1031-1662, 1039- |
| | 1302, 1039-1418, 1039-1593, 1051-1698, 1052-1416, 1059-1300, 1063-1600, 1065-1422, 1066-1404, 1067-1464, |
| | 1073-1409, 1073-1593, 1074-1369, 1074-1419, 1075-1544, 1079-1323, 1081-1668, 1086-1544, 1088-1697, 1093- |
| | 1378, 1099-1544, 1101-1424, 1103-1544, 1110-1460, 1117-1462, 1128-1630, 1133-1703, 1135-1702, 1138-1617, |
| | 1140-1703, 1143-1550, 1145-1369, 1147-1544, 1153-1690, 1155-1702, 1157-1698, 1159-1696, 1171-1335, 1178- |
| | 1752, 1192-1544, 1200-1547, 1209-1540, 1213-1506, 1224-1928, 1236-1704, 1243-1704, 1247-1705, 1256-1703, |
| | 1256-1705, 1261-1705, 1263-1561, 1270-1704, 1273-1704, 1288-1705, 1290-1705, 1293-1696, 1293-1944, 1294- |
| | 1704, 1295-1704, 1297-1704, 1308-1703, 1308-1704, 1311-1568, 1315-1666, 1317-1704, 1318-1705, 1322-1705, |
| | 1324-1705, 1327-1627, 1327-1702, 1328-1704, 1332-1704, 1333-1704, 1334-1693, 1337-1696, 1344-1613, 1353- |
| | 1722, 1355-1650, 1362-1705, 1366-1654, 1366-1660, 1366-1704, 1372-1804, 1380-1704, 1386-1705, 1388-1645, |
| | 1389-1704, 1396-1704, 1403-1704, 1426-1682, 1480-1693, 1483-1704, 1494-1717, 1494-1737, 1542-1705 |
| 60/7494391CB1/ | 1-669, 172-838, 295-743, 295-786, 295-843, 295-931, 295-934, 295-954, 295-1002, 295-1021, 299-774, 299-919, |
| 1992 | 300-582, 300-1127, 346-1138, 347-974, 350-1163, 414-1050, 430-1203, 435-1167, 439-642, 444-517, 481-1162, |
| | 532-1242, 557-1282, 587-1396, 652-1310, 674-1162, 755-1546, 756-1560, 804-1044, 805-1586, 822-1464, 822- |
| | 1614, 838-1064, 883-1583, 922-1557, 924-1710, 961-1686, 964-1657, 965-1207, 990-1471, 1025-1312, 1025-1665, |
| | 1032-1842, 1033-1648, 1072-1643, 1075-1507, 1087-1921, 1099-1602, 1114-1555, 1121-1356, 1121-1475, 1134- |
| | 1474, 1156-1376, 1156-1825, 1161-1707, 1168-1893, 1190-1814, 1229-1870, 1255-1903, 1258-1769, 1266-1869, |
| | 1282-1575, 1300-1806, 1325-1992, 1347-1540 |
| 61/6451054CB1/ | 1-24, 1-27, 1-36, 1-41, 1-42, 1-43, 1-53, 1-55, 1-64, 1-65, 1-71, 1-72, 1-75, 1-80, 1-83, 1-86, 1-88, 1-89, 1-90, 1-92, |
| 3906 | 1-94, 1-95, 1-109, 1-117, 1-123, 1-130, 1-131, 1-139, 1-140, 1-170, 1-172, 1-186, 1-189, 1-190, 1-191, 1-193, 1-399, |
| | 5-189, 6-186, 7-189, 10-159, 14-151, 22-193, 29-193, 38-192, 38-193, 41-112, 44-189, 59-90, 71-193, 78-193, 79- |
| | 193, 86-193, 87-193, 91-192, 96-192, 122-193, 126-193, 127-193, 133-186, 139-192, 151-192, 153-191, 158-193, |
| | 162-193, 192-680, 192-728, 433-998, 433-1007, 433-1039, 433-1040, 444-668, 453-1350, 503-782, 573-1070, 573- |
| | 1166, 573-3748, 633-984, 633-1139, 639-1306, 666-1172, 683-950, 684-1132, 703-1346, 755-1518, 790-1294, 814- |
| | 1423, 867-1641, 869-1468, 909-1501, 996-1644, 1009-1598, 1025-1654, 1090-1117, 1103-1320, 1103-1610, 1105- |
| | 1535, 1105-1550, 1131-1438, 1136-1465, 1153-1704, 1158-1465, 1177-1419, 1178-1253, 1198-1709, 1201-1552, |
| | 1210-1552, 1224-1839, 1227-1486, 1239-1540, 1240-1540, 1249-1788, 1269-1821, 1292-1804, 1317-1499, 1332- |
| | 1556, 1426-2015, 1465-1953, 1471-1707, 1487-1693, 1509-2141, 1519-1798, 1520-1609, 1570-1912, 1593-2047, |
| | 1621-1891, 1631-2218, 1723-1882, 1726-2213, 1762-2341, 1764-2348, 1764-2431, 1765-2431, 1785-2431, |
| | 1816-2078, 1822-2074, 1843-2044, 1861-2292, 1874-2214, 1891-2776, 1921-2298, 1943-2431, 1969-2494, 1990- |
| | 2610, 2003-2306, 2011-2255, 2029-2290, 2029-2442, 2078-2299, 2079-2599, 2094-2558, 2121-2320, 2121-2323, |
| | 2127-2428, 2128-2336, 2128-2623, 2129-2857, 2136-2731, 2140-2430, 2155-2620, 2191-2846, 2198-2731, 2200- |
| | 2731, 2206-2460, 2210-2444, 2211-2367, 2217-3127, 2227-2447, 2243-2806, 2320-2898, 2325-2981, 2340-2767, |
| | 2354-2493, 2356-2466, 2358-2592, 2358-2740, 2373-2485, 2374-2561, 2374-2857, 2374-2892, 2378-2661, 2380- |
| | 2652, 2383-2656, 2394-2976, 2396-3005, 2430-3135, 2447-2699, 2447-2706, 2470-3044, 2473-2759, 2474-3022, |
| | 2482-3005, 2492-3007, 2498-2986, 2502-2946, 2503-2634, 2510-2706, 2510-3005, 2515-2964, 2516-2747, 2522- |
| | 3007, 2524-2768, 2527-2656, 2535-3028, 2555-2858, 2555-3132, 2555-3133, 2556-2825, 2559-2819, 2573-2795, |
| | 2593-2863, 2595-2857, 2598-2836, 2603-2876, 2603-2926, 2617-3043, 2619-2887, 2620-3067, 2623-2761, 2635- |
| | 3021, 2646-2908, 2649-3215, 2651-3260, 2652-3135, 2653-3065, 2659-3420, 2665-2946, 2679-3186, 2683-3348, |
| | 2684-3235, 2686-3166, 2687-3162, 2689-3174, 2711-2945, 2716-2983, 2732-2988, 2737-3247, 2739-3202, |
| | 2776-3348, 2783-3290, 2789-3052, 2790-3247, 2796-3045, 2799-3054, 2802-3423, 2830-3094, 2834-3460, 2846- |
| | 3098, 2847-3046, 2862-3145, 2866-3107, 2867-3029, 2869-3162, 2871-3253, 2871-3410, 2875-3131, 2891-3193, |
| | 2893-3193, 2900-3149, 2900-3494, 2901-3485, 2914-3137, 2922-3185, 2922-3381, 2925-3444, 2927-3495, 2932- |
| | 3584, 2947-3420, 2957-3233, 2959-3210, 2966-2998, 2991-3259, 2994-3597, 2997-3580, 3004-3264, 3013-3571, |
| | 3016-3181, 3023-3228, 3025-3299, 3025-3348, 3025-3662, 3026-3224, 3046-3308, 3051-3315, 3053-3526, 3057- |
| | 3562, 3059-3739, 3063-3300, 3079-3640, 3081-3742, 3096-3564, 3104-3748, 3105-3738, 3116-3737, 3119-3680, |
| | 3121-3358, 3127-3307, 3127-3647, 3128-3432, 3128-3701, 3129-3295, 3133-3513, 3145-3733, 3146-3739, 3156- |
| | 3365, 3156-3417, 3156-3427, 3156-3616, 3156-3738, 3175-3733, 3184-3635, 3188-3418, 3190-3742, 3193-3386, |
| | 3200-3407, 3200-3697, 3214-3457, 3214-3742, 3216-3738, 3227-3676, 3235-3691, 3246-3742, 3246-3743, 3247- |
| | 3746, 3255-3531, 3255-3748, 3256-3742, 3263-3491, 3263-3751, 3269-3536, 3274-3345, 3277-3396, 3277-3549, |
| | 3291-3748, 3292-3742, 3294-3748, 3296-3327, 3296-3703, 3296-3760, 3297-3742, 3298-3748, 3300-3548, |
| | 3300-3559, 3300-3743, 3300-3748, 3303-3748, 3306-3746, 3311-3748, 3314-3748, 3315-3748, 3321-3756, 3322- |
| | 3755, 3322-3760, 3331-3754, 3333-3760, 3342-3749, 3343-3746, 3344-3747, 3345-3616, 3346-3742, 3351-3750, |
| | 3360-3746, 3363-3748, 3371-3713, 3371-3748, 3372-3524, 3375-3741, 3377-3746, 3378-3748, 3382-3740, 3389- |
| | 3748, 3395-3674, 3397-3742, 3409-3664, 3410-3746, 3413-3746, 3417-3688, 3419-3711, 3423-3746, 3428-3760, |
| | 3440-3746, 3443-3748, 3450-3746, 3450-3748, 3455-3746, 3461-3732, 3469-3760, 3480-3681, 3482-3543, 3496- |
| | 3735, 3496-3748, 3499-3732, 3504-3733, 3504-3735, 3504-3747, 3504-3760, 3519-3742, 3528-3748, 3535-3748, |
| | 3585-3748, 3603-3735, 3609-3748, 3612-3748, 3614-3906, 3636-3748, 3665-3739 |
| 62/7494592CB1/ | 1-234, 8-614, 30-557, 30-619, 31-226, 102-429, 127-689, 134-799, 150-400, 186-834, 238-571, 520-1276, 624- |
| 3236 | 1216, 688-1195, 721-851, 792-1518, 821-1162, 835-1154, 905-1626, 916-1626, 922-1626, 943-1222, 953-1499, |
| | 1086-1616, 1112-1702, 1246-1665, 1251-1676, 1313-1858, 1441-1854, 1465-1919, 1477-1620, 1642-2180, 1697- |
| | 1860, 1716-2387, 1778-2128, 1796-2083, 1822-2371, 1846-2158, 1905-2422, 1973-2308, 2075-2187, 2092-2626, |
| | 2092-2631, 2094-2362, 2101-2662, 2191-2810, 2243-2655, 2506-2824, 2570-3236, 2572-2827 |
| 63/5202657CB1/ | 1-237, 1-525, 2-270, 3-277, 3-300, 3-557, 3-567, 7-270, 7-275, 8-292, 8-529, 12-207, 12-248, 12-606, 26-274, 26- |
| 1906 | 541, 27-276, 28-572, 29-306, 33-244, 33-330, 76-714, 119-450, 119-451, 125-371, 272-537, 296-556, 324-467, 553- |
| | 990, 555-813, 555-961, 557-812, 557-814, 571-812, 605-799, 608-860, 656-884, 661-927, 669-931, 712-959, 712- |
| | 976, 712-992, 713-980, 716-1007, 777-1048, 798-1050, 884-1405, 890-1120, 902-1175, 929-1294, 998-1533, 1018- |
| | 1432, 1037-1198, 1055-1230, 1061-1563, 1062-1283, 1064-1326, 1066-1342, 1068-1333, 1075-1396, 1079-1305, |
| | 1085-1345, 1085-1370, 1094-1347, 1102-1313, 1102-1321, 1102-1658, 1102-1789, 1139-1396, 1151-1648, 1177- |
| | 1403, 1183-1867, 1198-1894, 1205-1491, 1210-1827, 1226-1563, 1230-1834, 1237-1663, 1259-1874, 1264-1838, |
| | 1265-1544, 1268-1882, 1275-1566, 1284-1586, 1296-1516, 1303-1496, 1344-1583, 1346-1906, 1353-1874, 1359- |
| | 1594, 1366-1748, 1373-1635, 1375-1656, 1375-1851, 1378-1618, 1378-1619, 1393-1594, 1393-1631, 1393-1656, |
| | 1398-1751, 1425-1894, 1441-1898, 1446-1872, 1450-1906, 1455-1855, 1459-1891, 1462-1718, 1462-1878, 1467- |
| | 1864, 1475-1898, 1520-1793, 1520-1890, 1529-1878, 1536-1760, 1536-1823, 1558-1891, 1560-1890, 1587-1792, |
| | 1602-1870, 1606-1851, 1622-1872, 1636-1906, 1697-1906, 1708-1886, 1755-1891, 1776-1874 |
| 64/2013529CB1/ | 1-153, 1-293, 1-303, 1-322, 1-383, 1-423, 1-469, 1-478, 1-483, 1-484, 1-490, 1-507, 1-560, 1-603, 1-611, 1-627, 1- |
| 1347 | 654, 1-659, 1-660, 2-554, 2-630, 15-313, 15-636, 25-303, 25-328, 48-793, 64-554, 108-793, 183-441, 254-777, 279- |
| | 567, 282-556, 331-616, 341-1071, 350-604, 350-889, 374-591, 374-665, 413-610, 415-683, 455-1085, 517-848, 517- |
| | 924, 518-1149, 529-1098, 573-1203, 618-998, 634-1303, 647-1224, 675-1318, 681-895, 681-1040, 681-1057, 681- |
| | 1077, 681-1090, 681-1091, 681-1094, 681-1199, 681-1201, 681-1205, 681-1215, 681-1223, 681-1303, 681-1312, |
| | 684-1127, 684-1308, 751-1347, 761-1347, 771-1347, 816-1312, 908-1312, 916-1334, 929-1310, 1062-1347, 1089- |
| | 1310, 1128-1312, 1172-1310 |
| 65/3841351CB1/ | 1-585, 1-1854, 21-594, 29-721, 31-630, 34-302, 46-582, 49-289, 74-312, 474-875, 632-884, 632-1014, 632-1230, |
| 1854 | 632-1287, 675-1276, 761-1254, 814-1392, 850-1273, 864-1103, 904-1208, 906-1431, 945-1276, 949-1268, 1207- |
| | 1854, 1208-1608, 1316-1854, 1378-1603, 1410-1800, 1473-1560 |
| 66/152116CB1/ | 1-568, 74-694, 81-697, 93-579, 105-775, 108-291, 239-902, 468-718, 468-869, 515-761, 579-838, 583-1327, 670- |
| 1327 | 1289, 685-1291, 700-896, 872-1287 |
| 67/2381031CB1/ | 1-225, 1-515, 1-604, 2-254, 3-272, 7-185, 7-266, 7-286, 7-290, 7-291, 7-294, 7-296, 8-207, 9-301, 11-307, 11-319, |
| 627 | 15-264, 16-250, 17-278, 17-290, 17-295, 17-572, 18-246, 20-373, 22-301, 29-271, 29-287, 32-202, 32-257, 32-273, |
| | 32-291, 32-338, 32-343, 35-293, 35-295, 35-348, 37-194, 37-397, 37-452, 43-360, 51-327, 154-435, 155-617, 156- |
| | 433, 157-627, 158-607, 158-612, 162-378, 162-379, 164-615, 168-479, 169-627, 185-627, 188-606, 189-627, 204- |
| | 609, 223-607, 233-409, 254-615, 284-556, 292-573, 300-606, 344-529, 366-612, 418-627 |
| 68/2511371CB1/ | 1-577, 237-1628, 328-691, 328-892, 334-925, 373-975, 496-1062, 557-1106, 686-855, 921-978, 1002-1634, 1053- |
| 2564 | 1594, 1091-1766, 1102-1630, 1209-1827, 1210-1767, 1213-1642, 1213-1926, 1286-1943, 1305-1700, 1316-1945, |
| | 1336-1800, 1341-1981, 1341-2008, 1344-1908, 1345-1935, 1349-1899, 1464-2013, 1492-2061, 1503-1967, 1569- |
| | 2167, 1581-2170, 1662-2343, 1695-2351, 1746-2337, 1746-2400, 1750-2394, 1756-2418, 1772-2316, 1776-2497, |
| | 1902-2429, 1924-2453, 1930-2564 |
| 69/8068623CB1/ | 1-1457, 1292-1820, 1292-1831, 1292-1843, 1292-1908, 1292-1933, 1305-2006, 1306-1967, 1307-1900, 1307-1942, |
| 4134 | 1313-1790, 1319-1826, 1320-2234, 1322-1920, 1327-1889, 1331-1976, 1332-1610, 1352-1908, 1399-1856, 1439- |
| | 1929, 1485-1694, 1485-2040, 1501-1939, 1510-2089, 1516-2152, 1516-2156, 1516-2175, 1616-1985, 1695-1933, |
| | 1695-1942, 1738-1934, 1810-2017, 1810-2271, 1862-2339, 1882-2248, 1941-2478, 1961-2212, 1961-2214, 1970- |
| | 2218, 1975-2115, 1989-2719, 2009-2287, 2019-2457, 2019-2568, 2029-2269, 2046-2627, 2051-2697, 2055-2587, |
| | 2167-2434, 2185-2435, 2213-2829, 2258-2521, 2336-2525, 2408-2941, 2409-2620, 2430-2714, 2478-3024, 2547- |
| | 3147, 2670-2933, 2674-2921, 2674-3216, 2788-3037, 2867-3224, 2867-3445, 2908-3173, 2959-3159, 2959-3186, |
| | 2998-3252, 2998-3544, 3104-3358, 3195-3383, 3195-3432, 3285-3557, 3310-3502, 3368-3647, 3387-3642, 3410- |
| | 3598, 3491-3643, 3491-4021, 3499-3753, 3554-4134, 3610-3749, 3629-3859, 3652-3900, 3776-4011 |
| 70/677977CB1/ | 1-1548, 464-1093, 467-1093, 482-1093, 496-1093, 502-1093, 504-1093, 506-1093, 513-1093, 515-1093, 528-1093, |
| 2329 | 529-1093, 569-1093, 588-1093, 612-1088, 626-1093, 627-1093, 655-976, 678-1093, 694-1093, 903-1166, 927-1448, |
| | 1119-1860, 1161-1830, 1211-1741, 1253-1481, 1374-2024, 1378-1640, 1414-1546, 1414-1548, 1439-1704, 1461- |
| | 2042, 1467-1985, 1482-1548, 1512-1548, 1516-1548, 1519-1548, 1522-1548, 1548-1636, 1548-1733, 1548-1740, |
| | 1548-1763, 1548-1764, 1548-1788, 1548-2001, 1548-2080, 1548-2147, 1548-2195, 1548-2210, 1548-2220, 1548- |
| | 2329, 1578-1656, 1582-1851, 1585-2146, 1588-1849, 1588-2097 |
| 71/1661472CB1/ | 1-389, 2-389, 4-26, 18-2925, 96-815, 118-777, 333-923, 366-622, 369-907, 482-1135, 536-1004, 573-812, 661- |
| 2960 | 1021, 739-973, 739-1245, 743-1323, 774-1466, 793-1368, 882-1005, 927-1440, 964-1514, 1008-1582, 1035-1321, |
| | 1291-1719, 1305-1567, 1305-1721, 1369-1547, 1419-1666, 1419-1795, 1630-1685, 1675-2070, 1766-2529, 1781- |
| | 2148, 1781-2152, 1781-2164, 1781-2210, 1781-2221, 1781-2275, 1781-2292, 1781-2457, 1782-2446, 1799-2194, |
| | 1812-2291, 1839-2037, 1839-2318, 1843-2466, 1883-2459, 1893-2298, 1904-2271, 1935-2144, 1935-2150, 1939- |
| | 2197, 1941-2200, 1941-2541, 1941-2621, 1942-2327, 1949-2152, 1949-2206, 1994-2214, 1996-2151, 1997-2253, |
| | 2004-2298, 2005-2756, 2010-2248, 2028-2282, 2032-2262, 2032-2283, 2038-2258, 2038-2372, 2038-2489, 2039- |
| | 2723, 2046-2331, 2053-2544, 2055-2802, 2102-2744, 2111-2313, 2127-2345, 2127-2622, 2132-2914, 2140-2373, |
| | 2160-2754, 2161-2417, 2164-2392, 2166-2412, 2176-2445, 2203-2384, 2233-2883, 2239-2536, 2239-2548, 2259- |
| | 2497, 2270-2541, 2272-2520, 2285-2451, 2285-2761, 2306-2855, 2328-2760, 2341-2592, 2349-2619, 2349-2796, |
| | 2357-2810, 2372-2948, 2377-2628, 2385-2811, 2391-2811, 2403-2893, 2439-2857, 2443-2920, 2453-2926, |
| | 2468-2925, 2478-2921, 2483-2922, 2488-2960, 2497-2938, 2499-2923, 2500-2917, 2502-2920, 2504-2948, 2511- |
| | 2923, 2515-2923, 2520-2928, 2543-2926, 2549-2700, 2557-2920, 2568-2923, 2584-2934, 2595-2924, 2597-2914, |
| | 2726-2920, 2770-2924, 2773-2924 |
| 72/1748508CB1/ | 1-523, 57-749, 89-761, 392-889, 473-739, 545-1327, 592-1314, 640-1337, 678-1404, 706-1471, 741-868, 860-1366, |
| 2623 | 896-1143, 912-1385, 916-1156, 920-1172, 981-1578, 1067-1925, 1097-1796, 1150-1427, 1182-1797, 1240-1957, |
| | 1299-1529, 1322-1592, 1322-1603, 1322-1617, 1322-1737, 1322-1931, 1322-1962, 1345-1623, 1348-1770, 1352- |
| | 1619, 1353-1624, 1384-1750, 1384-1997, 1392-1661, 1439-1709, 1558-1769, 1558-1938, 1614-1899, 1725-2254, |
| | 1801-2046, 1859-2113, 1951-2195, 1951-2505, 1951-2603, 1953-2098, 1954-2495, 1956-2480, 1959-2116, 1963- |
| | 2501, 1963-2511, 1967-2209, 1980-2512, 1988-2601, 2010-2582, 2010-2613, 2046-2595, 2063-2584, 2064-2588, |
| | 2079-2616, 2085-2358, 2092-2257, 2098-2617, 2103-2502, 2106-2616, 2114-2616, 2122-2407, 2128-2592, 2138- |
| | 2592, 2141-2598, 2142-2595, 2142-2598, 2147-2599, 2148-2614, 2166-2616, 2167-2599, 2175-2307, 2182-2595, |
| | 2200-2595, 2201-2623, 2202-2595, 2207-2592, 2226-2597, 2229-2595, 2235-2505, 2244-2595, 2246-2616, 2263- |
| | 2595, 2264-2542, 2269-2565, 2288-2595, 2288-2597, 2297-2592, 2324-2578, 2344-2595, 2372-2615, 2424-2615, |
| | 2535-2599 |
| 73/2159545CB1/ | 1-625, 1-695, 9-773, 46-2189, 275-1051, 343-822, 704-4518, 987-1569, 990-1563, 992-1230, 992-1443, 1029-1388, |
| 4518 | 1260-1989, 1262-1572, 1263-1572, 1266-1569, 1266-1572, 1314-1570, 1363-1613, 1363-1912, 1520-2058, 1588- |
| | 2056, 1588-2086, 1590-1883, 1660-2224, 1736-2117, 1747-1939, 1781-2041, 1781-2324, 1798-2058, 1912-2216 |
| 74/8560269CB1/ | 1-263, 1-272, 1-329, 65-281, 65-332, 67-209, 90-274, 90-389, 91-270, 132-788, 331-583, 346-802, 348-620, 358- |
| 1238 | 949, 509-1075, 559-1024, 619-1030, 620-826, 620-879, 620-1031, 666-875, 666-1129, 678-1093, 736-1091, 764- |
| | 1057, 783-1107, 785-1045, 785-1078, 785-1088, 918-1184, 1020-1238 |
| 75/8710302CB1/ | 1-226, 82-368, 96-381, 125-423, 180-915, 184-395, 188-437, 189-379, 481-984, 493-989, 499-917, 500-980, 550- |
| 1771 | 869, 558-991, 561-973, 561-991, 563-991, 565-1159, 569-1250, 606-1103, 667-1226, 688-1356, 714-974, 714-1216, |
| | 718-989, 771-1028, 844-1142, 846-1402, 914-1493, 943-1371, 1032-1571, 1041-1598, 1041-1623, 1091-1369, 1091- |
| | 1372, 1091-1700, 1110-1383, 1154-1723, 1158-1444, 1273-1771, 1327-1598, 1330-1616 |
| 76/6778214CB1/ | 1-143, 60-830, 148-772, 165-441, 182-425, 207-373, 217-893, 342-479, 381-851, 400-885, 404-851, 407-764, 415- |
| 2909 | 886, 420-851, 431-851, 432-867, 438-1047, 455-851, 475-882, 501-851, 515-851, 541-851, 550-813, 551-859, 568- |
| | 851, 580-844, 621-880, 694-875, 871-1537, 882-1105, 883-1469, 884-1313, 888-1480, 891-1318, 891-1326, 891- |
| | 1348, 891-1575, 911-1111, 911-1312, 914-1221, 925-1362, 925-1394, 1001-1290, 1066-1374, 1088-1243, 1113- |
| | 1296, 1171-1645, 1213-1326, 1319-1556, 1319-1878, 1319-1919, 1351-1921, 1357-1592, 1376-1867, 1390-1921, |
| | 1406-1646, 1430-1671, 1478-1725, 1502-1701, 1522-1921, 1558-1917, 1560-1746, 1560-1921, 1587-1795, 1695- |
| | 1921, 1841-2143, 1847-1921, 1922-2129, 1922-2425, 2000-2203, 2009-2249, 2016-2218, 2016-2313, 2019-2155, |
| | 2055-2290, 2096-2381, 2166-2394, 2204-2485, 2204-2486, 2204-2795, 2208-2440, 2208-2444, 2241-2443, 2246- |
| | 2488, 2246-2831, 2286-2909 |
| 77/258383CB1/ | 1-226, 82-368, 82-513, 82-520, 82-540, 82-557, 82-571, 82-575, 82-610, 82-613, 82-619, 82-626, 82-628, 82-676, |
| 2216 | 82-734, 83-468, 83-571, 83-634, 88-548, 90-219, 96-381, 98-616, 122-423, 125-562, 168-785, 177-846, 182-513, |
| | 184-395, 188-437, 188-723, 188-748, 189-379, 189-663, 189-768, 201-747, 202-1007, 221-578, 234-785, 267-708, |
| | 354-910, 388-1073, 397-978, 411-979, 423-964, 437-928, 438-1036, 471-807, 480-1043, 529-962, 537-1184, 560- |
| | 1084, 574-1077, 592-1010, 593-1073, 651-1084, 654-1066, 654-1084, 656-1084, 658-1252, 662-1343, 699-1196, |
| | 760-1319, 781-1449, 807-1067, 807-1309, 811-1082, 864-1121, 937-1235, 939-1495, 1007-1586, 1036-1464, 1072- |
| | 1356, 1110-1417, 1125-1664, 1134-1691, 1134-1716, 1184-1462, 1184-1465, 1184-1793, 1203-1476, 1233-1424, |
| | 1247-1816, 1251-1537, 1366-1929, 1420-1691, 1423-1709, 1595-2216, 1643-2062, 1645-2216, 1669-2216, 1735- |
| | 2216 |
| 78/2804937CB1/ | 1-841, 4-671, 292-843, 292-847, 294-643, 329-4681, 436-801, 605-894, 1232-1669, 1471-1741, 1950-2226, 1950- |
| 5320 | 2439, 1950-2443, 1950-2520, 1950-2524, 2113-2740, 2115-2775, 2120-2793, 2213-2798, 2260-2857, 2323-2857, |
| | 2347-2870, 2383-3024, 2398-3079, 2429-3142, 2456-3035, 2459-3079, 2508-2838, 2532-3079, 2572-3160, 2599- |
| | 3079, 2607-3209, 2607-3232, 2620-3219, 2697-3259, 2701-2879, 2701-2994, 2701-3340, 2763-2817, 2779-3422, |
| | 2804-3070, 2811-3035, 2831-3243, 2831-3417, 2844-3133, 2861-3369, 2900-3064, 2914-3500, 2974-3256, 3001- |
| | 3609, 3021-3595, 3032-3535, 3060-3333, 3060-3553, 3065-3337, 3065-3565, 3085-3670, 3087-3678, 3094-3607, |
| | 3118-3607, 3118-3727, 3119-3762, 3129-3619, 3130-3693, 3135-3726, 3165-3841, 3166-3623, 3166-3766, 3214- |
| | 3444, 3219-3809, 3223-3819, 3235-3715, 3240-3879, 3264-3710, 3305-3845, 3306-3820, 3329-3979, 3336-3792, |
| | 3339-3972, 3342-4018, 3369-4032, 3370-3890, 3405-3946, 3422-3639, 3432-3927, 3447-3706, 3471-4052, 3471- |
| | 4124, 3493-4138, 3494-4126, 3496-4105, 3501-3762, 3501-3921, 3502-4028, 3503-4076, 3523-4138, 3556-3829, |
| | 3557-3772, 3569-4175, 3578-4188, 3591-3850, 3617-4269, 3621-3827, 3626-4175, 3637-3955, 3643-4208, |
| | 3647-4203, 3659-4214, 3663-4155, 3663-4275, 3678-4011, 3682-3950, 3682-4088, 3682-4214, 3695-4268, 3701- |
| | 4230, 3704-4293, 3717-3955, 3717-4286, 3726-4266, 3731-4312, 3734-4345, 3737-4355, 3749-4344, 3750-4006, |
| | 3750-4331, 3751-4011, 3756-4343, 3763-4293, 3780-4272, 3780-4450, 3781-4316, 3786-4056, 3807-4196, 3808- |
| | 3905, 3809-4469, 3810-4368, 3813-4209, 3816-4344, 3821-4013, 3824-4305, 3831-4422, 3832-4360, 3848-4565, |
| | 3855-4565, 3857-4401, 3863-4151, 3863-4216, 3863-4323, 3868-4458, 3870-4509, 3871-4565, 3883-4415, 3886- |
| | 4495, 3908-4535, 3914-4470, 3915-4384, 3924-4507, 3925-4182, 3934-4236, 3935-4427, 3937-4200, 3942-4356, |
| | 3942-4479, 3943-4218, 3946-4232, 3947-4591, 3948-4462, 3948-4591, 3952-4196, 3955-4129, 3961-4547, 3963- |
| | 4514, 3967-4606, 3974-4608, 3978-4440, 3994-4458, 3997-4385, 3997-4619, 3998-4458, 4007-4470, 4009-4517, |
| | 4014-4509, 4017-4625, 4017-4682, 4021-4449, 4029-4599, 4039-4646, 4061-4094, 4062-4609, 4067-4366, 4074- |
| | 4584, 4076-4659, 4081-4349, 4086-4313, 4086-4589, 4097-4659, 4120-4551, 4122-4741, 4124-4716, 4125-4591, |
| | 4135-4670, 4136-4645, 4137-4683, 4142-4709, 4146-4372, 4146-4634, 4146-4692, 4146-4729, 4147-4396, 4153- |
| | 4715, 4162-4679, 4164-4706, 4169-4639, 4169-4699, 4172-4686, 4180-4403, 4186-4718, 4188-4810, 4192-4842, |
| | 4211-4753, 4230-4681, 4244-4672, 4245-4836, 4258-4729, 4263-4622, 4263-4854, 4264-4699, 4275-4830, 4276- |
| | 4885, 4280-4888, 4288-4534, 4288-4723, 4288-4938, 4289-4895, 4297-4429, 4300-4871, 4312-4657, 4315-4760, |
| | 4315-4914, 4327-4753, 4327-4806, 4338-4888, 4340-4761, 4340-4903, 4355-4590, 4357-4565, 4361-4899, 4364- |
| | 5013, 4374-4899, 4389-4894, 4389-4898, 4390-4903, 4393-4964, 4399-4648, 4399-4895, 4406-4719, 4416-4654, |
| | 4416-4664, 4416-4879, 4421-4899, 4434-4902, 4438-4903, 4448-4978, 4471-4903, 4475-5125, 4477-4836, 4494- |
| | 4853, 4494-4958, 4496-4903, 4502-4901, 4503-4914, 4507-4903, 4510-4902, 4568-4974, 4570-4762, 4573-4903, |
| | 4575-4805, 4614-4898, 4651-4903, 4720-4943, 4720-4964, 4722-4893, 4867-5130, 4953-5320, 5018-5269, 5104- |
| | 5320, 5134-5320, 5155-5320 |
| 79/7494915CB1/ | 1-653, 51-602 |
| 653 | |
| 80/2073751CB1/ | 1-415, 14-312, 14-367, 14-496, 14-507, 14-510, 14-559, 14-560, 19-268, 19-463, 54-340, 62-542, 81-564, 102-560, |
| 1794 | 110-941, 111-372, 117-369, 117-599, 120-326, 191-508, 199-709, 239-550, 252-731, 532-599, 600-658, 788-1715, |
| | 790-1227, 792-1044, 1051-1229, 1082-1227, 1266-1794, 1280-1583, 1395-1544, 1716-1794 |
| 81/3178841CB1/ | 1-282, 1-510, 8-281, 53-404, 55-518, 55-519, 58-501, 73-519, 78-447, 86-348, 90-501, 97-886, 115-515, 120-324, |
| 2691 | 120-446, 120-519, 130-519, 136-387, 136-694, 148-519, 152-519, 160-515, 163-517, 163-519, 168-466, 179-464, |
| | 202-510, 265-954, 267-469, 467-1036, 485-1029, 512-970, 514-1021, 515-1164, 516-907, 516-981, 517-1116, 518- |
| | 1067, 521-905, 521-906, 521-931, 521-946, 521-991, 521-997, 521-1021, 521-1022, 521-1023, 521-1036, 521-1041, |
| | 521-1047, 521-1050, 521-1059, 521-1066, 521-1082, 521-1089, 521-1113, 521-1115, 521-1126, 521-1128, 521- |
| | 1130, 521-1136, 521-1145, 521-1154, 521-1158, 521-1167, 521-1172, 522-1116, 527-1206, 530-871, 530-1209, 543- |
| | 815, 543-921, 547-867, 547-977, 559-1076, 561-929, 585-1217, 599-882, 613-1267, 619-885, 620-1194, 647-1261, |
| | 653-1303, 673-1310, 692-1369, 698-1123, 720-1378, 733-1476, 734-889, 738-1379, 744-1025, 764-1217, 770-1470, |
| | 789-985, 793-1470, 794-1304, 800-1055, 813-1416, 834-1496, 871-1568, 883-1152, 883-1163, 883-1649, 905-1471, |
| | 925-1475, 937-1203, 1011-1630, 1012-1589, 1031-1512, 1054-1639, 1066-1719, 1120-1606, 1120-1755, 1156- |
| | 1396, 1157-1755, 1159-1431, 1177-1755, 1184-1388, 1238-1733, 1247-1737, 1248-1805, |
| | 1273-1512, 1273-1519, 1273-1527, 1273-1528, 1303-1769, 1346-1906, 1357-1525, 1361-1755, 1430-1727, 1453- |
| | 1966, 1459-1762, 1466-1755, 1492-2116, 1499-1784, 1501-1805, 1544-2110, 1565-1947, 1580-1967, 1582-1816, |
| | 1588-1765, 1605-1771, 1622-2150, 1631-2200, 1655-2263, 1672-1961, 1672-2161, 1717-2229, 1718-1921, 1718- |
| | 2180, 1730-2225, 1749-2302, 1749-2382, 1751-2005, 1751-2007, 1766-1992, 1776-2037, 1803-2200, 1822-2083, |
| | 1856-2292, 1869-2440, 1918-2081, 1939-2549, 1949-2601, 1953-2601, 1968-2397, 1989-2574, 2004-2394, 2009- |
| | 2536, 2043-2332, 2067-2346, 2068-2333, 2068-2425, 2069-2318, 2069-2417, 2074-2327, 2083-2341, 2112-2378, |
| | 2112-2648, 2112-2687, 2132-2442, 2136-2682, 2151-2662, 2167-2380, 2187-2691, 2200-2462, 2207-2486, 2234- |
| | 2531, 2238-2467, 2417-2579 |
| 82/3674807CB1/ | 1-290, 1-436, 1-453, 1-456, 1-488, 1-491, 1-524, 1-558, 1-563, 1-581, 1-591, 1-598, 1-600, 1-613, 1-641, 1-652, 1- |
| 2056 | 686, 1-754, 2-575, 41-736, 58-502, 83-748, 105-854, 126-911, 133-395, 157-874, 167-650, 197-807, 226-703, 230- |
| | 807, 265-716, 323-891, 388-1071, 406-708, 417-1080, 440-1053, 441-682, 443-1099, 444-1024, 445-1075, 497-850, |
| | 504-1064, 527-1165, 547-1182, 573-1124, 631-1179, 635-1204, 636-1165, 660-1163, 664-1218, 688-864, 716-1206, |
| | 719-1339, 723-1023, 752-1255, 759-1295, 771-1325, 773-1382, 778-1378, 795-1382, 800-1455, 804-1354, 811- |
| | 1272, 823-1461, 834-1202, 837-1525, 845-1438, 846-1296, 850-1224, 857-1058, 866-983, 866-1282, 866-1362, 918- |
| | 1530, 976-1553, 983-1497, 1009-1446, 1046-1509, 1251-1882, 1388-1888, 1424-1711, 1721-2056, 1754-2056, 1807- |
| | 2056 |
| 83/1794922CB1/ | 1-283, 1-407, 169-592, 169-957, 579-972, 838-1488, 850-1634, 896-1138, 896-1385, 1031-1642, 1084-1347, 1084- |
| 2275 | 1544, 1166-1411, 1357-1649, 1357-1662, 1417-2118, 1459-1655, 1459-1722, 1464-1719, 1464-1996, 1472-2167, |
| | 1574-2254, 1575-1878, 1643-1791, 1684-2275, 1739-1994, 1739-2183, 1739-2199, 1765-2246, 1808-2266, 1810- |
| | 2269, 1816-2265, 1821-2257, 1825-2275, 1844-2265, 1876-2266, 1926-2265, 2042-2275 |
| 84/1795509CB1/ | 1-282, 1-284, 62-313, 71-271, 72-325, 72-512, 72-826, 100-415, 108-345, 145-648, 167-555, 238-443, 238-943, 240- |
| 1219 | 788, 293-552, 322-845, 337-887, 370-887, 397-685, 443-683, 443-921, 443-943, 469-694, 499-1096, 512-745, 526- |
| | 717, 530-845, 571-1009, 574-1034, 575-889, 579-1038, 579-1051, 579-1061, 583-1052, 584-845, 651-878, 661- |
| | 1210, 661-1219, 668-1219, 674-1190, 677-845, 740-1208, 859-1219, 944-1206 |
| 85/2017180CB1/ | 1-264, 1-305, 1-447, 6-433, 7-515, 7-517, 9-330, 11-615, 15-308, 16-235, 16-293, 18-211, 18-279, 18-295, 20-665, |
| 1015 | 21-224, 22-375, 26-718, 33-259, 35-300, 35-812, 36-290, 39-504, 40-334, 51-328, 52-287, 52-747, 58-317, 72-686, |
| | 79-527, 80-344, 114-677, 145-308, 174-444, 196-465, 228-463, 252-753, 362-768, 439-717, 459-1015, 479-748, 520- |
| | 657, 527-1012, 557-862, 602-884, 673-1012, 703-994, 706-987, 722-964, 725-976, 725-1015, 731-999, 743-991 |
| 86/219442CB1/ | 1-408, 1-416, 1-460, 2-384, 4-297, 7-283, 118-745, 158-447, 350-855, 376-855, 381-612, 394-664, 419-855, 441- |
| 2392 | 855, 493-659, 525-1114, 592-855, 629-1263, 635-953, 665-1113, 774-1133, 801-998, 820-1269, 883-1239, 913- |
| | 1150, 916-1044, 937-1114, 963-1061, 963-1115, 1035-1269, 1096-1628, 1120-1616, 1437-1941, 1617-2266, 1619- |
| | 1890, 1668-1976, 1691-2045, 1707-2253, 1713-2392, 1787-2080, 1798-2096, 1816-1946, 1818-2392, 1836-2392, |
| | 1879-2345, 1901-2392, 1935-2328, 1939-2392, 1944-2328, 1953-2059, 1964-2382 |
| 87/2597459CB1/ | 1-578, 148-611, 404-1009, 421-824, 427-884, 432-612, 517-1000, 643-847, 643-909, 643-1098, 643-1123, 643- |
| 1799 | 1130, 643-1136, 643-1139, 643-1143, 643-1149, 643-1150, 643-1164, 643-1169, 643-1178, 643-1180, 643-1181, |
| | 643-1182, 643-1189, 643-1200, 643-1210, 643-1219, 643-1249, 643-1277, 643-1290, 674-1283, 701-1279, 705- |
| | 1413, 755-1305, 898-1080, 966-1231, 966-1425, 1018-1711, 1019-1588, 1022-1409, 1052-1630, 1066-1552, 1069- |
| | 1442, 1119-1713, 1133-1696, 1145-1555, 1165-1754, 1190-1795, 1194-1767, 1202-1743, 1251-1761, 1255-1381, |
| | 1262-1789, 1262-1799, 1283-1799, 1314-1440, 1314-1767, 1379-1634, 1384-1599, 1384-1799, 1437-1706, 1444- |
| | 1727 |
| 88/2783863CB1/ | 1-556, 227-772, 270-767, 279-574, 279-823, 279-909, 300-863, 387-977, 468-959, 600-1232, 884-1201, 884-1341, |
| 3718 | 884-1455, 927-1069, 962-1117, 962-1562, 985-1742, 1007-1525, 1007-1592, 1045-1763, 1066-1448, 1088-1339, |
| | 1171-2106, 1183-1451, 1183-1742, 1189-1967, 1250-1897, 1267-1954, 1296-2124, 1301-1449, 1433-2059, 1466- |
| | 2059, 1560-2297, 1571-1823, 1571-1828, 1606-2363, 1625-1911, 1658-2015, 1664-1905, 1664-1919, 1664-2146, |
| | 1676-2352, 1681-1994, 1683-2031, 1686-2246, 1839-2104, 1846-2049, 1850-2069, 1856-2826, 1870-2666, 2056- |
| | 2233, 2113-2365, 2216-2491, 2218-2823, 2241-2473, 2256-2834, 2256-2881, 2266-2546, 2298-2564, 2330-2618, |
| | 2336-2618, 2341-2784, 2363-2604, 2377-2897, 2387-2902, 2402-2646, 2402-2671, 2402-2954, 2450-2733, 2456- |
| | 2875, 2503-2721, 2512-2973, 2627-2889, 2644-2908, 2741-2973, 2755-3289, 2816-3209, 2836-3387, 2847-3655, |
| | 2895-3151, 2896-3466, 2912-3158, 2945-3180, 2970-3262, 2971-3224, 2985-3710, 2987-3274, 3063-3718, 3064- |
| | 3675, 3106-3636, 3116-3710, 3117-3695, 3139-3704, 3143-3712, 3192-3700, 3200-3496, 3201-3712, 3215-3664, |
| | 3227-3699, 3228-3645, 3248-3711, 3249-3711, 3252-3710, 3258-3710, 3305-3665, 3356-3599, 3356-3696, 3356- |
| | 3710, 3372-3707, 3407-3594, 3407-3605, 3407-3707, 3413-3664, 3426-3711, 3429-3707 |
| 89/2902971CB1/ | 1-105, 1-622, 1-644, 2-200, 2-285, 2-287, 2-821, 4-273, 8-232, 8-254, 8-256, 8-282, 8-635, 13-251, 16-236, 18-138, |
| 3250 | 18-284, 18-303, 18-313, 19-812, 29-673, 34-231, 34-232, 34-282, 34-842, 43-482, 61-274, 64-266, 65-266, 68-266, |
| | 102-650, 200-274, 208-258, 276-582, 327-501, 575-1157, 665-838, 665-1384, 784-932, 803-1417, 848-1665, 855- |
| | 1472, 908-1530, 1013-1615, 1030-1345, 1041-1266, 1123-1527, 1157-1611, 1160-1737, 1293-1535, 1403-1721, |
| | 1450-2069, 1466-1710, 1631-1749, 1631-2071, 1700-1983, 1739-2028, 1739-2180, 1809-2254, 1844-2278, 1897- |
| | 2310, 1959-2199, 1959-2477, 1959-2491, 1971-2250, 2001-2477, 2043-2477, 2043-2516, 2050-2477, 2063-2477, |
| | 2165-2362, 2165-2603, 2165-2783, 2285-2980, 2363-2748, 2364-2975, 2394-2659, 2467-3133, 2477-3163, 2478- |
| | 2936, 2478-2978, 2536-2807, 2604-2987, 2623-2875, 2623-2907, 2623-2911, 2623-2924, 2625-2944, 2678-3224, |
| | 2698-3219, 2755-3204, 2766-3237, 2775-3250, 2789-3062, 2789-3217, 2789-3222, 2792-3245, 2801-3242, 2812- |
| | 3237, 2828-3237, 2845-3235, 2849-3228, 2897-3231, 2923-3131, 2953-3250 |
| 90/368660CB1/ | 1-584, 4-273, 7-286, 33-410, 34-790, 35-811, 41-548, 529-1215, 530-935, 584-1232, 648-1227, 653-1394, 654- |
| 2295 | 1118, 684-1187, 795-1048, 923-1158, 928-1227, 928-1415, 958-1196, 1016-1265, 1017-1325, 1038-1309, 1163- |
| | 1368, 1172-1542, 1388-1705, 1473-1557, 1477-1691, 1482-1774, 1485-2117, 1489-1747, 1526-2130, 1569-1748, |
| | 1658-2186, 1712-1917, 1712-1931, 1712-1934, 1712-1936, 1712-1947, 1712-1965, 1712-1982, 1714-2190, 1720- |
| | 1853, 1728-2295, 1740-1996, 1754-2252, 1777-1984, 1779-2042, 1791-2051, 1853-2031, 1863-1997, 1920-2293, |
| | 1922-2295 |
| 91/2804990CB1/ | 1-738, 1-780, 210-758, 214-1734, 414-722, 484-517, 495-743, 498-884, 518-801, 641-1240, 679-975, 735-1131, 743- |
| 1777 | 1234, 763-1285, 847-1458, 868-1191, 918-1186, 973-1353, 1041-1348, 1041-1452, 1094-1432, 1154-1594, 1202- |
| | 1395, 1214-1614, 1214-1718, 1218-1688, 1218-1710, 1218-1747, 1222-1487, 1222-1689, 1222-1746, 1253-1478, |
| | 1255-1777, 1266-1720, 1270-1594, 1271-1594, 1273-1594, 1279-1550, 1282-1552 |
| 92/168571CB1/ | 1-564, 1-591, 50-722, 177-780, 382-983, 404-661, 426-1056, 444-979, 543-1149, 599-1147, 648-1148, 682-1146, |
| 3181 | 687-1146, 689-1139, 909-1605, 1150-1412, 1150-1567, 1150-1611, 1152-1417, 1157-1765, 1159-1624, 1159-1666, |
| | 1159-1759, 1159-1760, 1168-1625, 1196-1449, 1216-1550, 1291-1505, 1291-1541, 1322-1737, 1331-1542, 1579- |
| | 1793, 1601-1967, 1707-2028, 1717-1942, 1781-2282, 1800-2145, 1836-2063, 1839-2077, 1839-2409, 1865-2430, |
| | 1875-2369, 1952-2369, 1960-2574, 1960-2593, 1961-2169, 1968-2611, 1971-2361, 1975-2558, 1979-2167, 1999- |
| | 2656, 2019-2298, 2021-2406, 2021-2476, 2022-2241, 2022-2559, 2062-2304, 2079-2271, 2112-2382, 2128-2715, |
| | 2133-2685, 2160-2672, 2185-2509, 2204-2808, 2222-2644, 2223-2814, 2293-2571, 2303-2523, 2305-2868, 2329- |
| | 2576, 2334-2606, 2335-2628, 2341-2856, 2379-2693, 2417-2638, 2425-2727, 2472-2896, 2494-3097, 2495-2724, |
| | 2500-2696, 2500-2772, 2500-2781, 2500-2835, 2500-3078, 2501-2773, 2504-3008, 2519-3171, 2540-2851, 2544- |
| | 2861, 2565-2827, 2567-2786, 2579-3101, 2583-2744, 2586-2885, 2586-3094, 2588-3102, 2602-2863, 2607-2859, |
| | 2648-2863, 2649-2814, 2664-3165, 2671-3161, 2708-3002, 2749-3094, 2800-3180, 2821-3098, 2821-3116, 2902- |
| | 3149, 3025-3181 |
| 93/1286391CB1/ | 1-650, 1-2676, 152-347, 266-860, 266-968, 362-528, 442-4155, 634-1105, 660-901, 823-1005, 823-1263, 1004- |
| 5987 | 1581, 1104-1337, 1126-1735, 1207-1630, 1229-1926, 1324-2013, 1415-1659, 1415-1728, 1838-2104, 1872-2353, |
| | 1959-2529, 2093-2721, 2111-2647, 2132-2438, 2177-2678, 2233-2705, 2331-3122, 2352-2542, 2447-2678, 2491- |
| | 3289, 2537-3129, 2704-3268, 2729-3009, 2729-3192, 2729-3194, 2729-3279, 2729-3292, 2805-3377, 2822-3285, |
| | 2875-3573, 2893-3549, 3028-3710, 3060-3611, 3079-3290, 3114-3527, 3256-3902, 3281-3856, 3297-3848, 3331- |
| | 3838, 3336-3851, 3364-3863, 3438-3903, 3439-4143, 3545-4169, 3603-4056, 3603-4252, 3698-4456, 3701-4276, |
| | 3742-4452, 3743-4167, 3743-4326, 3743-4360, 3743-4365, 3743-4411, 3743-4424, 3743-4427, 3743-4458, 3743- |
| | 4487, 3747-3967, 3763-4251, 3780-4419, 3790-4053, 3790-4220, 3804-4498, 3807-4424, 3887-4391, 3888-4502, |
| | 3894-4451, 3896-4502, 3928-4074, 3928-4156, 3928-4499, 3930-4380, 3933-4609, 3961-4662, 3986-4244, 3991- |
| | 4459, 3992-4629, 3999-4529, 4025-4577, 4054-4691, 4054-4709, 4145-4184, 4193-4757, 4350-4878, 4350-5055, |
| | 4350-5067, 4351-5002, 4490-5197, 4490-5265, 4532-5209, 4545-5117, 4563-5157, 4592-5083, 4646-5278, |
| | 4650-5278, 4652-5315, 4683-5357, 4684-5339, 4709-5193, 4715-5341, 4720-5399, 4731-5400, 4732-5395, 4744- |
| | 5220, 4756-5348, 4763-5378, 4785-5413, 4788-5078, 4788-5267, 4788-5281, 4788-5309, 4788-5383, 4788-5472, |
| | 4788-5491, 4789-5386, 4789-5395, 4791-5425, 4792-5440, 4798-5064, 4798-5363, 4799-5383, 4799-5491, 4799- |
| | 5584, 4800-5349, 4801-5607, 4818-5087, 4823-5300, 4831-5288, 4844-5125, 4844-5320, 4845-5032, 4861-5409, |
| | 4897-5514, 4898-5493, 4899-5527, 4906-5384, 4910-5163, 4910-5578, 4914-5480, 4914-5493, 4915-5158, 4915- |
| | 5503, 4920-5527, 4921-5496, 4936-5650, 4939-5221, 4942-5449, 4946-5535, 4949-5559, 4950-5527, 4954-5455, |
| | 4954-5536, 4956-5501, 4964-5597, 4965-5565, 4967-5599, 4980-5564, 4999-5483, 5001-5457, 5002-5527, 5018- |
| | 5556, 5021-5274, 5024-5534, 5040-5564, 5042-5514, 5061-5522, 5068-5539, 5070-5696, 5140-5371, 5232-5942, |
| | 5311-5977, 5327-5981, 5331-5987, 5333-5977, 5336-5955, 5485-5987, 5572-5981, 5629-5743, 5629-5920, 5684- |
| | 5905, 5688-5920, 5753-5965, 5766-5920 |
| 94/2007684CB1/ | 1-275, 1-536, 23-227, 55-580, 250-765, 345-610, 420-765, 451-759, 452-613, 474-613 |
| 765 | |
| 95/2227040CB1/ | 1-418, 123-418, 162-484, 167-671, 187-391, 428-651, 524-764, 524-769, 524-1060, 552-667, 601-952, 797-1050, |
| 2674 | 797-1336, 840-1091, 1023-1444, 1034-1555, 1104-1684, 1149-1369, 1149-1626, 1301-1416, 1310-1545, 1318- |
| | 1546, 1506-2099, 1661-2031, 1662-1944, 1677-2331, 1705-1968, 1771-2277, 1799-2106, 1901-2385, 1936-2142, |
| | 1936-2190, 1936-2208, 1941-2209, 1944-2242, 1944-2247, 1944-2400, 1945-2150, 1968-2192, 1968-2474, 2002- |
| | 2233, 2035-2674, 2039-2295, 2064-2401, 2071-2639, 2083-2256, 2097-2248, 2101-2671, 2160-2419, 2166-2330, |
| | 2166-2377, 2168-2389, 2278-2377, 2290-2377 |
| 96/4346130CB1/ | 1-479, 2-493, 2-533, 2-535, 2-561, 21-272, 24-400, 30-558, 32-402, 37-480, 179-705, 471-800, 492-789, 536-672, |
| 5920 | 536-910, 536-1141, 784-1423, 860-1379, 971-1215, 971-1226, 971-1358, 971-1565, 1053-1324, 1137-1485, 1349- |
| | 1817, 1368-1624, 1368-1841, 1435-1987, 1435-2009, 1561-2121, 1588-1817, 1590-2121, 1600-1817, 1720-1817, |
| | 1722-1817, 1768-2170, 2013-2504, 2079-2317, 2079-2346, 2079-2398, 2155-2682, 2155-2738, 2310-5524, 2373- |
| | 2857, 2451-2684, 2544-3072, 2771-3257, 2948-3187, 3369-4024, 3537-4052, 4366-4776, 4377-4732, 4378-4682, |
| | 4413-4693, 4752-5051, 4919-5161, 4945-5574, 5013-5385, 5055-5646, 5070-5319, 5241-5501, 5303-5864, 5392- |
| | 5780, 5392-5802, 5393-5627, 5431-5684, 5431-5920, 5433-5837, 5469-5873 |
| 97/55117040CB1/ | 1-341, 1-347, 1-385, 1-418, 1-560, 1-661, 4-661, 5-661, 58-637, 89-157, 89-167, 89-171, 114-823, 200-268, 200- |
| 1689 | 282, 201-282, 225-268, 325-877, 805-1689, 817-1532 |
| 98/7472392CB1/ | 1-157, 1-158, 1-195, 1-333, 1-364, 1-438, 1-508, 1-510, 1-513, 1-546, 1-620, 1-624, 1-657, 1-684, 1-721, 1-735, 1- |
| 1314 | 744, 1-764, 1-831, 1-912, 1-1048, 1-1307, 4-548, 14-811, 20-661, 24-920, 76-1044, 80-141, 231-778, 231-782, 342- |
| | 761, 502-1031, 536-1035, 536-1314, 561-657, 576-973, 648-788 |
| 99/4028960CB1/ | 1-824, 11-856, 19-545, 97-209, 115-790, 174-777, 174-850, 187-655, 209-534, 396-678, 434-650, 434-950, 657- |
| 3322 | 935, 657-1053, 783-1046, 783-1383, 834-970, 879-1067, 885-1496, 945-1592, 1071-1491, 1176-1337, 1234-1892, |
| | 1284-1663, 1293-1801, 1314-1534, 1320-1704, 1341-1953, 1454-1760, 1684-1919, 1684-1920, 1684-1933, 1684- |
| | 2140, 1690-2148, 1850-2042, 1873-2116, 1875-2098, 1890-2348, 1963-2703, 2027-2518, 2043-2329, 2101-2324, |
| | 2127-2372, 2137-2776, 2169-2577, 2218-2493, 2322-2911, 2324-2491, 2336-2994, 2344-2558, 2395-2924, 2403- |
| | 3035, 2409-2592, 2414-2978, 2415-2688, 2496-2757, 2498-2724, 2505-3038, 2529-2864, 2536-2980, 2562-2692, |
| | 2632-3052, 2654-3051, 2743-3233, 2767-3227, 2784-3224, 2787-2921, 2794-3016, 2808-3218, 2817-3050, 2906- |
| | 3322 |
| 100/8227004CB1/ | 1-250, 1-434, 1-492, 1-611, 1-612, 1-676, 1-860, 100-900, 114-772, 238-680, 247-879, 273-494, 288-685, 341-1108, |
| 3621 | 396-813, 402-813, 425-1104, 467-1245, 507-1224, 526-896, 650-1364, 655-1231, 656-1302, 661-1301, 668-1423, |
| | 671-1042, 679-1258, 711-1301, 760-1301, 763-1131, 782-1542, 787-1323, 794-1224, 818-1271, 852-1355, 874- |
| | 1462, 904-1036, 916-1301, 961-1624, 980-1454, 1011-1397, 1025-1592, 1028-1255, 1031-1728, 1065-1298, 1065- |
| | 1605, 1149-1842, 1168-1548, 1202-1682, 1209-2160, 1217-1697, 1229-1757, 1282-1511, 1313-1804, 1334-2037, |
| | 1362-2087, 1372-1967, 1400-2375, 1407-2144, 1497-2162, 1621-2285, 1627-2092, 1627-2569, 1629-2068, 1630- |
| | 2318, 1652-1880, 1673-2409, 1683-2354, 1708-1979, 1733-1995, 1742-2449, 1743-2000, 1745-2189, 1746-2419, |
| | 1751-2439, 1751-2497, 1759-2530, 1792-2108, 1795-2614, 1816-2097, 1834-2105, 1845-2598, 1893-2144, 1893- |
| | 2485, 1903-2586, 1904-2715, 1904-2732, 1949-2203, 1951-2207, 1975-2296, 1977-2878, 1979-2214, 1980-2645, |
| | 1989-2407, 1996-2256, 1999-2668, 2006-2722, 2025-2560, 2032-2514, 2046-2745, 2061-2540, 2079-2707, 2094- |
| | 2348, 2094-2895, 2106-2296, 2106-2510, 2109-2289, 2109-2597, 2112-2372, 2119-2385, 2138-2789, |
| | 2150-2447, 2174-2333, 2188-2889, 2195-2841, 2209-2802, 2216-2546, 2251-2596, 2251-2604, 2272-2527, 2296- |
| | 2583, 2297-2575, 2312-2811, 2321-2574, 2335-2664, 2343-2950, 2350-2865, 2351-2879, 2353-2827, 2355-2567, |
| | 2363-2759, 2390-2588, 2394-2664, 2421-2920, 2426-2740, 2427-2942, 2447-2862, 2458-2846, 2462-3141, 2466- |
| | 2720, 2470-3192, 2483-2699, 2483-2997, 2798-3256, 2859-3106, 2908-3175, 2921-3178, 3049-3309, 3049-3621 |
| 101/3044763CB1/ | 1-283, 41-819, 66-368, 88-204, 215-856, 218-734, 303-647, 389-661, 421-706, 465-713, 482-1060, 490-610, 490- |
| 2704 | 952, 511-1108, 516-1026, 569-1199, 576-1240, 585-1112, 673-1246, 677-1221, 697-974, 732-978, 742-1290, 755- |
| | 1049, 780-1430, 824-997, 844-1079, 872-1392, 913-1188, 946-1561, 979-1529, 1035-1304, 1035-1755, 1035-1818, |
| | 1039-1294, 1056-1207, 1056-1290, 1076-1758, 1111-1489, 1113-1375, 1117-1433, 1147-1515, 1162-1458, 1166- |
| | 1744, 1171-1427, 1172-1518, 1173-1393, 1191-1461, 1208-1511, 1217-1365, 1227-1537, 1249-1800, 1273-1923, |
| | 1290-1513, 1296-1547, 1302-1495, 1321-1409, 1324-1798, 1332-1627, 1341-1494, 1341-1623, 1343-1818, 1363- |
| | 1682, 1363-1886, 1380-1649, 1388-1631, 1390-1635, 1390-1822, 1392-1911, 1397-1818, 1400-1818, 1402-1734, |
| | 1419-1791, 1420-1799, 1429-1818, 1438-1714, 1460-1646, 1461-1677, 1503-1918, 1511-1897, 1522-1608, 1556- |
| | 1799, 1571-1818, 1577-2418, 1596-1818, 1637-1918, 1643-1878, 1658-1808, 1658-1818, 1660-2116, 1754-1860, |
| | 1757-1813, 1757-1818, 1760-1918, 2110-2704 |
| 102/4044519CB1/ | 1-146, 1-291, 1-490, 1-574, 1-583, 1-598, 1-627, 1-631, 1-657, 1-679, 1-680, 1-808, 92-804, 98-820, 159-495, 479- |
| 1345 | 1252, 509-1289, 615-1345, 674-1329, 684-1341, 764-1342 |
| 103/71351918CB1/ | 1-246, 27-253, 29-80, 30-304, 33-278, 33-376, 33-561, 34-225, 36-571, 38-280, 43-324, 60-315, 61-286, 169-738, |
| 1607 | 308-578, 419-947, 423-659, 424-695, 441-1112, 444-972, 466-994, 468-793, 478-829, 490-798, 498-1124, 506-725, |
| | 512-1016, 516-946, 519-791, 545-1040, 547-997, 565-1145, 568-849, 570-813, 570-956, 571-965, 576-736, 579- |
| | 1111, 581-1028, 582-1167, 587-1142, 588-1208, 595-1145, 595-1378, 596-857, 600-1109, 614-1186, 624-1220, 628- |
| | 848, 633-1213, 638-1225, 640-1172, 641-1244, 648-822, 648-1403, 649-955, 659-756, 672-1106, 676-1403, 678- |
| | 949, 680-1192, 683-931, 684-1206, 684-1228, 693-976, 699-1040, 700-960, 711-1305, 715-1263, 726-1304, 730- |
| | 987, 730-1358, 742-1339, 748-1399, 757-1264, 760-1244, 770-992, 771-1131, 787-1301, 788-1014, 796-1301, 798- |
| | 1336, 814-1374, 825-1536, 832-1068, 838-1396, 843-1044, 846-1093, 846-1482, 858-1114, 860-1494, 866-1149, |
| | 866-1428, 867-1306, 875-1471, 876-1120, 878-1146, 879-1238, 883-1528, 890-1533, 893-1090, 897-1393, 899- |
| | 1127, 899-1546, 900-1203, 904-1281, 917-1364, 918-1430, 928-1371, 930-1544, 937-1540, 949-1444, 952-1414, |
| | 952-1537, 955-1527, 956-1081, 963-1533, 988-1106, 990-1264, 999-1540, 1003-1459, 1006-1539, |
| | 1014-1473, 1015-1415, 1025-1288, 1028-1543, 1028-1550, 1030-1539, 1043-1527, 1046-1317, 1047-1358, 1047- |
| | 1367, 1047-1607, 1050-1546, 1051-1475, 1058-1353, 1070-1316 |
| 104/8109363CB1/ | 1-521, 125-498, 246-543, 268-746, 315-549, 367-885, 403-1139, 464-717, 634-903, 662-1255, 704-925, 742-1371, |
| 2622 | 845-1124, 864-1385, 966-1471, 1041-1444, 1045-1444, 1105-1415, 1105-1618, 1320-1624, 1320-1888, 1361-1648, |
| | 1373-1884, 1448-1688, 1556-1811, 1584-2049, 1591-1840, 1596-2174, 1690-1962, 1697-1956, 1697-2207, 1701- |
| | 1826, 1772-2051, 1790-2055, 1935-2190, 1937-2617, 2003-2253, 2003-2302, 2003-2490, 2010-2236, 2010-2309, |
| | 2011-2622, 2012-2593, 2052-2318, 2063-2292, 2063-2296, 2087-2622, 2094-2616, 2096-2340, 2161-2424, 2254- |
| | 2510 |
| 105/1272746CB1/ | 1-214, 1-254, 1-595, 3-267, 7-248, 41-267, 46-275, 154-268, 202-394, 202-775, 202-841, 207-392, 207-534, 207- |
| 3489 | 918, 340-632, 340-866, 401-1133, 463-1095, 506-1225, 592-864, 746-1357, 748-1382, 772-1095, 929-1193, 954- |
| | 1410, 959-1425, 1059-1554, 1064-1421, 1166-1419, 1336-1669, 1449-1951, 1493-2128, 1519-1968, 1519-2004, |
| | 1519-2045, 1519-2055, 1519-2096, 1519-2113, 1519-2155, 1519-2161, 1519-2202, 1520-2009, 1521-1786, 1560- |
| | 2144, 1569-2178, 1625-2247, 1642-1783, 1663-1912, 1700-2520, 1710-2140, 1734-2263, 1813-2446, 1860-2519, |
| | 1880-2572, 1937-2098, 1951-2552, 1953-2706, 1955-2186, 1955-2219, 1955-2641, 1955-2763, 1956-2325, 1959- |
| | 2420, 1965-2559, 1976-2243, 1982-2480, 1994-2072, 2023-2579, 2024-2647, 2031-2642, 2052-2697, 2053-2742, |
| | 2126-2775, 2127-2699, 2135-2420, 2139-2833, 2173-2730, 2177-2840, 2182-2783, 2183-2869, 2215-2456, 2215- |
| | 2830, 2229-2785, 2236-2935, 2242-2935, 2246-2852, 2256-3027, 2257-2763, 2288-2848, 2294-2924, 2296-2932, |
| | 2301-2525, 2314-2824, 2322-2524, 2322-2792, 2324-2935, 2341-2935, 2344-2914, 2354-2935, 2358-2935, 2373- |
| | 2932, 2379-2932, 2382-2935, 2384-2918, 2386-2599, 2386-2935, 2389-2935, 2391-3001, 2397-2693, 2413-2639, |
| | 2422-2935, 2424-2935, 2431-2679, 2443-3079, 2449-2935, 2468-3177, 2477-2941, 2487-2856, 2495-2935, 2499- |
| | 2932, 2516-2932, 2524-3136, 2526-2932, 2530-2938, 2558-2935, 2561-2935, 2609-3294, 2620-3315, 2621-2940, |
| | 2624-2907, 2648-2773, 2658-3455, 2674-3489, 2708-3463, 2756-3191, 2768-2977, 2768-3252, 2768-3265, 2768- |
| | 3267, 2768-3278, 2768-3288, 2768-3372, 2830-3068, 2830-3195, 2833-3452, 2867-3421, 2889-3431, 2916-3366, |
| | 2941-3196, 2962-3443, 2977-3197, 2992-3444, 3020-3285, 3105-3349, 3105-3434, 3105-3452, 3122-3330, 3125- |
| | 3443, 3126-3449, 3137-3306, 3228-3443, 3230-3443, 3305-3449 |
| 106/1839974CB1/ | 1-747, 1-757, 24-624, 25-529, 25-530, 87-694, 150-944, 179-571, 313-528, 458-1022, 469-1197, 588-930, 820- |
| 2269 | 1097, 893-1455, 1174-1547, 1174-1604, 1179-1519, 1362-1636, 1447-1743, 1513-1804, 1521-1764, 1654-2269, |
| | 1658-1892, 1658-2148, 1661-2231, 1812-2269, 1905-2103, 1912-2175, 1949-2240, 2008-2098 |
| 107/1877336CB1/ | 1-194, 1-197, 1-399, 3-412, 4-295, 7-222, 9-240, 13-569, 13-608, 15-558, 15-642, 17-218, 19-628, 25-277, 25-549, |
| 3075 | 33-305, 34-614, 38-621, 43-698, 102-510, 103-770, 103-800, 112-738, 137-754, 138-429, 144-664, 173-730, 241- |
| | 514, 259-535, 262-614, 262-662, 286-594, 421-648, 573-1015, 641-1229, 691-1147, 713-942, 725-1421, 757-1368, |
| | 803-1368, 806-1309, 820-1492, 835-1360, 835-1368, 837-1459, 840-1325, 844-1339, 872-1439, 877-1464, 883- |
| | 1467, 889-1366, 893-1557, 895-1366, 898-1370, 926-1593, 959-1455, 971-1387, 1007-1678, 1014-1567, 1045- |
| | 1572, 1075-1707, 1097-1225, 1104-1471, 1104-1662, 1104-1698, 1106-1800, 1154-1653, 1156-1735, 1160-1850, |
| | 1176-1709, 1176-1859, 1177-1806, 1180-1801, 1183-1718, 1196-1800, 1215-1736, 1224-1878, 1252-1897, 1265- |
| | 1829, 1272-1801, 1283-1639, 1291-1815, 1295-1830, 1314-1866, 1316-1807, 1318-1861, 1319-1795, 1319-1908, |
| | 1325-1836, 1335-1848, 1335-1851, 1338-1941, 1340-1905, 1346-1784, 1347-1867, 1355-1820, 1359-1851, 1363- |
| | 1859, 1379-1947, 1384-1891, 1406-1927, 1417-1902, 1436-2104, 1500-2058, 1519-1981, 1520-2025, 1520-2034, |
| | 1521-1859, 1521-1860, 1526-2061, 1528-2160, 1529-2025, 1548-2212, 1557-2158, 1562-2158, 1567-2301, |
| | 1591-2113, 1598-2183, 1606-2125, 1610-2247, 1612-2157, 1612-2186, 1628-2291, 1647-2255, 1675-2326, 1684- |
| | 2069, 1684-2301, 1687-2349, 1699-2148, 1700-2215, 1700-2252, 1704-2212, 1705-2205, 1705-2360, 1738-2370, |
| | 1743-2076, 1750-1776, 1752-2278, 1762-2337, 1763-2190, 1763-2285, 1764-2236, 1777-2503, 1791-2295, 1793- |
| | 2186, 1794-2364, 1800-2361, 1805-2226, 1815-2085, 1819-2382, 1831-2323, 1842-2302, 1873-2325, 1988-2212, |
| | 2005-2608, 2039-2706, 2085-2673, 2323-2357, 2365-2695, 2397-2962, 2402-2686, 2418-2677, 2419-2999, 2420- |
| | 2670, 2430-2668, 2448-2678, 2449-2612, 2457-2660, 2603-2663, 2702-2789, 2704-3056, 2716-2984, 2723-3056, |
| | 2729-3057, 2736-2912, 2738-2981, 2762-2981, 2762-2997, 2769-2974, 2769-3013, 2769-3027, 2777-3057, 2784- |
| | 3024, 2785-3058, 2789-3029, 2801-3018, 2811-3056, 2811-3075, 2822-3049, 2823-3049, 2829-3055, 2831-3075, |
| | 2833-3039, 2834-3059, 2845-3058, 2851-3061, 2851-3075, 2852-3027, 2852-3046, 2861-3075, 2865-3059, 2865- |
| | 3075, 2867-3037, 2868-3075, 2876-3046, 2876-3056, 2876-3059, 2876-3073, 2877-3056, 2878-3039, 2879-3058, |
| | 2881-2987, 2883-3063, 2885-3032, 2885-3075, 2887-3056, 2887-3058, 2887-3059, 2887-3073, 2887-3075, |
| | 2890-2989, 2892-3058, 2894-3075, 2895-3042, 2908-3060, 2949-3075, 2998-3057, 2998-3060, 2998-3063, 2998- |
| | 3064, 2999-3075 |
| 108/2321054CB1/ | 1-96, 1-109, 1-134, 1-273, 100-364, 100-637, 100-656, 100-691, 100-700, 100-831, 100-834, 100-848, 101-453, 132- |
| 849 | 842, 174-823, 200-731, 200-784, 213-780, 246-833, 273-573, 278-849, 287-796, 297-831, 323-537, 323-553, 334- |
| | 838, 355-642, 360-587, 361-696, 382-652, 554-776 |
| 109/2796034CB1/ | 1-790, 49-773, 85-773, 118-773, 300-773, 326-773, 498-773, 544-799, 563-804, 608-810, 709-1077, 801-1077, 947- |
| 2659 | 1142, 958-1273, 958-1538, 991-1142, 1008-1219, 1262-2658, 1263-1551, 1408-1997, 1479-1860, 1715-2350, 2017- |
| | 2618, 2156-2306, 2187-2659 |
| 110/4413112CB1/ | 1-268, 1-289, 1-622, 10-245, 10-254, 10-358, 10-433, 10-438, 15-263, 367-623, 367-779, 367-816, 367-884, 367- |
| 2205 | 889, 367-897, 367-942, 367-944, 367-947, 367-955, 367-1001, 367-1010, 367-1026, 367-1064, 367-1089, 370-982, |
| | 372-464, 382-1075, 458-737, 458-1128, 653-1316, 713-981, 769-1212, 802-1467, 823-1343, 901-1468, 949-1164, |
| | 949-1167, 950-1323, 964-1578, 1031-1397, 1034-1650, 1040-1500, 1094-1394, 1129-1804, 1310-1503, 1362-1621, |
| | 1416-1512, 1436-1691, 1462-1667, 1462-1717, 1462-1964, 1467-1778, 1478-1619, 1504-1800, 1545-1912, 1611- |
| | 1837, 1611-2115, 1657-1911, 1703-2205, 1762-2018 |
| 111/7654832CB1/ | 1-391, 1-567, 29-488, 29-678, 97-699, 104-846, 115-227, 153-627, 153-680, 153-684, 153-726, 153-769, 153-776, |
| 3042 | 648-942, 649-814, 649-1187, 713-1409, 733-1267, 951-1546, 1107-1800, 1207-1869, 1207-1901, 1207-1938, 1207- |
| | 1945, 1207-1947, 1207-1981, 1207-1982, 1207-1987, 1207-1994, 1207-1996, 1207-1999, 1208-1938, 1208-1943, |
| | 1208-2086, 1274-2012, 1600-1872, 1668-1940, 1668-2141, 1668-2353, 1669-2432, 1819-2113, 1858-2031, 2050- |
| | 2567, 2051-2327, 2120-2973, 2121-2428, 2135-2973, 2139-2975, 2311-2951, 2393-3009, 2412-3042, 2416-3042, |
| | 2423-2949, 2439-3039, 2446-2973, 2460-3009, 2483-2728, 2552-2800, 2559-3042, 2630-2862, 2770-3027 |
| 112/7503849CB1/ | 1-214, 1-216, 1-249, 1-273, 1-293, 2-239, 4-783, 6-301, 6-343, 8-2112, 10-556, 13-311, 14-303, 14-495, 16-264, 16- |
| 2112 | 633, 17-295, 18-248, 18-269, 18-605, 18-707, 19-252, 19-258, 19-287, 19-536, 19-712, 20-272, 20-374, 21-268, 21- |
| | 292, 21-328, 21-337, 21-343, 21-444, 22-325, 22-416, 22-703, 23-298, 23-540, 23-641, 24-219, 24-456, 25-687, 25- |
| | 709, 27-335, 28-304, 29-597, 35-626, 37-665, 37-674, 38-538, 38-704, 39-570, 39-625, 40-631, 40-645, 41-685, 41- |
| | 784, 42-657, 42-757, 43-301, 45-592, 48-630, 48-708, 51-344, 51-418, 51-421, 53-337, 57-489, 58-386, 59-340, 60- |
| | 311, 61-418, 62-307, 67-387, 89-333, 115-735, 139-404, 141-338, 141-410, 145-383, 164-440, 165-386, 167-414, |
| | 180-464, 186-546, 186-607, 211-466, 218-413, 218-721, 219-539, 220-688, 221-310, 221-446, 230-520, 230-556, |
| | 232-485, 235-531, 236-770, 239-874, 278-497, 288-765, 297-748, 314-737, 329-508, 334-580, 384-510, 531-1073, |
| | 563-833, 656-945, 674-907, 698-1310, 882-1475, 948-1177, 950-1399, 969-1314, 980-1554, 1012-1252, 1016-1515, |
| | 1028-1678, 1030-1298, 1035-1615, 1045-1301, 1047-1501, 1048-1284, 1048-1344, 1056-1684, 1058-1328, 1058- |
| | 1330, 1058-1336, 1060-1298, 1062-1799, 1066-1582, 1080-1643, 1083-1800, 1091-1668, 1091-1712, |
| | 1092-1335, 1098-1657, 1101-1826, 1109-1560, 1109-1624, 1126-1590, 1134-1753, 1137-1590, 1148-1587, 1152- |
| | 1586, 1153-1715, 1159-1762, 1168-1314, 1170-1437, 1170-1735, 1172-1789, 1172-1871, 1177-1431, 1177-1474, |
| | 1177-1791, 1179-1651, 1190-1636, 1192-1713, 1204-1466, 1205-1628, 1206-1470, 1206-1586, 1219-1584, 1221- |
| | 1489, 1225-1628, 1226-1468, 1232-1590, 1234-1467, 1234-1632, 1240-1590, 1240-1800, 1241-1497, 1241-1537, |
| | 1241-1587, 1242-1712, 1244-1485, 1246-1427, 1246-1491, 1248-1836, 1249-1708, 1249-1873, 1253-1712, 1255- |
| | 1865, 1260-1546, 1266-1712, 1268-1592, 1268-1873, 1270-1497, 1270-1712, 1275-1717, 1275-1718, 1277-1647, |
| | 1284-1541, 1295-1826, 1298-1873, 1299-1873, 1300-1715, 1300-1871, 1300-1873, 1302-1873, 1303-1710, 1303- |
| | 1785, 1307-1871, 1308-1870, 1316-1536, 1316-1868, 1319-1718, 1321-1858, 1324-1590, 1324-1864, 1325-1866, |
| | 1343-1872, 1344-1463, 1345-1583, 1360-1586, 1360-1715, 1379-1676, 1382-1873, 1402-1873, 1415-1873, 1424- |
| | 1494, 1424-1873, 1428-1590, 1428-1729, 1428-1873, 1429-1873, 1441-1712, 1441-1872, 1453-1673, 1456-1873, |
| | 1458-1873, 1461-2112, 1462-1873, 1463-1872, 1464-1850, 1464-1873, 1476-1871, 1476-1872, 1479-1736, 1483- |
| | 1834, 1490-1570, 1490-1873, 1493-1811, 1496-1872, 1502-1861, 1520-1796, 1521-1820, 1528-1658, 1530-1873, |
| | 1533-1873, 1534-1822, 1534-1830, 1534-1872, 1548-1872, 1554-1873, 1556-1813, 1557-1872, 1563-1704, 1564- |
| | 1872, 1571-1872, 1573-1873, 1599-1868, 1601-1844, 1601-1845, 1624-1873, 1701-1873, 1748-1859, 1814-1864 |

TABLE 5

| Polynucleotide | Incyte | Representative |
|---|---|---|
| SEQ ID NO: | Project ID: | Library |
| 57 | 2867236CB1 | KIDNNOT20 |
| 58 | 1294096CB1 | PROSTUS23 |
| 59 | 7238537CB1 | BRAITUT03 |
| 60 | 7494391CB1 | SINTNOT18 |
| 61 | 6451054CB1 | CORPNOT02 |
| 62 | 7494592CB1 | UTRSTMR01 |
| 63 | 5202657CB1 | HEARFET01 |
| 64 | 2013529CB1 | TESTNOT03 |
| 65 | 3841351CB1 | LIVRNOT21 |
| 66 | 152116CB1 | BRAITDR02 |
| 67 | 2381031CB1 | PROSBPS05 |
| 68 | 2511371CB1 | CONUTUT01 |
| 69 | 8068623CB1 | TONSDIC01 |
| 70 | 677977CB1 | BRABDIE02 |
| 71 | 1661472CB1 | MLP000032 |
| 72 | 1748508CB1 | PROSNON01 |
| 73 | 2159545CB1 | PLACFEB01 |
| 74 | 8560269CB1 | NEUTFMT01 |
| 75 | 8710302CB1 | THYMNOE02 |
| 76 | 6778214CB1 | THYRTUT03 |
| 77 | 258383CB1 | BRSTNOT17 |
| 78 | 2804937CB1 | STOMTDE01 |
| 80 | 2073751CB1 | TLYMUNT01 |
| 81 | 3178841CB1 | UTRSTUE01 |
| 82 | 3674807CB1 | PLACNOT07 |
| 83 | 1794922CB1 | PROSTUT05 |
| 84 | 1795509CB1 | PROSTUT05 |
| 85 | 2017180CB1 | BLADTUT08 |
| 86 | 219442CB1 | THYMNOT05 |
| 87 | 2597459CB1 | BRACNOK02 |
| 88 | 2783863CB1 | BRAHTDR03 |
| 89 | 2902971CB1 | BRAYDIN03 |
| 90 | 368660CB1 | BRSTNOT01 |
| 91 | 2804990CB1 | BRAITUT02 |
| 92 | 168571CB1 | BRAUNOR01 |
| 93 | 1286391CB1 | MENITUT03 |
| 94 | 2007684CB1 | LUNGNON03 |
| 95 | 2227040CB1 | BRSTNOT03 |
| 96 | 4346130CB1 | BRAUNOR01 |
| 98 | 7472392CB1 | BRALNON02 |
| 99 | 4028960CB1 | KIDNFEC01 |
| 100 | 8227004CB1 | PROSNOT19 |
| 101 | 3044763CB1 | HEAANOT01 |
| 102 | 4044519CB1 | LUNGNOT35 |
| 103 | 71351918CB1 | BRSTTUT01 |
| 104 | 8109363CB1 | BRAINOT19 |
| 105 | 1272746CB1 | TESTTUT02 |
| 106 | 1839974CB1 | OVARDIR01 |
| 107 | 1877336CB1 | ADRETUR01 |
| 108 | 2321054CB1 | BRSTNOT13 |
| 109 | 2796034CB1 | LUNGDIN02 |
| 110 | 4413112CB1 | MONOTXT01 |
| 111 | 7654832CB1 | BLADTUT06 |
| 112 | 7503849CB1 | KIDNNOT09 |

TABLE 6

| Library | Vector | Library Description |
|---|---|---|
| ADRETUR01 | PCDNA2.1 | This random primed library was constructed using RNA isolated from left upper pole, adrenal |
| | | gland tumor tissue removed from a 52-year-old Caucasian male during nephroureterectomy and |
| | | local destruction of renal lesion. Pathology indicated grade 3 adrenal cortical carcinoma |
| | | forming a mass that infiltrated almost the whole adrenal parenchyma and extended to adjacent |
| | | adipose tissue. A metastatic tumor nodule was identified in the hilar region. The renal |
| | | vein was infiltrated by tumor and the neoplastic process was present at the resection |
| | | margin of the renal vein. Fragments of adrenal cortical carcinoma and thrombus were found |
| | | in the inferior vena cava. Patient history included abnormal weight loss. Family history |
| | | included skin cancer, type I diabetes, and neurotic depression. |
| BLADTUT06 | pINCY | Library was constructed using RNA isolated from bladder tumor tissue removed from the |
| | | posterior bladder wall of a 58-year-old Caucasian male during a radical cystectomy, radical |
| | | prostatectomy, and gastrostomy. Pathology indicated grade 3 transitional cell carcinoma in |
| | | the left lateral bladder wall. The remaining bladder showed marked cystitis with scattered |
| | | microscopic foci of transitional cell carcinoma in situ. Patient history included angina, |
| | | emphysema and tobacco use. Family history included acute myocardial infarction, |
| | | atherosclerotic coronary artery disease, and type II diabetes. |
| BLADTUT08 | pINCY | Library was constructed using RNA isolated from bladder tumor tissue removed from a 72- |
| | | year-old Caucasian male during a radical cystectomy and prostatectomy. Pathology indicated |
| | | an invasive grade 3 (of 3) transitional cell carcinoma in the right bladder base. Patient |
| | | history included pure hypercholesterolemia and tobacco abuse. Family history included |
| | | myocardial infarction, cerebrovascular disease, and brain cancer. |
| BRABDIE02 | pINCY | This 5′ biased random primed library was constructed using RNA isolated from diseased |
| | | cerebellum tissue removed from the brain of a 57-year-old Caucasian male who died from a |
| | | cerebrovascular accident. Serologies were negative. Patient history included Huntington's |
| | | disease, emphysema, and tobacco abuse (3-4 packs per day, for 40 years). |
| BRACNOK02 | PSPORT1 | This amplified and normalized library was constructed using RNA isolated from posterior |
| | | cingulate tissue removed from an 85-year-old Caucasian female who died from myocardial |
| | | infarction and retroperitoneal hemorrhage. Pathology indicated atherosclerosis, moderate to |
| | | severe, involving the circle of Willis, middle cerebral, basilar and vertebral arteries; |
| | | infarction, remote, left dentate nucleus; and amyloid plaque deposition consistent with age. |
| | | There was mild to moderate leptomeningeal fibrosis, especially over the convexity of the |
| | | frontal lobe. There was mild generalized atrophy involving all lobes. The white matter was |
| | | mildly thinned. Cortical thickness in the temporal lobes, both maximal and minimal, was |
| | | slightly reduced. The substantia nigra pars compacta appeared mildly depigmented. Patient |
| | | history included COPD, hypertension, and recurrent deep venous thrombosis. 6.4 million |
| | | independent clones from this amplified library were normalized in one round using |
| | | conditions adapted from Soares et al., PNAS (1994) 91: 9228-9232 and Bonaldo et al., |
| | | Genome Research 6 (1996): 791. |
| BRAHTDR03 | PCDNA2.1 | This random primed library was constructed using RNA isolated from archaecortex, anterior |
| | | hippocampus tissue removed from a 55-year-old Caucasian female who died from cholangio- |
| | | carcinoma. Pathology indicated mild meningeal fibrosis predominately over the convexities, |
| | | scattered axonal spheroids in the white matter of the cingulate cortex and the thalamus, |
| | | and a few scattered neurofibrillary tangles in the entorhinal cortex and the periaqueductal |
| | | gray region. Pathology for the associated tumor tissue indicated well-differentiated |
| | | cholangiocarcinoma of the liver with residual or relapsed tumor. Patient history included |
| | | cholangiocarcinoma, post-operative Budd-Chiari syndrome, biliary ascites, hydrothorax, |
| | | dehydration, malnutrition, oliguria and acute renal failure. Previous surgeries included |
| | | cholecystectomy and resection of 85% of the liver. |
| BRAINOT19 | pINCY | Library was constructed using RNA isolated from diseased brain tissue removed from the |
| | | left frontal lobe of a 27-year-old Caucasian male during a brain lobectomy. Pathology |
| | | indicated a focal deep white matter lesion, characterized by marked gliosis, calcifi- |
| | | cations, and hemosiderin-laden macrophages, consistent with a remote perinatal injury. |
| | | This tissue also showed mild to moderate generalized gliosis, predominantly subpial and |
| | | subcortical, consistent with chronic seizure disorder. The left temporal lobe, including |
| | | the mesial temporal structures, showed focal, marked pyramidal cell loss and gliosis in |
| | | hippocampal sector CA1, consistent with mesial temporal sclerosis. GFAP was positive for |
| | | astrocytes. The patient presented with intractable epilepsy, focal epilepsy, hemiplegia, |
| | | and an unspecified brain injury. Patient history included cerebral palsy, abnormality of |
| | | gait, and depressive disorder. Family history included brain cancer. |
| BRAITDR02 | PCDNA2.1 | This random primed library was constructed using RNA isolated from allocortex, neocortex, |
| | | anterior and frontal cingulate tissue removed from a 55-year-old Caucasian female who died |
| | | from cholangiocarcinoma. Pathology indicated mild meningeal fibrosis predominately over the |
| | | convexities, scattered axonal spheroids in the white matter of the cingulate cortex and the |
| | | thalamus, and a few scattered neurofibrillary tangles in the entorhinal cortex and the periaqueductal gray |
| | | region. Pathology for the associated tumor tissue indicated well-differentiated |
| | | cholangiocarcinoma of the liver with residual or relapsed tumor. Patient history included |
| | | cholangiocarcinoma, post-operative Budd-Chiari syndrome, biliary ascites, hydrothorax, |
| | | dehydration, malnutrition, oliguria and acute renal failure. Previous surgeries included |
| | | cholecystectomy and resection of 85% of the liver. |
| BRAITUT02 | PSPORT1 | Library was constructed using RNA isolated from brain tumor tissue removed from the frontal |
| | | lobe of a 58-year-old Caucasian male during excision of a cerebral meningeal lesion. |
| | | Pathology indicated a grade 2 metastatic hypernephroma. Patient history included a grade 2 |
| | | renal cell carcinoma, insomnia, and chronic airway obstruction. Family history included a |
| | | malignant neoplasm of the kidney. |
| BRAITUT03 | PSPORT1 | Library was constructed using RNA isolated from brain tumor tissue removed from the left |
| | | frontal lobe of a 17-year-old Caucasian female during excision of a cerebral meningeal |
| | | lesion. Pathology indicated a grade 4 fibrillary giant and small-cell astrocytoma. Family |
| | | history included benign hypertension and cerebrovascular disease. |
| BRALNON02 | pINCY | This thalamus tissue library was constructed from 4.24 million independent clones from a |
| | | thalamus tissue library. Starting RNA was made from thalamus tissue removed from a 35-year- |
| | | old Caucasian male who died from cardiac failure. Pathology indicated moderate |
| | | leptomeningeal fibrosis and multiple microinfarctions of the cerebral neocortex. |
| | | Microscopically, the cerebral hemisphere revealed moderate fibrosis of the leptomeninges |
| | | with focal calcifications. There was evidence of shrunken and slightly eosinophilic |
| | | pyramidal neurons throughout the cerebral hemispheres. Scattered throughout the cerebral |
| | | cortex, there were multiple small microscopic areas of cavitation with surrounding gliosis. |
| | | Patient history included dilated cardiomyopathy, congestive heart failure, cardiomegaly |
| | | and an enlarged spleen and liver. The library was normalized in two rounds using |
| | | conditions adapted from Soares et al., PNAS (1994) 91: 9228-9232 and Bonaldo et al., |
| | | Genome Research (1996) 6: 791, except that a significantly longer (48 hours/round) |
| | | reannealing hybridization was used. |
| BRAUNOR01 | pINCY | This random primed library was constructed using RNA isolated from striatum, globus |
| | | pallidus and posterior putamen tissue removed from an 81-year-old Caucasian female who |
| | | died from a hemorrhage and ruptured thoracic aorta due to atherosclerosis. Pathology |
| | | indicated moderate atherosclerosis involving the internal carotids, bilaterally; |
| | | microscopic infarcts of the frontal cortex and hippocampus; and scattered diffuse amyloid |
| | | plaques and neurofibrillary tangles, consistent with age. Grossly, the leptomeninges |
| | | showed only mild thickening and hyalinization along the superior sagittal sinus. The |
| | | remainder of the leptomeninges was thin and contained some congested blood vessels. Mild |
| | | atrophy was found mostly in the frontal poles and lobes, and temporal lobes, bilaterally. |
| | | Microscopically, there were pairs of Alzheimer type II astrocytes within the deep layers of |
| | | the neocortex. There was increased satellitosis around neurons in the deep gray matter |
| | | in the middle frontal cortex. The amygdala contained rare diffuse plaques and |
| | | neurofibrillary tangles. The posterior hippocampus contained a microscopic area of cystic |
| | | cavitation with hemosiderin-laden macrophages surrounded by |
| | | reactive gliosis. Patient history included sepsis, cholangitis, post-operative atelectasis, |
| | | pneumonia CAD, cardiomegaly due to left ventricular hypertrophy, splenomegaly, |
| | | arteriolonephrosclerosis, nodular colloidal goiter, emphysema, CHF, hypothyroidism, and |
| | | peripheral vascular disease. |
| BRAYDIN03 | pINCY | This normalized library was constructed from 6.7 million independent clones from a brain |
| | | tissue library. Starting RNA was made from RNA isolated from diseased hypothalamus tissue |
| | | removed from a 57-year-old Caucasian male who died from a cerebrovascular accident. |
| | | Patient history included Huntington's disease and emphysema. The library was normalized in |
| | | 2 rounds using conditions adapted from Soares et al., PNAS (1994) 91: 9228 and Bonaldo et |
| | | al., Genome Research (1996) 6: 791, except that a significantly longer (48-hours/round) |
| | | reannealing hybridization was used. The library was linearized and recircularized to |
| | | select for insert containing clones. |
| BRSTNOT01 | PBLUESCRIPT | Library was constructed using RNA isolated from the breast tissue of a 56-year-old |
| | | Caucasian female who died in a motor vehicle accident. |
| BRSTNOT03 | PSPORT1 | Library was constructed using RNA isolated from diseased breast tissue removed from a 54- |
| | | year-old Caucasian female during a bilateral radical mastectomy. Pathology for the |
| | | associated tumor tissue indicated residual invasive grade 3 mammary ductal adenocarcinoma. |
| | | Patient history included kidney infection and condyloma acuminatum. Family history |
| | | included benign hypertension, hyperlipidemia and a malignant neoplasm of the colon. |
| BRSTNOT13 | pINCY | Library was constructed using RNA isolated from breast tissue removed from a 36-year-old |
| | | Caucasian female during bilateral simple mastectomy. Patient history included a breast |
| | | neoplasm, depressive disorder, hyperlipidemia, and a chronic stomach ulcer. Family history |
| | | included a cardiovascular and cerebrovascular disease; hyperlipidemia; skin, breast, |
| | | esophageal, bladder, and bone cancer; and Hodgkin's lymphoma. |
| BRSTNOT17 | pINCY | Library was constructed using RNA isolated from breast tissue removed from a 46-year-old |
| | | Caucasian female during a unilateral extended simple mastectomy. Pathology for the |
| | | associated tumor tissue indicated invasive grade 3, nuclear grade 2 adenocarcinoma, ductal |
| | | type. An intraductal carcinoma component, non-comedo, comprised approximately 50% of the |
| | | neoplasm, including the lactiferous ducts. Angiolymphatic involvement was present, and |
| | | metastatic adenocarcinoma was present in 7 of 10 axillary lymph nodes. The largest nodal |
| | | metastasis measured 3 cm, and focal extracapsular extension was identified. Family history |
| | | included atherosclerotic coronary artery disease, type II diabetes, cerebrovascular |
| | | disease, and depressive disorder. |
| BRSTTUT01 | PSPORT1 | Library was constructed using RNA isolated from breast tumor tissue removed from a 55-year- |
| | | old Caucasian female during a unilateral extended simple mastectomy. Pathology indicated |
| | | invasive grade 4 mammary adenocarcinoma of mixed lobular and ductal type, extensively |
| | | involving the left breast. The tumor was identified in the deep dermis near the |
| | | lactiferous ducts with extracapsular extension. Seven mid and low and five high axillary |
| | | lymph nodes were positive for tumor. Proliferative fibrocysytic changes were characterized |
| | | by apocrine metaplasia, sclerosing adenosis, cyst formation, and ductal hyperplasia |
| | | without atypia. Patient history included atrial tachycardia, blood in the stool, and a |
| | | benign breast neoplasm. Family history included benign hypertension, atherosclerotic |
| | | coronary artery disease, cerebrovascular disease, and depressive disorder. |
| CONUTUT01 | pINCY | Library was constructed using RNA isolated from sigmoid mesentery tumor tissue obtained |
| | | from a 61-year-old female during a total abdominal hysterectomy and bilateral salpingo- |
| | | oophorectomy with regional lymph node excision. Pathology indicated a metastatic grade 4 |
| | | malignant mixed mullerian tumor present in the sigmoid mesentery at two sites. |
| CORPNOT02 | pINCY | Library was constructed using RNA isolated from diseased corpus callosum tissue removed |
| | | from the brain of a 74-year-old Caucasian male who died from Alzheimer's disease. |
| HEAANOT01 | pINCY | Library was constructed using RNA isolated from right coronary and right circumflex |
| | | coronary artery tissue removed from the explanted heart of a 46-year-old Caucasian male |
| | | during a heart transplantation. Patient history included myocardial infarction from total |
| | | occlusion of the left anterior descending coronary artery, atherosclerotic coronary artery |
| | | disease, hyperlipidemia, myocardial ischemia, dilated cardiomyopathy, left ventricular |
| | | dysfunction, and tobacco abuse. Previous surgeries included cardiac catheterization. |
| | | Family history included atherosclerotic coronary artery disease. |
| HEARFET01 | pINCY | Library was constructed using RNA isolated from heart tissue removed from a Hispanic male |
| | | fetus, who died at 18 weeks' gestation. |
| KIDNFEC01 | PBLUESCRIPT | Library was constructed using RNA isolated from kidney tissue removed from a pool of |
| | | twelve Caucasian male and female fetuses that were spontaneously aborted at 19-23 |
| | | weeks' gestation. |
| KIDNNOT09 | pINCY | Library was constructed using RNA isolated from the kidney tissue of a Caucasian male |
| | | fetus, who died at 23 weeks' gestation. |
| KIDNNOT20 | pINCY | Library was constructed using RNA isolated from left kidney tissue removed from a 43-year- |
| | | old Caucasian male during nephroureterectomy, regional lymph node excision, and unilateral |
| | | left adrenalectomy. Pathology for the associated tumor tissue indicated a grade 2 renal |
| | | cell carcinoma. Family history included atherosclerotic coronary artery disease. |
| LIVRNOT21 | pINCY | Library was constructed using RNA isolated from liver tissue removed from a 29-year-old |
| | | Caucasian male who died from massive head injury due to a motor vehicle accident. Serology |
| | | was positive for cytomegalovirus. |
| LUNGDIN02 | pINCY | This normalized lung tissue library was constructed from 7.6 million independent clones |
| | | from a diseased lung tissue library. Starting RNA was made from RNA isolated from diseased |
| | | lung tissue. Pathology indicated ideopathic pulmonary disease. The library was normalized |
| | | in 2 rounds using conditions adapted from Soares et al., PNAS (1994) 91: 9228-9232 |
| | | and Bonaldo et al., Genome Research 6 (1996): 791, except that a significantly longer |
| | | (48 hours/round) reannealing hybridization was used. |
| LUNGNON03 | PSPORT1 | This normalized library was constructed from 2.56 million independent clones from a lung |
| | | tissue library. RNA was made from lung tissue removed from the left lobe of a 58-year-old |
| | | Caucasian male during a segmental lung resection. Pathology for the associated tumor |
| | | tissue indicated a metastatic grade 3 (of 4) osteosarcoma. Patient history included soft |
| | | tissue cancer, secondary cancer of the lung, prostate cancer, and an acute duodenal ulcer |
| | | with hemorrhage. Patient also received radiation therapy to the retroperitoneum. Family |
| | | history included prostate cancer, breast cancer, and acute leukemia. The normalization and |
| | | hybridization conditions were adapted from Scares et al., PNAS (1994) 91: 9228; Swaroop et |
| | | al., NAR (1991) 19: 1954; and Bonaldo et al., Genome Research (1996) 6: 791. |
| LUNGNOT35 | pINCY | Library was constructed using RNA isolated from lung tissue removed from a 62-year-old |
| | | Caucasian female. Pathology for the associated tumor tissue indicated a grade 1 spindle |
| | | cell carcinoid forming a nodule. Patient history included depression, thrombophlebitis, |
| | | and hyperlipidemia. Family history included cerebrovascular disease, atherosclerotic |
| | | coronary artery disease, breast cancer, colon cancer, type II diabetes, and malignant skin |
| | | melanoma. |
| MENITUT03 | pINCY | Library was constructed using RNA isolated from brain meningioma tissue removed from a 35- |
| | | year-old Caucasian female during excision of a cerebral meningeal lesion. Pathology |
| | | indicated a benign neoplasm in the right cerebellopontine angle of the brain. Patient |
| | | history included hypothyroidism. Family history included myocardial infarction and breast |
| | | cancer. |
| MLP000032 | PCR2-TOPOTA | Library was constructed using pooled cDNA from different donors. cDNA was generated using |
| | | mRNA isolated from the following: aorta, cerebellum, lymph nodes, muscle, tonsil (lymphoid |
| | | hyperplasia), bladder tumor (invasive grade 3 transitional cell carcinoma.), breast |
| | | (proliferative fibrocystic changes without atypia characterized by epithelial ductal |
| | | hyperplasia, testicle tumor (embryonal carcinoma), spleen, ovary, parathyroid, ileum, |
| | | breast skin, sigmoid colon, penis tumor (fungating invasive grade 4 squamous cell |
| | | carcinoma), fetal lung,, breast, fetal small intestine, fetal liver, fetal pancreas, fetal |
| | | lung, fetal skin, fetal penis, fetal bone, fetal ribs, frontal brain tumor (grade 4 |
| | | gemistocytic astrocytoma), ovary (stromal hyperthecosis), bladder, bladder tumor (invasive |
| | | grade 3 transitional cell carcinoma), stomach, lymph node tumor (metastatic basaloid |
| | | squamous cell carcinoma), tonsil (reactive lymphoid hyperplasia), periosteum from the |
| | | tibia, fetal brain, fetal spleen, uterus tumor, endometrial (grade 3 adenosquamous |
| | | carcinoma), seminal vesicle, liver, aorta, adrenal gland, lymph node (metastatic grade 3 |
| | | squamous cell carcinoma), glossal muscle, esophagus, |
| | | esophagus tumor (invasive grade 3 adenocarcinoma), ileum, pancreas, soft tissue tumor from |
| | | the skull (grade 3 ependymoma), transverse colon, (benign familial polyposis), rectum |
| | | tumor (grade 3 colonic adenocarcinoma), rib tumor, (metastatic grade 3 osteosarcoma), |
| | | lung, heart, placenta, thymus, stomach, spleen (splenomegaly with congestion), uterus, |
| | | cervix (mild chronic cervicitis with focal squamous metaplasia), spleen tumor (malignant |
| | | lymphoma, diffuse large cell type, B-cell phenotype with abundant reactive T-cells and |
| | | marked granulomatous response), umbilical cord blood mononuclear cells, upper lobe lung |
| | | tumor, (grade 3 squamous cell carcinoma), endometrium (secretory phase), liver, liver |
| | | tumor (metastatic grade 2 neuroendocrine carcinoma), colon, umbilical cord blood, Th1 |
| | | cells, nonactivated, umbilical cord blood, Th2 cells, nonactivated, coronary artery |
| | | endothelial cells (untreated), coronary artery smooth muscle cells, (untreated), |
| | | coronary artery smooth muscle cells (treated with TNF & IL-1 10 ng/ml each for 20 hours), |
| | | bladder (mild chronic cystitis), epiglottis, breast skin, small intestine, fetal prostate |
| | | stroma fibroblasts, prostate epithelial cells (PrEC cells), |
| | | fetal adrenal glands, fetal liver, kidney transformed embryonal cell line (293-EBNA) |
| | | (untreated), kidney transformed embryonal cell line (293-EBNA) (treated with |
| | | 5Aza-2deoxycytidine for 72 hours), mammary epithelial cells, (HMEC cells), peripheral |
| | | blood monocytes (treated with IL-10 at time 0, 10 ng/ml, LPS was added at 1 hour at 5 |
| | | ng/ml. Incubation 24 hours), peripheral blood monocytes (treated with anti-IL-10 at time |
| | | 0, 10 ng/ml, LPS was added at 1 hour at 5 ng/ml. Incubation 24 hours), spinal cord, base |
| | | of medulla (Huntington's chorea), thigh and arm muscle (ALS), breast skin fibroblast |
| | | (untreated), breast skin fibroblast (treated with 9CIS Retinoic Acid 1 μM for 20 hours), |
| | | breast skin fibroblast (treated with TNF-alpha & IL-1 beta, 10 ng/ml each for 20 hours), |
| | | fetal liver mast cells, hematopoietic (Mast cells prepared from human fetal liver |
| | | hematopoietic progenitor cells (CD34+ stem cells) cultured in the presence of hIL-6 and |
| | | hSCF for 18 days), epithelial layer of colon, bronchial epithelial cells (treated for 20 |
| | | hours with 20% smoke conditioned media), lymph node, pooled peripheral blood mononuclear |
| | | cells (untreated), pooled brain segments: striatum, globus |
| | | pallidus and posterior putamen (Alzheimer's Disease), pituitary gland, umbilical cord |
| | | blood, CD34+ derived dendritic cells (treated with SCF, GM-CSF & TNF alpha, 13 days), |
| | | umbilical cord blood, CD34+ derived dendritic cells (treated with SCF, GM-CSF & TNF |
| | | alpha, 13 days followed by PMA/Ionomycin for 5 hours), small intestine, rectum, bone |
| | | marrow neuroblastoma cell line (SH-SY5Y cells, treated with 6-Hydroxydopamine 100 uM for 8 |
| | | hours), bone marrow, neuroblastoma cell line (SH-SY5Y cells, untreated), brain segments |
| | | from one donor: amygdala, entorhinal cortex, globus pallidus, substantia innominata, |
| | | striatum, dorsal caudate nucleus, dorsal putamen, ventral nucleus accumbens, archaecortex |
| | | (hippocampus anterior and posterior), thalamus, nucleus raphe magnus, periaqueductal gray, |
| | | midbrain, substantia nigra, and dentate nucleus, pineal gland (Alzheimer's Disease), |
| | | preadipocytes (untreated), preadipocytes (treated with a peroxisome proliferator-activated |
| | | receptor gamma agonist, 1 microM, 4 hours), pooled prostate (adenofibromatous |
| | | hyperplasia), pooled kidney, pooled adipocytes (untreated), pooled adipocytes (treated |
| | | with human insulin), |
| | | pooled mesentaric and abdomenal fat, pooled adrenal glands, pooled thyroid (normal and |
| | | adenomatous hyperplasia), pooled spleen (normal and with changes consistent with |
| | | idiopathic thrombocytopenic purpura), pooled right and left breast, pooled lung, pooled |
| | | nasal polyps, pooled fat, pooled synovium (normal and rhumatoid arthritis), pooled brain |
| | | (meningioma, gemistocytic astrocytoma. and Alzheimer's disease), pooled fetal colon, |
| | | pooled colon: ascending, descending (chronic ulcerative colitis), and rectal tumor |
| | | (adenocarcinoma), pooled esophagus, normal and tumor (invasive grade 3 adenocarcinoma), |
| | | pooled breast skin fibroblast (one treated w/9CIS Retinoic Acid and the other with |
| | | TNF-alpha & IL-1 beta), pooled gallbladder (acute necrotizing cholecystitis with |
| | | cholelithiasis (clinically hydrops), acute hemorrhagic cholecystitis with cholelithiasis, |
| | | chronic cholecystitis and cholelithiasis), pooled fetal heart, (Patau's and fetal demise), |
| | | pooled neurogenic tumor cell line, SK-N-MC, (neuroepitelioma, metastasis to supra-orbital |
| | | area, untreated) and neuron, NT-2 cell line, (treated with mouse leptin at 1 μg/ml and |
| | | 9cis retinoic acid at 3.3 μM |
| | | for 6 days), pooled ovary (normal and polycystic ovarian disease), pooled prostate, |
| | | (adenofibromatous hyperplasia), pooled seminal vesicle, pooled small intestine, pooled |
| | | fetal small intestine, pooled stomach and fetal stomach, prostate epithelial cells, pooled |
| | | testis (normal and embryonal carcinoma), pooled uterus, pooled uterus tumor (grade 3 |
| | | adenosquamous carcinoma and leiomyoma), pooled uterus, endometrium, and myometrium, |
| | | (normal and adenomatous hyperplasia with squamous metaplasia and focal atypia), pooled |
| | | brain: (temporal lobe meningioma, cerebellum and hippocampus (Alzheimer's Disease), pooled |
| | | skin, fetal lung, adrenal tumor (adrenal cortical carcinoma), prostate tumor |
| | | (adenocarcinoma), fetal heart, fetal small intestine, ovary tumor (mucinous cystadenoma), |
| | | ovary, ovary tumor (transitional cell carcinoma), disease prostate (adenofibromatous |
| | | hyperplasia), fetal colon, uterus tumor (leiomyoma), temporal brain, submandibular gland, |
| | | colon tumor (adenocarcinoma), ascending and transverse colon, ovary tumor (endometrioid |
| | | carcinoma), lung tumor (squamous cell carcinoma), fetal brain, fetal lung, ureter tumor |
| | | (transitional cell carcinoma), |
| | | untreated HNT cells, para-aortic soft tissue, testis, seminal vesicle, diseased ovary |
| | | (endometriosis), temporal lobe, myometrium, diseased gallbladder (cholecystitis, |
| | | cholelithiasis), placenta, breast tumor (ductal adenocarcinoma), breast, lung tumor |
| | | (liposarcoma), endometrium, abdominal fat, cervical spine dorsal root ganglion, thoracic |
| | | spine dorsal root ganglion, diseased thyroid (adenomatous hyperplasia), liver, kidney, |
| | | fetal liver, NT-2 cells (treated with mouse leptin and 9cis RA), K562 cells (treated with |
| | | 9cis RA), cerebellum, corpus callosum, hypothalamus, fetal brain astrocytes (treated with |
| | | TNFa and IL-1b), inferior parietal cortex, posterior hippocampus, pons, thalamus, C3A |
| | | cells (untreated), C3A cells (treated with 3-methylcholanthrene), testis, colon epithelial |
| | | layer, pooled prostate, pooled liver, substantia nigra, thigh muscle, rib bone, fallopian |
| | | tube tumor (endometrioid and serous adenocarcinoma), diseased lung (idiopathic pulmonary |
| | | disease), cingulate anterior allocortex and neocortex, cingulate posterior allocortex, |
| | | auditory neocortex, frontal neocortex, orbital inferior neocortex, parietal superior |
| | | neocortex, visual primary neocortex, dentate nucleus, posterior cingulate, |
| | | cerebellum, vermis, inferior temporal cortex, medulla, posterior parietal cortex, colon |
| | | polyp, pooled breast, anterior and posterior hippocampus, mesenteric and abdominal fat, |
| | | pooled esophagus, pooled fetal kidney, pooled fetal liver, ileum, small intestine, pooled |
| | | gallbladder, frontal and superior temporal cortex, pooled ovary, pooled endometrium, |
| | | pooled prostate, pooled kidney, fetal femur, sacrum tumor (giant cell tumor), pooled |
| | | kidney and kidney tumor (renal cell carcinoma clear-cell type), pooled liver and liver |
| | | tumor (neuroendocrine carcinoma), pooled fetal liver, pooled lung, fetal pancreas, |
| | | pancreas, parotid gland, parotid tumor (sebaceous lymphadenoma), retroperitoneal and |
| | | suprglottic soft tissue, spleen, fetal spleen, spleen tumor (malignant lymphoma), diseased |
| | | spleen (idiopathic thrombocytopenic purpura), parathyroid, thyroid, thymus, tonsil ureter |
| | | tumor (transitional cell carcinoma), pooled adrenal gland and adrenal tumor |
| | | (pheochromocytoma), pooled lymph node tumor (Hodgkin's disease and metastatic |
| | | adenocarcinoma), pooled neck and calf muscles, and pooled bladder |
| MONOTXT01 | pINCY | Library was constructed using RNA isolated from treated monocytes from peripheral blood |
| | | obtained from a 42-year-old female. The cells were treated with anti IL-10 and LPS. |
| NEUTFMT01 | PBLUESCRIPT | Library was constructed using total RNA isolated from peripheral blood granulocytes |
| | | collected by density gradient centrifugation through Ficoll-Hypaque. The cells were |
| | | isolated from buffy coat units obtained from unrelated male and female donors. Cells were |
| | | cultured in 10 nM fMLP for 30 minutes, lysed in GuSCN, and spun through CsCl to obtain RNA |
| | | for library construction. Because this library was made from total RNA, it has an unusually |
| | | high proportion of unique singleton sequences, which may not all come from polyA RNA |
| | | species. |
| OVARDIR01 | PCDNA2.1 | This random primed library was constructed using RNA isolated from right ovary tissue |
| | | removed from a 45-year-old Caucasian female during total abdominal hysterectomy, bilateral |
| | | salpingo-oophorectomy, vaginal suspension and fixation, and incidental appendectomy. |
| | | Pathology indicated stromal hyperthecosis of the right and left ovaries. Pathology for the |
| | | matched tumor tissue indicated a dermoid cyst (benign cystic teratoma) in the left ovary. |
| | | Multiple (3) intramural leiomyomata were identified. The cervix showed squamous metaplasia. |
| | | Patient history included metrorrhagia, female stress incontinence, alopecia, depressive |
| | | disorder, pneumonia, normal delivery, and deficiency anemia. Family history included |
| | | benign hypertension, atherosclerotic coronary artery disease, hyperlipidemia, and primary |
| | | tuberculous complex. |
| PLACFEB01 | pINCY | Library was constructed using pooled cDNA from two different donors. cDNA was generated |
| | | using RNA isolated from placenta tissue removed from a Caucasian fetus (donor A), who died |
| | | after 16 weeks' gestation from fetal demise and hydrocephalus; and a Caucasian male fetus |
| | | (donor B), who died after 18 weeks' gestation from fetal demise. Patient history included |
| | | umbilical cord wrapped around the head (3 times) and the shoulders (1 time) in donor A. |
| | | Serology was positive for anti-CMV in donor A. Family history included multiple pregnancies |
| | | and live births, and an abortion in donor A. |
| PLACNOT07 | pINCY | Library was constructed using RNA isolated from placental tissue removed from a Caucasian |
| | | fetus, who died after 16 weeks' gestation from fetal demise and hydrocephalus. Serology was |
| | | positive for anti-CMV (cytomegalovirus). |
| PROSBPS05 | pINCY | This subtracted prostate tissue library was constructed using 4.48 × 10e5 clones from |
| | | diseased prostate tissue and was subjected to two rounds of subtraction hybridization with |
| | | 1.56 million clones from a breast tissue library. The starting library for subtraction was |
| | | constructed using RNA isolated from diseased prostate tissue removed from a 70-year-old |
| | | Caucasian male during a radical prostatectomy and closed prostatic biopsy. Pathology |
| | | indicated benign prostatic hypertrophy. Pathology for the matched tumor tissue indicated |
| | | adenocarcinoma. The patient presented with elevated prostate specific antigen and |
| | | induration. Patient history included benign hypertension, gastrointestinal bleed, cardiac |
| | | dysrhythmia, cardiac arrest, hyperlipidemia, alcohol abuse and fractured mandible. Previous |
| | | surgeries included splenectomy, cholecystectomy and inguinal hernia repair. Patient |
| | | medications included Verapamil and antacids. Family history included benign hypertension, |
| | | myocardial infarction and coronary atherosclerosis in the mother; tobacco abuse and lung |
| | | cancer in the father; tobacco abuse, cerebrovascular accident and lung cancer in the |
| | | sibling(s). The |
| | | hybridization probe for subtraction was derived from a similarly constructed library from |
| | | RNA isolated from nontumorous breast tissue from a different donor. Subtractive |
| | | hybridization conditions were based on the methodologies of Swaroop et al., NAR 19 |
| | | (1991): 1954 and Bonaldo, et al. Genome Research 6 (1996): 791. |
| PROSNON01 | PSPORT1 | This normalized prostate library was constructed from 4.4 M independent clones from a |
| | | prostate library. Starting RNA was made from prostate tissue removed from a 28-year-old |
| | | Caucasian male who died from a self-inflicted gunshot wound. The normalization and |
| | | hybridization conditions were adapted from Soares, M. B. et al. (1994) Proc. Natl. Acad. |
| | | Sci. USA 91: 9228-9232, using a longer (19 hour) reannealing hybridization period. |
| PROSNOT19 | pINCY | Library was constructed using RNA isolated from diseased prostate tissue removed from a 59- |
| | | year-old Caucasian male during a radical prostatectomy with regional lymph node excision. |
| | | Pathology indicated adenofibromatous hyperplasia. Pathology for the associated tumor tissue |
| | | indicated an adenocarcinoma (Gleason grade 3 + 3). The patient presented with elevated |
| | | prostate-specific antigen (PSA). Patient history included colon diverticuli, asbestosis, |
| | | and thrombophlebitis. Previous surgeries included a partial colectomy. Family history |
| | | included benign hypertension, multiple myeloma, hyperlipidemia and rheumatoid arthritis. |
| PROSTUS23 | pINCY | This subtracted prostate tumor library was constructed using 10 million clones from a pooled |
| | | prostate tumor library that was subjected to 2 rounds of subtractive hybridization with 10 |
| | | million clones from a pooled prostate tissue library. The starting library for subtraction |
| | | was constructed by pooling equal numbers of clones from 4 prostate tumor libraries using |
| | | mRNA isolated from prostate tumor removed from Caucasian males at ages 58 (A), 61 (B), 66 |
| | | (C), and 68 (D) during prostatectomy with lymph node excision. Pathology indicated |
| | | adenocarcinoma in all donors. History included elevated PSA, induration and tobacco abuse |
| | | in donor A; elevated PSA, induration, prostate hyperplasia, renal failure, osteoarthritis, |
| | | renal artery stenosis, benign HTN, thrombocytopenia, hyperlipidemia, tobacco/alcohol abuse |
| | | and hepatitis C (carrier) in donor B; elevated PSA, induration, and tobacco abuse in donor C; |
| | | and elevated PSA, induration, hypercholesterolemia, and kidney calculus in donor D. The |
| | | hybridization probe for subtraction was constructed by pooling equal numbers of cDNA |
| | | clones from 3 prostate tissue libraries derived from prostate tissue, prostate epithelial |
| | | cells, |
| | | and fibroblasts from prostate stroma from 3 different donors. Subtractive hybridization |
| | | conditions were based on the methodologies of Swaroop et al., NAR 19 (1991): 1954 and |
| | | Bonaldo, et al. Genome Research 6 (1996): 791. |
| PROSTUT05 | PSPORT1 | Library was constructed using RNA isolated from prostate tumor tissue removed from a 69- |
| | | year-old Caucasian male during a radical prostatectomy. Pathology indicated adenocarcinoma |
| | | (Gleason grade 3 + 4). Adenofibromatous hyperplasia was also present. Family history |
| | | included congestive heart failure, multiple myeloma, hyperlipidemia, and rheumatoid arthritis. |
| SINTNOT18 | pINCY | Library was constructed using RNA isolated from small intestine tissue obtained from a 59- |
| | | year-old male. |
| STOMTDE01 | PCDNA2.1 | This 5′ biased random primed library was constructed using RNA isolated from stomach |
| | | tissue removed from a 61-year-old Caucasian male during a partial esophagectomy, proximal |
| | | gastrectomy, pyloromyotomy, and regional lymph node excision. Pathology for the associated |
| | | tumor indicated an invasive grade 3 adenocarcinoma in the esophagus, extending distally to |
| | | involve the gastroesophageal junction. The tumor extended through the muscularis to involve |
| | | periesophageal and perigastric soft tissues. One perigastric and two periesophageal lymph |
| | | nodes were positive for tumor. There were multiple perigastric and periesophageal tumor |
| | | implants. The patient presented with deficiency anemia and myelodysplasia. Patient history |
| | | included hyperlipidemia, and tobacco and alcohol abuse in remission. Previous surgeries |
| | | included adenotonsillectomy, rhinoplasty, vasectomy, and hemorrhoidectomy. A previous bone |
| | | marrow aspiration found the marrow to be hypercellular for age and had a cellularity-to-fat |
| | | ratio of 95:5. The marrow was focally densely fibrotic. Granulocytic precursors were |
| | | slightly increased with normal maturation. The estimate of blast cells was greater than 5%. |
| | | Megakaryocytes were increased and appeared atypical in clusters. Storage cells and |
| | | granulomata were absent. Patient medications included Epoetin, Danocrine, Berocca Plus |
| | | tablets, Selenium, vitamin B6 phosphate, vitamins E & C, and beta carotene. Family history |
| | | included alcohol abuse, atherosclerotic coronary artery disease, type II diabetes, chronic |
| | | liver disease, and primary cardiomyopathy in the father; and benign hypertension and |
| | | cerebrovascular disease in the mother. |
| TESTNOT03 | PBLUESCRIPT | Library was constructed using RNA isolated from testicular tissue removed from a 37-year- |
| | | old Caucasian male, who died from liver disease. Patient history included cirrhosis, |
| | | jaundice, and liver failure. |
| TESTTUT02 | pINCY | Library was constructed using RNA isolated from testicular tumor removed from a 31-year-old |
| | | Caucasian male during unilateral orchiectomy. Pathology indicated embryonal carcinoma. |
| THYMNOE02 | PCDNA2.1 | This 5′ biased random primed library was constructed using RNA isolated from thymus |
| | | tissue removed from a 3-year-old Hispanic male during a thymectomy and closure of a patent |
| | | ductus arteriosus. The patient presented with severe pulmonary stenosis and cyanosis. |
| | | Patient history included a cardiac catheterization and echocardiogram. Previous surgeries |
| | | included Blalock-Taussig shunt and pulmonary valvotomy. The patient was not taking any |
| | | medications. Family history included benign hypertension, osteoarthritis, depressive |
| | | disorder, and extrinsic asthma in the grandparent(s). |
| THYMNOT05 | pINCY | Library was constructed using RNA isolated from thymus tissue removed from a 3-year-old |
| | | Hispanic male during a thymectomy and closure of a patent ductus arteriosus. The patient |
| | | presented with severe pulmonary stenosis and cyanosis. Patient history included a cardiac |
| | | catheterization and echocardiogram. Previous surgeries included Blalock-Taussig shunt |
| | | and pulmonary valvotomy. The patient was not taking any medications. Family history |
| | | included benign hypertension, osteoarthritis, depressive disorder, and extrinsic asthma in |
| | | the grandparent(s). |
| THYRTUT03 | pINCY | Library was constructed using RNA isolated from benign thyroid tumor tissue removed from a |
| | | 17-year-old Caucasian male during a thyroidectomy. Pathology indicated encapsulated |
| | | follicular adenoma forming a circumscribed mass. |
| TLYMUNT01 | pINCY | Library was constructed using RNA isolated from resting allogenic T-lymphocyte tissue |
| | | removed from an adult (40-50-year old) Caucasian male. |
| TONSDIC01 | PSPORT1 | This large size fractionated library was constructed using pooled cDNA from two donors. |
| | | cDNA was generated using mRNA isolated from diseased left tonsil tissue removed from a 6- |
| | | year-old Caucasian male (donor A) during adenotonsillectomy and from diseased right tonsil |
| | | tissue removed from a 9-year-old Caucasian female (donor B) during adenotonsillectomy. |
| | | Pathology indicated reactive lymphoid hyperplasia, bilaterally (A) and lymphoid hyperplasia |
| | | (B). The patients presented with sleep apnea (A) and hypertrophy of tonsils, cough, and |
| | | unspecified nasal and sinus disease (B). Patient history included a bacterial infection |
| | | (A). Previous surgeries included myringotomy with tube insertion (A). Donor A was not |
| | | taking any medications and donor B was taking Vancenase. Family history included benign |
| | | hypertension, myocardial infarction, and atherosclerotic coronary artery disease in the |
| | | grandparent(s) of donor A; and extrinsic asthma and unspecified allergy in the mother; |
| | | unspecified allergy in the father; benign hypertension, deficiency anemia, |
| | | osteoarthritis, extrinsic asthma and unspecified allergy in the grandparent(s) of donor B. |
| UTRSTMR01 | pINCY | Library was constructed using RNA isolated from uterine myometrial tissue removed from a 41- |
| | | year-old Caucasian female during a vaginal hysterectomy. The endometrium was secretory and |
| | | contained fragments of endometrial polyps. Pathology for associated tumor tissue indicated |
| | | uterine leiomyoma. Patient history included ventral hernia and a benign ovarian neoplasm. |
| UTRSTUE01 | PCDNA2.1 | This 5′ biased random primed library was constructed using RNA isolated from uterus |
| | | tumor tissue removed a 37-year-old Black female during myomectomy, dilation and curettage, |
| | | right fimbrial region biopsy, and incidental appendectomy. Pathology indicated multiple |
| | | (12) uterine leiomyomata. A fimbrial cyst was identified. The patient presented with |
| | | deficiency anemia, an umbilical hernia, and premenopausal menorrhagia. Patient history |
| | | included premenopausal menorrhagia and sarcoidosis of the lung. Previous surgeries included |
| | | hysteroscopy, dilation and curettage, and an endoscopic lung biopsy. Patient medications |
| | | included Chromagen and Claritin. Family history included acute myocardial infarction and |
| | | atherosclerotic coronary artery disease in the father. |

TABLE 7

| | | | Parameter |
|---|---|---|---|
| Program | Description | Reference | Threshold |
| ABI | A program that removes vector sequences and masks | Applied Biosystems, Foster City, CA. | |
| FACTURA | ambiguous bases in nucleic acid sequences. | | |
| ABI/ | A Fast Data Finder useful in comparing and | Applied Biosystems, Foster City, CA; | Mismatch <50% |
| PARACEL FDF | annotating amino acid or nucleic acid sequences. | Paracel Inc., Pasadena, CA. | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: Probability |
| | sequence similarity search for amino acid and nucleic | 215: 403-410; Altschul, S. F. et al. (1997) | value = 1.0E−8 |
| | acid sequences. BLAST includes five functions: | Nucleic Acids Res. 25: 3389-3402. | or less |
| | blastp, blastn, blastx, tblastn, and tblastx. | | Full Length |
| | | | sequences: |
| | | | Probability value = |
| | | | 1.0E−10 or less |
| FASTA | A Pearson and Lipman algorithm that searches for | Pearson, W. R. and D. J. Lipman (1988) Proc. | ESTs: fasta E |
| | similarity between a query sequence and a group of | Natl. Acad Sci. USA 85: 2444-2448; Pearson, | value = 1.06E−6 |
| | sequences of the same type. FASTA comprises as | W. R. (1990) Methods Enzymol. 183: 63-98; | Assembled ESTs: |
| | least five functions: fasta, tfasta, fastx, tfastx, and | and Smith, T. F. and M. S. Waterman (1981) | fasta Identity = |
| | ssearch. | Adv. Appl. Math. 2: 482-489. | 95% or greater |
| | | | and Match length = |
| | | | 200 bases or greater; |
| | | | fastx E value = |
| | | | 1.0E−8 or less |
| | | | Full Length |
| | | | sequences: fastx |
| | | | score = 100 |
| | | | or greater |
| BLIMPS | A BLocks IMProved Searcher that matches a | Henikoff, S. and J. G. Henikoff (1991) | Probability value = |
| | sequence against those in BLOCKS, PRINTS, | Nucleic Acids Res. 19: 6565-6572; Henikoff, | 1.0E−3 or less |
| | DOMO, PRODOM, and PFAM databases to search | J. G. and S. Henikoff (1996) Methods | |
| | for gene families, sequence homology, and structural | Enzymol. 266: 88-105; and Attwood, T. K. et | |
| | fingerprint regions. | al. (1997) J. Chem. Inf. Comput. Sci. 37: 417- | |
| | | 424. | |
| HMMER | An algorithm for searching a query sequence against | Krogh, A. et al. (1994) J. Mol. Biol. | PFAM, INCY, SMART |
| | hidden Markov model (HMM)-based databases of | 235: 1501-1531; Sonnhammer, E. L. L. et al. | or TIGRFAM hits: |
| | protein family consensus sequences, such as PFAM, | (1988) Nucleic Acids Res. 26: 320-322; | Probability value = |
| | INCY, SMART and TIGRFAM. | Durbin, R. et al. (1998) Our World View, in | 1.0E−3 or less |
| | | a Nutshell, Cambridge Univ. Press, pp. 1- | Signal peptide |
| | | 350. | hits: Score = 0 |
| | | | or greater |
| ProfileScan | An algorithm that searches for structural and | Gribskov, M. et al. (1988) CABIOS 4: 61-66; | Normalized quality |
| | sequence motifs in protein sequences that match | Gribskov, M. et al. (1989) Methods | score ≧ GCG- |
| | sequence patterns defined in Prosite. | Enzymol. 183: 146-159; Bairoch, A. et al. | specified “HIGH” |
| | | (1997) Nucleic Acids Res. 25: 217-221. | value for that |
| | | | particular Prosite |
| | | | motif. Generally, |
| | | | score = 1.4-2.1. |
| Phred | A base-calling algorithm that examines automated | Ewing, B. et al. (1998) Genome Res. 8: 175- | |
| | sequencer traces with high sensitivity and probability. | 185; Ewing, B. and P. Green (1998) Genome | |
| | | Res. 8: 186-194. | |
| Phrap | A Phils Revised Assembly Program including | Smith, T. F. and M. S. Waterman (1981) Adv. | Score = 120 |
| | SWAT and CrossMatch, programs based on efficient | Appl. Math. 2: 482-489; Smith, T. F. and | or greater; |
| | implementation of the Smith-Waterman algorithm, | M. S. Waterman (1981) J. Mol. Biol. 147: 195- | Match |
| | useful in searching sequence homology and | 197; and Green, P., University of | length = 56 |
| | assembling DNA sequences. | Washington, Seattle, WA. | or greater |
| Consed | A graphical tool for viewing and editing Phrap | Gordon, D. et al. (1998) Genome Res. 8: 195- | |
| | assemblies. | 202. | |
| SPScan | A weight matrix analysis program that scans protein | Nielson, H. et al. (1997) Protein Engineering | Score = 3.5 |
| | sequences for the presence of secretory signal | 10: 1-6; Claverie, J. M. and S. Audic (1997) | or greater |
| | peptides. | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos | |
| | determine orientation. | (1996) Protein Sci. 5: 363-371. | |
| TMHMMER | A program that uses a hidden Markov model (HMM) | Sonnhammer, E. L. et al. (1998) Proc. Sixth | |
| | to delineate transmembrane segments on protein | Intl. Conf. on Intelligent Systems for Mol. | |
| | sequences and determine orientation. | Biol., Glasgow et al., eds., The Am. Assoc. | |
| | | for Artificial Intelligence Press, | |
| | | Menlo Park, CA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

TABLE 8

| | |
|---|---|
| Asian | Hispanic |

| | | | | | | | |
|---|---|---|---|---|---|---|---|
| SEQ | EST | Al- | Al- | Caucasian | African | Allele 1 | Allele 1 |

| | | | | | | | | | |
|---|---|---|---|---|---|---|---|---|---|
| ID | EST | CB1 | Al- | lele | lele | Allele 1 | Allele 1 | fre- | fre- |

| | | | | | | | | | | | | | |
|---|---|---|---|---|---|---|---|---|---|---|---|---|---|
| NO: | PID | EST ID | SNP ID | SNP | SNP | lele | 1 | 2 | Amino Acid | frequency | frequency | quency | quency |

| | | | | | | | | | | | | | |
|---|---|---|---|---|---|---|---|---|---|---|---|---|---|
| 112 | 7503849 | 1339126H1 | SNP00112738 | 151 | 315 | A | G | A | T80 | n/a | n/a | n/a | n/a |
| 112 | 7503849 | 1376303H1 | SNP00009519 | 85 | 38 | C | C | A | noncoding | n/a | n/a | n/a | n/a |
| 112 | 7503849 | 1376303H1 | SNP00037704 | 88 | 35 | G | G | A | noncoding | n/a | n/a | n/a | n/a |
| 112 | 7503849 | 1422323H1 | SNP00061236 | 167 | 384 | T | C | T | stop103 | n/d | n/d | n/d | n/d |
| 112 | 7503849 | 1520683H1 | SNP00009519 | 131 | 39 | C | C | A | noncoding | n/a | n/a | n/a | n/a |
| 112 | 7503849 | 1520683H1 | SNP00037704 | 134 | 36 | G | G | A | noncoding | n/a | n/a | n/a | n/a |
| 112 | 7503849 | 2211818H1 | SNP00097061 | 219 | 1021 | C | C | T | noncoding | n/a | n/a | n/a | n/a |
| 112 | 7503849 | 2346103H1 | SNP00037704 | 106 | 27 | G | G | A | noncoding | n/a | n/a | n/a | n/a |
| 112 | 7503849 | 2497486H1 | SNP00009519 | 84 | 41 | C | C | A | noncoding | n/a | n/a | n/a | n/a |
| 112 | 7503849 | 2497486H1 | SNP00037704 | 87 | 38 | G | G | A | noncoding | n/a | n/a | n/a | n/a |
| 112 | 7503849 | 2688833H1 | SNP00009519 | 103 | 42 | C | C | A | noncoding | n/a | n/a | n/a | n/a |
| 112 | 7503849 | 2688833H1 | SNP00037704 | 106 | 39 | G | G | A | noncoding | n/a | n/a | n/a | n/a |
| 112 | 7503849 | 2691537H1 | SNP00009519 | 228 | 106 | G | G | T | R10 | n/a | n/a | n/a | n/a |
| 112 | 7503849 | 2691537H1 | SNP00037704 | 225 | 109 | C | C | T | P11 | n/a | n/a | n/a | n/a |
| 112 | 7503849 | 2984764H1 | SNP00037704 | 84 | 100 | C | C | T | P8 | n/a | n/a | n/a | n/a |
| 112 | 7503849 | 2998532H1 | SNP00092683 | 42 | 1103 | C | C | T | noncoding | n/a | n/a | n/a | n/a |
| 112 | 7503849 | 3083817H1 | SNP00092683 | 97 | 1102 | T | C | T | noncoding | n/a | n/a | n/a | n/a |
| 112 | 7503849 | 3083817H1 | SNP00097061 | 15 | 1020 | C | C | T | noncoding | n/a | n/a | n/a | n/a |
| 112 | 7503849 | 3163442H1 | SNP00097061 | 252 | 1015 | C | C | T | noncoding | n/a | n/a | n/a | n/a |
| 112 | 7503849 | 3184509H1 | SNP00009519 | 79 | 94 | G | G | T | G6 | n/a | n/a | n/a | n/a |
| 112 | 7503849 | 3184509H1 | SNP00037704 | 82 | 97 | C | C | T | P7 | n/a | n/a | n/a | n/a |
| 112 | 7503849 | 3184509H1 | SNP00112738 | 288 | 305 | G | G | A | S76 | n/a | n/a | n/a | n/a |
| 112 | 7503849 | 3319727H1 | SNP00092683 | 71 | 1101 | C | C | T | noncoding | n/a | n/a | n/a | n/a |
| 112 | 7503849 | 3360460H1 | SNP00009519 | 85 | 44 | C | C | A | noncoding | n/a | n/a | n/a | n/a |
| 112 | 7503849 | 3501445H1 | SNP00009519 | 97 | 102 | T | G | T | C9 | n/a | n/a | n/a | n/a |
| 112 | 7503849 | 3692522H1 | SNP00061236 | 202 | 381 | C | C | T | P102 | n/d | n/d | n/d | n/d |
| 112 | 7503849 | 3748594H1 | SNP00112738 | 84 | 313 | A | G | A | H79 | n/a | n/a | n/a | n/a |
| 112 | 7503849 | 3777748H1 | SNP00112738 | 239 | 291 | G | G | A | G72 | n/a | n/a | n/a | n/a |
| 112 | 7503849 | 3877579H1 | SNP00009519 | 47 | 96 | T | G | T | S7 | n/a | n/a | n/a | n/a |
| 112 | 7503849 | 4205658H1 | SNP00061236 | 49 | 382 | C | C | T | A102 | n/d | n/d | n/d | n/d |
| 112 | 7503849 | 4541534H1 | SNP00112739 | 225 | 609 | C | C | T | H178 | n/d | n/d | n/d | n/d |
| 112 | 7503849 | 4575407H1 | SNP00092683 | 54 | 1100 | C | C | T | noncoding | n/a | n/a | n/a | n/a |
| 112 | 7503849 | 4640189H1 | SNP00112739 | 206 | 611 | C | C | T | Y178 | n/d | n/d | n/d | n/d |
| 112 | 7503849 | 4916293H1 | SNP00092683 | 43 | 1099 | C | C | T | noncoding | n/a | n/a | n/a | n/a |
| 112 | 7503849 | 4956713H1 | SNP00097061 | 8 | 1019 | C | C | T | noncoding | n/a | n/a | n/a | n/a |
| 112 | 7503849 | 5026038H1 | SNP00112739 | 75 | 602 | C | C | T | R175 | n/d | n/d | n/d | n/d |
| 112 | 7503849 | 5206042H1 | SNP00097061 | 135 | 851 | C | C | T | noncoding | n/a | n/a | n/a | n/a |
| 112 | 7503849 | 5872016H1 | SNP00061236 | 159 | 375 | C | C | T | L100 | n/d | n/d | n/d | n/d |
| 112 | 7503849 | 6732363H1 | SNP00112740 | 410 | 627 | C | C | T | L184 | n/a | n/a | n/a | n/a |

1. An isolated polypeptide selected from the group consisting of:
a) a polypeptide comprising an amino acid sequence selected from the group consisting of SEQ ID NO:1-55,
b) a polypeptide consisting essentially of the amino acid sequence of SEQ ID NO:56,
c) a polypeptide comprising a naturally occurring amino acid sequence at least 90% identical to an amino acid sequence selected from the group consisting of SEQ ID NO:2, SEQ ID NO:4, SEQ ID NO:7-12, SEQ ID NO:14-20, SEQ ID NO:23-24, SEQ ID NO:26-36, SEQ ID NO:38-43, SEQ ID NO:45-46 and SEQ ID NO:48-54,
d) a polypeptide comprising a naturally occurring amino acid sequence at least 93% identical to the amino acid sequence of SEQ ID NO:37,
e) a polypeptide comprising a naturally occurring amino acid sequence at least 94% identical to an amino acid sequence selected from the group consisting of SEQ ID NO:3, SEQ ID NO:22 and SEQ ID NO:55,
f) a polypeptide comprising a naturally occurring amino acid sequence at least 95% identical to the amino acid sequence of SEQ ID NO:13,
g) a polypeptide comprising a naturally occurring amino acid sequence at least 97% identical to an amino acid sequence selected from the group consisting of SEQ ID NO:6 and SEQ ID NO:44,
h) a polypeptide comprising a naturally occurring amino acid sequence at least 98% identical to an amino acid sequence selected from the group consisting of SEQ ID NO:5, SEQ ID NO:21 and SEQ ID NO:25,
i) a polypeptide comprising a naturally occurring amino acid sequence at least 99% identical to the amino acid sequence of SEQ ID NO:1,
j) a biologically active fragment of a polypeptide having an amino acid sequence selected from the group consisting of SEQ ID NO:1-56, and
k) an immunogenic fragment of a polypeptide having an amino acid sequence selected from the group consisting of SEQ ID NO:1-56.

2. An isolated polypeptide of claim 1 selected from the group consisting of:
a) a polypeptide comprising an amino acid sequence selected from the group consisting of SEQ ID NO:1-55 and
b) a polypeptide consisting essentially of the amino acid sequence of SEQ ID NO:56.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:57-112.

6. A recombinant polynucleotide comprising a promoter sequence operably linked to a polynucleotide of claim 3.

7. A cell transformed with a recombinant polynucleotide of claim 6.

8. (CANCELED)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. (CANCELED)

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:57-112,
b) a polynucleotide comprising a naturally occurring polynucleotide sequence at least 90% identical to a polynucleotide sequence selected from the group consisting of SEQ ID NO:57-112,
c) a polynucleotide complementary to a polynucleotide of a),
d) a polynucleotide complementary to a polynucleotide of b), and
e) an RNA equivalent of a)-d).

13. An isolated polynucleotide comprising at least 60 contiguous nucleotides of a polynucleotide of claim 12.

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. A method of claim 14, wherein the probe comprises at least 60 contiguous nucleotides.

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18-19. (CANCELED)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21-22. (CANCELED)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24-26. (CANCELED)

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. (CANCELED)

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-45. (CANCELED)

46. A microarray wherein at least one element of the microarray is a polynucleotide of claim 13.

47-167. (CANCELED)
